(19) United States
(12) Patent Application Publication
(10) Pub. No.: US 2022/0107322 A1
(43) Pub. Date: Apr. 7, 2022

(54) LIPID, PROTEIN, AND METABOLITE MARKERS FOR THE DIAGNOSIS AND TREATMENT OF PROSTATE CANCER

(71) Applicants: Berg LLC, Framingham, MA (US); The Henry M. Jackson Foundation for the Advancement of Military Medicine, Inc., Bethesda, MD (US)

(72) Inventors: Michael Andrew Kiebish, Millis, MA (US); Niven Rajin Narain, Cambridge, MA (US); Rangaprasad Sarangarajan, Boylston, MA (US); Viatcheslav R. Akmaev, Sudbury, MA (US); Leonardo Rodrigues, Ashland, MA (US); Yezhou Sun, Framingham, MA (US); Shiv Srivastava, Potomac, MD (US); Albert Dobi, Rockville, MD (US)

(21) Appl. No.: 17/343,538

(22) Filed: Jun. 9, 2021

Related U.S. Application Data
(63) Continuation of application No. 15/644,095, filed on Jul. 7, 2017, now abandoned.
(60) Provisional application No. 62/359,657, filed on Jul. 7, 2016.

(51) Int. Cl. G01N 33/574; A61K 31/00; C12Q 1/6886; G01R 33/46; G01N 21/31; G01N 30/02; C07C 229/12

(52) U.S. Cl. CPC G01N 33/57434; A61K 31/00; C12Q 1/6886; G01R 33/46; G01N 2500/10; G01N 2800/60; G01N 2030/027; C07C 229/12; G01N 2021/3155

(57) ABSTRACT

Methods for diagnosing the presence of prostate cancer in a subject are provided, such methods including the detection of levels of a variety of biomarkers diagnostic of prostate cancer. The invention also provides methods of treating prostate cancer by administering a biomarker or an agent that modulates a biomarker of prostate cancer. Compositions in the form of kits and panels of reagents for detecting the biomarkers of the invention are also provided.

### CROSS REFERENCE TO RELATED APPLICATIONS

This application is a continuation of U.S. patent application Ser. No. 15/644,095, filed Jul. 7, 2017 which, in turn, claims priority to U.S. Provisional Application Ser. No. 62/359,657, filed Jul. 7, 2016, the content of which is incorporated herein by reference in its entirety.

### GOVERNMENT SUPPORT

This invention was made with government support under HU0001-10-2-0002 awarded by the Uniformed Services University of the Health Sciences. The Government has certain rights in the invention.

### INCORPORATION BY REFERENCE

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the invention.

### BACKGROUND

### A. Field of the Invention

The invention relates generally to novel biomarkers and combinations thereof which can be used to diagnose, prognose, monitor, and treat prostate cancer. The invention also generally relates to methods for diagnosing, prognosing, monitoring, and treating prostate cancer involving the detection of biomarkers of the invention.

### B. Background of the Invention

Prostate cancer is a leading cause of male cancer-related deaths-second only to lung cancer—and afflicts one out of nine men over the age of 65. According to the American Cancer Society, 241,000 new cases of prostate cancer were reported with about 30,000 prostate cancer-related deaths that same year. Although the disease is typically diagnosed in men over the age of 65, its impact is still significant in that the average life span of a man who dies from prostate cancer is reduced by nearly a decade on average. However, if prostate cancer is discovered early, 90% of the cases may be cured with surgery. Once the tumor spreads outside the area of the prostate gland and forms distant metastases, the disease is more difficult to treat. Therefore, early detection is of critical importance to the success of interventional therapies, and for reducing the mortality rate associated with prostate cancer.

Prostate cancer typically develops in the various tissues of the prostate, a gland in the male reproductive system. Most prostate cancers are slow growing. However, there are also a significant number of cases per year of aggressive prostate cancers, in which the cancer cells may metastasize from the prostate to other parts of the body, particularly to the bones and lymph nodes. Prostate cancer may cause pain, difficulty in urinating, problems during sexual intercourse, or erectile dysfunction. Other symptoms can potentially develop during later stages of the disease.

Currently, prostate cancer is screened using only a limited number of detection means, including the digital rectal exam (DRE) and/or the measurement of the levels of prostate specific antigen (PSA). However, these approaches have an unacceptably high rate of false-positives. Indeed, most men (75%) with an elevated PSA level turn out not to have prostate cancer as determined by subsequent confirmatory prostate biopsies.

As such, the current screening tests are not specific enough to robustly screen for prostate cancer. Each year, based on the results of the DRE and PSA screens, about one million prostate biopsies are performed in the U.S. alone. Only 25% of these biopsies confirm the presence of cancer. PSA is secreted from epithelial cells of the prostate gland and is higher in blood due to increased number of prostate epithelial cells. When prostate cancers develop, PSA levels in the blood can start to climb. In the United States, the FDA has approved the PSA test for annual screening of prostate cancer in men of age 50 and older. PSA levels between 4 and 10 ng/nL are considered to be suspicious and consideration should be given to confirming the abnormal PSA with a repeat test. If indicated, a prostate biopsy is performed to obtain a tissue sample for histopathological analysis. Complications-such as infection, internal bleeding, allergic reactions, impotence, and urinary incontinence-induced by needless biopsies and treatments injure many more men than are potentially helped by early detection of cancers.

Indeed, the U.S. Preventative Services Task Force (USPSTF) estimates that about 90% of diagnosed men are treated and 2 in 1000 men will develop serious cardiovascular events, 1 in 1000 men will develop deep venous thrombosis, 29 in 1000 men will develop erectile dysfunction, 18 in 1000 men will develop urinary incontinence, and 1 in 1000 men will die due to treatment. A large majority of these men would have have remained asymptomatic for life if left untreated. As such, most cancers found through PSA tests are not, in fact, dangerous. Nevertheless, given the lack of more effective predictors of prostate cancer, the field takes a more conservative approach in the use of biopsies and treatment, erring on the side of precaution but risking significant harm to otherwise healthy men.

Despite the current drawbacks in prostate cancer detection, the USPSTF estimates that one life will be saved for every 1,000 men screened every 1-4 years over a 10-year period. This overall outlook can be further improved by limiting unnecessary biopsies with the use of improved pre-biopsy screening methods that are associated with fewer false-positive results. With fewer unnecessary biopsies, fewer men will suffer the associated biopsy complications. In addition, fewer complications will also lead to an overall cost reduction to the healthcare system in the management of prostate cancer. Accordingly, there is an unmet need for improved prostate cancer screening tools that improve the accuracy of prostate cancer prognosis and detection.

Prostate cancer incidence rates vary depending on race and/or ethnicity. For example, African-American men are nearly 1.6 times more likely to be diagnosed with prostate cancer than Caucasian men and 2.4 times more likely to die from the disease (Prostate Cancer Foundation, Oct. 5, 2012; http//www.pcf.org). Thus, there is also an unmet need for improved prostate cancer screening tools that improve the accuracy of prostate cancer prognosis and detection in diverse populations. Moreover, there is an unmet need and to determine activation status such as ERG-positive or ERG-negative tumors, and stratification along Gleason grades in prostate cancers. Molecular-based biomarkers other than PSA, such as lipids, proteins, and metabolites, may address this need. However, while lipid molecular species have been studied in recent years as potential biomarkers for the diagnosis of prostate cancer (e.g., Zhou, X., et al. PLoS One, Vol. 7, Issue 11, e48889 (2012) and Min H. K., et al., Anal. Bioanal. Chem., Vol. 399, Issue 2, pp. 823-30 (2011), to date there has no viable alternative to the DRE/PSA standard of care.

### SUMMARY OF THE INVENTION

In view of the fact that prostate cancer remains a life threatening disease reaching a significant portion of the male population across various racial and ethnic populations, there remains a need for efficient, accurate, and rapid molecular prognosis and diagnosis means, particularly which do not suffer from a high proportion of false results. The development of molecular tests for the accurate prognosis and detection of prostate cancer will also lead to improved management of appropriate therapies, and an overall improved survival rate. Thus, there remains a need to provide an improved diagnostic test for the detection of prostate cancer which is more reliable and accurate than PSA and other current screening tests. The present invention addresses this need by providing the use of biomarkers, i.e., one or more markers selected from Tables 1-31, which are, in some embodiments, associated with race or other clinical phenotypes, such as body mass index (BMI), ERG status, or Gleason stratification, for the accurate and reliable prognosis and/or detection of prostate cancer.

The present invention is based, at least in part, on the discovery that the markers in Tables 1-31 are differentially regulated in prostate cancer cells. In particular, the invention is based on the surprising discovery that the markers in Tables 1-31 are either elevated or depressed in the serum of patients with prostate cancer. It is also surprisingly discovered that certain markers of the invention are differentially expressed in populations of different races, for example, in African Americans (AA) or Caucasian Americans (CA), and are also differentally expressed in subjects with different types of prostate cancer, such as ERG-positive and ERG-negative prostate cancers, Gleason scores, or in subjects having different BMI indexes. Accordingly, the invention provides methods for diagnosing and/or monitoring (e.g., monitoring of disease progression or treatment) and/or prognosing an oncological disease state, e.g., prostate cancer, in a subject. In some embodiments, the subject is selected from a general population. In other embodiments, the subject is selected from a population of Caucasians. In yet another embodiment, the subject is selected from a population of African Americans. In some embodiments, the subject has an ERG-positive prostate cancer. In other embodiments, the subject has an ERG-negative prostate cancer. In a further embodiment, the subject has a BMI index equal or greater than 30.

The invention also provides methods for treating or for adjusting treatment regimens based on diagnostic information relating to the levels of the one or more markers selected from Tables 1-31 in the serum of a subject with an oncological disease state, e.g., prostate cancer. The invention further provides panels and kits for practicing the methods of the invention.

Accordingly, in one aspect, the present invention provides methods for diagnosing the presence of prostate cancer in a subject. The methods comprise (a) detecting the level of a prostate cancer marker in a biological sample from the subject, wherein the prostate cancer marker comprises one or more markers selected from Tables 1-31; and (b) comparing the level of the prostate cancer marker in the biological sample with a predetermined threshold value; wherein the level of the prostate cancer marker above or below the predetermined threshold value indicates a diagnosis that prostate cancer is present in the subject.

In another aspect, the present invention provides methods for diagnosing the presence of prostate cancer in a subject selected from a population of Caucasians. The methods comprise (a) detecting the level of a prostate cancer marker in a biological sample from the subject, wherein the prostate cancer marker comprises one or more markers selected from Tables 1, 4, 8, 11, 13, 16, 19, 22, 26, 29 and 30; and (b) comparing the level of the prostate cancer marker in the biological sample with a predetermined threshold value; wherein the level of the prostate cancer marker above or below the predetermined threshold value indicates a diagnosis that prostate cancer is present in the subject.

In yet another aspect, the present invention provides methods for diagnosing the presence of prostate cancer in a subject selected from a population of African Americans. The methods comprise (a) detecting the level of a prostate cancer marker in a biological sample from the subject, wherein the prostate cancer marker comprises one or more markers selected from Tables 2, 5, 9, 12, 14, 17, 20, 23, 27 and 31; and (b) comparing the level of the prostate cancer marker in the biological sample with a predetermined threshold value; wherein the level of the prostate cancer marker above or below the predetermined threshold value indicates a diagnosis that prostate cancer is present in the subject.

In one aspect, the present invention provides methods for diagnosing the presence of ERG-positive prostate cancer in a subject. The methods comprise (a) detecting the level of an ERG-positive prostate cancer marker in a biological sample from the subject, wherein the ERG-positive prostate cancer marker comprises one or more markers selected from Tables 6, 30 and 31; and (b) comparing the level of the ERG-positive prostate cancer marker in the biological sample with a predetermined threshold value; wherein the level of the ERG-positive prostate cancer marker above or below the predetermined threshold value indicates a diagnosis that ERG-positive prostate cancer is present in the subject.

In another aspect, the present invention provides methods for diagnosing the presence of prostate cancer in a subject with a BMI index equal or greater than 30. The methods comprise (a) detecting the level of a high BMI prostate cancer marker in a biological sample from the subject, wherein the high BMI prostate cancer marker comprises one or more markers selected from Tables 7, 18 and 25; and (b) comparing the level of the high BMI prostate cancer marker in the biological sample with a predetermined threshold value; wherein the level of the high BMI prostate cancer marker above or below the predetermined threshold value indicates a diagnosis that prostate cancer is present in the subject.

In another aspect, the present invention provides methods for diagnosing the presence of prostate cancer in a subject comprising (a) detecting the level of one or more prostate cancer marker in a biological sample from the subject, wherein the prostate cancer markers comprise one or more of nicotinamide, eicosenoic acid, and a decanoylcarnitate, e.g., dodecanoylcarnitine; and (b) comparing the level of the prostate cancer marker in the biological sample with a predetermined threshold value; wherein the level of the one or more prostate cancer markers above or below the predetermined threshold value indicates a diagnosis that prostate cancer is present in the subject.

In yet another aspect, the present invention provides methods for diagnosing the presence of ERG-negative prostate cancer in a Caucasian subject with a BMI index equal or greater than 30. The methods comprise (a) detecting the level of mercapto-succinyl-carnitine in a biological sample from the subject; and (b) comparing the level of mercapto-succinyl-carnitine in the biological sample with a predetermined threshold value; wherein the level of mercapto-succinyl-carnitine above the predetermined threshold value indicates a diagnosis that ERG-negative prostate cancer is present in the subject.

In some embodiments of the foregoing aspects, the biological sample is selected from the group consisting of blood, serum, plasma, urine, organ tissue, biopsy tissue, and seminal fluid. In other embodiments of the foregoing aspects, the organ tissue or biopsy tissue is prostate tissue.

In some embodiments of the foregoing aspects, the prostate cancer marker comprises at least two or more markers, wherein each of the two of more markers are selected from the structural lipids set forth in Tables 1-7, the signaling lipids set forth in Tables 8-12, the proteins set forth in Tables 13-18, the metabolites set forth in Tables 19-25, and the markers set forth in Tables 26-28.

In some embodiments of the foregoing aspects, the prostate cancer marker comprises at least two or more markers, wherein each of the two of more markers are selected from the a structural lipids set forth in Tables 1-3, the signaling lipids set forth in Tables 8-10, the proteins set forth in Tables 13-15, the metabolites set forth in Tables 19-21, and the markers set forth in Tables 26-28.

In some embodiments of the foregoing aspects, the prostate cancer marker is selected form the group consisting of nicotinamide, eicosenoic acid, and a decanoylcarnitate, e.g., ketodecanoylcarnitine.

In some embodiments of the foregoing aspects, the prostate cancer marker is a structural lipid selected from Tables 1-7. In other embodiments of the foregoing aspects, the prostate cancer marker is a structural lipid selected from Tables 1-3. In some embodiments of the foregoing aspects, the prostate cancer marker is a signaling lipid selected from Tables 8-12. In other embodiments of the foregoing aspects, the prostate cancer marker is a signaling lipid selected from Tables 8-10. In some embodiments of the foregoing aspects, the prostate cancer marker is a protein selected from Tables 13-18. In other embodiments of the foregoing aspects, the prostate cancer marker is a protein selected from Tables 13-15. In some embodiments of the foregoing aspects, the prostate cancer marker is a metabolite selected from Tables 19-25. In other embodiments of the foregoing aspects, the prostate cancer marker is a metabolite selected from Tables 19-21. In some embodiments of the foregoing aspects, the prostate cancer marker is selected from Tables 26-28.

In some embodiments of the foregoing aspects, the level of the prostate cancer marker is increased when compared to the predetermined threshold value in the subject. In other embodiments of the foregoing aspects, the level of the prostate cancer marker is decreased when compared to the predetermined threshold value in the subject. In some embodiments of the foregoing aspects, the prostate cancer marker comprises one or more markers with an increased level when compared to the predetermined threshold value in the subject, and/or one or more markers with a decreased level when compared to the predetermined threshold value in the subject.

In some embodiments of the foregoing aspects, the prostate cancer marker is selected from the group consisting of FFA_18:3, TAG_54:7+NH4, TAG_54:6+NH4, PA_18:1/20:2, FFA_18:3, FFA_20:1, TAG_54:7+NH4, TAG_54:6+NH4, PA_18:1/18:3, 6-KETO-PGF1A, TXB2, 13-HOTRE/13-HOTRE(R), 9-HOTRE, TXB2, 12-HEPE, 12-HETE, 13-HODE, APOC, APOB, ADIPOQ, SEPP1, CST3, F5, B2M, nicotinamide, eicosenoic acid, glycerylphosphorylethanolamine, nicotinamide, eicosenoic acid, 3-hydroxybutyric acid and 2-keto-isovalerate.

In some embodiments of the foregoing aspects, the prostate cancer marker is selected from the group consisting of CE_22:2+NH4, CE_20:0+NH4, CE_22:3+NH4, DAG_40:1+NH4, CE_20:1+NH4, PI 18:0/20:5, CE_22:1+NH4, TAG_54:0+NH4, PI 18:0/20:4, PI_16:0/18:3, PI_16:0/20:4, CE_20:0+NH4, CE_24:0+NH4, CE_22:2+NH4, DAG_42:2+NH4, PE 36:2, 5-HETE, LXA4, 15-OXOETE, 5-HEPE, 8-HETE, LTB4, 5-HEPE, 5-HETE, LTB4, PGE2/PGD2, GPLD1, SERPING1, C3, A2M, SERPINA6, APOA4, APCS, ITIH2, CLU, APOA2, PPBP, C3, APOA4, C4BPA, MMRN2, APOA2, FGA, ABI3BP, APOA1, PROS1, COMP, CDH5, SERPINA6, glu-leu, 6-ketodecanoylcarnitine, myo-inositol, chenodeoxyglycocholate, 2-hydroxy-2-methylbutanedioic acid, nonanedioic acid, 6-ketodecanoylcarnitine, glu-leu, ethanolamine, and nonanoylcarnitine.

In some embodiments of the foregoing aspects, the prostate cancer marker comprises one or more markers selected from Tables 4-7, 11, 12, 16-18, 22-25, 30 and 31, wherein the one or more markers have a FC ratio greater than 1, or a Log FC value greater than 0. In other embodiments of the foregoing aspects, the prostate cancer marker comprises one or more markers selected from Tables 4-7, 11, 12, 16-18, 22-25, 30 and 31, wherein the one or more markers have a FC ratio less than 1, or a Log FC value less than 0.

In some embodiments of the foregoing aspects, the prostate cancer marker comprises at least two or more markers, wherein each of the two or more markers are selected from the structural lipids set forth in Tables 1 and 4, the signaling lipids set forth in Tables 8 and 11, the proteins set forth in Tables 13 and 16, the metabolites set forth in Tables 19 and 22, and the markers set forth in Table 26.

In some embodiments of the foregoing aspects, the prostate cancer marker comprises at least two or more markers, wherein each of the two or more markers are selected from the structural lipids set forth in Table 1, the signaling lipids set forth in Table 8, the proteins set forth in Table 13, the metabolites set forth in Table 19, and the markers set forth in Table 26.

In some embodiments of the foregoing aspects, the prostate cancer marker is a structural lipid selected from Tables 1, 4 and 30. In other embodiments of the foregoing aspects, the prostate cancer marker is a structural lipid selected from Table 1. In some embodiments of the foregoing aspects, the prostate cancer marker is a signaling lipid selected from Tables 8 and 11. In other embodiments of the foregoing aspects, the prostate cancer marker is a signaling lipid selected from Table 8. In some embodiments of the foregoing aspects, the prostate cancer marker is a protein selected from Tables 13 and 16. In other embodiments of the foregoing aspects, the prostate cancer marker is a protein selected from Table 13. In some embodiments of the foregoing aspects, the prostate cancer marker is a metabolite selected from Tables 19 and 22. In other embodiments of the foregoing aspects, the prostate cancer marker is a metabolite selected from Table 19. In some embodiments of the foregoing aspects, the prostate cancer marker is selected from Tables 26 and 29.

In some embodiments of the foregoing aspects, the level of the prostate cancer marker is increased when compared to the predetermined threshold value in the subject. In other embodiments of the foregoing aspects, the level of the prostate cancer marker is decreased when compared to the predetermined threshold value in the subject. In some embodiments of the foregoing aspects, the prostate cancer marker comprises one or more markers with an increased level when compared to the predetermined threshold value in the subject, and/or one or more markers with a decreased level when compared to the predetermined threshold value in the subject.

In some embodiments of the foregoing aspects, the prostate cancer marker is selected from the group consisting of FFA_18:3, TAG_54:7+NH4, TAG_54:6+NH4, PA_18:1/20:2, 6-KETO-PGF1A, TXB2, APOC, APOB, ADIPOQ, SEPP1, nicotinamide, eicosenoic acid, glycerylphosphorylethanolamine.

In some embodiments of the foregoing aspects, the prostate cancer marker is selected from the group consisting of CE_22:2+NH4, CE_20:0+NH4, CE_22:3+NH4, DAG_40:1+NH4, CE_20:1+NH4, PI 18:0/20:5, CE_22:1+NH4, TAG_54:0+NH4, PI 18:0/20:4, PI_16:0/18:3, PI_16:0/20:4, 5-HETE, LXA4, 15-OXOETE, 5-HEPE, 8-HETE, LTB4, GPLD1, SERPING1, C3, A2M, SERPINA6, APOA4, APCS, ITIH2, CLU, APOA2, PPBP, glu-leu, 6-ketodecanoylcarnitine, myo-inositol, chenodeoxyglycocholate, 2-hydroxy-2-methylbutanedioic acid, nonanedioic acid.

In some embodiments of the foregoing aspects, the prostate cancer marker comprises one or more markers selected from Tables 4, 11, 16, 22 and 30, wherein the one or more markers have a FC ratio greater than 1, or a Log FC value greater than 0. In other embodiments of the foregoing aspects, the prostate cancer marker comprises one or more markers selected from Tables 4, 11, 16, 22 and 30, wherein the one or more markers have a FC ratio less than 1, or a Log FC value less than 0.

In some embodiments of the foregoing aspects, the prostate cancer marker comprises at least two or more markers, wherein each of the two or more markers are selected from the structural lipids set forth in Tables 2 and 5, the signaling lipids set forth in Tables 9 and 12, the proteins set forth in Tables 14 and 17, the metabolites set forth in Tables 20 and 23, and the markers set forth in Table 27.

In some embodiments of the foregoing aspects, the prostate cancer marker comprises at least two or more markers, wherein each of the two or more markers are selected from the structural lipids set forth in Table 2, the signaling lipids set forth in Table 9, the proteins set forth in Table 14, the metabolites set forth in Table 20, and the markers set forth in Table 27.

In some embodiments of the foregoing aspects, the prostate cancer marker is a structural lipid selected from Tables 2, 5 and 31. In other embodiments of the foregoing aspects, the prostate cancer marker is a structural lipid selected from Table 2. In some embodiments of the foregoing aspects, the prostate cancer marker is a signaling lipid selected from Tables 9 and 12. In other embodiments of the foregoing aspects, the prostate cancer marker is a signaling lipid selected from Table 9. In some embodiments of the foregoing aspects, the prostate cancer marker is a protein selected from Tables 14 and 17. In other embodiments of the foregoing aspects, the prostate cancer marker is a protein selected from Table 14. In some embodiments of the foregoing aspects, the prostate cancer marker is a metabolite selected from Tables 20 and 23. In other embodiments of the foregoing aspects, the prostate cancer marker is a metabolite selected from Table 20. In some embodiments of the foregoing aspects, the prostate cancer marker is selected from Table 27.

In some embodiments of the foregoing aspects, the level of the prostate cancer marker is increased when compared to the predetermined threshold value in the subject. In other embodiments of the foregoing aspects, the level of the prostate cancer marker is decreased when compared to the predetermined threshold value in the subject. In some embodiments of the foregoing aspects, the prostate cancer marker comprises one or more markers with an increased level when compared to the predetermined threshold value in the subject, and/or one or more markers with a decreased level when compared to the predetermined threshold value in the subject.

In some embodiments of the foregoing aspects, the prostate cancer marker is selected from the group consisting of FFA_18:3, FFA_20:1, TAG_54:7+NH4, TAG_54:6+NH4, PA_18:1/18:3, 13-HOTRE/13-HOTRE(R), 9-HOTRE, TXB2, 12-HEPE, 12-HETE, 13-HODE, CST3, F5, B2M, nicotinamide, eicosenoic acid, 3-hydroxybutyric acid, 2-keto-isovalerate and 2-octandioic-carnitine.

In some embodiments of the foregoing aspects, the prostate cancer marker is selected from the group consisting of CE_20:0+NH4, CE_24:0+NH4, CE_22:2+NH4, DAG_42:2+NH4, PE_36:2, 5-HEPE, 5-HETE, LTB4, PGE2/PGD2, C3, APOA4, C4BPA, MMRN2, APOA2, FGA, ABI3BP, APOA1, PROS1, COMP, CDH5, SERPINA6, 6-ketodecanoylcarnitine, glu-leu, ethanolamine, nonanoylcarnitine, and propionylcarnitine.

In some embodiments of the foregoing aspects, the prostate cancer marker comprises one or more markers selected from Tables 5, 12, 17, 23 and 31, wherein the one or more markers have a FC ratio greater than 1, or a Log FC value greater than 0. In other embodiments of the foregoing aspects, the prostate cancer marker comprises one or more markers selected from Tables 5, 12, 17, 23 and 31, wherein the one or more markers have a FC ratio less than 1, or a Log FC value less than 0.

In some embodiments of the foregoing aspects, the level of the ERG-positive prostate cancer marker is increased when compared to the predetermined threshold value in the subject. In other embodiments of the foregoing aspects, the level of the ERG-positive prostate cancer marker is decreased when compared to the predetermined threshold value in the subject. In some embodiments of the foregoing aspects, the ERG-positive prostate cancer marker comprises one or more markers with an increased level when compared to the predetermined threshold value in the subject, and/or one or more markers with a decreased level when compared to the predetermined threshold value in the subject.

In some embodiments of the foregoing aspects, the ERG-positive prostate cancer marker is selected from the group consisting of CE_20:4+NH4, PG_16:1/18:3, D18:0/16:1-MONOHEX, D18:1/22:1-MONOHEX, PG_16:1/20:3.

In some embodiments of the foregoing aspects, the ERG-positive prostate cancer marker is selected from the group consisting of LPC_0-14:1, LPC_22:1, LPC_10:0, LPC_0-22:0, LPC_24:0.

In some embodiments of the foregoing aspects, the ERG-positive prostate cancer marker comprises one or more markers selected from Tables 6, 30 and 31, wherein the one or more markers have a FC ratio greater than 1, or a Log FC value greater than 0. In other embodiments of the foregoing aspects, the ERG-positive prostate cancer marker comprises one or more markers selected from Tables 6, 30 and 31, wherein the one or more markers have a FC ratio less than 1, or a Log FC value less than 0.

In some embodiments of the foregoing aspects, the level of the high BMI prostate cancer marker is increased when compared to the predetermined threshold value in the subject. In other embodiments of the foregoing aspects, the level of the high BMI prostate cancer marker is decreased when compared to the predetermined threshold value in the subject. In some embodiments of the foregoing aspects, the high BMI prostate cancer marker comprises one or more markers with an increased level when compared to the predetermined threshold value in the subject, and/or one or more markers with a decreased level when compared to the predetermined threshold value in the subject.

In some embodiments of the foregoing aspects, the high BMI prostate cancer marker comprises one or more markers selected from Tables 7, 18 and 25, wherein the one or more markers have a FC ratio greater than 1, or a Log FC value greater than 0. In other embodiments of the foregoing aspects, the high BMI prostate cancer marker comprises one or more markers selected from Tables 7, 18 and 25, wherein the one or more markers have a FC ratio less than 1, or a Log FC value less than 0.

In some embodiments of the foregoing aspects, the level of the prostate cancer marker is detected by HPLC/UV-Vis spectroscopy, enzymatic analysis, mass spectrometry, NMR, immunoassay, ELISA, or any combination thereof. In some embodiments of the foregoing aspects, the level of the prostate cancer marker is detected by determining the level of its corresponding mRNA in the biological sample.

In some embodiments of the foregoing aspects, the level of the ERG-positive prostate cancer marker is detected by HPLC/UV-Vis spectroscopy, enzymatic analysis, mass spectrometry, NMR, immunoassay, ELISA, or any combination thereof. In some embodiments of the foregoing aspects, the level of the ERG-positive prostate cancer marker is detected by determining the level of its corresponding mRNA in the biological sample.

In some embodiments of the foregoing aspects, the level of the high BMI prostate cancer marker is detected by HPLC/UV-Vis spectroscopy, enzymatic analysis, mass spectrometry, NMR, immunoassay, ELISA, or any combination thereof. In some embodiments of the foregoing aspects, the level of the high BMI prostate cancer marker is detected by determining the level of its corresponding mRNA in the biological sample.

In some embodiments of the foregoing aspects, the level of mercapto-succinyl-carnitine is detected by HPLC/UV-Vis spectroscopy, enzymatic analysis, mass spectrometry, NMR, immunoassay, ELISA, or any combination thereof.

In some embodiments of the foregoing aspects, the methods further comprise detecting the level of one or more additional markers of prostate cancer. In some embodiments of the foregoing aspects, the one or more additional markers of prostate cancer is prostate specific antigen (PSA).

In some embodiments of the foregoing aspects, the methods described herein further comprise administering a therapeutic anti-cancer treatment where the diagnosis indicates the presence of prostate cancer in the subject. In other embodiments of the foregoing aspects, the methods described herein further comprise administering a therapeutic anti-cancer treatment where the diagnosis indicates the presence of ERG-positive prostate cancer in the subject. In some embodiments of the foregoing aspects, the methods described herein further comprise administering a therapeutic anti-cancer treatment where the diagnosis indicates the presence of ERG-negative prostate cancer in the subject.

In some embodiments of the foregoing aspects, the anti-cancer treatment is selected from the group consisting of (a) radiation therapy, (b) chemotherapy, (c) surgery, (d) hormone therapy, (e) antibody therapy, (f) immunotherapy, (g) cytokine therapy, (h) growth factor therapy, and (i) any combination of (a)-(h).

In some embodiments of the foregoing aspects, the methods described herein further comprise selecting a subject suspected of having or being at risk of having prostate cancer. In some embodiments of the foregoing aspects, the methods described herein further comprise obtaining a biological sample from a subject suspected of having or being at risk of having prostate cancer.

In some embodiments of the foregoing aspects, the subject is selected from a population of Caucasians. In some embodiments of the foregoing aspects, the subject is selected from a population of African Americans.

In one aspect, the present invention provides methods for identifying a subject as being at an increased risk for developing prostate cancer. The methods comprise (a) detecting the level of a prostate cancer marker in a biological sample from the subject, wherein the prostate cancer comprises one or more markers selected from Tables 1-31; and (b) comparing the level of the prostate cancer marker in the biological sample with a predetermined threshold value; wherein the level of the prostate cancer marker above or below the predetermined threshold value indicates that the subject is being at an increased risk for developing prostate cancer.

In another aspect, the present invention provides methods for identifying a Caucasian subject as being at an increased risk for developing prostate cancer. The methods comprise (a) detecting the level of a prostate cancer marker in a biological sample from the subject, wherein the prostate cancer comprises one or more markers selected from Tables 1, 4, 8, 11, 13, 16, 19, 22, 26, 29 and 30; and (b) comparing the level of the prostate cancer marker in the biological sample with a predetermined threshold value; wherein the level of the prostate cancer marker above or below the predetermined threshold value indicates that the subject is being at an increased risk for developing prostate cancer.

In yet another aspect, the present invention provides methods for identifying an African American subject as being at an increased risk for developing prostate cancer. The methods comprise (a) detecting the level of a prostate cancer marker in a biological sample from the subject, wherein the prostate cancer comprises one or more markers selected from Tables 2, 5, 9, 12, 14, 17, 20, 23, 27 and 31; and (b) comparing the level of the prostate cancer marker in the biological sample with a predetermined threshold value; wherein the level of the prostate cancer marker above or below the predetermined threshold value indicates that the subject is being at an increased risk for developing prostate cancer.

In one aspect, the present invention provides methods for identifying a subject as being at an increased risk for developing ERG-positive prostate cancer in a subject. The methods comprise (a) detecting the level of an ERG-positive prostate cancer marker selected from Tables 6, 30 and 31; and (b) comparing the level of the ERG-positive prostate cancer marker in the biological sample with a predetermined threshold value; wherein the level of the ERG-positive prostate cancer marker above or below the predetermined threshold value indicates that the subject is being at an increased risk for developing ERG-positive prostate cancer.

In another aspect, the present invention provides methods for identifying a subject with a BMI index equal or greater than 30 as being at an increased risk for developing prostate cancer. The methods comprise (a) detecting the level of a high BMI prostate cancer marker selected from Tables 7, 18 and 25; and (b) comparing the level of the high BMI prostate cancer marker in the biological sample with a predetermined threshold value; wherein the level of the high BMI prostate cancer marker above or below the predetermined threshold value indicates that the subject is being at an increased risk for developing prostate cancer.

In yet another aspect, the present invention provides methods for identifying a Caucasian subject with a BMI index equal or greater than 30 as being at an increased risk for developing ERG-negative prostate cancer. The methods comprise (a) detecting the level of mercapto-succinyl-carnitine in the biological sample from the subject; and (b) comparing the level of mercapto-succinyl-carnitine in the biological sample with a predetermined threshold value; wherein the level of mercapto-succinyl-carnitine above the predetermined threshold value indicates that the subject is being at an increased risk for developing ERG-negative prostate cancer.

In some embodiments of the foregoing aspects, the biological sample is selected from the group consisting of blood, serum, plasma, urine, organ tissue, biopsy tissue, and seminal fluid. In some embodiments of the foregoing aspects, the organ tissue or biopsy tissue is prostate tissue.

In some embodiments of the foregoing aspects, the prostate cancer marker comprises at least two or more markers, wherein each of the two of more markers are selected from the structural lipids set forth in Tables 1-7, the signaling lipids set forth in Tables 8-12, the proteins set forth in Tables 13-18, the metabolites set forth in Tables 19-25, and the markers set forth in Tables 26-28.

In some embodiments of the foregoing aspects, the prostate cancer marker comprises at least two or more markers, wherein each of the two of more markers are selected from the a structural lipids set forth in Tables 1-3, the signaling lipids set forth in Tables 8-10, the proteins set forth in Tables 13-15, the metabolites set forth in Tables 19-21, and the markers set forth in Tables 26-28.

In some embodiments of the foregoing aspects, the prostate cancer marker is a structural lipid selected from Tables 1-7. In other embodiments of the foregoing aspects, the prostate cancer marker is a structural lipid selected from Tables 1-3. In some embodiments of the foregoing aspects, the prostate cancer marker is a signaling lipid selected from Tables 8-12. In other embodiments of the foregoing aspects, the prostate cancer marker is a signaling lipid selected from Tables 8-10. In some embodiments of the foregoing aspects, the prostate cancer marker is a protein selected from Tables 13-18. In other embodiments of the foregoing aspects, the prostate cancer marker is a protein selected from Tables 13-15. In some embodiments of the foregoing aspects, the prostate cancer marker is a metabolite selected from Tables 19-25. In other embodiments of the foregoing aspects, the prostate cancer marker is a metabolite selected from Tables 19-21. In some embodiments of the foregoing aspects, the prostate cancer marker is selected from Tables 26-28.

In some embodiments of the foregoing aspects, the level of the prostate cancer marker is increased when compared to the predetermined threshold value in the subject. In other embodiments of the foregoing aspects, the level of the prostate cancer marker is decreased when compared to the predetermined threshold value in the subject. In some embodiments of the foregoing aspects, the prostate cancer marker comprises one or more markers with an increased level when compared to the predetermined threshold value in the subject, and/or one or more markers with a decreased level when compared to the predetermined threshold value in the subject.

In some embodiments of the foregoing aspects, the prostate cancer marker is selected from the group consisting of FFA_18:3, TAG_54:7+NH4, TAG_54:6+NH4, PA_18:1/20:2, FFA_18:3, FFA_20:1, TAG_54:7+NH4, TAG_54:6+NH4, PA_18:1/18:3, 6-KETO-PGF1A, TXB2, 13-HOTRE/13-HOTRE(R), 9-HOTRE, TXB2, 12-HEPE, 12-HETE, 13-HODE, APOC, APOB, ADIPOQ, SEPP1, CST3, F5, B2M, nicotinamide, eicosenoic acid, glycerylphosphorylethanolamine, nicotinamide, eicosenoic acid, 3-hydroxybutyric acid and 2-keto-isovalerate.

In some embodiments of the foregoing aspects, the prostate cancer marker is selected from the group consisting of CE_22:2+NH4, CE_20:0+NH4, CE_22:3+NH4, DAG_40:1+NH4, CE_20:1+NH4, PI_18:0/20:5, CE_22:1+NH4, TAG_54:0+NH4, PI_18:0/20:4, PI_16:0/18:3, PI_16:0/20:4, CE_20:0+NH4, CE_24:0+NH4, CE_22:2+NH4, DAG 42:2+NH4, PE 36:2, 5-HETE, LXA4, 15-OXOETE, 5-HEPE, 8-HETE, LTB4, 5-HEPE, 5-HETE, LTB4, PGE2/PGD2, GPLD1, SERPING1, C3, A2M, SERPINA6, APOA4, APCS, ITIH2, CLU, APOA2, PPBP, C3, APOA4, C4BPA, MMRN2, APOA2, FGA, ABI3BP, APOA1, PROS1, COMP, CDH5, SERPINA6, glu-leu, 6-ketodecanoylcarnitine, myo-inositol, chenodeoxyglycocholate, 2-hydroxy-2-methylbutanedioic acid, nonanedioic acid, 6-ketodecanoylcarnitine, glu-leu, ethanolamine, and nonanoylcarnitine.

In some embodiments of the foregoing aspects, the prostate cancer marker comprises one or more markers selected from Tables 4-7, 11, 12, 16-18, 22-25, 30 and 31, wherein the one or more markers have a FC ratio greater than 1, or a Log FC value greater than 0. In other embodiments of the foregoing aspects, the prostate cancer marker comprises one or more markers selected from Tables 4-7, 11, 12, 16-18, 22-25, 30 and 31, wherein the one or more markers have a FC ratio less than 1, or a Log FC value less than 0.

In some embodiments of the foregoing aspects, the prostate cancer marker comprises at least two or more markers, wherein each of the two or more markers are selected from the structural lipids set forth in Tables 1 and 4, the signaling lipids set forth in Tables 8 and 11, the proteins set forth in Tables 13 and 16, the metabolites set forth in Tables 19 and 22, and the markers set forth in Table 26.

In some embodiments of the foregoing aspects, the prostate cancer marker comprises at least two or more markers, wherein each of the two or more markers are selected from the structural lipids set forth in Table 1, the signaling lipids set forth in Table 8, the proteins set forth in Table 13, the metabolites set forth in Table 19, and the markers set forth in Table 26.

In some embodiments of the foregoing aspects, the prostate cancer marker is a structural lipid selected from Tables 1, 4 and 30. In other embodiments of the foregoing aspects, the prostate cancer marker is a structural lipid selected from Table 1. In some embodiments of the foregoing aspects, the prostate cancer marker is a signaling lipid selected from Tables 8 and 11. In other embodiments of the foregoing aspects, the prostate cancer marker is a signaling lipid selected from Table 8. In some embodiments of the foregoing aspects, the prostate cancer marker is a protein selected from Tables 13 and 16. In other embodiments of the foregoing aspects, the prostate cancer marker is a protein selected from Table 13. In some embodiments of the foregoing aspects, the prostate cancer marker is a metabolite selected from Tables 19 and 22. In other embodiments of the foregoing aspects, the prostate cancer marker is a metabolite selected from Table 19. In some embodiments of the foregoing aspects, the prostate cancer marker is selected from Tables 26 and 29.

In some embodiments of the foregoing aspects, the level of the prostate cancer marker is increased when compared to the predetermined threshold value in the subject. In other embodiments of the foregoing aspects, the level of the prostate cancer marker is decreased when compared to the predetermined threshold value in the subject. In some embodiments of the foregoing aspects, the prostate cancer marker comprises one or more markers with an increased level when compared to the predetermined threshold value in the subject, and/or one or more markers with a decreased level when compared to the predetermined threshold value in the subject.

In some embodiments of the foregoing aspects, the prostate cancer marker is selected from the group consisting of FFA_18:3, TAG_54:7+NH4, TAG_54:6+NH4, PA_18:1/20:2, 6-KETO-PGF1A, TXB2, APOC, APOB, ADIPOQ, SEPP1, nicotinamide, eicosenoic acid, glycerylphosphorylethanolamine.

In some embodiments of the foregoing aspects, the prostate cancer marker is selected from the group consisting of CE_22:2+NH4, CE_20:0+NH4, CE_22:3+NH4, DAG_40:1+NH4, CE_20:1+NH4, PI_18:0/20:5, CE_22:1+NH4, TAG_54:0+NH4, PI_18:0/20:4, PI_16:0/18:3, PI_16:0/20:4, 5-HETE, LXA4, 15-OXOETE, 5-HEPE, 8-HETE, LTB4, GPLD1, SERPING1, C3, A2M, SERPINA6, APOA4, APCS, ITIH2, CLU, APOA2, PPBP, glu-leu, 6-ketodecanoylcarnitine, myo-inositol, chenodeoxyglycocholate, 2-hydroxy-2-methylbutanedioic acid, nonanedioic acid.

In some embodiments of the foregoing aspects, the prostate cancer marker comprises one or more markers selected from Tables 4, 11, 16, 22 and 30, wherein the one or more markers have a FC ratio greater than 1, or a Log FC value greater than 0. In other embodiments of the foregoing aspects, the prostate cancer marker comprises one or more markers selected from Tables 4, 11, 16, 22 and 30, wherein the one or more markers have a FC ratio less than 1, or a Log FC value less than 0.

In some embodiments of the foregoing aspects, the prostate cancer marker comprises at least two or more markers, wherein each of the two or more markers are selected from the structural lipids set forth in Tables 2 and 5, the signaling lipids set forth in Tables 9 and 12, the proteins set forth in Tables 14 and 17, the metabolites set forth in Tables 20 and 23, and the markers set forth in Table 27.

In some embodiments of the foregoing aspects, the prostate cancer marker comprises at least two or more markers, wherein each of the two or more markers are selected from the structural lipids set forth in Table 2, the signaling lipids set forth in Table 9, the proteins set forth in Table 14, the metabolites set forth in Table 20, and the markers set forth in Table 27.

In some embodiments of the foregoing aspects, the prostate cancer marker is a structural lipid selected from Tables 2, 5 and 31. In other embodiments of the foregoing aspects, the prostate cancer marker is a structural lipid selected from Table 2. In some embodiments of the foregoing aspects, the prostate cancer marker is a signaling lipid selected from Tables 9 and 12. In other embodiments of the foregoing aspects, the prostate cancer marker is a signaling lipid selected from Table 9. In some embodiments of the foregoing aspects, the prostate cancer marker is a protein selected from Tables 14 and 17. In other embodiments of the foregoing aspects, the prostate cancer marker is a protein selected from Table 14. In some embodiments of the foregoing aspects, the prostate cancer marker is a metabolite selected from Tables 20 and 23. In other embodiments of the foregoing aspects, the prostate cancer marker is a metabolite selected from Table 20. In some embodiments of the foregoing aspects, the prostate cancer marker is selected from Table 27.

In some embodiments of the foregoing aspects, the level of the prostate cancer marker is increased when compared to the predetermined threshold value in the subject. In other embodiments of the foregoing aspects, the level of the prostate cancer marker is decreased when compared to the predetermined threshold value in the subject. In some embodiments of the foregoing aspects, the prostate cancer marker comprises one or more markers with an increased level when compared to the predetermined threshold value in the subject, and/or one or more markers with a decreased level when compared to the predetermined threshold value in the subject.

In some embodiments of the foregoing aspects, the prostate cancer marker is selected from the group consisting of FFA_18:3, FFA_20:1, TAG_54:7+NH4, TAG_54:6+NH4, PA_18:1/18:3, 13-HOTRE/13-HOTRE(R), 9-HOTRE, TXB2, 12-HEPE, 12-HETE, 13-HODE, CST3, F5, B2M, nicotinamide, eicosenoic acid, 3-hydroxybutyric acid, 2-keto-isovalerate and 2-octandioic-carnitine.

In some embodiments of the foregoing aspects, the prostate cancer marker is selected from the group consisting of CE_20:0+NH4, CE_24:0+NH4, CE_22:2+NH4, DAG_42:2+NH4, PE_36:2, 5-HEPE, 5-HETE, LTB4, PGE2/PGD2, C3, APOA4, C4BPA, MMRN2, APOA2, FGA, ABI3BP, APOA1, PROS1, COMP, CDH5, SERPINA6, 6-ketodecanoylcarnitine, glu-leu, ethanolamine, nonanoylcarnitine, and propionylcarnitine.

In some embodiments of the foregoing aspects, the prostate cancer marker comprises one or more markers selected from Tables 5, 12, 17, 23 and 31, wherein the one or more markers have a FC ratio greater than 1, or a Log FC value greater than 0. In other embodiments of the foregoing aspects, the prostate cancer marker comprises one or more markers selected from Tables 5, 12, 17, 23 and 31, wherein the one or more markers have a FC ratio less than 1, or a Log FC value less than 0.

In some embodiments of the foregoing aspects, the level of the ERG-positive prostate cancer marker is increased when compared to the predetermined threshold value in the subject. In other embodiments of the foregoing aspects, the level of the ERG-positive prostate cancer marker is decreased when compared to the predetermined threshold value in the subject. In some embodiments of the foregoing aspects, the ERG-positive prostate cancer marker comprises one or more markers with an increased level when compared to the predetermined threshold value in the subject, and/or one or more markers with a decreased level when compared to the predetermined threshold value in the subject.

In some embodiments of the foregoing aspects, the ERG-positive prostate cancer marker is selected from the group consisting of CE_20:4+NH4, PG_16:1/18:3, D18:0/16:1-MONOHEX, D18:1/22:1-MONOHEX, PG_16:1/20:3.

In some embodiments of the foregoing aspects, the ERG-positive prostate cancer marker is selected from the group consisting of LPC_0-14:1, LPC_22:1, LPC_10:0, LPC_0-22:0, LPC_24:0.

In some embodiments of the foregoing aspects, the ERG-positive prostate cancer marker comprises one or more markers selected from Tables 6, 30 and 31, wherein the one or more markers have a FC ratio greater than 1, or a Log FC value greater than 0. In other embodiments of the foregoing aspects, the ERG-positive prostate cancer marker comprises one or more markers selected from Tables 6, 30 and 31, wherein the one or more markers have a FC ratio less than 1, or a Log FC value less than 0.

In some embodiments of the foregoing aspects, the level of the high BMI prostate cancer marker is increased when compared to the predetermined threshold value in the subject. In other embodiments of the foregoing aspects, the level of the high BMI prostate cancer marker is decreased when compared to the predetermined threshold value in the subject. In some embodiments of the foregoing aspects, the high BMI prostate cancer marker comprises one or more markers with an increased level when compared to the predetermined threshold value in the subject, and/or one or more markers with a decreased level when compared to the predetermined threshold value in the subject.

In some embodiments of the foregoing aspects, the high BMI prostate cancer marker comprises one or more markers selected from Tables 7, 18 and 25, wherein the one or more markers have a FC ratio greater than 1, or a Log FC value greater than 0. In other embodiments of the foregoing aspects, the high BMI prostate cancer marker comprises one or more markers selected from Tables 7, 18 and 25, wherein the one or more markers have a FC ratio less than 1, or a Log FC value less than 0.

In some embodiments of the foregoing aspects, the level of the prostate cancer marker is detected by HPLC/UV-Vis spectroscopy, enzymatic analysis, mass spectrometry, NMR, immunoassay, ELISA, or any combination thereof. In some embodiments of the foregoing aspects, the level of the prostate cancer marker is detected by determining the level of its corresponding mRNA in the biological sample.

In some embodiments of the foregoing aspects, the level of the ERG-positive prostate cancer marker is detected by HPLC/UV-Vis spectroscopy, enzymatic analysis, mass spectrometry, NMR, immunoassay, ELISA, or any combination thereof. In some embodiments of the foregoing aspects, the level of the ERG-positive prostate cancer marker is detected by determining the level of its corresponding mRNA in the biological sample.

In some embodiments of the foregoing aspects, the level of the high BMI prostate cancer marker is detected by HPLC/UV-Vis spectroscopy, enzymatic analysis, mass spectrometry, NMR, immunoassay, ELISA, or any combination thereof. In some embodiments of the foregoing aspects, the level of the high BMI prostate cancer marker is detected by determining the level of its corresponding mRNA in the biological sample.

In some embodiments of the foregoing aspects, the level of mercapto-succinyl-carnitine is detected by HPLC/UV-Vis spectroscopy, enzymatic analysis, mass spectrometry, NMR, immunoassay, ELISA, or any combination thereof.

In some embodiments of the foregoing aspects, the methods described herein further comprise detecting the level of one or more additional markers of prostate cancer. In some embodiments of the foregoing aspects, the one or more additional markers of prostate cancer is prostate specific antigen (PSA).

In some embodiments of the foregoing aspects, the methods described herein further comprise administering a therapeutic anti-cancer treatment to the subject based on the prognosis. In some embodiments of the foregoing aspects, the anti-cancer treatment is selected from the group consisting of (a) radiation therapy, (b) chemotherapy, (c) surgery, (d) hormone therapy, (e) antibody therapy, (f) immunotherapy, (g) cytokine therapy, (h) growth factor therapy, and (i) any combination of (a)-(h).

In some embodiments of the foregoing aspects, the biomarker reference level correlates with a Gleason Score in the range of from 2 to 10. In other embodiments of the foregoing aspects, the biomarker is at least one marker selected from Table 29.

In some embodiments of the foregoing aspects, the biomarker reference level correlates with a T stage classification selected from the group consisting of T1, T2, T3, and T4. In other embodiments of the foregoing aspects, the biomarker reference level correlates with a N stage classification selected from the group consisting of N0, N1, N2, and N3. In certain embodiments of the foregoing aspects, the biomarker reference level correlates with a M stage classification selected from the group consisting of M0 and M1.

In some embodiments of the foregoing aspects, the subject is selected from a population of Caucasians. In other embodiments of the foregoing aspects, the subject is selected from a population of African Americans.

In one aspect, the present invention provides methods for monitoring prostate cancer in a subject. The methods comprise (1) detecting the level of a prostate cancer marker in a first biological sample obtained at a first time from the subject having prostate cancer, wherein the prostate cancer marker comprises one or more markers selected from Tables 1-31; (2) detecting the level of the prostate cancer marker in a second biological sample obtained from the subject at a second time, wherein the second time is later than the first time; and (3) comparing the level of the prostate cancer marker in the second sample with the level of the prostate cancer marker in the first sample; wherein a change in the level of the prostate cancer marker is indicative of a change in prostate cancer status in the subject.

In another aspect, the present invention provides methods for monitoring prostate cancer in a subject selected from a population of Caucasians. The methods comprise (1) detecting the level of a prostate cancer marker in a first biological sample obtained at a first time from the subject having prostate cancer, wherein the prostate cancer marker comprises one or more markers selected from Tables 1, 4, 8, 11, 13, 16, 19, 22, 26, 29 and 30; (2) detecting the level of the prostate cancer marker in a second biological sample obtained from the subject at a second time, wherein the second time is later than the first time; and (3) comparing the level of the prostate cancer marker in the second sample with the level of the prostate cancer marker in the first sample; herein a change in the level of the prostate cancer marker is indicative of a change in prostate cancer status in the subject.

In one aspect, the present invention provides methods for monitoring prostate cancer in a subject selected from a population of African Americans. The methods comprise (1) detecting the level of a prostate cancer marker in a first biological sample obtained at a first time from the subject having prostate cancer, wherein the prostate cancer marker comprises one or more markers selected from Tables 2, 5, 9, 12, 14, 17, 20, 23, 27 and 31 in a first biological sample obtained at a first time from a subject having prostate cancer; (2) detecting the level of the prostate cancer marker in a second biological sample obtained from the subject at a second time, wherein the second time is later than the first time; and (3) comparing the level of the prostate cancer marker in the second sample with the level of the prostate cancer marker in the first sample; wherein a change in the level of the prostate cancer marker is indicative of a change in prostate cancer status in the subject.

In another aspect, the present invention provides methods for monitoring ERG-positive prostate cancer in a subject. The methods comprise (1) detecting the level of an ERG-positive prostate cancer marker in a first biological sample obtained at a first time from the subject having ERG-positive prostate cancer, wherein the ERG-positive prostate cancer marker comprises one or more markers selected from Tables 6, 30 and 31; (2) detecting the level of the ERG-positive prostate cancer marker in a second biological sample obtained from the subject at a second time, wherein the second time is later than the first time; and (3) comparing the level of the ERG-positive prostate cancer marker in the second sample with the level of the ERG-positive prostate cancer marker in the first sample; wherein a change in the level of the ERG-positive prostate cancer marker is indicative of a change in ERG-positive prostate cancer status in the subject.

In one aspect, the present invention provides methods for monitoring prostate cancer in a subject with a BMI index equal or greater than 30. The methods comprise (1) detecting the level of a high BMI prostate cancer marker in a first biological sample obtained at a first time from the subject having prostate cancer, wherein the high BMI prostate cancer marker comprises one or more markers selected from Tables 7, 18 and 25; (2) detecting the level of the high BMI prostate cancer marker in a second biological sample obtained from the subject at a second time, wherein the second time is later than the first time; and (3) comparing the level of the high BMI prostate cancer marker in the second sample with the level of the high BMI prostate cancer marker in the first sample; wherein a change in the level of the high BMI prostate cancer marker is indicative of a change in prostate cancer status in the subject.

In another aspect, the present invention provides methods for monitoring ERG-negative prostate cancer in a subject a Caucasian subject with a BMI index equal or greater than 30. The methods comprise (1) detecting the level of mercapto-succinyl-carnitine in a first biological sample obtained at a first time from a subject having ERG-negative prostate cancer; (2) detecting the level of mercapto-succinyl-carnitine in a second biological sample obtained from the subject at a second time, wherein the second time is later than the first time; and (3) comparing the level of mercapto-succinyl-carnitine in the second sample with the level of the at least one marker in the first sample; wherein a change in the level of mercapto-succinyl-carnitine is indicative of a change in prostate cancer status in the subject.

In some embodiments of the foregoing aspects, the biological sample is selected from the group consisting of blood, serum, plasma, urine, organ tissue, biopsy tissue, and seminal fluid.

In some embodiments of the foregoing aspects, steps (1) and (2) further comprise determining the amount of one or more additional markers of prostate cancer.

In some embodiments of the foregoing aspects, the subject is actively treated for prostate cancer prior to obtaining the second sample.

In some embodiments of the foregoing aspects, an increased or decreased level of the prostate cancer marker in the second biological sample as compared to the first biological sample is indicative of progression of the prostate cancer in the subject. In other embodiments of the foregoing aspects, an increased, decreased, or equivalent level of the prostate cancer marker in the second biological sample as compared to the first biological sample is indicative of non-progression of the prostate cancer in the subject.

In some embodiments of the foregoing aspects, an increased level of the prostate cancer marker selected from the group consisting of FFA_18:3, TAG_54:7+NH4, TAG 54:6+NH4, PA_18:1/20:2, FFA_18:3, FFA_20:1, TAG_54:7+NH4, TAG_54:6+NH4, PA_18:1/18:3, 6-KETO-PGF1A, TXB2, 13-HOTRE/13-HOTRE(R), 9-HOTRE, TXB2, 12-HEPE, 12-HETE, 13-HODE, APOC, APOB, ADIPOQ, SEPP1, CST3, F5, B2M, nicotinamide, eicosenoic acid, glycerylphosphorylethanolamine, nicotinamide, eicosenoic acid, 3-hydroxybutyric acid and 2-keto-isovalerate in the second biological sample as compared to the first biological sample is indicative of progression of the prostate cancer in the subject.

In some embodiments of the foregoing aspects, a decreased level of the prostate cancer marker selected from the group consisting of CE_22:2+NH4, CE_20:0+NH4, CE_22:3+NH4, DAG_40:1+NH4, CE_20:1+NH4, PI_18:0/20:5, CE_22:1+NH4, TAG_54:0+NH4, PI_18:0/20:4, PI_16:0/18:3, PI_16:0/20:4, CE_20:0+NH4, CE_24:0+NH4, CE_22:2+NH4, DAG_42:2+NH4, PE_36:2, 5-HETE, LXA4, 15-OXOETE, 5-HEPE, 8-HETE, LTB4, 5-HEPE, 5-HETE, LTB4, PGE2/PGD2, GPLD1, SERPING1, C3, A2M, SERPINA6, APOA4, APCS, ITIH2, CLU, APOA2, PPBP, C3, APOA4, C4BPA, MMRN2, APOA2, FGA, ABI3BP, APOA1, PROS1, COMP, CDH5, SERPINA6, glu-leu, 6-ketodecanoylcarnitine, myo-inositol, chenodeoxyglycocholate, 2-hydroxy-2-methylbutanedioic acid, nonanedioic acid, 6-ketodecanoylcarnitine, glu-leu, ethanolamine, and nonanoylcarnitine in the second biological sample as compared to the first biological sample is indicative of progression of the prostate cancer in the subject.

In some embodiments of the foregoing aspects, an increased level of the prostate cancer marker selected from Tables 4-7, 11, 12, 16-18, 22-25, 30 and 31 in the second biological sample as compared to the first biological sample is indicative of progression of the prostate cancer in the subject, wherein the prostate cancer marker comprises one or more markers having a FC ratio greater than 1, or a Log FC value greater than 0. In some embodiments of the foregoing aspects, a decreased level of the prostate cancer marker selected from Tables 4-7, 11, 12, 16-18, 22-25, 30 and 31 in the second biological sample as compared to the first biological sample is indicative of progression of the prostate cancer in the subject, wherein the prostate cancer marker comprises one or more markers having a FC ratio less than 1, or a Log FC value less than 0.

In some embodiments of the foregoing aspects, an increased level of the prostate cancer marker selected from the group consisting of FFA_18:3, TAG_54:7+NH4, TAG_54:6+NH4, PA_18:1/20:2, 6-KETO-PGF1A, TXB2, APOC, APOB, ADIPOQ, SEPP1, nicotinamide, eicosenoic acid, glycerylphosphorylethanolamine in the second biological sample as compared to the first biological sample is indicative of progression of the prostate cancer in the subject.

In some embodiments of the foregoing aspects, a decreased level of the prostate cancer marker selected from the group consisting of CE_22:2+NH4, CE_20:0+NH4, CE_22:3+NH4, DAG_40:1+NH4, CE_20:1+NH4, PI_18:0/20:5, CE_22:1+NH4, TAG_54:0+NH4, PI_18:0/20:4, PI_16:0/18:3, PI_16:0/20:4, 5-HETE, LXA4, 15-OXOETE, 5-HEPE, 8-HETE, LTB4, GPLD1, SERPING1, C3, A2M, SERPINA6, APOA4, APCS, ITIH2, CLU, APOA2, PPBP, glu-leu, 6-ketodecanoylcarnitine, myo-inositol, chenodeoxyglycocholate, 2-hydroxy-2-methylbutanedioic acid, nonanedioic acid in the second biological sample as compared to the first biological sample is indicative of progression of the prostate cancer in the subject.

In some embodiments of the foregoing aspects, an increased level of the prostate cancer marker selected from Tables 4, 11, 16, 22 and 30 in the second biological sample as compared to the first biological sample is indicative of progression of the prostate cancer in the subject, wherein the prostate cancer comprises one or more markers having a FC ratio greater than 1, or a Log FC value greater than 0. In some embodiments of the foregoing aspects, a decreased level of the prostate cancer marker selected from Tables 4, 11, 16, 22 and 30 in the second biological sample as compared to the first biological sample is indicative of progression of the prostate cancer in the subject, wherein the prostate cancer comprises one or more markers having a FC ratio less than 1, or a Log FC value less than 0.

In some embodiments of the foregoing aspects, an increased level of the prostate cancer marker selected from the group consisting of FFA_18:3, FFA_20:1, TAG_54:7+NH4, TAG 54:6+NH4, PA_18:1/18:3, 13-HOTRE/13-HOTRE(R), 9-HOTRE, TXB2, 12-HEPE, 12-HETE, 13-HODE, CST3, F5, B2M, nicotinamide, eicosenoic acid, 3-hydroxybutyric acid, 2-keto-isovalerate and 2-octandioic-carnitine in the second biological sample as compared to the first biological sample is indicative of progression of the prostate cancer in the subject.

In some embodiments of the foregoing aspects, a decreased level of the prostate cancer marker selected from the group consisting of CE_20:0+NH4, CE_24:0+NH4, CE_22:2+NH4, DAG_42:2+NH4, PE_36:2, 5-HEPE, 5-HETE, LTB4, PGE2/PGD2, C3, APOA4, C4BPA, MMRN2, APOA2, FGA, ABI3BP, APOA1, PROS1, COMP, CDH5, SERPINA6, 6-ketodecanoylcarnitine, glu-leu, ethanolamine, nonanoylcarnitine and propionylcarnitine in the second biological sample as compared to the first biological sample is indicative of progression of the prostate cancer in the subject.

In some embodiments of the foregoing aspects, an increased level of the prostate cancer marker selected from Tables 5, 12, 17, 23 and 31 in the second biological sample as compared to the first biological sample is indicative of progression of the prostate cancer in the subject, wherein the prostate cancer comprises one or more markers having a FC ratio greater than 1, or a Log FC value greater than 0. In some embodiments of the foregoing aspects, a decreased level of the prostate cancer marker selected from Tables 5, 12, 17, 23 and 31 in the second biological sample as compared to the first biological sample is indicative of progression of the prostate cancer in the subject, wherein the prostate cancer comprises one or more markers having a FC ratio less than 1, or a Log FC value less than 0.

In some embodiments of the foregoing aspects, an increased or decreased level of the ERG-positive prostate cancer marker in the second biological sample as compared to the first biological sample is indicative of progression of the ERG-positive prostate cancer in the subject. In some embodiments of the foregoing aspects, an increased, decreased, or equivalent level of the ERG-positive prostate cancer marker in the second biological sample as compared to the first biological sample is indicative of non-progression of the ERG-positive prostate cancer in the subject.

In some embodiments of the foregoing aspects, an increased level of the ERG-positive prostate cancer marker selected from the group consisting of CE_20:4+NH4, PG_16:1/18:3, D18:0/16:1-MONOHEX, D18:1/22:1-MONOHEX, PG_16:1/20:3 in the second biological sample as compared to the first biological sample is indicative of progression of the ERG-positive prostate cancer in the subject.

In some embodiments of the foregoing aspects, a decreased level of the ERG-positive prostate cancer marker selected from the group consisting of LPC_0-14:1, LPC_22:1, LPC_10:0, LPC_O-22:0, LPC_24:0 in the second biological sample as compared to the first biological sample is indicative of progression of the ERG-positive prostate cancer in the subject.

In some embodiments of the foregoing aspects, an increased level of the ERG-positive prostate cancer marker selected from selected from Tables 6, 30 and 31 in the second biological sample as compared to the first biological sample is indicative of progression of the ERG-positive prostate cancer in the subject, wherein the ERG-positive prostate cancer comprises one or more markers having a FC ratio greater than 1, or a Log FC value greater than 0. In some embodiments of the foregoing aspects, a decreased level of the ERG-positive prostate cancer marker selected from selected from Tables 6, 30 and 31 in the second biological sample as compared to the first biological sample is indicative of progression of the ERG-positive prostate cancer in the subject, wherein the ERG-positive prostate cancer comprises one or more markers having a FC ratio less than 1, or a Log FC value less than 0.

In some embodiments of the foregoing aspects, an increased or decreased level of the high BMI prostate cancer marker in the second biological sample as compared to the first biological sample is indicative of progression of the prostate cancer in the subject. In some embodiments of the foregoing aspects, an increased, decreased, or equivalent level of the high BMI prostate cancer marker in the second biological sample as compared to the first biological sample is indicative of non-progression of the prostate cancer in the subject.

In some embodiments of the foregoing aspects, an increased level of the high BMI prostate cancer marker selected from selected from Tables 7, 18 and 25 in the second biological sample as compared to the first biological sample is indicative of progression of the prostate cancer in the subject, wherein the high BMI prostate cancer marker comprises one or more markers having a FC ratio greater than 1, or a Log FC value greater than 0. In some embodiments of the foregoing aspects, a decreased level of the high BMI prostate cancer marker selected from selected from Tables 7, 18 and 25 in the second biological sample as compared to the first biological sample is indicative of progression of the prostate cancer in the subject, wherein the high BMI prostate cancer marker comprises one or more markers having a FC ratio less than 1, or a Log FC value less than 0.

In some embodiments of the foregoing aspects, an increased level of mercapto-succinyl-carnitine in the second biological sample as compared to the first biological sample is indicative of progression of the ERG-negative prostate cancer in the subject. In some embodiments of the foregoing aspects, a decreased, or equivalent level of mercapto-succinyl-carnitine in the second biological sample as compared to the first biological sample is indicative of non-progression of the ERG-negative prostate cancer in the subject.

In some embodiments of the foregoing aspects, the methods described herein further comprise selecting and/or administering a different treatment regimen for the subject based on progression of the prostate cancer in the subject.

In some embodiments of the foregoing aspects, the subject is selected from a population of Caucasians. In some embodiments of the foregoing aspects, the subject is selected from a population of African Americans.

In one aspect, the present invention provides methods for identifying an agent that modulates prostate cancer progression. The methods comprise (a) contacting a cell with a test compound, and (b) determining the expression and/or activity of a prostate cancer marker, wherein the prostate cancer marker comprises one or more markers selected from Tables 1-31.

In another aspect, the present invention provides methods for identifying an agent that modulates ERG-positive prostate cancer progression. The methods comprise (a) contacting a cell with at least one test compound, and (b) determining the expression and/or activity of an ERG-positive prostate cancer marker, wherein the ERG-positive prostate cancer marker comprises one or more markers selected from Tables 6, 30 and 31.

In some embodiments of the foregoing aspects, the cell is a prostate cancer cell. In some embodiments of the foregoing aspects, the cell is a ERG-positive prostate cancer cell.

In some embodiments of the foregoing aspects, the cell is engineered to produce the prostate cancer marker selected from Tables 1-31. In some embodiments of the foregoing aspects, the cell is engineered to produce the ERG-positive prostate cancer marker selected from Tables 6, 30 and 31.

In some embodiments of the foregoing aspects, the at least one test compound is selected from the group consisting of small molecules, antibodies, and nucleic acid inhibitors.

In one aspect, the present invention provides compounds identified by any one of the methods described herein.

In one aspect, the present invention provides methods of treating prostate cancer in a subject. The methods comprise administering to the subject a modulator of a prostate cancer marker, wherein the prostate cancer marker comprises one or more markers selected from Tables 1-31.

In another aspect, the present invention provides methods of treating prostate cancer in a subject selected from a population of Caucasians. The methods comprise administering to the subject a modulator of a prostate cancer marker, wherein the prostate cancer marker comprises one or more markers selected from Tables 1, 4, 8, 11, 13, 16, 19, 22, 26, 29 and 30.

In one aspect, the present invention provides methods of treating prostate cancer in a subject selected from a population of African Americans. The methods comprise administering to the subject a modulator of a prostate cancer marker, wherein the prostate cancer marker comprises one or more markers selected from Tables 2, 5, 9, 12, 14, 17, 20, 23, 27 and 31.

In another aspect, the present invention provides methods of treating ERG-positive prostate cancer in a subject. The methods comprise administering to the subject a modulator of an ERG-positive prostate cancer marker, wherein the ERG-positive prostate cancer marker comprises one or more markers selected from Tables 6, 30 and 31.

In one aspect, the present invention provides methods of treating prostate cancer in a subject with a BMI index equal or greater than 30. The methods comprise administering to the subject a modulator of a high BMI prostate cancer marker, wherein the high BMI prostate cancer marker comprises one or more markers selected from Tables 7, 18 and 25.

In another aspect, the present invention provides methods of treating ERG-negative prostate cancer in a Caucasian subject with a BMI index equal or greater than 30. The methods comprise administering to the subject a modulator of mercapto-succinyl-carnitine.

In some embodiments of the foregoing aspects, the modulator increases the level or activity of the prostate cancer marker. In some embodiments of the foregoing aspects, the prostate cancer marker comprises one or more markers selected from the group consisting of CE_22:2+NH4, CE_20:0+NH4, CE_22:3+NH4, DAG_40:1+NH4, CE_20:1+NH4, PI_18:0/20:5, CE_22:1+NH4, TAG 54:0+NH4, PI_18:0/20:4, PI_16:0/18:3, PI_16:0/20:4, CE_20:0+NH4, CE_24:0+NH4, CE_22:2+NH4, DAG_42:2+NH4, PE_36:2, 5-HETE, LXA4, 15-OXOETE, 5-HEPE, 8-HETE, LTB4, 5-HEPE, 5-HETE, LTB4, PGE2/PGD2, GPLD1, SERPING1, C3, A2M, SERPINA6, APOA4, APCS, ITIH2, CLU, APOA2, PPBP, C3, APOA4, C4BPA, MMRN2, APOA2, FGA, ABI3BP, APOA1, PROS1, COMP, CDH5, SERPINA6, glu-leu, 6-ketodecanoylcarnitine, myo-inositol, chenodeoxyglycocholate, 2-hydroxy-2-methylbutanedioic acid, nonanedioic acid, 6-ketodecanoylcarnitine, glu-leu, ethanolamine, and nonanoylcarnitine. In some embodiments of the foregoing aspects, the prostate cancer marker comprises one or more markers selected from Tables 4-7, 11, 12, 16-18, 22-25, 30 and 31, wherein the one or more markers have a FC ratio less than 1, or a Log FC value less than 0. In some embodiments of the foregoing aspects, the modulator decreases the level or activity of the prostate cancer marker. In some embodiments of the foregoing aspects, the prostate cancer marker is selected from the group consisting of FFA_18:3, TAG_54:7+NH4, TAG_54:6+NH4, PA_18:1/20:2, FFA_18:3, FFA_20:1, TAG_54:7+NH4, TAG_54:6+NH4, PA_18:1/18:3, 6-KETO-PGF1A, TXB2, 13-HOTRE/13-HOTRE(R), 9-HOTRE, TXB2, 12-HEPE, 12-HETE, 13-HODE, APOC, APOB, ADIPOQ, SEPP1, CST3, F5, B2M, nicotinamide, eicosenoic acid, glycerylphosphorylethanolamine, nicotinamide, eicosenoic acid, 3-hydroxybutyric acid and 2-keto-isovalerate. In some embodiments of the foregoing aspects, the prostate cancer marker comprises one or more markers selected from Tables 4-7, 11, 12, 16-18, 22-25, 30 and 31, wherein the one or more markers have a FC ratio greater than 1, or a Log FC value greater than 0.

In some embodiments of the foregoing aspects, the modulator increases the level or activity of the prostate cancer marker. In some embodiments of the foregoing aspects, the prostate cancer marker comprises one or more markers selected from the group consisting of CE_22:2+NH4, CE_20:0+NH4, CE_22:3+NH4, DAG_40:1+NH4, CE_20:1+NH4, PI_18:0/20:5, CE_22:1+NH4, TAG 54:0+NH4, PI_18:0/20:4, PI_16:0/18:3, PI_16:0/20:4, 5-HETE, LXA4, 15-OXOETE, 5-HEPE, 8-HETE, LTB4, GPLD1, SERPING1, C3, A2M, SERPINA6, APOA4, APCS, ITIH2, CLU, APOA2, PPBP, glu-leu, 6-ketodecanoylcarnitine, myo-inositol, chenodeoxyglycocholate, 2-hydroxy-2-methylbutanedioic acid, nonanedioic acid. In some embodiments of the foregoing aspects, the prostate cancer marker comprises one or more markers selected from Tables 4, 11, 16, 22 and 30, wherein the one or more markers have a FC ratio less than 1, or a Log FC value less than 0.

In some embodiments of the foregoing aspects, the modulator decreases the level or activity of the prostate cancer marker. In some embodiments of the foregoing aspects, the prostate cancer marker is selected from the group consisting of FFA_18:3, TAG_54:7+NH4, TAG_54:6+NH4, PA_18:1/20:2, 6-KETO-PGF1A, TXB2, APOC, APOB, ADIPOQ, SEPP1, nicotinamide, eicosenoic acid, glycerylphosphorylethanolamine. In some embodiments of the foregoing aspects, the prostate cancer marker comprises one or more markers selected from Tables 4, 11, 16, 22 and 30, wherein the one or more markers have a FC ratio greater than 1, or a Log FC value greater than 0.

In some embodiments of the foregoing aspects, the modulator increases the level or activity of the prostate cancer marker. In some embodiments of the foregoing aspects, the prostate cancer marker comprises one or more markers selected from the group consisting of CE_20:0+NH4, CE_24:0+NH4, CE_22:2+NH4, DAG_42:2+NH4, PE_36:2, 5-HEPE, 5-HETE, LTB4, PGE2/PGD2, C3, APOA4, C4BPA, MMRN2, APOA2, FGA, ABI3BP, APOA1, PROS1, COMP, CDH5, SERPINA6, 6-ketodecanoylcarnitine, glu-leu, ethanolamine, nonanoylcarnitine and propionylcarnitine. In some embodiments of the foregoing aspects, the prostate cancer marker comprises one or more markers selected from Tables 5, 12, 17, 23 and 31, wherein the one or more markers have a FC ratio less than 1, or a Log FC value less than 0.

In some embodiments of the foregoing aspects, the modulator decreases the level or activity of the prostate cancer marker. In some embodiments of the foregoing aspects, the prostate cancer marker is selected from the group consisting of FFA_18:3, FFA_20:1, TAG_54:7+NH4, TAG_54:6+NH4, PA_18:1/18:3, 13-HOTRE/13-HOTRE(R), 9-HOTRE, TXB2, 12-HEPE, 12-HETE, 13-HODE, CST3, F5, B2M, nicotinamide, eicosenoic acid, 3-hydroxybutyric acid, 2-keto-isovalerate and 2-octandioic-carnitine. In some embodiments of the foregoing aspects, the prostate cancer marker comprises one or more markers selected from Tables 5, 12, 17, 23 and 31, wherein the one or more markers have a FC ratio greater than 1, or a Log FC value greater than 0.

In some embodiments of the foregoing aspects, the modulator increases the level or activity of the ERG-positive prostate cancer marker. In some embodiments of the foregoing aspects, the ERG-positive prostate cancer marker comprises one or more markers selected from the group consisting of LPC_O-14:1, LPC_22:1, LPC_10:0, LPC_O-22:0, LPC_24:0. In some embodiments of the foregoing aspects, the ERG-positive prostate cancer marker comprises one or more markers selected from Tables 6, 30 and 31, wherein the one or more markers have a FC ratio less than 1, or a Log FC value less than 0.

In some embodiments of the foregoing aspects, the modulator decreases the level or activity of the ERG-positive prostate cancer marker. In some embodiments of the foregoing aspects, the ERG-positive prostate cancer marker is selected from the group consisting of CE_20:4+NH4, PG_16:1/18:3, D18:0/16:1-MONOHEX, D18:1/22:1-MONOHEX, PG_16:1/20:3. In some embodiments of the foregoing aspects, the ERG-positive prostate cancer marker comprises one or more markers selected from Tables 6, 30 and 31, wherein the one or more markers have a FC ratio greater than 1, or a Log FC value greater than 0.

In some embodiments of the foregoing aspects, the modulator increases the level or activity of the high BMI prostate cancer marker. In some embodiments of the foregoing aspects, the high BMI prostate cancer marker comprises one or more markers selected from Tables 7, 18 and 25, wherein the one or more markers have a FC ratio less than 1, or a Log FC value less than 0.

In some embodiments of the foregoing aspects, the modulator decreases the level or activity of the high BMI prostate cancer marker. In some embodiments of the foregoing aspects, the high BMI prostate cancer marker comprises one or more markers selected from Tables 7, 18 and 25, wherein the one or more markers have a FC ratio greater than 1, or a Log FC value greater than 0.

In some embodiments of the foregoing aspects, the modulator decreases the level or activity of mercapto-succinyl-carnitine. In some embodiments of the foregoing aspects, the prostate cancer marker comprises one or more markers selected from Table 29.

In one aspect, the present invention provides kits for detecting a prostate cancer marker in a biological sample from a subject having, suspected of having, or at risk for having prostate cancer. The kits comprise one or more reagents for measuring the level of the prostate cancer marker in the biological sample from the subject, wherein the prostate cancer marker comprises one or more markers selected from Tables 1-31 and a set of instructions for measuring the level of the prostate cancer marker.

In some embodiments of the foregoing aspects, the reagent is an antibody. In some embodiments of the foregoing aspects, the kit further comprise a means to detect the antibody. In some embodiments of the foregoing aspects, the reagent is an oligonucleotide that is complementary to the corresponding mRNA of the prostate cancer marker.

In some embodiments of the foregoing aspects, the instructions set forth an immunoassay, ELISA, or mass spectrometry assay for detecting the level of the prostate cancer marker in the biological sample. In some embodiments of the foregoing aspects, the instructions set forth an amplification reaction for assaying the level of the mRNA in the biological sample corresponding to the prostate cancer marker. In some embodiments of the foregoing aspects, the instructions set forth a hybridization assay for detecting the level of the mRNA in the biological sample corresponding to the prostate cancer marker. In some embodiments of the foregoing aspects, the instructions further set forth comparing the level of the prostate cancer marker in the biological sample from the subject to a predetermined threshold value of the prostate cancer marker. In some embodiments of the foregoing aspects, the instructions further set forth making a diagnosis of prostate cancer based on the level of the prostate cancer marker in the biological sample from the subject as compared to a predetermined threshold value of the prostate cancer marker.

In some embodiments of the foregoing aspects, the subject is selected from a population of Caucasians. In some embodiments of the foregoing aspects, the subject is selected from a population of African Americans.

In another aspect, the present invention provides panels for use in a method of monitoring the treatment of prostate cancer. The panels comprise one or more detection reagents, wherein each detection reagent is specific for the detection of a prostate cancer marker, wherein the prostate cancer marker comprises one or more markers selected from Tables 1-31.

In some embodiments of the foregoing aspects, the prostate cancer marker comprises at least two or more markers, wherein each of the two or more markers are selected from the structural lipids set forth in Tables 1-7, the signaling lipids set forth in Tables 8-12, the proteins set forth in Tables 13-18, the metabolites set forth in Tables 19-25, and the markers set forth in Tables 26-28.

In some embodiments of the foregoing aspects, the prostate cancer marker comprises at least two or more markers, wherein each of the two or more markers are selected from the structural lipids set forth in Tables 1-3, the signaling lipids set forth in Tables 8-10, the proteins set forth in Tables 13-15, the metabolites set forth in Tables 19-21, and the markers set forth in Tables 26-28.

In another aspect, the present invention provides kits comprising the panels as described herein and a set of instructions for obtaining diagnostic information based on a level of the prostate cancer marker.

In some embodiments of the foregoing aspects, the level of the prostate cancer marker is increased when compared to a predetermined threshold value. In some embodiments of the foregoing aspects, the level of the prostate cancer marker is decreased when compared to a predetermined threshold value.

In some embodiments of the foregoing aspects, the prostate cancer marker comprises one or more markers with an increased level when compared to a predetermined threshold value, and/or one or more markers with a decreased level when compared to a predetermined threshold value.

In some embodiments of the foregoing aspects, the prostate cancer marker is selected from the group consisting of FFA_18:3, TAG_54:7+NH4, TAG_54:6+NH4, PA_18:1/20:2, FFA_18:3, FFA_20:1, TAG_54:7+NH4, TAG_54:6+NH4, PA_18:1/18:3, 6-KETO-PGF1A, TXB2, 13-HOTRE/13-HOTRE(R), 9-HOTRE, TXB2, 12-HEPE, 12-HETE, 13-HODE, APOC, APOB, ADIPOQ, SEPP1, CST3, F5, B2M, nicotinamide, eicosenoic acid, glycerylphosphorylethanolamine, nicotinamide, eicosenoic acid, 3-hydroxybutyric acid and 2-keto-isovalerate.

In some embodiments of the foregoing aspects, the prostate cancer marker is selected from the group consisting of CE_22:2+NH4, CE_20:0+NH4, CE_22:3+NH4, DAG_40:1+NH4, CE_20:1+NH4, PI_18:0/20:5, CE_22:1+NH4, TAG_54:0+NH4, PI_18:0/20:4, PI_16:0/18:3, PI_16:0/20:4, CE_20:0+NH4, CE_24:0+NH4, CE_22:2+NH4, DAG_42:2+NH4, PE 36:2, 5-HETE, LXA4, 15-OXOETE, 5-HEPE, 8-HETE, LTB4, 5-HEPE, 5-HETE, LTB4, PGE2/PGD2, GPLD1, SERPING1, C3, A2M, SERPINA6, APOA4, APCS, ITIH2, CLU, APOA2, PPBP, C3, APOA4, C4BPA, MMRN2, APOA2, FGA, ABI3BP, APOA1, PROS1, COMP, CDH5, SERPINA6, glu-leu, 6-ketodecanoylcarnitine, myo-inositol, chenodeoxyglycocholate, 2-hydroxy-2-methylbutanedioic acid, nonanedioic acid, 6-ketodecanoylcarnitine, glu-leu, ethanolamine, and nonanoylcarnitine.

In some embodiments of the foregoing aspects, the prostate cancer marker comprises one or more markers selected from Tables 4-7, 11, 12, 16-18, 22-25, 30 and 31, wherein the one or more markers have a FC ratio greater than 1, or a Log FC value greater than 0.

In some embodiments of the foregoing aspects, the prostate cancer marker comprises one or more markers selected from Tables 4-7, 11, 12, 16-18, 22-25, 30 and 31, wherein the one or more markers have a FC ratio less than 1, or a Log FC value less than 0.

In another aspect, the present invention provides uses of a panel comprising a plurality of detection reagents specific for detecting a prostate cancer marker in a method for diagnosing and/or treating prostate cancer, wherein each detection reagent of the panel is specific for the detection of a prostate cancer marker, wherein the prostate cancer marker comprises at least two markers selected from Tables 1-31.

Where applicable or not specifically disclaimed, any one of the embodiments described herein are contemplated to be able to combine with any other one or more embodiments, even though the embodiments are described under different aspects of the invention.

These and other embodiments are disclosed or are obvious from and encompassed by, the following Detailed Description.

### BRIEF DESCRIPTION OF THE DRAWINGS

FIG. 1 is a box plot depicting a direct comparison of normalized expression levels of individual structural lipid markers identified in Table 1 between Caucasian prostate cancer patients and negative controls.

FIG. 2 is a box plot depicting a direct comparison of normalized expression levels of individual structural lipid markers identified in Table 2 between American African prostate cancer patients and negative controls.

FIG. 3 is a box plot depicting a direct comparison of normalized expression levels of individual structural lipid markers identified in Table 3 between Caucasian and African American prostate cancer patients and negative controls.

FIG. 4 depicts a ROC curve with a predictive diagnostic value of 0.942 for a set of structural lipid markers for Caucasian prostate cancer patients.

FIG. 5 depicts a ROC curve with a predictive diagnostic value of 0.847 for a set of structural lipid markers for African American prostate cancer patients.

FIG. 6 depicts a ROC curve with a predictive diagnostic value of 0.891 for a set of structural lipid markers for Caucasian and African American prostate cancer patients.

FIG. 7 is a box plot depicting a direct comparison of normalized expression levels of individual signaling lipid markers identified in Table 8 between Caucasian prostate cancer patients and negative controls.

FIG. 8 is a box plot depicting a direct comparison of normalized expression levels of individual signaling lipid markers identified in Table 9 between American African prostate cancer patients and negative controls.

FIG. 9 is a box plot depicting a direct comparison of normalized expression levels of individual signaling lipid markers identified in Table 10 between Caucasian and African American prostate cancer patients and negative controls.

FIG. 10 depicts a ROC curve with a predictive diagnostic value of 0.987 for a set of signaling lipid markers for Caucasian prostate cancer patients.

FIG. 11 depicts a ROC curve with a predictive diagnostic value of 0.94 for a set of signaling lipid markers for African American prostate cancer patients.

FIG. 12 depicts a ROC curve with a predictive diagnostic value of 0.957 for a set of signaling lipid markers for Caucasian and African American prostate cancer patients.

FIG. 13 is a box plot depicting a direct comparison of normalized expression levels of individual protein markers identified in Table 13 between Caucasian prostate cancer patients and negative controls.

FIG. 14 is a box plot depicting a direct comparison of normalized expression levels of individual protein markers identified in Table 14 between American African prostate cancer patients and negative controls.

FIG. 15 is a box plot depicting a direct comparison of normalized expression levels of individual protein markers identified in Table 15 between Caucasian and African American prostate cancer patients and negative controls.

FIG. 16 depicts a ROC curve with a predictive diagnostic value of 0.879 for a set of protein markers for Caucasian prostate cancer patients.

FIG. 17 depicts a ROC curve with a predictive diagnostic value of 0.868 for a set of protein markers for African American prostate cancer patients.

FIG. 18 depicts a ROC curve with a predictive diagnostic value of 0.856 for a set of protein markers for Caucasian and African American prostate cancer patients.

FIG. 19 is a box plot depicting a direct comparison of normalized expression levels of individual metabolite markers identified in Table 19 between Caucasian prostate cancer patients and negative controls.

FIG. 20 is a box plot depicting a direct comparison of normalized expression levels of individual metabolite markers identified in Table 20 between American African prostate cancer patients and negative controls.

FIG. 21 is a box plot depicting a direct comparison of normalized expression levels of individual metabolite markers identified in Table 21 between Caucasian and African American prostate cancer patients and negative controls.

FIG. 22 depicts a ROC curve with a predictive diagnostic value of 0.99 for a set of metabolite markers for Caucasian prostate cancer patients.

FIG. 23 depicts a ROC curve with a predictive diagnostic value of 0.991 for a set of metabolite markers for African American prostate cancer patients.

FIG. 24 depicts a ROC curve with a predictive diagnostic value of 0.988 for a set of metabolite markers for Caucasian and African American prostate cancer patients.

FIG. 25 is a box plot depicting an association of a high serum level of mercapto-succinyl-carnitine metabolite with the presence of ERG-index tumors in obese Caucasian prostate cancer patients.

FIG. 26 is a box plot depicting a direct comparison of normalized expression levels of individual omics markers identified in Table 26 between Caucasian prostate cancer patients and negative controls.

FIG. 27 is a box plot depicting a direct comparison of normalized expression levels of individual omics markers identified in Table 27 between American African prostate cancer patients and negative controls.

FIG. 28 is a box plot depicting a direct comparison of normalized expression levels of individual omics markers identified in Table 28 between Caucasian and African American prostate cancer patients and negative controls.

FIG. 29 depicts a ROC curve with a predictive diagnostic value of 0.992 for a set of omics markers for Caucasian prostate cancer patients.

FIG. 30 depicts a ROC curve with a predictive diagnostic value of 0.995 for a set of omics markers for African American prostate cancer patients.

FIG. 31 depicts a ROC curve with a predictive diagnostic value of 0.994 for a set of omics markers for Caucasian and African American prostate cancer patients.

FIG. 32 depicts a ROC curve for omics markers selected from Table 29 in Caucasian prostate cancer patients for Gleason score class predication.

FIG. 33 depicts a ROC curve for the combination of patient age and the omics markers selected from Table 29 in Caucasian prostate cancer patients for Gleason score class predication.

FIG. 34 depicts a ROC curve for the combination of patient age, patient diagnostic PSA level, and the omics markers selected from Table 29 in Caucasian prostate cancer patients for Gleason score class predication.

FIG. 35 is a box plot depicting a direct comparison of normalized expression levels of individual structural lipid markers identified in Table 30 between Caucasian ERG positive and ERG negative prostate cancer patients.

FIG. 36 is a box plot depicting a direct comparison of normalized expression levels of individual structural lipid markers identified in Table 31 between African American ERG positive and ERG negative prostate cancer patients.

### DETAILED DESCRIPTION OF THE INVENTION

### A. Overview

The identification of tumor markers or antigens associated with prostate cancer has stimulated considerable interest as promising tools for the screening, diagnosis, prognosis, clinical management, and potential treatment of prostate cancer, and in particular, prognosis and early detection of prostate cancer. Indeed, early detection mitigates the risk that the cancer will metastasize. Non-metastasized, local prostate tumors can often be cured by radical prostatectomy or radiation therapy, however for patients with distantly spread disease, no curative treatment is available. This emphasizes the need for new prostate (cancer) specific prognosic and diagnostic tools that may improve the chances for accurate prediction and early detection of prostate cancer across various populations and clinical phenotypes.

While some prostate-specific markers are known, e.g., prostate-specific antigen and prostate stem cell antigen, very few biomarkers are in widespread or routine use as molecular diagnostics for prostate cancer. Accordingly, there remains a need for efficient, accurate, and rapid molecular prognosis and diagnosis means, particularly which do not suffer from a high proportion of false results. The development of molecular tests for the accurate prognosis, i.e., prediction of one's risk for the development of prostate cancer and detection of prostate cancer will also lead to improved management of appropriate therapies, and an overall improved survival rate. Thus, there remains a need to provide an improved prognostic and/or diagnostic test for the prediction or detection of prostate cancer which is more reliable and accurate than PSA and other current screening tests. The present invention addresses this need by providing the use of biomarkers, i.e., one or more markers selected from Tables 1-31, for the accurate and reliable detection of prostate cancer.

As presently described herein, the invention at hand is based, at least in part, on the discovery that the one or more markers selected from Tables 1-31 are differentially regulated in prostate cancer cells and serve as useful biomarkers of prostate cancer. In particular, the invention is based on the surprising discovery that the markers in Tables 1-31 differentially expressed, e.g., either increased or decreased as compared to a control, in the serum of patients with prostate cancer, and are thus useful in the diagnosis and/or prognosis of prostate cancer.

It is also surprisingly discovered that markers for the prognosis and/or diagnosis of prostate cancer are differentially expressed based on race or clinical phenotype. For example, in one embodiment, markers of the invention are differentially expressed among different populations, for example, in African American (AA) or Caucasian American (CA) populations.

In another embodiment, markers of the invention are also differentally expressed in subjects with different types of prostate cancer, such as ERG-positive and ERG-negative tumors, or tumors having different Gleason scores. In another embodiment, markers of the invention are also differentally expressed in subjects having different BMIs.

Accordingly, the invention provides methods for diagnosing and/or monitoring (e.g., monitoring of disease progression or treatment) and/or prognosing an oncological disease state, e.g., prostate cancer, in a subject. In some embodiments, the subject is selected from a general population. In other embodiments, the subject is selected from a population of Caucasians. In yet another embodiment, the subject is selected from a population of African American. In some embodiments, the subject has an ERG-positive prostate cancer. In other embodiments, the subject has an ERG-negative prostate cancer. In a further embodiment, the subject has a BMI index equal or greater than 30.

In one embodiment, these one or more markers selected from Tables 1-31 can serve as useful diagnostic biomarkers to predict and/or detect the presence of prostate cancer in a subject. In another embodiment, these one or more markers selected from Tables 1-31 can serve as useful prognostic biomarkers, serving to inform on the likely development or progression of prostate cancer in a subject with or without treatment. In still another embodiment, these one or more markers selected from Tables 1-31 can serve as useful predictive biomarkers for helping to assess the likely response of prostate cancer to a particular treatment. Accordingly, the invention provides methods that use the one or more markers selected from Tables 1-31 in the diagnosis of prostate cancer (e.g., prediction of the presence of prostate cancer in a subject), in the prognosis of prostate cancer (e.g., prediction of the development of prostate cancer, or the course or outcome of prostate cancer with or without treatment), and in the assessment of therapies intended to treat prostate cancer (e.g., the one or more markers selected from Tables 1-31 as a theragnostic or predictive marker).

The following is a detailed description of the invention provided to aid those skilled in the art in practicing the present invention. Those of ordinary skill in the art may make modifications and variations in the embodiments described herein without departing from the spirit or scope of the present invention. Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, figures and other references mentioned herein are expressly incorporated by reference in their entirety.

Although any methods and materials similar or equivalent to those described herein can also be used in the practice or testing of the present invention, the preferred methods and materials are now described. All publications mentioned herein are incorporated herein by reference to disclose and described the methods and/or materials in connection with which the publications are cited.

### B. Definitions

Unless defined otherwise, all technical and scientific terms used herein have the meaning commonly understood by a person skilled in the art to which this invention belongs. The following references, the entire disclosures of which are incorporated herein by reference, provide one of skill with a general definition of many of the terms (unless defined otherwise herein) used in this invention: Singleton et al., Dictionary of Microbiology and Molecular Biology (2^{nd} ed. 1994); The Cambridge Dictionary of Science and Technology (Walker ed., 1988); The Glossary of Genetics, 5^{th} Ed., R. Rieger et al. (eds.), Springer Verlag (1991); and Hale & Marham, the Harper Collins Dictionary of Biology (1991). Generally, the procedures of molecular biology methods described or inherent herein and the like are common methods used in the art. Such standard techniques can be found in reference manuals such as for example Sambrook et al., (2000, Molecular Cloning—A Laboratory Manual, Third Edition, Cold Spring Harbor Laboratories); and Ausubel et al., (1994, Current Protocols in Molecular Biology, John Wiley & Sons, New-York).

The following terms may have meanings ascribed to them below, unless specified otherwise. However, it should be understood that other meanings that are known or understood by those having ordinary skill in the art are also possible, and within the scope of the present invention. All publications, patent applications, patents, and other references mentioned herein are incorporated by reference in their entirety. In the case of conflict, the present specification, including definitions, will control. In addition, the materials, methods, and examples are illustrative only and not intended to be limiting.

As used herein, the singular forms “a”, “and”, and “the” include plural references unless the context clearly dictates otherwise. All technical and scientific terms used herein have the same meaning.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

As used herein, the term “amplification” refers to any known in vitro procedure for obtaining multiple copies (“amplicons”) of a target nucleic acid sequence or its complement or fragments thereof. In vitro amplification refers to production of an amplified nucleic acid that may contain less than the complete target region sequence or its complement. Known in vitro amplification methods include, e.g., transcription-mediated amplification, replicase-mediated amplification, polymerase chain reaction (PCR) amplification, ligase chain reaction (LCR) amplification and strand-displacement amplification (SDA including multiple strand-displacement amplification method (MSDA)). Replicase-mediated amplification uses self-replicating RNA molecules, and a replicase such as Q-O-replicase (e.g., Kramer et al., U.S. Pat. No. 4,786,600). PCR amplification is well known and uses DNA polymerase, primers and thermal cycling to synthesize multiple copies of the two complementary strands of DNA or cDNA (e.g., Mullis et al., U.S. Pat. Nos. 4,683,195, 4,683,202, and 4,800,159). LCR amplification uses at least four separate oligonucleotides to amplify a target and its complementary strand by using multiple cycles of hybridization, ligation, and denaturation (e.g., EP Pat. App. Pub. No. 0 320 308). SDA is a method in which a primer contains a recognition site for a restriction endonuclease that permits the endonuclease to nick one strand of a hemimodified DNA duplex that includes the target sequence, followed by amplification in a series of primer extension and strand displacement steps (e.g., Walker et al., U.S. Pat. No. 5,422,252). Two other known strand-displacement amplification methods do not require endonuclease nicking (Dattagupta et al., U.S. Pat. Nos. 6,087,133 and 6,124,120 (MSDA)). Those skilled in the art will understand that the oligonucleotide primer sequences of the present invention may be readily used in any in vitro amplification method based on primer extension by a polymerase. (see generally Kwoh et al., 1990, Am. Biotechnol. Lab. 8:14-25 and (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 2000, Molecular Cloning—A Laboratory Manual, Third Edition, CSH Laboratories). As commonly known in the art, the oligos are designed to bind to a complementary sequence under selected conditions.

As used herein, the term “antigen” refers to a molecule, e.g., a peptide, polypeptide, protein, fragment, or other biological moiety, which elicits an antibody response in a subject, or is recognized and bound by an antibody.

As used herein, the term “marker” is a biological molecule, or a panel of biological molecules, whose altered level in a tissue or cell as compared to its level in normal or healthy tissue or cell is associated with a disease state, such as an abnormal prostate state, including disease in an early stage, e.g., prior to the detection of one or more symptoms associated with the disease. In a preferred embodiment, the marker is detected in a blood sample, e.g., serum or plasma. In one embodiment, the marker is detected in serum. In one embodiment, the marker is detected in plasma. In certain embodiments, the serum or plasma can be further processed to remove abundant blood proteins (e.g., albumin) or proteins that are not marker proteins prior to analysis. Examples of biomarkers include, for example, polypeptides, peptides, polypeptide fragments, proteins, antibodies, hormones, polynucleotides, RNA or RNA fragments, microRNA (miRNAs), lipids, metabolites, or polysaccharides.

As used herein, the term “prostate cancer marker” is a “marker” as set forth above, which is associated with an abnormal prostate state, such as, for example, prostate cancer. As used herein, a prostate cancer marker includes one or more of the markers set forth in Tables 1-31.

As used herein, the term “ERG-positive prostate cancer marker” is a “marker” as set forth above, which is associated with ERG-positive prostate cancer. As used herein, an ERG-positive prostate cancer marker includes one or more of the markers set forth in Tables 6, 30 and 31.

As used herein, the term “ERG-negative prostate cancer marker” is a “marker” as set forth above, which is associated with ERG-negative prostate cancer. As used herein, an ERG-negative prostate cancer marker includes mercapto-succinyl-carnitine and one or more of the markers set forth in Tables 6, 30 and 31.

As used herein, the term “high BMI prostate cancer marker” is a “marker” as set forth above, which is associated an abnormal prostate state, e.g., prostate cancer, in an individual with a high BMI index, e.g., a BMI index equal or greater than 30. As used herein, a high BMI prostate cancer marker includes one or more of the markers set forth in Tables 7, 18 and 25.

As used herein, the term “Gleason Score marker” is a “marker” as set forth above, which is useful for predication of Gleason Score in a subject associated with an abnormal prostate state, e.g., prostate cancer. As used herein, a Gleason Score marker includes one or more of the markers set forth in Table 29.

Preferably, a marker of the present invention, e.g., a prostate cancer marker, an ERG-positive prostate cancer marker, an ERG-negative prostate cancer marker, a Gleason Score marker, or a high BMI prostate cancer marker, is modulated (e.g., increased or decreased level) in a biological sample from a subject or a group of subjects having a first phenotype (e.g., having a disease) as compared to a biological sample from a subject or group of subjects having a second phenotype (e.g., not having the disease, e.g., a control). A biomarker may be differentially present at any level, but is generally present at a level that is increased relative to normal or control levels by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased relative to normal or control levels by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent). A biomarker is preferably differentially present at a level that is statistically significant (e.g., a p-value less than 0.05 and/or a q-value of less than 0.10 as determined using either Welch's T-test or Wilcoxon's rank-sum Test).

As used herein, the term “BMI” or “body mass index” refers to a value derived from the mass (weight) and height of an individual. BMI is defined as the body mass divided by the square of the body height, and is universally expressed in units of kg/m², resulting from mass in kilograms and height in metres. BMI is an attempt to quantify the amount of tissue mass (muscle, fat, and bone) in an individual, and then categorize that person as underweight, normal weight, overweight, or obese based on that value. Commonly accepted BMI ranges are underweight: under 18.5, normal weight: 18.5 to 25, overweight: 25 to 30, obese: equal or over 30.

As used herein, the term “ERG” or “ETS-related gene”) refers to an oncogene which encodes a protein that typically is mutated in cancer. (Reddy E S, et al., 1987, Proceedings of the National Academy of Sciences of the United States of America 84 (17): 6131-5). ERG is a member of the ETS (erythroblast transformation-specific) family of transcription factors and encodes the ERG protein that functions as a transcriptional regulator. Genes in the ETS family regulate embryonic development, cell proliferation, differentiation, angiogenesis, inflammation, and apoptosis.

As used herein, the term “ERG-positive prostate cancer” refers to a type of prostate cancer which develops and progresses due to an underlying genetic defect in which an androgen hormone regulated gene, TMPRSS2, fuses with the oncogene ERG. These gene fusions drive the over-expression of ERG leading eventually to uncontrolled growth of prostate cancer. ERG gene fusions have gained significant recognition as a prostate cancer specific biomarker. This biomarker is seldom found in normal tissue or in non-prostatic tumors. ERG alteration is seen in 50% of prostate cancers and 20% of high-grade prostatic intraepithelial neoplasia, a neoplastic precursor lesion that intermingles with prostate carcinoma. Among PSA-screened men in the United States, TMPRSS2-ERG fusion prostate cancer has a prevalence of 46% in prostate needle biopsies. Therefore, early detection of ERG over-expression may provide significant diagnostic and prognostic value.

As used herein, the term “ERG-negative prostate cancer” refers to a type of prostate cancer which does not express a TMPRSS2-ERG fusion.

As used herein, the term “biopsy” or “biopsy tissue” refers to a sample of tissue (e.g., prostate tissue) that is removed from a subject for the purpose of determining if the sample contains cancerous tissue. The biopsy tissue is then examined (e.g., by microscopy) for the presence or absence of cancer.

As used herein, the term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “control sample” or “control,” as used herein, refers to any clinically relevant comparative sample, including, for example, a sample from a healthy subject not afflicted with an oncological disorder, e.g., prostate cancer, or a sample from a subject from an earlier time point, e.g., prior to treatment, an earlier tumor assessment time point, at an earlier stage of treatment. A control sample can be a purified sample, protein, and/or nucleic acid provided with a kit. Such control samples can be diluted, for example, in a dilution series to allow for quantitative measurement of levels of analytes, e.g., markers, in test samples. A control sample may include a sample derived from one or more subjects. A control sample may also be a sample made at an earlier time point from the subject to be assessed. For example, the control sample could be a sample taken from the subject to be assessed before the onset of an oncological disorder, e.g., prostate cancer, at an earlier stage of disease, or before the administration of treatment or of a portion of treatment. The control sample may also be a sample from an animal model, or from a tissue or cell line derived from the animal model of oncological disorder, e.g., prostate cancer. The level of activity or expression of one or more markers (e.g., 1, 2, 3, 4, 5, 6, 7, 8, or 9 or more markers) in a control sample consists of a group of measurements that may be determined, e.g., based on any appropriate statistical measurement, such as, for example, measures of central tendency including average, median, or modal values. In one embodiment, “different from a control” is preferably statistically significantly different from a control.

As used herein, “changed, altered, increased or decreased as compared to a control” sample or subject is understood as having a level of the analyte or diagnostic or therapeutic indicator (e.g., marker) to be detected at a level that is statistically different, e.g., increased or decreased, as compared to a sample from a normal, untreated, or abnormal state control sample. Changed as compared to control can also include a difference in the rate of change of the level of one or more markers obtained in a series of at least two subject samples obtained over time. Determination of statistical significance is within the ability of those skilled in the art and can include any acceptable means for determining and/or measuring statistical significance, such as, for example, the number of standard deviations from the mean that constitute a positive or negative result, an increase in the detected level of a biomarker in a sample (e.g., prostate cancer sample) versus a control or healthy sample, wherein the increase is above some threshold value, or a decrease in the detected level of a biomarker in a sample (e.g., prostate cancer sample) versus a control or healthy sample, wherein the decrease is below some threshold value. The threshold value can be determine by any suitable means by measuring the biomarker levels in a plurality of tissues or samples known to have a disease, e.g., prostate cancer, and comparing those levels to a normal sample and calculating a statistically significant threshold value.

The term “control level” refers to an accepted or pre-determined level of a marker in a subject sample. A control level can be a range of values. Marker levels can be compared to a single control value, to a range of control values, to the upper level of normal, or to the lower level of normal as appropriate for the assay.

In one embodiment, the control is a standardized control, such as, for example, a control which is predetermined using an average of the levels of expression of one or more markers from a population of subjects having no cancer, especially subjects having no prostate cancer. In still other embodiments of the invention, a control level of a marker is the level of the marker in a non-cancerous sample(s) derived from the subject having cancer. For example, when a biopsy or other medical procedure reveals the presence of cancer in one portion of the tissue, the control level of a marker may be determined using the non-affected portion of the tissue, and this control level may be compared with the level of the marker in an affected portion of the tissue.

In certain embodiments, the control can be from a subject, or a population of subject, having an abnormal prostate state. For example, the control can be from a subject suffering from benign prostate hyperplasia (BPH), androgen sensitive prostate cancer, androgen insensitive or resistant prostate cancer, aggressive prostate cancer, non-aggressive prostate cancer, metastatic prostate cancer, or non-metastatic prostate cancer. It is understood that not all markers will have different levels for each of the abnormal prostate states listed. It is understood that a combination of marker levels may be most useful to distinguish between abnormal prostate states, possibly in combination with other diagnostic methods. Further, marker levels in biological samples can be compared to more than one control sample (e.g., normal, abnormal, from the same subject, from a population control). Marker levels can be used in combination with other signs or symptoms of an abnormal prostate state to provide a diagnosis for the subject.

A control can also be a sample from a subject at an earlier time point, e.g., a baseline level prior to suspected presence of disease, before the diagnosis of a disease, at an earlier assessment time point during watchful waiting, before the treatment with a specific agent (e.g., chemotherapy, hormone therapy) or intervention (e.g., radiation, surgery). In certain embodiments, a change in the level of the marker in a subject can be more significant than the absolute level of a marker, e.g., as compared to control.

As used herein, “detecting”, “detection”, “determining”, and the like are understood to refer to an assay performed for identification of one or more markers selected from Tables 1-31. The amount of marker expression or activity detected in the sample can be none or below the level of detection of the assay or method.

As used herein, the term “DNA” or “RNA” molecule or sequence (as well as sometimes the term “oligonucleotide”) refers to a molecule comprised generally of the deoxyribonucleotides adenine (A), guanine (G), thymine (T) and/or cytosine (C). In “RNA”, T is replaced by uracil (U).

The terms “disorders”, “diseases”, and “abnormal state” are used inclusively and refer to any deviation from the normal structure or function of any part, organ, or system of the body (or any combination thereof). A specific disease is manifested by characteristic symptoms and signs, including biological, chemical, and physical changes, and is often associated with a variety of other factors including, but not limited to, demographic, environmental, employment, genetic, and medically historical factors. An early stage disease state includes a state wherein one or more physical symptoms are not yet detectable. Certain characteristic signs, symptoms, and related factors can be quantitated through a variety of methods to yield important diagnostic information. As used herein the disorder, disease, or abnormal state is an abnormal prostate state, including benign prostate hyperplasia and cancer, particularly prostate cancer. The abnormal prostate state of prostate cancer can be further subdivided into stages and grades of prostate cancer as provided, for example in Prostate. In: Edge S B, Byrd D R, Compton C C, et al., eds.: AJCC Cancer Staging Manual. 7th ed. New York, N.Y.: Springer, 2010, pp 457-68 (incorporated herein by reference in its entirety). Further, abnormal prostate states can be classified as one or more of benign prostate hyperplasia (BPH), androgen sensitive prostate cancer, androgen insensitive or resistant prostate cancer, aggressive prostate cancer, non-aggressive prostate cancer, metastatic prostate cancer, and non-metastatic prostate cancer.

As used herein, a sample obtained at an “earlier time point” is a sample that was obtained at a sufficient time in the past such that clinically relevant information could be obtained in the sample from the earlier time point as compared to the later time point. In certain embodiments, an earlier time point is at least four weeks earlier. In certain embodiments, an earlier time point is at least six weeks earlier. In certain embodiments, an earlier time point is at least two months earlier. In certain embodiments, an earlier time point is at least three months earlier. In certain embodiments, an earlier time point is at least six months earlier. In certain embodiments, an earlier time point is at least nine months earlier. In certain embodiments, an earlier time point is at least one year earlier. Multiple subject samples (e.g., 3, 4, 5, 6, 7, or more) can be obtained at regular or irregular intervals over time and analyzed for trends in changes in marker levels. Appropriate intervals for testing for a particular subject can be determined by one of skill in the art based on ordinary considerations.

The term “expression” is used herein to mean the process by which a polypeptide is produced from DNA. The process involves the transcription of the gene into mRNA and the translation of this mRNA into a polypeptide. Depending on the context in which used, “expression” may refer to the production of RNA, or protein, or both.

As used herein, “fold change ratio” or “FC ratio” refers to a change, e.g., increase or decrease, of the expression or level of a marker, e.g., one or more marker selected from Tables 1-31. In some embodiments, the FC ratio is greater than 1, which indicates an up-regulation or increase in the expression or level of the marker. In other embodiments, the FC ratio is less than 1, indicating a down-regulation or decrease in the expression or level of the marker. FC ratio can also be calculated and expressed as a Log unit. When the FC ratio is expressed as a Log FC value, a Log FC value greater than 0 is equivalent to an FC ratio greater than 1, indicating an up-regulation or increase in the expression or level of the marker. Alternatively, a Log FC value less than 0 is equivalent to an FC ratio less than 1, indicating a down-regulation or decrease in the expression or level of the marker.

As used herein, “greater predictive value” is understood as an assay that has significantly greater sensitivity and/or specificity, preferably greater sensitivity and specificity, than the test to which it is compared. The predictive value of a test can be determined using an ROC analysis. In an ROC analysis, a test that provides perfect discrimination or accuracy between normal and disease states would have an area under the curve (AUC)=1, whereas a very poor test that provides no better discrimination than random chance would have AUC=0.5. As used herein, a test with a greater predictive value will have a statistically improved AUC as compared to another assay. The assays are preformed in an appropriate subject population.

A “higher level of expression”, “higher level”, “increased level,” and the like of a marker refers to an expression level in a test sample that is greater than the standard error of the assay employed to assess expression, and is preferably at least 25% more, at least 50% more, at least 75% more, at least two, at least three, at least four, at least five, at least six, at least seven, at least eight, at least nine, or at least ten times the expression level of the marker in a control sample (e.g., sample from a healthy subject not having the marker associated disease, i.e., an abnormal prostate state) and preferably, the average expression level of the marker or markers in several control samples.

As used herein, the term “hybridization,” as in “nucleic acid hybridization,” refers generally to the hybridization of two single-stranded nucleic acid molecules having complementary base sequences, which under appropriate conditions will form a thermodynamically favored double-stranded structure. Examples of hybridization conditions can be found in the two laboratory manuals referred above (Sambrook et al., 2000, supra and Ausubel et al., 1994, supra, or further in Higgins and Hames (Eds.) “Nucleic acid hybridization, a practical approach” TRL Press Oxford, Washington D.C., (1985)) and are commonly known in the art. In the case of a hybridization to a nitrocellulose filter (or other such support like nylon), as for example in the well-known Southern blotting procedure, a nitrocellulose filter can be incubated overnight at a temperature representative of the desired stringency condition (60-65° C. for high stringency, 50-60° C. for moderate stringency and 40-45° C. for low stringency conditions) with a labeled probe in a solution containing high salt (6×SSC or 5×SSPE), 5×Denhardt's solution, 0.5% SDS, and 100 μg/ml denatured carrier DNA (e.g., salmon sperm DNA). The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency). The salt and SDS concentration of the washing solutions may also be adjusted to accommodate for the desired stringency. The selected temperature and salt concentration is based on the melting temperature (Tm) of the DNA hybrid. Of course, RNA-DNA hybrids can also be formed and detected. In such cases, the conditions of hybridization and washing can be adapted according to well-known methods by the person of ordinary skill. Stringent conditions will be preferably used (Sambrook et al., 2000, supra). Other protocols or commercially available hybridization kits (e.g., ExpressHyb® from BD Biosciences Clonetech) using different annealing and washing solutions can also be used as well known in the art. As is well known, the length of the probe and the composition of the nucleic acid to be determined constitute further parameters of the hybridization conditions. Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility. Hybridizing nucleic acid molecules also comprise fragments of the above described molecules. Furthermore, nucleic acid molecules which hybridize with any of the aforementioned nucleic acid molecules also include complementary fragments, derivatives and allelic variants of these molecules. Additionally, a hybridization complex refers to a complex between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., Cot or Rot analysis) or between one nucleic acid sequence present in solution and another nucleic acid sequence immobilized on a solid support (e.g., membranes, filters, chips, pins or glass slides to which, e.g., cells have been fixed).

As used herein, the term “identical” or “percent identity” in the context of two or more nucleic acid or amino acid sequences, refers to two or more sequences or subsequences that are the same, or that have a specified percentage of amino acid residues or nucleotides that are the same (e.g., 60% or 65% identity, preferably, 70-95% identity, more preferably at least 95% identity), when compared and aligned for maximum correspondence over a window of comparison, or over a designated region as measured using a sequence comparison algorithm as known in the art, or by manual alignment and visual inspection. Sequences having, for example, 60% to 95% or greater sequence identity are considered to be substantially identical. Such a definition also applies to the complement of a test sequence. Preferably the described identity exists over a region that is at least about 15 to 25 amino acids or nucleotides in length, more preferably, over a region that is about 50 to 100 amino acids or nucleotides in length. Those having skill in the art will know how to determine percent identity between/among sequences using, for example, algorithms such as those based on CLUSTALW computer program (Thompson Nucl. Acids Res. 2 (1994), 4673-4680) or FASTDB (Brutlag Comp. App. Biosci. 6 (1990), 237-245), as known in the art. Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations. Also available to those having skill in this art are the BLAST and BLAST 2.0 algorithms (Altschul Nucl. Acids Res. 25 (1977), 3389-3402). The BLASTN program for nucleic acid sequences uses as defaults a word length (W) of 11, an expectation (E) of 10, M=5, N=4, and a comparison of both strands. For amino acid sequences, the BLASTP program uses as defaults a wordlength (W) of 3, and an expectation (E) of 10. The BLOSUM62 scoring matrix (Henikoff Proc. Natl. Acad. Sci., USA, 89, (1989), 10915) uses alignments (B) of 50, expectation (E) of 10, M=5, N=4, and a comparison of both strands. Moreover, the present invention also relates to nucleic acid molecules the sequence of which is degenerate in comparison with the sequence of an above-described hybridizing molecule. When used in accordance with the present invention the term “being degenerate as a result of the genetic code” means that due to the redundancy of the genetic code different nucleotide sequences code for the same amino acid. The present invention also relates to nucleic acid molecules which comprise one or more mutations or deletions, and to nucleic acid molecules which hybridize to one of the herein described nucleic acid molecules, which show (a) mutation(s) or (a) deletion(s).

The term “including” is used herein to mean, and is used interchangeably with, the phrase “including but not limited to.”

A subject at “increased risk for developing prostate cancer” may or may not develop prostate cancer. Identification of a subject at increased risk for developing prostate cancer should be monitored for additional signs or symptoms of prostate cancer. The methods provided herein for identifying a subject with increased risk for developing prostate cancer can be used in combination with assessment of other known risk factors or signs of prostate cancer including, but not limited to decreased urinary stream, urgency, hesitancy, nocturia, incomplete bladder emptying, and age.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

As used herein, a “label” refers to a molecular moiety or compound that can be detected or can lead to a detectable signal. A label is joined, directly or indirectly, to a molecule, such as an antibody, a nucleic acid probe or the protein/antigen or nucleic acid to be detected (e.g., an amplified sequence). Direct labeling can occur through bonds or interactions that link the label to the nucleic acid (e.g., covalent bonds or non-covalent interactions), whereas indirect labeling can occur through the use of a “linker” or bridging moiety, such as oligonucleotide(s) or small molecule carbon chains, which is either directly or indirectly labeled. Bridging moieties may amplify a detectable signal. Labels can include any detectable moiety (e.g., a radionuclide, ligand such as biotin or avidin, enzyme or enzyme substrate, reactive group, chromophore such as a dye or colored particle, luminescent compound including a bioluminescent, phosphorescent or chemiluminescent compound, and fluorescent compound). Preferably, the label on a labeled probe is detectable in a homogeneous assay system, i.e., in a mixture, the bound label exhibits a detectable change compared to an unbound label.

The terms “level of expression of a gene”, “gene expression level”, “level of a marker”, and the like refer to the level of mRNA, as well as pre-mRNA nascent transcript(s), transcript processing intermediates, mature mRNA(s) and degradation products, or the level of protein, encoded by the gene in the cell. The “level” of one of more biomarkers means the absolute or relative amount or concentration of the biomarker in the sample.

A “lower level of expression” or “lower level” or “decreased level” of a marker refers to an expression level in a test sample that is less than 90%, 85%, 80%, 75%, 70%, 65%, 60%, 55%, 50%, 45%, 40%, 35%, 30%, 25%, 20%, 15%, or 10% of the expression level of the marker in a control sample (e.g., sample from a healthy subjects not having the marker associated disease, i.e., an abnormal prostate state) and preferably, the average expression level of the marker in several control samples.

The term “modulation” refers to upregulation (i.e., activation or stimulation), down-regulation (i.e., inhibition or suppression) of a response (e.g., level of a marker), or the two in combination or apart. A “modulator” is a compound or molecule that modulates, and may be, e.g., an agonist, antagonist, activator, stimulator, suppressor, or inhibitor.

As used herein, “nucleic acid molecule” or “polynucleotides”, refers to a polymer of nucleotides. Non-limiting examples thereof include DNA (e.g., genomic DNA, cDNA), RNA molecules (e.g., mRNA) and chimeras thereof. The nucleic acid molecule can be obtained by cloning techniques or synthesized. DNA can be double-stranded or single-stranded (coding strand or non-coding strand [antisense]). Conventional ribonucleic acid (RNA) and deoxyribonucleic acid (DNA) are included in the term “nucleic acid” and polynucleotides as are analogs thereof Δ nucleic acid backbone may comprise a variety of linkages known in the art, including one or more of sugar-phosphodiester linkages, peptide-nucleic acid bonds (referred to as “peptide nucleic acids” (PNA); Hydig-Hielsen et al., PCT Intl Pub. No. WO 95/32305), phosphorothioate linkages, methylphosphonate linkages or combinations thereof. Sugar moieties of the nucleic acid may be ribose or deoxyribose, or similar compounds having known substitutions, e.g., 2′ methoxy substitutions (containing a 2′-O-methylribofuranosyl moiety; see PCT No. WO 98/02582) and/or 2′ halide substitutions. Nitrogenous bases may be conventional bases (A, G, C, T, U), known analogs thereof (e.g., inosine or others; see The Biochemistry of the Nucleic Acids 5-36, Adams et al., ed., 11th ed., 1992), or known derivatives of purine or pyrimidine bases (see, Cook, PCT Int'l Pub. No. WO 93/13121) or “abasic” residues in which the backbone includes no nitrogenous base for one or more residues (Arnold et al., U.S. Pat. No. 5,585,481). A nucleic acid may comprise only conventional sugars, bases and linkages, as found in RNA and DNA, or may include both conventional components and substitutions (e.g., conventional bases linked via a methoxy backbone, or a nucleic acid including conventional bases and one or more base analogs). An “isolated nucleic acid molecule”, as is generally understood and used herein, refers to a polymer of nucleotides, and includes, but should not limited to DNA and RNA. The “isolated” nucleic acid molecule is purified from its natural in vivo state, obtained by cloning or chemically synthesized.

As used herein, the term “obtaining” is understood herein as manufacturing purchasing, or otherwise coming into possession of.

As used herein, “oligonucleotides” or “oligos” define a molecule having two or more nucleotides (ribo or deoxyribonucleotides). The size of the oligo will be dictated by the particular situation and ultimately on the particular use thereof and adapted accordingly by the person of ordinary skill. An oligonucleotide can be synthesized chemically or derived by cloning according to well-known methods. While they are usually in a single-stranded form, they can be in a double-stranded form and even contain a “regulatory region”. They can contain natural rare or synthetic nucleotides. They can be designed to enhance a chosen criteria like stability for example. Chimeras of deoxyribonucleotides and ribonucleotides may also be within the scope of the present invention.

As used herein, “one or more” is understood as each value 1, 2, 3, 4, 5, 6, 7, 8, 9, 10, and any value greater than 10.

The term “or” is used inclusively herein to mean, and is used interchangeably with, the term “and/or,” unless context clearly indicates otherwise.

As used herein, “patient” or “subject” can mean either a human or non-human animal, preferably a mammal. By “subject” is meant any animal, including horses, dogs, cats, pigs, goats, rabbits, hamsters, monkeys, guinea pigs, rats, mice, lizards, snakes, sheep, cattle, fish, and birds. A human subject may be referred to as a patient. It should be noted that clinical observations described herein were made with human subjects and, in at least some embodiments, the subjects are human. In some embodiments, the subject is selected from a population of Caucasians. In other embodiments, the subject is selected from a population of African Americans.

As used herein, “preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease). Prevention does not require that the disease or condition never occurs in the subject. Prevention includes delaying the onset or severity of the disease or condition.

As used herein, a “predetermined threshold value” or “threshold value” of a biomarker refers to the level of the biomarker (e.g., the expression level or quantity (e.g., ng/ml) in a biological sample) in a corresponding control/normal sample or group of control/normal samples obtained from normal or healthy subjects, e.g., those males that do not have prostate cancer. The predetermined threshold value may be determined prior to or concurrently with measurement of marker levels in a biological sample. The control sample may be from the same subject at a previous time or from different subjects.

As used herein, a “probe” is meant to include a nucleic acid oligomer or oligonucleotide that hybridizes specifically to a target sequence in a nucleic acid or its complement, under conditions that promote hybridization, thereby allowing detection of the target sequence or its amplified nucleic acid. Detection may either be direct (i.e., resulting from a probe hybridizing directly to the target or amplified sequence) or indirect (i.e., resulting from a probe hybridizing to an intermediate molecular structure that links the probe to the target or amplified sequence). A probe's “target” generally refers to a sequence within an amplified nucleic acid sequence (i.e., a subset of the amplified sequence) that hybridizes specifically to at least a portion of the probe sequence by standard hydrogen bonding or “base pairing.” Sequences that are “sufficiently complementary” allow stable hybridization of a probe sequence to a target sequence, even if the two sequences are not completely complementary. A probe may be labeled or unlabeled. A probe can be produced by molecular cloning of a specific DNA sequence or it can also be synthesized. Numerous primers and probes which can be designed and used in the context of the present invention can be readily determined by a person of ordinary skill in the art to which the present invention pertains.

As used herein, the terminology “prognosis”, “staging” and “determination of aggressiveness” are defined herein as the prediction of the degree of severity of the prostate cancer and of its evolution as well as the prospect of recovery as anticipated from usual course of the disease. According to the present invention, once the aggressiveness of the prostate cancer has been determined appropriate methods of treatments can be chosen.

As used herein, “prophylactic” or “therapeutic” treatment refers to administration to the subject of one or more agents or interventions to provide the desired clinical effect. If it is administered prior to clinical manifestation of the unwanted condition (e.g., disease or other unwanted state of the host animal) then the treatment is prophylactic, i.e., it protects the host against developing at least one sign or symptom of the unwanted condition, whereas if administered after manifestation of the unwanted condition, the treatment is therapeutic (i.e., it is intended to diminish, ameliorate, or maintain at least one sign or symptom of the existing unwanted condition or side effects therefrom).

As used herein, “prostate cancer,” refers to any malignant or pre-malignant form of cancer of the prostate. The term includes prostate in situ carcinomas, invasive carcinomas, metastatic carcimomas and pre-malignant conditions. The term also encompasses any stage or grade of cancer in the prostate. Where the prostate cancer is “metastatic,” the cancer has spread or metastasized beyond the prostate gland to a distant site, such as a lymph node or to the bone. In some embodiments, the prostate cancer is an ERG-positive prostate cancer. In other embodiments, the prostate cancer is an ERG-negative prostate cancer.

As used herein, a “reference level” of a biomarker means a level of the biomarker that is indicative of a particular disease state, phenotype, or lack thereof, as well as combinations of disease states, phenotypes, or lack thereof Δ “positive” reference level of a biomarker means a level that is indicative of a particular disease state or phenotype. A “negative” reference level of a biomarker means a level that is indicative of a lack of a particular disease state or phenotype. For example, a “prostate cancer-positive reference level” of a biomarker means a level of a biomarker that is indicative of a positive diagnosis of prostate cancer in a subject, and a “prostate cancer-negative reference level” of a biomarker means a level of a biomarker that is indicative of a negative diagnosis of prostate cancer in a subject. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched so that comparisons may be made between biomarker levels in samples from subjects of a certain age and reference levels for a particular disease state, phenotype, or lack thereof in a certain age group). Such reference levels may also be tailored to specific techniques that are used to measure levels of biomarkers in biological samples (e.g., LC-MS, GC-MS, etc.), where the levels of biomarkers may differ based on the specific technique that is used.

As used herein, “sample” or “biological sample” includes a specimen or culture obtained from any source. Biological samples can be obtained from blood (including any blood product, such as whole blood, plasma, serum, or specific types of cells of the blood), urine, saliva, seminal fluid, and the like. Biological samples also include tissue samples, such as biopsy tissues or pathological tissues that have previously been fixed (e.g., formaline snap frozen, cytological processing etc.). In an embodiment, the biological sample is from blood. In another embodiment, the biological sample is a biopsy tissue from the prostate gland.

As use herein, the phrase “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabeled A) in a reaction containing labeled “A” and the antibody will reduce the amount of labeled Abound to the antibody.

The phrase “specific identification” is understood as detection of a marker of interest with sufficiently low background of the assay and cross-reactivity of the reagents used such that the detection method is diagnostically useful. In certain embodiments, reagents for specific identification of a marker bind to only one isoform of the marker. In certain embodiments, reagents for specific identification of a marker bind to more than one isoform of the marker. In certain embodiments, reagents for specific identification of a marker bind to all known isoforms of the marker.

As used herein, the phrase “subject suspected of having cancer” refers to a subject that presents one or more symptoms indicative of a cancer or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received an initial diagnosis (e.g., a CT scan showing a mass or increased PSA level) but for whom the stage of cancer is not known. The term further includes people who once had cancer (e.g., an individual in remission).

The term “such as” is used herein to mean, and is used interchangeably, with the phrase “such as but not limited to.”

As used herein, the term “stage of cancer” refers to a qualitative or quantitative assessment of the level of advancement of a cancer. Criteria used to determine the stage of a cancer include, but are not limited to, the size of the tumor, whether the tumor has spread to other parts of the body and where the cancer has spread (e.g., within the same organ or region of the body or to another organ).

As used herein, the term “staging” refers to commonly used systems for grading/stating cancer, e.g., prostate cancer. In one aspect, staging can take the form of the “Gleason Score”, as well known in the art, is the most commonly used system for the grading/staging and prognosis of adenocarcinoma. The system describes a score between 2 and 10, with 2 being the least aggressive and 10 being the most aggressive. The score is the sum of the two most common patterns (grade 1-5) of tumor growth found. To be counted a pattern (grade) needs to occupy more than 5% of the biopsy specimen. The scoring system requires biopsy material (core biopsy or operative specimens) in order to be accurate; cytological preparations cannot be used. The “Gleason Grade” is the most commonly used prostate cancer grading system. It involves assigning numbers to cancerous prostate tissue, ranging from 1 through 5, based on how much the arrangement of the cancer cells mimics the way normal prostate cells form glands. Two grades are assigned to the most common patterns of cells that appear; these two grades (they can be the same or different) are then added together to determine the Gleason score (a number from 1 to 10). The Gleason system is based exclusively on the architectural pattern of the glands of the prostate tumor. It evaluates how effectively the cells of any particular cancer are able to structure themselves into glands resembling those of the normal prostate. The ability of a tumor to mimic normal gland architecture is called its differentiation, and experience has shown that a tumor whose structure is nearly normal (well differentiated) will probably have a biological behavior relatively close to normal, i.e., that is not very aggressively malignant.

A Gleason grading from very well differentiated (grade 1) to very poorly differentiated (grade 5) is usually done for the most part by viewing the low magnification microscopic image of the cancer. There are important additional details which require higher magnification, and an ability to accurately grade any tumor is achieved only through much training and experience in pathology. Gleason grades 1 and 2: These two grades closely resemble normal prostate. They are the least important grades because they seldom occur in the general population and because they confer a prognostic benefit which is only slightly better than grade 3. Both of these grades are composed by mass; in grade 2 they are more loosely aggregated, and some glands wander (invade) into the surrounding muscle (stroma). Gleason grade 3 is the most common grade and is also considered well differentiated (like grades 1 and 2). This is because all three grades have a normal “gland unit” like that of a normal prostate; that is, every cell is part of a circular row which forms the lining of a central space (the lumen). The lumen contains prostatic secretion like normal prostate, and each gland unit is surrounded by prostate muscle which keeps the gland units apart. In contrast to grade 2, wandering of glands (invading) into the stroma (muscle) is very prominent and is the main defining feature. The cells are dark rather than pale and the glands often have more variable shapes.

Gleason Grade 4 is probably the most important grade because it is fairly common and because of the fact that if a lot of it is present, patient prognosis is usually (but not always) worsened by a considerable degree. Grade 4 also shows a considerable loss of architecture. For the first time, disruption and loss of the normal gland unit is observed. In fact, grade 4 is identified almost entirely by loss of the ability to form individual, separate gland units, each with its separate lumen (secretory space). This important distinction is simple in concept but complex in practice. The reason is that there are a variety of different-appearing ways in which the cancer's effort to form gland units can be distorted. Each cancer has its own partial set of tools with which it builds part of the normal structure. Grade 4 is like the branches of a large tree, reaching in a number of directions from the (well differentiated) trunk of grades 1, 2, and 3. Much experience is required for this diagnosis, and not all patterns are easily distinguished from grade 3. This is the main class of poorly differentiated prostate cancer, and its distinction from grade 3 is the most commonly important grading decision.

Gleason grade 5 is an important grade because it usually predicts another significant step towards poor prognosis. Its overall importance for the general population is reduced by the fact that it is less common than grade 4, and it is seldom seen in men whose prostate cancer is diagnosed early in its development. This grade too shows a variety of patterns, all of which demonstrate no evidence of any attempt to form gland units. This grade is often called undifferentiated, because its features are not significantly distinguishing to make it look any different from undifferentiated cancers which occur in other organs. When a pathologist looks at prostate cancer specimens under the microscope and gives them a Gleason grade, an attempt to identify two architectural patterns and assign a Gleason grade to each one is made. There may be a primary or most common pattern and then a secondary or second most common pattern which the pathologist will seek to describe for each specimen; alternatively, there may often be only a single pure grade. In developing his system, Dr. Gleason discovered that by giving a combination of the grades of the two most common patterns he could see in any particular patient's specimens, that he was better able to predict the likelihood that a particular patient would do well or badly. Therefore, although it may seem confusing the Gleason score which a physician usually gives to a patient, is actually a combination or sum of two numbers which is accurate enough to be very widely used. These combined Gleason sums or scores may be determined as follows:

The lowest possible Gleason score is 2 (1+1), where both the primary and secondary patterns have a Gleason grade of 1 and therefore when added together their combined sum is 2.

Very typical Gleason scores might be 5 (2+3), where the primary pattern has a Gleason grade of 2 and the secondary pattern has a grade of 3, or 6 (3+3), a pure pattern.

Another typical Gleason score might be 7 (4+3), where the primary pattern has a Gleason grade of 4 and the secondary pattern has a grade of 3.

Finally, the highest possible Gleason score is 10 (5+5), when the primary and secondary patterns both have the most disordered Gleason grades of 5.

Example of markers for that are predictive of any particular stage or phase of prostate cancer, e.g., Gleason grade 1, grade 2, grade 3, grade 4, or grade 5 prostate cancer, include one or more marker selected from Table 29.

Another way of staging prostate cancer is by using the TNM System. It describes the extent of the primary tumor (T stage), the absence or presence of spread to nearby lymph nodes (N stage) and the absence or presence of distant spread, or metastasis (M stage). Each category of the TNM classification is divided into subcategories representative of its particular state. For example, primary tumors (T stage) may be classified into:

T1: The tumor cannot be felt during a digital rectal exam, or seen by imaging studies, but cancer cells are found in a biopsy specimen;

T2: The tumor can be felt during a DRE and the cancer is confined within the prostate gland;

T3: The tumor has extended through the prostatic capsule (a layer of fibrous tissue surrounding the prostate gland) and/or to the seminal vesicles (two small sacs next to the prostate that store semen), but no other organs are affected;

T4: The tumor has spread or attached to tissues next to the prostate (other than the seminal vesicles).

Lymph node involvement is divided into the following 4 categories:

N0: Cancer has not spread to any lymph nodes;

N1: Cancer has spread to a single regional lymph node (inside the pelvis) and is not larger than 2 centimeters;

N2: Cancer has spread to one or more regional lymph nodes and is larger than 2 centimeters, but not larger than 5 centimeters; and

N3: Cancer has spread to a lymph node and is larger than 5 centimeters (2 inches).

Metastasis is generally divided into the following two categories:

M0: The cancer has not metastasized (spread) beyond the regional lymph nodes; and

M1: The cancer has metastasized to distant lymph nodes (outside of the pelvis), bones, or other distant organs such as lungs, liver, or brain.

In addition, the T stage is further divided into subcategories T1a-c T2a-c, T3a-c and T4a-b. The characteristics of each of these subcategories are well known in the art and can be found in a number of textbooks.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using the screening methods of the present invention. In some embodiments of the present invention, test compounds include antisense compounds.

The term “therapeutic effect” refers to a local or systemic effect in animals, particularly mammals, and more particularly humans caused by a pharmacologically active substance. The term thus means any substance intended for use in the diagnosis, cure, mitigation, treatment, or prevention of disease, or in the enhancement of desirable physical or mental development and conditions in an animal or human. A therapeutic effect can be understood as a decrease in tumor growth, decrease in tumor growth rate, stabilization or decrease in tumor burden, stabilization or reduction in tumor size, stabilization or decrease in tumor malignancy, increase in tumor apoptosis, and/or a decrease in tumor angiogenesis.

As used herein, “therapeutically effective amount” means the amount of a compound that, when administered to a patient for treating a disease, is sufficient to effect such treatment for the disease, e.g., the amount of such a substance that produces some desired local or systemic effect at a reasonable benefit/risk ratio applicable to any treatment, e.g., is sufficient to ameliorate at least one sign or symptom of the disease, e.g., to prevent progression of the disease or condition, e.g., prevent tumor growth, decrease tumor size, induce tumor cell apoptosis, reduce tumor angiogenesis, prevent metastasis. When administered for preventing a disease, the amount is sufficient to avoid or delay onset of the disease. The “therapeutically effective amount” will vary depending on the compound, its therapeutic index, solubility, the disease and its severity and the age, weight, etc., of the patient to be treated, and the like. For example, certain compounds discovered by the methods of the present invention may be administered in a sufficient amount to produce a reasonable benefit/risk ratio applicable to such treatment. Administration of a therapeutically effective amount of a compound may require the administration of more than one dose of the compound.

A “transcribed polynucleotide” or “nucleotide transcript” is a polynucleotide (e.g. an mRNA, hnRNA, a cDNA, or an analog of such RNA or cDNA) which is complementary to or having a high percentage of identity (e.g., at least 80% identity) with all or a portion of a mature mRNA made by transcription of a marker of the invention and normal post-transcriptional processing (e.g. splicing), if any, of the RNA transcript, and reverse transcription of the RNA transcript.

As used herein, “treatment,” particularly “active treatment,” refers to performing an intervention to treat prostate cancer in a subject, e.g., reduce at least one of the growth rate, reduction of tumor burden, reduce or maintain the tumor size, or the malignancy (e.g., likelihood of metastasis) of the tumor; or to increase apoptosis in the tumor by one or more of administration of a therapeutic agent, e.g., chemotherapy or hormone therapy; administration of radiation therapy (e.g., pellet implantation, brachytherapy), or surgical resection of the tumor, or any combination thereof appropriate for treatment of the subject based on grade and stage of the tumor and other routine considerations. Active treatment is distinguished from “watchful waiting” (i.e., not active treatment) in which the subject and tumor are monitored, but no interventions are performed to affect the tumor. Watchful waiting can include administration of agents that alter effects caused by the tumor (e.g., incontinence, erectile dysfunction) that are not administered to alter the growth or pathology of the tumor itself.

The recitation of a listing of chemical group(s) in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Reference will now be made in detail to exemplary embodiments of the invention. While the invention will be described in conjunction with the exemplary embodiments, it will be understood that it is not intended to limit the invention to those embodiments. To the contrary, it is intended to cover alternatives, modifications, and equivalents as may be included within the spirit and scope of the invention as defined by the appended claims.

Exemplary compositions and methods of the present invention are described in more detail in the following sections: (C) Biomarkers of the invention; (D) Prostate tissue samples; (E) Detection and/or measurement of the biomarkers of the invention; (F) Isolated biomarkers; (G) Applications of biomarkers of the invention; (H) Therapeutics; (I) Drug screening and (J) Kits/panels.

### C. Biomarkers of the Invention

The present invention is based, at least in part, on the discovery that the markers (hereinafter “biomarkers”, “markers” or “markers of the invention”) in Tables 1-31 are differentially regulated in prostate cancer cells. In particular, the invention is based on the surprising discovery that the markers in Tables 1-31 are either elevated or depressed in the serum of patients with prostate cancer. The invention is also based on the surprising discovery that certain markers of the invention for the prognosis and/or diagnosis of prostate cancer are differentially expressed based on race or clinical phenotype. For example, in one embodiment, markers of the invention are differentially expressed among different populations, for example, in African American (AA) or Caucasian American (CA) populations. In another embodiment, markers of the invention are also differentally expressed in subjects with different types of prostate cancer, such as ERG-positive and ERG-negative tumors, and with different Gleason scores. In another embodiment, markers of the invention are also differentally expressed in subjects having different BMIs. Accordingly, the invention provides methods for prognosing, diagnosing and/or monitoring (e.g., monitoring of disease progression or treatment) and/or prognosing prostate cancer, in a subject. Specifically, the markers of the invention, e.g., one or more markers selected from Tables 1, 4, 8, 11, 13, 16, 19, 22, 26, 29 and 30, are diagnostic and/or indicative and/or predicative of prostate cancer in the Caucasian population. The markers of the invention, e.g., one or more markers selected from Tables 2, 5, 9, 12, 14, 17, 20, 23, 27 and 31, are diagnostic and/or indicative and/or predicative of prostate cancer in the African American population. The markers of the invention, e.g., one or more markers selected from Tables 6, 30 and 31, are diagnostic and/or indicative and/or predicative of the ERG status of a prostate tumor. The markers of the invention, e.g., one or more markers selected from Tables 7, 18 and 25, are diagnostic and/or indicative and/or predicative of prostate cancer in patients with a high BMI index equal or greater than 30. The markers of the invention, e.g., one or more markers selected from Table 29, are diagnostic and/or indicative and/or predicative of clinical state, i.e., Gleason score, of a prostate tumor.

Accordingly, the invention provides methods for diagnosing and/or monitoring (e.g., monitoring of disease progression or treatment) and/or prognosing an oncological disease state, e.g., prostate cancer, in a subject. In some embodiments, the subject is selected from the general population. In other embodiments, the subject is selected from a population of Caucasians. In yet another embodiment, the subject is selected from a population of African Americans. In some embodiments, the subject has an ERG-positive prostate cancer. In other embodiments, the subject has an ERG-negative prostate cancer. In a further embodiment, the subject has a BMI equal to or greater than 30.

The invention also provides methods for treating or for adjusting treatment regimens based on diagnostic information relating to the levels of one or more of the markers in Tables 1-31 in the serum of a subject with an oncological disease state, e.g., prostate cancer. The invention further provides panels and kits for practicing the methods of the invention.

The present invention provides new markers and combinations of markers for use in diagnosing and/or prognosing an oncological disorder, and in particular, markers for use in diagnosing and/or prognosing prostate cancer. These markers are particularly useful in screening for the presence of an altered prostate state, e.g., prostate cancer, in assessing aggressiveness and metastatic potential of an oncologic disorder, e.g., prostate cancer, assessing whether a subject is afflicted with an oncological disorder, identifying a composition for treating an oncological disorder, assessing the efficacy of a compound for treating an oncological disorder, monitoring the progression of an oncological disorder, prognosing the aggressiveness of an oncological disorder, prognosing the survival of a subject with an oncological disorder, prognosing the recurrence of an oncological disorder, and prognosing whether a subject is predisposed to developing an oncological disorder.

The markers of the invention include, but are not limited to, one or more prostate cancer markers selected from Tables 1-31, one or more ERG-positive prostate cancer markers selected from Tables 6, 30 and 31, ERG-negative prostate cancer marker selected from Table 24, one or more Gleason Score markers selected from Table 29, and one or more high BMI prostate cancer markers selected from Tables 7, 18 and 25.

In some embodiments of the present invention, other biomarkers can be used in connection with the methods of the present invention. As used herein, the term “one or more biomarkers” is intended to mean that one or more (e.g., 1, 2, 3, 4, 5, 6, 7, 8, 9, 10, 11, 12, 13, 14, 15, 16, 17, 18, 19, 20, or more) markers selected from Tables 1-31, are assayed, optionally in combination with PSA, or another prostate cancer marker, and, in various embodiments, more than one other biomarker may be assayed, such as one or more biomarkers from Tables 1-31 may be assayed.

Methods, kits, and panels provided herein include any combination of e.g., 1, 2, 3, 4, 5, 6, 7, 8, 9, 10, 11, 12, 13, 14, 15, 16, 17, 18, 19, 20, or more markers selected from Tables 1-31. Any one marker or any combination of more than one marker selected from Tables 1-31 can be use din combination with PSA or another prostate cancer marker.

The markers of the invention are meant to encompass any measurable characteristic that reflects in a quantitative or qualitative manner the physiological state of an organism, e.g., whether the organism has prostate cancer. The physiological state of an organism is inclusive of any disease or non-disease state, e.g., a subject having prostate cancer or a subject who is otherwise healthy. Said another way, the markers of the invention include characteristics that can be objectively measured and evaluated as indicators of normal processes, pathogenic processes, or pharmacologic responses to a therapeutic intervention, including, in particular, prostate cancer. Examples of markers include, for example, polypeptides, peptides, polypeptide fragments, proteins, antibodies, hormones, polynucleotides, RNA or RNA fragments, microRNA (miRNAs), lipids (e.g. structural lipids or signaling lipids), polysaccharides, and other bodily metabolites that are diagnostic and/or indicative and/or predictive of an oncological disease, e.g., prostate cancer, including one or more of the markers of Tables 1-31. Examples of markers also include polypeptides, peptides, polypeptide fragments, proteins, antibodies, hormones, polynucleotides, RNA or RNA fragments, microRNA (miRNAs), lipids (e.g. structural lipids or signaling lipids), polysaccharides, and other bodily metabolites which are diagnostic and/or indicative and/or predictive of any stage or clinical phase of a disease, such as prostate cancer, including one or more of the markers of Tables 1-31.

The markers of the invention, e.g., one or more markers selected from Tables 1-31, are diagnostic and/or indicative and/or predictive of prostate cancer in a subject. In one embodiment, the markers of the invention, e.g., one or more markers selected from Tables 1, 4, 8, 11, 13, 16, 19, 22, 26, 29 and 30, are diagnostic and/or indicative and/or predicative of prostate cancer in the Caucasian population. In another embodiment, the markers of the invention, e.g., one or more markers selected from Tables 2, 5, 9, 12, 14, 17, 20, 23, 27 and 31, are diagnostic and/or indicative and/or predicative of prostate cancer in the African American population. In still another embodiment, the markers of the invention, e.g., one or more markers selected from Tables 6, 30 and 31, are diagnostic and/or indicative and/or predicative of the ERG status of a prostate tumor. In another embodiment, the markers of the invention, e.g., one or more markers selected from Tables 7, 18 and 25, are diagnostic and/or indicative and/or predicative of prostate cancer in patients with a high BMI index equal or greater than 30. In yet another embodiment, the markers of the invention, e.g., one or more markers selected from Table 29, are diagnostic and/or indicative and/or predicative of clinical state, i.e., Gleason score, of a prostate tumor. Clinical stage or phase can be represented by any means known in the art, for example, based on the Gleason Score system, e.g., Gleason grade 1, grade 2, grade 3, grade 4, or grade 5 prostate cancer.

In one aspect, the present invention relates to using, measuring, detecting, and the like of one or more of the markers in Tables 1-31 for diagnosis of the presence of prostate cancer in a subject. In some embodiments, the subject is selected from a general population. In other embodiments, the subject is selected from a population of Caucasians. In yet another embodiment, the subject is selected from a population of African Americans. In some embodiments, the subject has an ERG-positive prostate cancer. In other embodiments, the subject has an ERG-negative prostate cancer. In a further embodiment, the subject has a BMI equal to or greater than 30.

Accordingly, in one aspect, the present invention provides methods for diagnosing the presence of prostate cancer in a subject selected from a population of Caucasians. In another aspect, the present invention provides methods for diagnosing the presence of prostate cancer in a subject selected from a population of African Americans. In one aspect, the present invention provides methods for diagnosing the presence of ERG-positive prostate cancer in a subject. In another aspect, the present invention provides methods for diagnosing the presence of prostate cancer in a subject with a BMI equal to or greater than 30. In a further aspect, the present invention provides methods for diagnosing the presence of ERG-negative prostate cancer in a Caucasian subject with a BMI index equal or greater than 30.

In another aspect, the present invention relates to using measuring, detecting and the like of one or more of the markers in Tables 1-31 alone, or together with one or more additional markers of prostate cancer. Other markers that may be used in combination with the one or more markers in Tables 1-31 include any measurable characteristic described herein that reflects in a quantitative or qualitative manner the physiological state of an organism, e.g., whether the organism has prostate cancer. The physiological state of an organism is inclusive of any disease or non-disease state, e.g., a subject having prostate cancer or a subject who is otherwise healthy. The markers of the invention that may be used in combination with the markers in Tables 1-31 include characteristics that can be objectively measured and evaluated as indicators of normal processes, pathogenic processes, or pharmacologic responses to a therapeutic intervention, including in particular, prostate cancer. Such combination markers can be clinical parameters (e.g., age, performance status), laboratory measures (e.g., molecular markers, such as prostate specific antigen), imaging-based measures, or genetic or other molecular determinants. Examples of markers for use in combination with the markers in Tables 1-31 include, for example, polypeptides, peptides, polypeptide fragments, proteins, antibodies, hormones, polynucleotides, RNA or RNA fragments, microRNA (miRNAs), lipids, polysaccharides, and other bodily metabolites that are diagnostic and/or indicative and/or predictive of prostate cancer, or any particular stage or phase of prostate cancer, e.g., Gleason grade 1, grade 2, grade 3, grade 4, or grade 5 prostate cancer or TNM classifications. An example of a marker that is predictive of any particular stage or phase of prostate cancer, e.g., Gleason grade 1, grade 2, grade 3, grade 4, or grade 5 prostate cancer includes one or more markers selected from Table 29. In other embodiments, the present invention also involves the analysis and consideration of any clinical and/or patient-related health data, for example, data obtained from an Electronic Medical Record (e.g., collection of electronic health information about individual patients or populations relating to various types of data, such as, demographics, medical history, medication and allergies, immunization status, laboratory test results, radiology images, vital signs, personal statistics like age and weight, and billing information).

The present invention also contemplates the use of particular combinations of the markers of Tables 1-31. In one embodiment, the invention contemplates marker sets with at least two (2) members, which may include any two of the markers in Tables 1-31. In another embodiment, the invention contemplates marker sets with at least three (3) members, which may include any three of the markers in Tables 1-31. In another embodiment, the invention contemplates marker sets with at least four (4) members, which may include any four of the markers in Tables 1-31. In another embodiment, the invention contemplates marker sets with at least five (5) members, which may include any five of the markers in Tables 1-31. In another embodiment, the invention contemplates marker sets with at least six (6) members, which may include any six of the markers in Tables 1-31. In another embodiment, the invention contemplates marker sets with at least seven (7) members, which may include any seven of the markers in Tables 1-31. In another embodiment, the invention contemplates marker sets with at least eight (8) members, which may include any eight of the markers in Tables 1-31. In another embodiment, the invention contemplates marker sets with at least nine (9) members, which may include any nine of the markers in Tables 1-31. In another embodiment, the invention contemplates marker sets with at least ten (10) members, which may include any ten of the markers in Tables 1-31. In another embodiment, the invention contemplates marker sets with at least eleven (11) members, which may include any ten of the markers in Tables 1-31. In another embodiment, the invention contemplates marker sets with at least twelve (12) members, which may include any ten of the markers in Tables 1-31. In other embodiments, the invention contemplates a marker set comprising at least 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 80, 90, 100, 110, or 120 of the markers listed in Tables 1-31.

In one embodiment, markers used in the methods of the invention which are diagnostic and/or indicative and/or predictive of prostate cancer comprise one or both of nicotinamide and eicosenoic acid. In one embodiment, nicotinamide and/or eicosenoic acid are increased in a subject having prostate cancer. In another embodiment, the marker used in the methods of the invention which is diagnostic and/or indicative and/or predictive of prostate cancer comprises a decanoylcarnitate, e.g., ketodecanoylcarnitine. In one embodiment, the decanoylcarnitate, e.g., ketodecanoylcarnitine, is decreased in a subject having prostate cancer. In another embodiment, markers used in the methods of the invention which are diagnostic and/or indicative and/or predictive of prostate cancer comprise one or more of nicotinamide, eicosenoic acid, and a decanoylcarnitate, e.g., ketodecanoylcarnitine.

In certain embodiments, the markers in Tables 1-31 may be used in combination with at least one other marker, or more preferably, with at least two other markers, or still more preferably, with at least three other markers, or even more preferably with at least four other markers. Still further, the markers in Tables 1-31 in certain embodiments, may be used in combination with at least five other markers, or at least six other markers, or at least seven other markers, or at least eight other markers, or at least nine other markers, or at least ten other markers, or at least eleven other markers, or at least twelve other markers, or at least thirteen other markers, or at least fourteen other markers, or at least fifteen other markers, or at least sixteen other markers, or at least seventeen other markers, or at least eighteen other markers, or at least nineteen other markers, or at least twenty other markers. Further, the markers in Tables 1-31 may be used in combination with a multitude of other markers, including, for example, with between about 20-50 other markers, or between 50-100, or between 100-500, or between 500-1000, or between 1000-10,000 or markers or more.

In certain embodiments, the at least one other marker is any prostate cancer marker previously known in the art. In certain other embodiments, the at least one other marker can include genes that have been described in the literature as being specifically expressed in the prostate. These genes can include, for example, prostate-specific membrane antigen (PSM) (Fair et al., 1997, Prostate-specific membrane antigen. Prostate 32:140-148), prostate stem cell antigen (PSCA) (Reiter et al., 1998, Prostate stem cell antigen: a cell surface marker overexpressed in prostate cancer. Proc. Natl. Acad. Sci. USA 95:1735-1740), TMPRSS2 (Lin et al., 1999. Prostate-localized and androgen-regulated expression of the membrane-bound serine protease TMPRSS2. Cancer Res. 59:4180-4184), PDEF (Oettgen et al., 2000, PDEF, a novel prostate epithelium-specific ETS transcription factor, interacts with the androgen receptor and activates prostate-specific antigen gene expression. J. Biol. Chem. 275:1216-1225), prostate-specific gene-1 (Herness, 2003. A novel human prostate-specific gene-1 (HPG-1): molecular cloning, sequencing, and its potential involvement in prostate carcinogenesis. Cancer Res. 63:329-336), and even various non-coding RNA's (ncRNA's), like PCA3 (Bussemakers et al., 1999. DD3: a new prostate-specific gene, highly overexpressed in prostate cancer, Cancer Res. 59:5975-5979), PCGEM1 (Srikantan et al., 2000. PCGEM1, a prostate-specific gene, is overexpressed in prostate cancer. Proc. Natl. Acad. Sci. USA 97:12216-12221) and the gene cluster P704P, P712P, and P775P(Stolk et al., 2004. P704P, P712P, and P775P: A genomic cluster of prostate-specific genes. Prostate 60:214-226). Only a fraction of these markers have been associated with prostate cancer prognosis, progression and/or metastatic capacity and as such, their potential as valuable biomarkers and/or therapeutic targets is largely unknown.

In certain other embodiments, the at least one other marker is prostate-specific antigen (PSA), also known as kallikrein-3, seminin, P-30 antigen, semenogelase, gamma-seminoprotein, APS, hK3, and KLK2A1. Kallikreins are a subgroup of serine proteases having diverse physiological functions. Growing evidence suggests that many kallikreins are implicated in carcinogenesis and some have potential as novel cancer and other disease biomarkers. This gene is one of the fifteen kallikrein subfamily members located in a cluster on chromosome 19. Its protein product is a protease present in seminal plasma. It is thought to function normally in the liquefaction of seminal coagulum, presumably by hydrolysis of the high molecular mass seminal vesicle protein. Serum level of this protein, called PSA in the clinical setting, is useful in the diagnosis and monitoring of prostatic carcinoma. Alternate splicing of this gene generates several transcript variants encoding different isoforms.

As used herein, PSA refers to both the gene and the protein, in both processed and unprocessed forms, unless clearly indicated otherwise by context. The NCBI gene ID for PSA is 354 and detailed information can be found at the NCBI website (incorporated herein by reference in the version available on the filing date of the application to which this application claims priority).

Homo sapiens PSA is located on chromosome 19 at 19q13.41Sequence: NC_000019.9 (51358171 . . . 51364020). Four splice variants of human PSA are known. Prostate-specific antigen isoform 3 preproprotein, NM_001030047.1, prostate-specific antigen isoform 4 preproprotein, NM_001030048.1, prostate-specific antigen isoform 6 preproprotein, NM_001030050.1, and prostate-specific antigen isoform 1 preproprotein, NM 001648.2. (Each GenBank number is incorporated herein by reference in the version available on the filing date of the application to which this application claims priority).

It is understood that the invention includes the use of any combination of one or more of the PSA sequences provided in the sequence listing or any fragments thereof as long as the fragment can allow for the specific identification of PSA. Methods of the invention and reagents can be used to detect single isoforms of PSA, combinations of PSA isoforms, or all of the PSA isoforms simultaneously. Unless specified, PSA can be considered to refer to one or more isoforms of PSA, including total PSA. Moreover, it is understood that there are naturally occurring variants of PSA, which may or may not be associated with a specific disease state, the use of which are also included in the instant application.

In addition, it is understood that the invention includes the use of any fragments of PSA polypeptide as long as the fragment allow for the specific identification of PSA by a detection method of the invention. For example, an ELISA antibody must be able to bind to the PSA fragment so that detection is possible. Moreover, it is understood that there are naturally occurring variants of PSA which may or may not be associated with a specific disease state, the use of which are also included in this application. Accordingly, the present inventions also contemplates fragments and variants of PSA which may be associated with a disease state, e.g., prostate cancer, and/or a particular stage or phase of a disease state, e.g., grades 1-5 of prostate cancer. It is also understood that the invention encompasses the use of nucleic acid molecules encoding PSA, including for example, PSA-encoding DNA, PSA mRNA, and fragments and/or variants thereof. Reference to “PSA” may refer to PSA polypeptide or to the PSA gene, unless otherwise indicated.

The specific marker identified herein as prostate-specific membrane antigen (PSM) is further described in Sokoll et al., 1997, Prostate-specific antigen—Its discovery and biochemical characteristics, Urol. Clin. North Am., 24:253-259, which is incorporated herein by reference.

The specific marker identified herein as prostate stem cell antigen (PSCA) is further described in Fair et al., 1997, Prostate-specific membrane antigen, Prostate, 32:140-148, which is incorporated herein by reference.

The specific marker identified herein as TMPRSS2 is further described in Lin et al., 1999, Prostate-localized and androgen-regulated expression of the membrane-bound serine protease TMPRSS2, Cancer Res., 59:4180-4184, which is incorporated herein by reference.

The specific marker identified herein as PDEF is further described in Oettgen et al., PDEF, a novel prostate epithelium-specific ETS transcription factor interacts with the androgen receptor and activates prostate-specific antigen gene expression, J. Biol. Chem., 275: 1216-1225, which is incorporated herein by reference.

The specific marker identified herein as prostate-specific gene-1 (HPG-1) is further described in Herness, A novel human prostate-specific gene-1 (HPG-1): molecular cloning, sequencing, and its potential involvement in prostate carcinogenesis, 2003, Cancer Res. 63:329-336, which is incorporated herein by reference.

The non-coding RNA's (ncRNA's) identified as PCA3 is further described in Bussemakers et al., 1999, DD3: a new prostate-specific gene, highly overexpressed in prostate cancer, Cancer Res. 59:5975-5979, which is incorporated herein by reference.

The non-coding RNA identified as PCGEM1 is further described in Srikantan et al., 2000. PCGEM1, a prostate-specific gene, is overexpressed in prostate cancer. Proc. Natl. Acad. Sci. USA 97:12216-12221, which is incorporated herein by reference.

The gene cluster P704P, P712P, and P775P is further described in Stolk et al., 2004. P704P, P712P, and P775P: A genomic cluster of prostate-specific genes. Prostate 60:214-226), which is incorporated herein by reference.

In certain embodiments, the marker, e.g., a prostate cancer marker, is a structural lipid, for example, a structural lipid listed in Tables 1-7. In some embodiments, the invention also relates to a marker set comprising one or more of the structural lipids listed in Tables 1-7. In certain embodiments, the marker, e.g., a prostate cancer marker, is a signaling lipid, for example, a signaling lipid listed in Tables 8-12. In some embodiments, the invention also relates to a marker comprising one or more of the signaling lipids listed in Tables 8-12. In certain embodiments, the marker, e.g., a prostate cancer marker, is a protein, for example, a protein listed in Tables 13-18. In some embodiments, the invention also relates to a marker comprising one or more of the protein listed in Tables 13-18. In certain embodiments, the marker, e.g., a prostate cancer marker, is a metabolite, for example, a metabolite listed in Tables 19-25. In some embodiments, the invention also relates to a marker set comprising one or more of the metabolites listed in Tables 19-25. In certain embodiments, the marker, e.g., a prostate cancer marker, is selected from Tables 26-28. In some embodiments, the invention also relates to a marker comprising one or more of the markers listed in Tables 26-28.

In some embodiments, the marker, e.g., a prostate cancer marker, comprises at least two or more markers, wherein each of the two of more markers are selected from the structural lipids set forth in Tables 1-7, the signaling lipids set forth in Tables 8-12, the proteins set forth in Tables 13-18, the metabolites set forth in Tables 19-25, and/or the markers set forth in Tables 26-28.

In certain embodiments, the level of the marker, e.g., a prostate cancer marker, is increased when compared to the predetermined threshold value in the subject. In some embodiments, the marker, e.g., a prostate cancer marker, is selected from the group consisting of FFA_18:3, TAG_54:7+NH4, TAG_54:6+NH4, PA_18:1/20:2, FFA_18:3, FFA_20:1, TAG 54:7+NH4, TAG_54:6+NH4, PA_18:1/18:3, 6-KETO-PGF1A, TXB2, 13-HOTRE/13-HOTRE(R), 9-HOTRE, TXB2, 12-HEPE, 12-HETE, 13-HODE, APOC, APOB, ADIPOQ, SEPP1, CST3, F5, B2M, nicotinamide, eicosenoic acid, glycerylphosphorylethanolamine, nicotinamide, eicosenoic acid, 3-hydroxybutyric acid and 2-keto-isovalerate. In other embodiments, the marker, e.g., a prostate cancer marker, comprises one or more markers selected from Tables 4-7, 11, 12, 16-18, 22-25, 30 and 31, wherein the one or more markers have a FC ratio greater than 1, or a Log FC value greater than 0. In yet another embodiment, the marker, e.g., a prostate cancer marker, is selected from the group consisting of 13-HOTRE/13-HOTRE(R), nicotinamide, eicosenoic acid, glycerylphosphorylethanolamine, and 3-hydroxybutyric acid.

In other embodiments, the level of the marker, e.g., a prostate cancer marker, is decreased when compared to the predetermined threshold value in the subject. In some embodiments, the marker, e.g., a prostate cancer marker, is selected from the group consisting of CE_22:2+NH4, CE_20:0+NH4, CE_22:3+NH4, DAG_40:1+NH4, CE_20:1+NH4, PI_18:0/20:5, CE_22:1+NH4, TAG_54:0+NH4, PI_18:0/20:4, PI_16:0/18:3, PI_16:0/20:4, CE_20:0+NH4, CE_24:0+NH4, CE_22:2+NH4, DAG 42:2+NH4, PE_36:2, 5-HETE, LXA4, 15-OXOETE, 5-HEPE, 8-HETE, LTB4, 5-HEPE, 5-HETE, LTB4, PGE2/PGD2, GPLD1, SERPING1, C3, A2M, SERPINA6, APOA4, APCS, ITIH2, CLU, APOA2, PPBP, C3, APOA4, C4BPA, MMRN2, APOA2, FGA, ABI3BP, APOA1, PROS1, COMP, CDH5, SERPINA6, glu-leu, 6-ketodecanoylcarnitine, myo-inositol, chenodeoxyglycocholate, 2-hydroxy-2-methylbutanedioic acid, nonanedioic acid, 6-ketodecanoylcarnitine, glu-leu, ethanolamine, and nonanoylcarnitine. In other embodiments, the marker, e.g., a prostate cancer marker, comprises one or more markers selected from Tables 4-7, 11, 12, 16-18, 22-25, 30 and 31, wherein the one or more markers have a FC ratio less than 1, or a Log FC value less than 0. In yet another embodiment, the marker, e.g., a prostate cancer marker, is selected from the group consisting of 15-OXOETE, 5-HEPE, 5-HETE, 6-ketodecanoylcarnitine, glu-leu, ethanolamine, and nonanoylcarnitine.

In some embodiments, markers of the invention for predicting the risk of developing prostate cancer or the diagnosis of prostate cancer are differentially expressed populations of different races, for example, in African Americans (AA) or Caucasian Americans (CA). Accordingly, in one aspect, the present invention provides methods for predicting or diagnosing prostate cancer in a subject selected from a population of Caucasians. In certain embodiments, the prostate cancer marker for diagnosing or predicting prostate cancer in a subject selected from a population of Caucasians comprises one or more markers selected from Tables 1, 4, 8, 11, 13, 16, 19, 22, 26, 29 and 30. In certain embodiments, the prostate cancer marker comprises at least two or more markers, wherein each of the two or more markers are selected from the structural lipids set forth in Tables 1 and 4, the signaling lipids set forth in Tables 8 and 11, the proteins set forth in Tables 13 and 16, the metabolites set forth in Tables 19 and 22, and/or the markers set forth in Table 26. In certain embodiments, the prostate cancer marker for diagnosing or predicting prostate cancer in a subject selected from a population of Caucasians comprises one or more markers selected from Table 29. In other embodiments, markers for that are predictive of any particular stage or phase of prostate cancer, e.g., Gleason grade 1, grade 2, grade 3, grade 4, or grade 5 prostate cancer, i.e., Gleason score markers, include one or more marker selected from Table 29.

In certain embodiments, the level of the marker, e.g., a prostate cancer marker, for diagnosing or prognosing prostate cancer in a subject selected from a population of Caucasians, is increased when compared to the predetermined threshold value in the subject. In some embodiments, the prostate cancer marker is selected from the group consisting of FFA_18:3, TAG 54:7+NH4, TAG_54:6+NH4, PA_18:1/20:2, 6-KETO-PGF1A, TXB2, APOC, APOB, ADIPOQ, SEPP1, nicotinamide, eicosenoic acid, glycerylphosphorylethanolamine. In other embodiments, the prostate cancer marker comprises one or more markers selected from Tables 4, 11, 16, 22 and 30, wherein the one or more markers have a FC ratio greater than 1, or a Log FC value greater than 0. In certain embodiments, the prostate cancer marker is nicotinamide.

In other embodiments, the level of the marker, e.g., a prostate cancer marker, for diagnosing or prognosing prostate cancer in a subject selected from a population of Caucasians, is decreased when compared to the predetermined threshold value in the subject. In some embodiments, the prostate cancer marker is selected from the group consisting of CE_22:2+NH4, CE_20:0+NH4, CE_22:3+NH4, DAG_40:1+NH4, CE_20:1+NH4, PI_18:0/20:5, CE_22:1+NH4, TAG_54:0+NH4, PI_18:0/20:4, PI_16:0/18:3, PI_16:0/20:4, 5-HETE, LXA4, 15-OXOETE, 5-HEPE, 8-HETE, LTB4, GPLD1, SERPING1, C3, A2M, SERPINA6, APOA4, APCS, ITIH2, CLU, APOA2, PPBP, glu-leu, 6-ketodecanoylcarnitine, myo-inositol, chenodeoxyglycocholate, 2-hydroxy-2-methylbutanedioic acid, nonanedioic acid. In other embodiments, the prostate cancer marker comprises one or more markers selected from Tables 4, 11, 16, 22 and 30, wherein the one or more markers have a FC ratio less than 1, or a Log FC value less than 0. In yet another embodiment, the prostate cancer marker is selected from the group consisting of glu-leu, 5-HETE, 15-OXOETE, 5-HEPE, 8-HETE, and 6-ketodecanoylcarnitine.

In another aspect, the present invention provides methods for diagnosing or prognosing prostate cancer in a subject selected from a population of African Americans. In certain embodiments, the prostate cancer marker for diagnosis of the presence of prostate cancer in a subject selected from a population of African Americans comprises one or more markers selected from Tables 2, 5, 9, 12, 14, 17, 20, 23, 27 and 31. In certain embodiments, the prostate cancer marker comprises at least two or more markers, wherein each of the two or more markers are selected from the structural lipids set forth in Table 2, the signaling lipids set forth in Table 9, the proteins set forth in Table 14, the metabolites set forth in Table 20, and/or the markers set forth in Table 27.

In certain embodiments, the level of the marker, e.g., a prostate cancer marker, for diagnosing or prognosing prostate cancer in a subject selected from a population of African Americans, is increased when compared to the predetermined threshold value in the subject. In some embodiments, the prostate cancer marker is selected from the group consisting of FFA_18:3, FFA_20:1, TAG_54:7+NH4, TAG_54:6+NH4, PA_18:1/18:3, 13-HOTRE/13-HOTRE(R), 9-HOTRE, TXB2, 12-HEPE, 12-HETE, 13-HODE, CST3, F5, B2M, nicotinamide, eicosenoic acid, 3-hydroxybutyric acid and 2-keto-isovalerate. In other embodiments, the prostate cancer marker comprises one or more markers selected from Tables 5, 12, 17, 23 and 31, wherein the one or more markers have a FC ratio greater than 1, or a Log FC value greater than 0. In yet another embodiment, the prostate cancer marker is selected from the group consisting of FFA_18:3, 13-HOTRE/13-HOTRE(R), 9-HOTRE, nicotinamide, eicosenoic acid, 3-hydroxybutyric acid, 2-keto-isovalerate and 2-octandioic-carnitine.

In other embodiments, the level of the marker, e.g., a prostate cancer marker, for diagnosing or prognosing prostate cancer in a subject selected from a population of African Americans, is decreased when compared to the predetermined threshold value in the subject. In some embodiments, the prostate cancer marker is selected from the group consisting of CE_20:0+NH4, CE_24:0+NH4, CE_22:2+NH4, DAG_42:2+NH4, PE_36:2, 5-HEPE, 5-HETE, LTB4, PGE2/PGD2, C3, APOA4, C4BPA, MMRN2, APOA2, FGA, ABI3BP, APOA1, PROS1, COMP, CDH5, SERPINA6, 6-ketodecanoylcarnitine, glu-leu, ethanolamine, and nonanoylcarnitine. In other embodiments, the prostate cancer marker comprises one or more markers selected from Tables 5, 12, 17, 23 and 31, wherein the one or more markers have a FC ratio less than 1, or a Log FC value less than 0. In yet another embodiment, the prostate cancer marker is selected from the group consisting of 6-ketodecanoylcarnitine, glu-leu, ethanolamine, and nonanoylcarnitine and propionylcarnitine.

In one aspect, the present invention provides methods for diagnosing the presence of ERG-positive prostate cancer in a subject. In certain embodiments, the ERG-positive prostate cancer marker for diagnosis of the presence of ERG-positive prostate cancer in a subject comprises one or more markers selected from Tables 6, 30 and 31. In certain embodiments, the ERG-positive prostate cancer marker comprises at least two or more markers, wherein each of the two or more markers are selected from Tables 6, 30 and 31.

In certain embodiments, the level of the marker, e.g., an ERG-positive prostate cancer marker, for diagnosis of the presence of ERG-positive prostate cancer in a subject, is increased when compared to the predetermined threshold value in the subject. In some embodiments, the ERG-positive prostate cancer marker is selected from the group consisting of CE_20:4+NH4, PG_16:1/18:3, D18:0/16:1-MONOHEX, D18:1/22:1-MONOHEX, PG_16:1/20:3. In other embodiments, the ERG-positive prostate cancer marker comprises one or more markers selected from Tables 6, 30 and 31, wherein the one or more markers have a FC ratio greater than 1, or a Log FC value greater than 0.

In other embodiments, the level of the marker, e.g., an ERG-positive prostate cancer marker, for diagnosis of the presence of ERG-positive prostate cancer in a subject, is decreased when compared to the predetermined threshold value in the subject. In some embodiments, the ERG-positive prostate cancer marker is selected from the group consisting of LPC_0-14:1, LPC_22:1, LPC_10:0, LPC_0-22:0, LPC_24:0. In other embodiments, the ERG-positive prostate cancer marker comprises one or more markers selected from Tables 6, 30 and 31, wherein the one or more markers have a FC ratio less than 1, or a Log FC value less than 0.

In another aspect, the present invention provides methods for diagnosing the presence of prostate cancer in a subject with a BMI index equal or greater than 30. In certain embodiments, the high BMI prostate cancer marker for diagnosis of the presence of prostate cancer in a subject comprises one or more markers selected from Tables 7, 18 and 25. In certain embodiments, the high BMI prostate cancer marker comprises at least two or more markers, wherein each of the two or more markers are selected from Tables 7, 18 and 25.

In certain embodiments, the level of the marker, e.g., a high BMI prostate cancer marker, for diagnosis of the presence of prostate cancer in a subject with a BMI index equal or greater than 30, is increased when compared to the predetermined threshold value in the subject. In some embodiments, the high BMI prostate cancer marker comprises one or more markers selected from Tables 7, 18 and 25, wherein the one or more markers have a FC ratio greater than 1, or a Log FC value greater than 0.

In other embodiments, the level of the marker, e.g., a high BMI prostate cancer marker, for diagnosis of the presence of prostate cancer in a subject with a BMI index equal or greater than 30, is decreased when compared to the predetermined threshold value in the subject. In some embodiments, the high BMI prostate cancer marker comprises one or more markers selected from Tables 7, 18 and 25, wherein the one or more markers have a FC ratio less than 1, or a Log FC value less than 0.

In certain embodiments, the marker, e.g., a prostate cancer marker, a ERG-positive prostate cancer marker, or a high BMI prostate cancer marker, comprises one or more markers with an increased level when compared to the predetermined threshold value in the subject, and/or one or more markers with a decreased level when compared to the predetermined threshold value in the subject.

In a further aspect, the present invention provides methods for diagnosing the presence of ERG-negative prostate cancer in a Caucasian subject with a BMI index equal or greater than 30. The methods include detecting the level of mercapto-succinyl-carnitine in a biological sample from the subject; and comparing the level of mercapto-succinyl-carnitine in the biological sample with a predetermined threshold value; wherein the level of mercapto-succinyl-carnitine above the predetermined threshold value indicates a diagnosis that ERG-negative prostate cancer is present in the subject.

In another aspect, the present invention provides for the identification of a “diagnostic signature” or “disease profile” based on the levels of the markers of the invention in a biological sample, including in a diseased tissue or directly from the serum or blood, that correlates with the presence and/or risk and/or prognosis of prostate cancer. The “levels of the markers” can refer to the level of a marker lipid, protein, or metabolite in a biological sample, e.g., plasma or serum. The “levels of the markers” can also refer to the expression level of the genes corresponding to the proteins, e.g., by measuring the expression levels of the corresponding marker mRNAs. The collection or totality of levels of markers provide a diagnostic signature that correlates with the presence and/or diagnosis and/or progression of prostate cancer. The methods for obtaining a diagnostic signature or disease profile of the invention are meant to encompass any measurable characteristic that reflects in a quantitative or qualitative manner the physiological state of an organism, e.g., whether the organism has prostate cancer. The physiological state of an organism is inclusive of any disease or non-disease state, e.g., a subject having prostate cancer or a subject who is otherwise healthy. Said another way, the methods used for identifying a diagnostic signature or disease profile of the invention include determining characteristics that can be objectively measured and evaluated as indicators of normal processes, pathogenic processes, or pharmacologic responses to a therapeutic intervention, including, in particular, prostate cancer. These characteristics can be clinical parameters (e.g., age, performance status), laboratory measures (e.g., molecular markers, such as proteins, lipids, or metabolites), imaging-based measures, or genetic or other molecular determinants. Examples of markers include, for example, polypeptides, peptides, polypeptide fragments, proteins, antibodies, hormones, polynucleotides, RNA or RNA fragments, microRNA (miRNAs), lipids, polysaccharides, and other metabolites that are diagnostic and/or indicative and/or predictive of prostate cancer. Examples of markers also include polypeptides, peptides, polypeptide fragments, proteins, antibodies, hormones, polynucleotides, RNA or RNA fragments, microRNA (miRNAs), lipids, polysaccharides, and other metabolites which are diagnostic and/or indicative and/or predictive of any stage or clinical phase of prostate cancer, e.g., Gleason grade 1, grade 2, grade 3, grade 4, or grade 5 prostate cancer.

In a particular embodiment, a prostate cancer profile or diagnostic signature is determined on the basis of the combination of the markers in Tables 1-31 together with one or more additional markers of prostate cancer. Other markers that may be used in combination with the markers in Tables 1-31 include any measurable characteristic that reflects in a quantitative or qualitative manner the physiological state of an organism, e.g., whether the organism has prostate cancer. The physiological state of an organism is inclusive of any disease or non-disease state, e.g., a subject having prostate cancer or a subject who is otherwise healthy. Said another way, the markers of the invention that may be used in combination with the markers in Tables 1-31 include characteristics that can be objectively measured and evaluated as indicators of normal processes, pathogenic processes, or pharmacologic responses to a therapeutic intervention, including, in particular, prostate cancer. Such combination markers can be clinical parameters (e.g., age, performance status), laboratory measures (e.g., molecular markers), imaging-based measures, or genetic or other molecular determinants. Example of markers for use in combination with the markers in Tables 1-31 include, for example, polypeptides, peptides, polypeptide fragments, proteins, antibodies, hormones, polynucleotides, RNA or RNA fragments, microRNA (miRNAs), lipids, polysaccharides, and other metabolites that are diagnostic and/or indicative and/or predictive of prostate cancer, or any particular stage or phase of prostate cancer, e.g., Gleason grade 1, grade 2, grade 3, grade 4, or grade 5 prostate cancer. In certain embodiments, markers for use in combination with the markers in Tables 1-31 include polypeptides, peptides, polypeptide fragments, proteins, antibodies, hormones, polynucleotides, RNA or RNA fragments, microRNA (miRNAs), lipids, polysaccharides, and other bodily metabolites which are diagnostic and/or indicative and/or predictive of prostate cancer, or any stage or clinical phase thereof. In other embodiments, the present invention also involves the analysis and consideration of any clinical and/or patient-related health data, for example, data obtained from an Electronic Medical Record (e.g., collection of electronic health information about individual patients or populations relating to various types of data, such as, demographics, medical history, medication and allergies, immunization status, laboratory test results, radiology images, vital signs, personal statistics like age and weight, and billing information).

In certain embodiments, the diagnostic signature is obtained by (1) detecting the level of at least one of the markers in Tables 1-31 in a biological sample, (2) comparing the level of the at least one marker in Tables 1-31 to the levels of the same marker from a control sample, and (3) determining if the at least one marker in Tables 1-31 is above or below a certain threshold level. If the at least one marker in Tables 1-31 is above or below the threshold level, then the diagnostic signature is indicative of prostate cancer in the biological sample. In certain embodiments, the diagnostic signature can be determined based on an algorithm or computer program that predicts whether the biological sample is from a subject with prostate cancer based on the level of the at least one marker in Tables 1-31.

In certain other embodiments, the diagnostic signature is obtained by (1) detecting the level of at least two markers in Tables 1-31 in a biological sample, (2) comparing the levels of the at least two markers in Tables 1-31 to the levels of the same markers from a control sample, and (3) determining if the at least two markers in Tables 1-31 detected in the biological sample are above or below a certain threshold level. If the at least two markers in Tables 1-31 are above or below the threshold level, then the diagnostic signature is indicative of prostate cancer in the biological sample. In certain embodiments, the diagnostic signature can be determined based on an algorithm or computer program that predicts whether the biological sample is from a subject with prostate cancer based on the levels of the at least two markers in Tables 1-31.

In certain other embodiments, the diagnostic signature is obtained by (1) detecting the level of at least three markers in Tables 1-31 in a biological sample, (2) comparing the levels of the at least three markers in Tables 1-31 to the levels of the same markers from a control sample, and (3) determining if the at least three markers in Tables 1-31 detected in the biological sample are above or below a certain threshold level. If the at least three markers in Tables 1-31 are above the threshold level, then the diagnostic signature is indicative of prostate cancer in the biological sample. In certain embodiments, the diagnostic signature can be determined based on an algorithm or computer program that predicts whether the biological sample is from a subject with prostate cancer based on the levels of the at least three markers in Tables 1-31.

In certain other embodiments, the diagnostic signature is obtained by (1) detecting the level of at least four markers in Tables 1-31 in a biological sample, (2) comparing the levels of the at least four markers in Tables 1-31 to the levels of the same markers from a control sample, and (3) determining if the at least four markers in Tables 1-31 detected in the biological sample are above or below a certain threshold level. If the at least four markers in Tables 1-31 are above the threshold level, then the diagnostic signature is indicative of prostate cancer in the biological sample. In certain embodiments, the diagnostic signature can be determined based on an algorithm or computer program that predicts whether the biological sample is from a subject with prostate cancer based on the levels of the at least four markers in Tables 1-31.

In certain other embodiments, the diagnostic signature is obtained by (1) detecting the level of at least five markers in Tables 1-31 in a biological sample, (2) comparing the levels of the at least five markers in Tables 1-31 to the levels of the same markers from a control sample, and (3) determining if the at least five markers in Tables 1-31 detected in the biological sample are above or below a certain threshold level. If the at least five markers in Tables 1-31 are above the threshold level, then the diagnostic signature is indicative of prostate cancer in the biological sample. In certain embodiments, the diagnostic signature can be determined based on an algorithm or computer program that predicts whether the biological sample is from a subject with prostate cancer based on the levels of the at least five markers in Tables 1-31.

In certain other embodiments, the diagnostic signature is obtained by (1) detecting the level of at least six markers in Tables 1-31 in a biological sample, (2) comparing the levels of the at least six markers in Tables 1-31 to the levels of the same markers from a control sample, and (3) determining if the at least six markers in Tables 1-31 detected in the biological sample are above or below a certain threshold level. If the at least six markers in Tables 1-31 are above the threshold level, then the diagnostic signature is indicative of prostate cancer in the biological sample. In certain embodiments, the diagnostic signature can be determined based on an algorithm or computer program that predicts whether the biological sample is from a subject with prostate cancer based on the levels of the at least six markers in Tables 1-31.

In certain other embodiments, the diagnostic signature is obtained by (1) detecting the level of at least seven markers in Tables 1-31 in a biological sample, (2) comparing the levels of the at least seven markers in Tables 1-31 to the levels of the same markers from a control sample, and (3) determining if the at least seven markers in Tables 1-31 detected in the biological sample are above or below a certain threshold level. If the at least seven markers in Tables 1-31 are above the threshold level, then the diagnostic signature is indicative of prostate cancer in the biological sample. In certain embodiments, the diagnostic signature can be determined based on an algorithm or computer program that predicts whether the biological sample is from a subject with prostate cancer based on the levels of the at least seven markers in Tables 1-31.

In certain other embodiments, the diagnostic signature is obtained by (1) detecting the level of at least eight markers in Tables 1-31 in a biological sample, (2) comparing the levels of the at least eight markers in Tables 1-31 to the levels of the same markers from a control sample, and (3) determining if the at least eight markers in Tables 1-31 detected in the biological sample are above or below a certain threshold level. If the at least eight markers in Tables 1-31 are above the threshold level, then the diagnostic signature is indicative of prostate cancer in the biological sample. In certain embodiments, the diagnostic signature can be determined based on an algorithm or computer program that predicts whether the biological sample is from a subject with prostate cancer based on the levels of the at least eight markers in Tables 1-31.

In certain other embodiments, the diagnostic signature is obtained by (1) detecting the level of at least nine markers in Tables 1-31 in a biological sample, (2) comparing the levels of the at least nine markers in Tables 1-31 to the levels of the same markers from a control sample, and (3) determining if the at least nine markers in Tables 1-31 detected in the biological sample are above or below a certain threshold level. If the at least nine markers in Tables 1-31 are above the threshold level, then the diagnostic signature is indicative of prostate cancer in the biological sample. In certain embodiments, the diagnostic signature can be determined based on an algorithm or computer program that predicts whether the biological sample is from a subject with prostate cancer based on the levels of the at least nine markers in Tables 1-31.

In certain other embodiments, the diagnostic signature is obtained by (1) detecting the level of at least ten markers in Tables 1-31 in a biological sample, (2) comparing the levels of the at least ten markers in Tables 1-31 to the levels of the same markers from a control sample, and (3) determining if the at least ten markers in Tables 1-31 detected in the biological sample are above or below a certain threshold level. If the at least ten markers in Tables 1-31 are above the threshold level, then the diagnostic signature is indicative of prostate cancer in the biological sample. In certain embodiments, the diagnostic signature can be determined based on an algorithm or computer program that predicts whether the biological sample is from a subject with prostate cancer based on the levels of the at least ten markers in Tables 1-31.

In certain embodiments, the marker, e.g., a prostate cancer marker is a structural lipid, for example, a structural lipid listed in Tables 1-7. In some embodiments, the invention relates to a marker comprising one or more of the structural lipids listed in Tables 1-7. In certain embodiments, the marker, e.g., a prostate cancer marker, is a signaling lipid, for example, a signaling lipid listed in Tables 8-12. In some embodiments, the invention relates to a marker comprising one or more of the signaling lipids listed in Tables 8-12. In certain embodiments, the marker, e.g., a prostate cancer marker, is a protein, for example, a protein listed in Tables 13-18. In some embodiments, the invention relates to a marker comprising one or more of the protein listed in Tables 13-18. In certain embodiments, the marker, e.g., a prostate cancer marker is a metabolite, for example, a metabolite listed in Tables 19-25. In some embodiments, the invention relates to a marker comprising one or more of the metabolites listed in Tables 19-25. In certain embodiments, the marker, e.g., a prostate cancer marker, is selected from Tables 26-28. In some embodiments, the invention relates to a marker comprising one or more of the markers listed in Tables 26-28.

In some embodiments, the marker, e.g., a prostate cancer marker, comprises at least two or more markers, wherein each of the two of more markers are selected from the structural lipids set forth in Tables 1-7, the signaling lipids set forth in Tables 8-12, the proteins set forth in Tables 13-18, the metabolites set forth in Tables 19-25, and/or the markers set forth in Tables 26-28.

In some embodiments, the marker, e.g., one or more prostate cancer marker is selected from nicotinamide, eicosenoic acid, and a decanoylcarnitate, e.g., ketodecanoylcarnitine.

In certain embodiments, the level of the marker, e.g., a prostate cancer marker, is increased when compared to the predetermined threshold value in the subject. In some embodiments, the marker, e.g., a prostate cancer marker, is selected from the group consisting of FFA_18:3, TAG_54:7+NH4, TAG_54:6+NH4, PA_18:1/20:2, FFA_18:3, FFA_20:1, TAG_54:7+NH4, TAG_54:6+NH4, PA_18:1/18:3, 6-KETO-PGF1A, TXB2, 13-HOTRE/13-HOTRE(R), 9-HOTRE, TXB2, 12-HEPE, 12-HETE, 13-HODE, APOC, APOB, ADIPOQ, SEPP1, CST3, F5, B2M, nicotinamide, eicosenoic acid, glycerylphosphorylethanolamine, nicotinamide, eicosenoic acid, 3-hydroxybutyric acid and 2-keto-isovalerate. In other embodiments, the marker, e.g., a prostate cancer marker, comprises one or more markers selected from Tables 4-7, 11, 12, 16-18, 22-25, 30 and 31, wherein the one or more markers have a FC ratio greater than 1, or a Log FC value greater than 0. In yet another embodiment, the marker, e.g., a prostate cancer marker, is selected from the group consisting of 13-HOTRE/13-HOTRE(R), nicotinamide, eicosenoic acid, glycerylphosphorylethanolamine, and 3-hydroxybutyric acid.

In other embodiments, the level of the marker, e.g., a prostate cancer marker, is decreased when compared to the predetermined threshold value in the subject. In some embodiments, the marker, e.g., a prostate cancer marker, is selected from the group consisting of CE_22:2+NH4, CE_20:0+NH4, CE_22:3+NH4, DAG_40:1+NH4, CE_20:1+NH4, PI_18:0/20:5, CE_22:1+NH4, TAG_54:0+NH4, PI_18:0/20:4, PI_16:0/18:3, PI_16:0/20:4, CE_20:0+NH4, CE_24:0+NH4, CE_22:2+NH4, DAG 42:2+NH4, PE_36:2, 5-HETE, LXA4, 15-OXOETE, 5-HEPE, 8-HETE, LTB4, 5-HEPE, 5-HETE, LTB4, PGE2/PGD2, GPLD1, SERPING1, C3, A2M, SERPINA6, APOA4, APCS, ITIH2, CLU, APOA2, PPBP, C3, APOA4, C4BPA, MMRN2, APOA2, FGA, ABI3BP, APOA1, PROS1, COMP, CDH5, SERPINA6, glu-leu, 6-ketodecanoylcarnitine, myo-inositol, chenodeoxyglycocholate, 2-hydroxy-2-methylbutanedioic acid, nonanedioic acid, 6-ketodecanoylcarnitine, glu-leu, ethanolamine, and nonanoylcarnitine. In other embodiments, the marker, e.g., a prostate cancer marker, comprises one or more markers selected from Tables 4-7, 11, 12, 16-18, 22-25, 30 and 31, wherein the one or more markers have a FC ratio less than 1, or a Log FC value less than 0. In yet another embodiment, the marker, e.g., a prostate cancer marker, is selected from the group consisting of 15-OXOETE, 5-HEPE, 5-HETE, 6-ketodecanoylcarnitine, glu-leu, ethanolamine, and nonanoylcarnitine.

In certain embodiments, the prostate cancer marker for diagnosis of the presence of prostate cancer in a subject selected from a population of Caucasians comprises one or more markers selected from Tables 1, 4, 8, 11, 13, 16, 19, 22, 26, 29 and 30. In certain embodiments, the prostate cancer marker comprises at least two or more markers, wherein each of the two or more markers are selected from the structural lipids set forth in Tables 1 and 4, the signaling lipids set forth in Tables 8 and 11, the proteins set forth in Tables 13 and 16, the metabolites set forth in Tables 19 and 22, and/or the markers set forth in Table 26. In certain embodiments, the prostate cancer marker for diagnosis of the presence of prostate cancer in a subject selected from a population of Caucasians comprises one or more markers selected from Table 29. In other embodiments, markers for that are predictive of any particular stage or phase of prostate cancer, e.g., Gleason grade 1, grade 2, grade 3, grade 4, or grade 5 prostate cancer, i.e., Gleason score markers, include one or more marker selected from Table 29.

In certain embodiments, the level of the marker, e.g., a prostate cancer marker, for diagnosis of the presence of prostate cancer in a subject selected from a population of Caucasians, is increased when compared to the predetermined threshold value in the subject. In some embodiments, the prostate cancer marker is selected from the group consisting of FFA_18:3, TAG 54:7+NH4, TAG 54:6+NH4, PA_18:1/20:2, 6-KETO-PGF1A, TXB2, APOC, APOB, ADIPOQ, SEPP1, nicotinamide, eicosenoic acid, glycerylphosphorylethanolamine. In other embodiments, the prostate cancer marker comprises one or more markers selected from Tables 4, 11, 16, 22 and 30, wherein the one or more markers have a FC ratio greater than 1, or a Log FC value greater than 0. In certain embodiments, the prostate cancer marker is nicotinamide.

In other embodiments, the level of the marker, e.g., a prostate cancer marker, for diagnosis of the presence of prostate cancer in a subject selected from a population of Caucasians, is decreased when compared to the predetermined threshold value in the subject. In some embodiments, the prostate cancer marker is selected from the group consisting of CE_22:2+NH4, CE_20:0+NH4, CE_22:3+NH4, DAG_40:1+NH4, CE_20:1+NH4, PI_18:0/20:5, CE_22:1+NH4, TAG 54:0+NH4, PI_18:0/20:4, PI_16:0/18:3, PI_16:0/20:4, 5-HETE, LXA4, 15-OXOETE, 5-HEPE, 8-HETE, LTB4, GPLD1, SERPING1, C3, A2M, SERPINA6, APOA4, APCS, ITIH2, CLU, APOA2, PPBP, glu-leu, 6-ketodecanoylcarnitine, myo-inositol, chenodeoxyglycocholate, 2-hydroxy-2-methylbutanedioic acid, nonanedioic acid. In other embodiments, the prostate cancer marker comprises one or more markers selected from Tables 4, 11, 16, 22 and 30, wherein the one or more markers have a FC ratio less than 1, or a Log FC value less than 0. In yet another embodiment, the prostate cancer marker is selected from the group consisting of glu-leu, 5-HETE, 15-OXOETE, 5-HEPE, 8-HETE, and 6-ketodecanoylcarnitine.

In certain embodiments, the prostate cancer marker for diagnosis of the presence of prostate cancer in a subject selected from a population of African Americans comprises one or more markers selected from Tables 2, 5, 9, 12, 14, 17, 20, 23, 27 and 31. In certain embodiments, the prostate cancer marker comprises at least two or more markers, wherein each of the two or more markers are selected from the structural lipids set forth in Table 2, the signaling lipids set forth in Table 9, the proteins set forth in Table 14, the metabolites set forth in Table 20, and/or the markers set forth in Table 27.

In certain embodiments, the level of the marker, e.g., a prostate cancer marker, for diagnosis of the presence of prostate cancer in a subject selected from a population of African Americans, is increased when compared to the predetermined threshold value in the subject. In some embodiments, the prostate cancer marker is selected from the group consisting of FFA_18:3, FFA_20:1, TAG 54:7+NH4, TAG 54:6+NH4, PA_18:1/18:3, 13-HOTRE/13-HOTRE(R), 9-HOTRE, TXB2, 12-HEPE, 12-HETE, 13-HODE, CST3, F5, B2M, nicotinamide, eicosenoic acid, 3-hydroxybutyric acid and 2-keto-isovalerate. In other embodiments, the prostate cancer marker comprises one or more markers selected from Tables 5, 12, 17, 23 and 31, wherein the one or more markers have a FC ratio greater than 1, or a Log FC value greater than 0. In yet another embodiment, the prostate cancer marker is selected from the group consisting of FFA_18:3, 13-HOTRE/13-HOTRE(R), 9-HOTRE, nicotinamide, eicosenoic acid, 3-hydroxybutyric acid, 2-keto-isovalerate and 2-octandioic-carnitine.

In other embodiments, the level of the marker, e.g., a prostate cancer marker, for diagnosis of the presence of prostate cancer in a subject selected from a population of African Americans, is decreased when compared to the predetermined threshold value in the subject. In some embodiments, the prostate cancer marker is selected from the group consisting of CE_20:0+NH4, CE_24:0+NH4, CE_22:2+NH4, DAG_42:2+NH4, PE_36:2, 5-HEPE, 5-HETE, LTB4, PGE2/PGD2, C3, APOA4, C4BPA, MMRN2, APOA2, FGA, ABI3BP, APOA1, PROS1, COMP, CDH5, SERPINA6, 6-ketodecanoylcarnitine, glu-leu, ethanolamine, and nonanoylcarnitine. In other embodiments, the prostate cancer marker comprises one or more markers selected from Tables 5, 12, 17, 23 and 31, wherein the one or more markers have a FC ratio less than 1, or a Log FC value less than 0. In yet another embodiment, the prostate cancer marker is selected from the group consisting of 6-ketodecanoylcarnitine, glu-leu, ethanolamine, and nonanoylcarnitine and propionylcarnitine.

In certain embodiments, the ERG-positive prostate cancer marker for diagnosis of the presence of ERG-positive prostate cancer in a subject comprises one or more markers selected from Tables 6, 30 and 31. In certain embodiments, the ERG-positive prostate cancer marker comprises at least two or more markers, wherein each of the two or more markers are selected from Tables 6, 30 and 31.

In certain embodiments, the level of the marker, e.g., an ERG-positive prostate cancer marker, for diagnosis of the presence of ERG-positive prostate cancer in a subject, is increased when compared to the predetermined threshold value in the subject. In some embodiments, the ERG-positive prostate cancer marker is selected from the group consisting of CE_20:4+NH4, PG_16:1/18:3, D18:0/16:1-MONOHEX, D18:1/22:1-MONOHEX, PG_16:1/20:3. In other embodiments, the ERG-positive prostate cancer marker comprises one or more markers selected from Tables 6, 30 and 31, wherein the one or more markers have a FC ratio greater than 1, or a Log FC value greater than 0.

In other embodiments, the level of the marker, e.g., an ERG-positive prostate cancer marker, for diagnosis of the presence of ERG-positive prostate cancer in a subject, is decreased when compared to the predetermined threshold value in the subject. In some embodiments, the ERG-positive prostate cancer marker is selected from the group consisting of LPC_0-14:1, LPC_22:1, LPC_10:0, LPC_0-22:0, LPC_24:0. In other embodiments, the ERG-positive prostate cancer marker comprises one or more markers selected from Tables 6, 30 and 31, wherein the one or more markers have a FC ratio less than 1, or a Log FC value less than 0.

In certain embodiments, the high BMI prostate cancer marker for diagnosis of the presence of prostate cancer in a subject with a BMI index equal or greater than 30 comprises one or more markers selected from Tables 7, 18 and 25. In certain embodiments, the high BMI prostate cancer marker comprises at least two or more markers, wherein each of the two or more markers are selected from Tables 7, 18 and 25.

In certain embodiments, the level of the marker, e.g., a high BMI prostate cancer marker, for diagnosis of the presence of prostate cancer in a subject with a BMI index equal or greater than 30, is increased when compared to the predetermined threshold value in the subject. In some embodiments, the high BMI prostate cancer marker comprises one or more markers selected from Tables 7, 18 and 25, wherein the one or more markers have a FC ratio greater than 1, or a Log FC value greater than 0.

In other embodiments, the level of the marker, e.g., a high BMI prostate cancer marker, for diagnosis of the presence of prostate cancer in a subject with a BMI index equal or greater than 30, is decreased when compared to the predetermined threshold value in the subject. In some embodiments, the high BMI prostate cancer marker comprises one or more markers selected from Tables 7, 18 and 25, wherein the one or more markers have a FC ratio less than 1, or a Log FC value less than 0.

In certain embodiments, the marker, e.g., a prostate cancer marker, a ERG-positive prostate cancer marker, a ERG-negative prostate cancer marker, a high BMI prostate cancer marker, or a Gleason Score marker, comprises one or more markers with an increased level when compared to the predetermined threshold value in the subject, and/or one or more markers with a decreased level when compared to the predetermined threshold value in the subject.

In accordance with various embodiments, algorithms may be employed to predict whether or not a biological sample is likely to be diseased, e.g., have prostate cancer. The skilled artisan will appreciate that an algorithm can be any computation, formula, statistical survey, nomogram, look-up Tables, decision tree method, or computer program which processes a set of input variables (e.g., number of markers (n) which have been detected at a level exceeding some threshold level, or number of markers (n) which have been detected at a level below some threshold level) through a number of well-defined successive steps to eventually produce a score or “output,” e.g., a diagnosis of prostate cancer. Any suitable algorithm-whether computer-based or manual-based (e.g., look-up Tables)—is contemplated herein.

In certain embodiments, an algorithm of the invention is used to predict whether a biological sample is from a subject that has prostate cancer by producing a score on the basis of the detected level of at least 1, 2, 3, 4, 5, 6, 7, 8, 9, 10, 11, 12, 13, 14, 15, 20, 30, 40, 50, 60, 70, or 80 of the markers in Tables 1-31 in the sample, wherein if the score is above or below a certain threshold score, then the biological sample is from a subject that has prostate cancer.

Moreover, a prostate cancer profile or signature may be obtained by detecting at least one of the markers in Tables 1-31 in combination with at least one other marker, or more preferably, with at least two other markers, or still more preferably, with at least three other markers, or even more preferably with at least four other markers. Still further, the markers in Tables 1-31 in certain embodiments, may be used in combination with at least five other markers, or at least six other markers, or at least seven other markers, or at least eight other markers, or at least nine other markers, or at least ten other markers, or at least eleven other markers, or at least twelve other markers, or at least thirteen other markers, or at least fourteen other markers, or at least fifteen other markers, or at least sixteen other markers, or at least seventeen other markers, or at least eighteen other markers, or at least nineteen other markers, or at least twenty other markers. Further still, the markers in Tables 1-31 may be used in combination with a multitude of other markers, including, for example, with between about 20-50 other markers, or between 50-100, or between 100-500, or between 500-1000, or between 1000-10,000 or markers or more.

In certain embodiments, the markers of the invention can include variant sequences. More particularly, certain binding agents/reagents used for detecting certain of the markers of the invention can bind and/or identify variants of these certain markers of the invention. As used herein, the term “variant” encompasses nucleotide or amino acid sequences different from the specifically identified sequences, wherein one or more nucleotides or amino acid residues is deleted, substituted, or added. Variants may be naturally occurring allelic variants, or non-naturally occurring variants. Variant sequences (polynucleotide or polypeptide) preferably exhibit at least 80%, 85%, 90%, 95%, 96%, 97%, 98% or 99% identity to a sequence disclosed herein. The percentage identity is determined by aligning the two sequences to be compared as described below, determining the number of identical residues in the aligned portion, dividing that number by the total number of residues in the inventive (queried) sequence, and multiplying the result by 100.

In addition to exhibiting the recited level of sequence identity, variants of the disclosed protein markers are preferably themselves expressed in subjects with prostate cancer at levels that are higher or lower than the levels of expression in normal, healthy individuals.

Variant sequences generally differ from the specifically identified sequence only by conservative substitutions, deletions or modifications. As used herein, a “conservative substitution” is one in which an amino acid is substituted for another amino acid that has similar properties, such that one skilled in the art of peptide chemistry would expect the secondary structure and hydropathic nature of the polypeptide to be substantially unchanged. In general, the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. Variants may also, or alternatively, contain other modifications, including the deletion or addition of amino acids that have minimal influence on the antigenic properties, secondary structure and hydropathic nature of the polypeptide. For example, a polypeptide may be conjugated to a signal (or leader) sequence at the N-terminal end of the protein which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

Polypeptide and polynucleotide sequences may be aligned, and percentages of identical amino acids or nucleotides in a specified region may be determined against another polypeptide or polynucleotide sequence, using computer algorithms that are publicly available. The percentage identity of a polynucleotide or polypeptide sequence is determined by aligning polynucleotide and polypeptide sequences using appropriate algorithms, such as BLASTN or BLASTP, respectively, set to default parameters; identifying the number of identical nucleic or amino acids over the aligned portions; dividing the number of identical nucleic or amino acids by the total number of nucleic or amino acids of the polynucleotide or polypeptide of the present invention; and then multiplying by 100 to determine the percentage identity.

Two exemplary algorithms for aligning and identifying the identity of polynucleotide sequences are the BLASTN and FASTA algorithms. The alignment and identity of polypeptide sequences may be examined using the BLASTP algorithm. BLASTX and FASTX algorithms compare nucleotide query sequences translated in all reading frames against polypeptide sequences. The FASTA and FASTX algorithms are described in Pearson and Lipman, Proc. Natl. Acad. Sci. USA 85:2444-2448, 1988; and in Pearson, Methods in Enzymol. 183:63-98, 1990. The FASTA software package is available from the University of Virginia, Charlottesville, Va. 22906-9025. The FASTA algorithm, set to the default parameters described in the documentation and distributed with the algorithm, may be used in the determination of polynucleotide variants. The readme files for FASTA and FASTX Version 2.0× that are distributed with the algorithms describe the use of the algorithms and describe the default parameters.

The BLASTN software is available on the NCBI anonymous FTP server and is available from the National Center for Biotechnology Information (NCBI), National Library of Medicine, Building 38A, Room 8N805, Bethesda, Md. 20894. The BLASTN algorithm Version 2.0.6 [Sep. 10, 1998] and Version 2.0.11 [Jan. 20, 2000] set to the default parameters described in the documentation and distributed with the algorithm, is preferred for use in the determination of variants according to the present invention. The use of the BLAST family of algorithms, including BLASTN, is described at NCBI's website and in the publication of Altschul, et al., “Gapped BLAST and PSI-BLAST: a new generation of protein database search programs,” Nucleic Acids Res. 25:3389-3402, 1997.

In an alternative embodiment, variant polypeptides are encoded by polynucleotide sequences that hybridize to a disclosed polynucleotide under stringent conditions. Stringent hybridization conditions for determining complementarity include salt conditions of less than about 1 M, more usually less than about 500 mM, and preferably less than about 200 mM. Hybridization temperatures can be as low as 5° C., but are generally greater than about 22° C., more preferably greater than about 30° C., and most preferably greater than about 37° C. Longer DNA fragments may require higher hybridization temperatures for specific hybridization. Since the stringency of hybridization may be affected by other factors such as probe composition, presence of organic solvents and extent of base mismatching, the combination of parameters is more important than the absolute measure of any one alone. An example of “stringent conditions” is prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

The invention provides for the use of various combinations and sub-combinations of markers. It is understood that any single marker or combination of the markers provided herein can be used in the invention unless clearly indicated otherwise. Further, any single marker or combination of the markers of the invention can be used in conjunction with PSA.

### D. Tissue Samples

The present invention may be practiced with any suitable biological sample that potentially contains, expresses, includes, a detectable disease biomarker, e.g., a lipid biomarker, a polypeptide biomarker, a nucleic acid biomarker, a mRNA biomarker, a microRNA biomarker. For example, the biological sample may be obtained from sources that include whole blood, serum, urine, diseased and/or healthy organ tissue, for example, biopsy of prostate tumor, and seminal fluid. In certain embodiments, the biological sample urine collected after a digital rectal exam, i.e., post-DRE urine. Preferably, the biological sample is serum or urine.

The methods of the invention may be applied to the study of any prostate tissue sample, i.e., a sample of prostate tissue or fluid, as well as cells (or their progeny) isolated from such tissue or fluid. In another embodiment, the present invention may be practiced with any suitable prostate tissue samples which are freshly isolated or which have been frozen or stored after having been collected from a subject, or archival tissue samples, for example, with known diagnosis, treatment, and/or outcome history. Prostate tissue may be collected by any non-invasive means, such as, for example, fine needle aspiration and needle biopsy, or alternatively, by an invasive method, including, for example, surgical biopsy.

The inventive methods may be performed at the single cell level (e.g., isolation and testing of cancerous cells from the prostate tissue sample). However, the inventive methods may also be performed using a sample comprising many cells, where the assay is “averaging” expression over the entire collection of cells and tissue present in the sample. Preferably, there is enough of the prostate tissue sample to accurately and reliably determine the expression levels of interest. In certain embodiments, multiple samples may be taken from the same prostate tissue in order to obtain a representative sampling of the tissue. In addition, sufficient biological material can be obtained in order to perform duplicate, triplicate or further rounds of testing.

Any commercial device or system for isolating and/or obtaining prostate tissue and/or blood or other biological products, and/or for processing said materials prior to conducting a detection reaction is contemplated.

In certain embodiments, the present invention relates to detecting biomarker nucleic acid molecules (e.g., mRNA encoding the protein markers of Tables 1-31). In such embodiments, RNA can be extracted from a biological sample, e.g., a prostate tissue sample, before analysis. Methods of RNA extraction are well known in the art (see, for example, J. Sambrook et al., “Molecular Cloning: A Laboratory Manual”, 1989, 2^{nd} Ed., Cold Spring Harbour Laboratory Press: New York). Most methods of RNA isolation from bodily fluids or tissues are based on the disruption of the tissue in the presence of protein denaturants to quickly and effectively inactivate RNases. Generally, RNA isolation reagents comprise, among other components, guanidinium thiocyanate and/or beta-mercaptoethanol, which are known to act as RNase inhibitors. Isolated total RNA is then further purified from the protein contaminants and concentrated by selective ethanol precipitations, phenol/chloroform extractions followed by isopropanol precipitation (see, for example, P. Chomczynski and N. Sacchi, Anal. Biochem., 1987, 162: 156-159) or cesium chloride, lithium chloride or cesium trifluoroacetate gradient centrifugations.

Numerous different and versatile kits can be used to extract RNA (i.e., total RNA or mRNA) from bodily fluids or tissues (e.g., prostate tissue samples) and are commercially available from, for example, Ambion, Inc. (Austin, Tex.), Amersham Biosciences (Piscataway, N.J.), BD Biosciences Clontech (Palo Alto, Calif.), BioRad Laboratories (Hercules, Calif.), GIBCO BRL (Gaithersburg, Md.), and Giagen, Inc. (Valencia, Calif.). User Guides that describe in great detail the protocol to be followed are usually included in all these kits. Sensitivity, processing time and cost may be different from one kit to another. One of ordinary skill in the art can easily select the kit(s) most appropriate for a particular situation.

In certain embodiments, after extraction, mRNA is amplified, and transcribed into cDNA, which can then serve as template for multiple rounds of transcription by the appropriate RNA polymerase. Amplification methods are well known in the art (see, for example, A. R. Kimmel and S. L. Berger, Methods Enzymol. 1987, 152: 307-316; J. Sambrook et al., “Molecular Cloning: A Laboratory Manual”, 1989, 2.sup.nd Ed., Cold Spring Harbour Laboratory Press: New York; “Short Protocols in Molecular Biology”, F. M. Ausubel (Ed.), 2002, 5.sup.th Ed., John Wiley & Sons; U.S. Pat. Nos. 4,683,195; 4,683,202 and 4,800,159). Reverse transcription reactions may be carried out using non-specific primers, such as an anchored oligo-dT primer, or random sequence primers, or using a target-specific primer complementary to the RNA for each genetic probe being monitored, or using thermostable DNA polymerases (such as avian myeloblastosis virus reverse transcriptase or Moloney murine leukemia virus reverse transcriptase).

In certain embodiments, the RNA isolated from the prostate tissue sample (for example, after amplification and/or conversion to cDNA or cRNA) is labeled with a detectable agent before being analyzed. The role of a detectable agent is to facilitate detection of RNA or to allow visualization of hybridized nucleic acid fragments (e.g., nucleic acid fragments hybridized to genetic probes in an array-based assay). Preferably, the detectable agent is selected such that it generates a signal which can be measured and whose intensity is related to the amount of labeled nucleic acids present in the sample being analyzed. In array-based analysis methods, the detectable agent is also preferably selected such that it generates a localized signal, thereby allowing spatial resolution of the signal from each spot on the array.

Methods for labeling nucleic acid molecules are well-known in the art. For a review of labeling protocols, label detection techniques and recent developments in the field, see, for example, L. J. Kricka, Ann. Clin. Biochem. 2002, 39: 114-129; R. P. van Gijlswijk et al., Expert Rev. Mol. Diagn. 2001, 1: 81-91; and S. Joos et al., J. Biotechnol. 1994, 35: 135-153. Standard nucleic acid labeling methods include: incorporation of radioactive agents, direct attachment of fluorescent dyes (see, for example, L. M. Smith et al., Nucl. Acids Res. 1985, 13: 2399-2412) or of enzymes (see, for example, B. A. Connoly and P. Rider, Nucl. Acids. Res. 1985, 13: 4485-4502); chemical modifications of nucleic acid fragments making them detectable immunochemically or by other affinity reactions (see, for example, T. R. Broker et al., Nucl. Acids Res. 1978, 5: 363-384; E. A. Bayer et al., Methods of Biochem. Analysis, 1980, 26: 1-45; R. Langer et al., Proc. Natl. Acad. Sci. USA, 1981, 78: 6633-6637; R. W. Richardson et al., Nucl. Acids Res. 1983, 11: 6167-6184; D. J. Brigati et al., Virol. 1983, 126: 32-50; P. Tchen et al., Proc. Natl Acad. Sci. USA, 1984, 81: 3466-3470; J. E. Landegent et al., Exp. Cell Res. 1984, 15: 61-72; and A. H. Hopman et al., Exp. Cell Res. 1987, 169: 357-368); and enzyme-mediated labeling methods, such as random priming nick translation, PCR and tailing with terminal transferase (for a review on enzymatic labeling see, for example, J. Temsamani and S. Agrawal, Mol. Biotechnol. 1996, 5: 223-232).

Any of a wide variety of detectable agents can be used in the practice of the present invention. Suitable detectable agents include, but are not limited to: various ligands, radionuclides, fluorescent dyes, chemiluminescent agents, microparticles (such as, for example, quantum dots, nanocrystals, phosphors and the like), enzymes (such as, for example, those used in an ELISA, i.e., horseradish peroxidase, beta-galactosidase, luciferase, alkaline phosphatase), colorimetric labels, magnetic labels, and biotin, dioxigenin or other haptens and proteins for which antisera or monoclonal antibodies are available.

However, in some embodiments, the expression levels are determined by detecting the expression of a gene product (e.g., protein) thereby eliminating the need to obtain a genetic sample (e.g., RNA) from the prostate tissue sample.

In still other embodiments, the present invention relates to preparing a prediction model for prostate and/or the likelihood of relapse of prostate cancer by preparing a model for prostate cancer based on measuring the biomarkers of the invention in known control samples. More particularly, the present invention relates in some embodiments to preparing a predictive model by evaluating the biomarkers of the invention, i.e., the markers of Tables 1-31.

The skilled person will appreciate that patient tissue samples containing prostate cells or prostate cancer cells may be used in the methods of the present invention including but not limited to those aimed at predicting relapse probability. In these embodiments, the level of expression of the signature gene can be assessed by assessing the amount, e.g. absolute amount or concentration, of a signature gene product, e.g., protein and RNA transcript encoded by the signature gene and fragments of the protein and RNA transcript) in a sample, e.g., stool and/or blood obtained from a patient. The sample can, of course, be subjected to a variety of well-known post-collection preparative and storage techniques (e.g. fixation, storage, freezing lysis, homogenization, DNA or RNA extraction, ultrafiltration, concentration, evaporation, centrifugation, etc.) prior to assessing the amount of the signature gene product in the sample.

The invention further relates to the preparation of a model for prostate cancer or prostate cancer relapse by evaluating the biomarkers of the invention in known samples of prostate cancer. More particularly, the present invention relates to a prostate cancer model for diagnosing and/or monitoring and/or prognosing prostate cancer or prostate cancer relapse using the biomarkers of the invention, i.e., the markers of Tables 1-31.

In the methods of the invention aimed at preparing a model for prostate cancer and/or prostate cancer relapse prediction, it is understood that the particular clinical outcome associated with each sample contributing to the model preferably should be known. Consequently, the model can be established using archived tissue samples. In the methods of the invention aimed at preparing a model for prostate cancer and/or prostate cancer relapse prediction, total RNA can be generally extracted from the source material of interest, generally an archived tissue such as a formalin-fixed, paraffin-embedded tissue, and subsequently purified. Methods for obtaining robust and reproducible gene expression patterns from archived tissues, including formalin-fixed, paraffin-embedded (FFPE) tissues are taught in U.S. Publ. No. 2004/0259105, which is incorporated herein by reference in its entirety. Commercial kits and protocols for RNA extraction from FFPE tissues are available including for example, ROCHE High Pure RNA Paraffin Kit (Roche) MasterPure™ Complete DNA and RNA Purification Kit (EPICENTRE®Madison, Wis.); Paraffin Block RNA Isolation Kit (Ambion, Inc.) and RNeasy™ Mini kit (Qiagen, Chatsworth, Calif.).

The use of FFPE tissues as a source of RNA for RT-PCR has been described previously (Stanta et al., Biotechniques 11:304-308 (1991); Stanta et al., Methods Mol. Biol. 86:23-26 (1998); Jackson et al., Lancet 1:1391 (1989); Jackson et al., J. Clin. Pathol. 43:499-504 (1999); Finke et al., Biotechniques 14:448-453 (1993); Goldsworthy et al., Mol. Carcinog. 25:86-91 (1999); Stanta and Bonin, Biotechniques 24:271-276 (1998); Godfrey et al., J. Mol. Diagnostics 2:84 (2000); Specht et al., J. Mol. Med. 78B27 (2000); Specht et al., Am. J. Pathol. 158:419-429 (2001)). For quick analysis of the RNA quality, RT-PCR can be performed utilizing a pair of primers targeting a short fragment in a highly expressed gene, for example, actin, ubiquitin, gapdh or other well-described commonly used housekeeping gene. If the cDNA synthesized from the RNA sample can be amplified using this pair of primers, then the sample is suitable for the a quantitative measurements of RNA target sequences by any method preferred, for example, the DASL assay, which requires only a short cDNA fragment for the annealing of query oligonucleotides.

There are numerous tissue banks and collections including exhaustive samples from all stages of a wide variety of disease states, most notably cancer and in particular, prostate cancer. The ability to perform genotyping and/or gene expression analysis, including both qualitative and quantitative analysis on these samples enables the application of this methodology to the methods of the invention. In particular, the ability to establish a correlation of gene expression and a known predictor of disease extent and/or outcome by probing the genetic state of tissue samples for which clinical outcome is already known, allows for the establishment of a correlation between a particular molecular signature and the known predictor, such as a Gleason score, to derive a score that allows for a more sensitive prognosis than that based on the known predictor alone. The skilled person will appreciate that by building databases of molecular signatures from tissue samples of known outcomes, many such correlations can be established, thus allowing both diagnosis and prognosis of any condition. Thus, such approaches may be used to correlate the expression levels of the biomarkers of the invention, i.e., the markers of Tables 1-31.

Tissue samples useful for preparing a model for prostate cancer prediction include, for example, paraffin and polymer embedded samples, ethanol embedded samples and/or formalin and formaldehyde embedded tissues, although any suitable sample may be used. In general, nucleic acids isolated from archived samples can be highly degraded and the quality of nucleic preparation can depend on several factors, including the sample shelf life, fixation technique and isolation method. However, using the methodologies taught in U.S. Publ. No. 2004/0259105, which have the significant advantage that short or degraded targets can be used for analysis as long as the sequence is long enough to hybridize with the oligonucleotide probes, highly reproducible results can be obtained that closely mimic results found in fresh samples.

Archived tissue samples, which can be used for all methods of the invention, typically have been obtained from a source and preserved. Preferred methods of preservation include, but are not limited to paraflin embedding, ethanol fixation and formalin, including formaldehyde and other derivatives, fixation as are known in the art. A tissue sample may be temporally “old”, e.g. months or years old, or recently fixed. For example, post-surgical procedures generally include a fixation step on excised tissue for histological analysis. In a preferred embodiment, the tissue sample is a diseased tissue sample, particularly a prostate cancer tissue, including primary and secondary tumor tissues as well as lymph node tissue and metastatic tissue.

Thus, an archived sample can be heterogeneous and encompass more than one cell or tissue type, for example, tumor and non-tumor tissue. Preferred tissue samples include solid tumor samples including, but not limited to, tumors of the prostate. It is understood that in applications of the present invention to conditions other than prostate cancer, the tumor source can be brain, bone, heart, breast, ovaries, prostate, uterus, spleen, pancreas, liver, kidneys, bladder, stomach and muscle. Similarly, depending on the condition, suitable tissue samples include, but are not limited to, bodily fluids (including, but not limited to, blood, urine, serum, lymph, saliva, anal and vaginal secretions, perspiration and semen, of virtually any organism, with mammalian samples being preferred and human samples being particularly preferred). In embodiments directed to methods of establishing a model for prostate cancer relapse prediction, the tissue sample is one for which patient history and outcome is known. Generally, the invention methods can be practiced with the signature gene sequence contained in an archived sample or can be practiced with signature gene sequences that have been physically separated from the sample prior to performing a method of the invention.

### E. Detection and/or Measurement of Biomarkers

The present invention contemplates any suitable means, techniques, and/or procedures for detecting and/or measuring the biomarkers of the invention. The skilled artisan will appreciate that the methodologies employed to measure the biomarkers of the invention will depend at least on the type of biomarker being detected or measured (e.g., lipid or polypeptide biomarker) and the source of the biological sample (e.g., whole blood versus prostate biopsy tissue). Certain biological samples may also require certain specialized treatments prior to measuring the biomarkers of the invention, e.g., the extraction of lipids from a serum in the case of lipid markers being measured.

1. Detection of Lipid Markers

A lipid sample may be extracted from a biological sample using any method known in the art such as chloroform-methanol based methods, isopropanol-hexane methods, the Bligh & Dyer lipid extraction method or a modified version thereof, or any combination thereof. Suitable modifications to the Bligh & Dyer method include treatment of crude lipid extracts with lithium methoxide followed by subsequent liquid-liquid extraction to remove generated free fatty acids, fatty acid methyl esters, cholesterol, and water-soluble components that may hinder the shotgun analysis of sphingolipidomes. Since sphingolipids are inert to the described base-treatment, the global analysis and accurate quantitation to assess low and even very low abundant sphingolipids is possible by using a modified Bligh & Dyer method. Following lipid extraction, it may be beneficial to separate the lipids prior to mass spectrometric analysis. Methods for separating lipids are known in the art. Suitable methods include, but are not limited to, chromatography methods such as solid-phase extraction, high performance liquid chromatography (HPLC), normal-phase HPLC, or reverse-phase HPLC. The resultant lipid extracts are then analyzed by mass spectrometric techniques commonly known in the art.

2. Detection of Protein Markers

The present invention contemplates any suitable method for detecting polypeptide biomarkers of the invention, i.e., the proteins of Tables 13-18. In certain embodiments, the detection method is an immunodetection method involving an antibody that specifically binds to one or more of the proteins of Tables 13-18. The steps of various useful immunodetection methods have been described in the scientific literature, such as, e.g., Nakamura et al. (1987), which is incorporated herein by reference.

In general, the immunobinding methods include obtaining a sample suspected of containing a biomarker protein, peptide or antibody, and contacting the sample with an antibody or protein or peptide in accordance with the present invention, as the case may be, under conditions effective to allow the formation of immunocomplexes.

The immunobinding methods include methods for detecting or quantifying the amount of a reactive component in a sample, which methods require the detection or quantitation of any immune complexes formed during the binding process. Here, one would obtain a sample suspected of containing a prostate specific protein, peptide or a corresponding antibody, and contact the sample with an antibody or encoded protein or peptide, as the case may be, and then detect or quantify the amount of immune complexes formed under the specific conditions.

In terms of biomarker detection, the biological sample analyzed may be any sample that is suspected of containing one more proteins of Tables 13-18. The biological sample may be, for example, a prostate or lymph node tissue section or specimen, a homogenized tissue extract, an isolated cell, a cell membrane preparation, separated or purified forms of any of the above protein-containing compositions, or even any biological fluid that comes into contact with prostate tissues, including blood or lymphatic fluid.

Contacting the chosen biological sample with the protein under conditions effective and for a period of time sufficient to allow the formation of immune complexes (primary immune complexes). Generally, complex formation is a matter of simply adding the composition to the biological sample and incubating the mixture for a period of time long enough for the antibodies to form immune complexes with, i.e., to bind to, any antigens present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any radioactive, fluorescent, biological or enzymatic tags or labels of standard use in the art. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody or a biotin/avidin ligand binding arrangement, as is known in the art.

The protein employed in the detection may itself be linked to a detectable label, wherein one would then simply detect this label, thereby allowing the amount of the primary immune complexes in the composition to be determined.

Alternatively, the first added component that becomes bound within the primary immune complexes may be detected by means of a second binding ligand that has binding affinity for the encoded protein, peptide or corresponding antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the encoded protein, peptide or corresponding antibody is used to form secondary immune complexes, as described above. After washing, the secondary immune complexes are contacted with a third binding ligand or antibody that has binding affinity for the second antibody, again under conditions effective and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

The immunodetection methods of the present invention have evident utility in the diagnosis of conditions such as prostate cancer. Here, a biological or clinical sample suspected of containing either the encoded protein or peptide or corresponding antibody is used. However, these embodiments also have applications to non-clinical samples, such as in the tittering of antigen or antibody samples, in the selection of hybridomas, and the like.

The present invention, in particular, contemplates the use of ELISAs as a type of immunodetection assay. It is contemplated that the biomarker proteins or peptides of the invention will find utility as immunogens in ELISA assays in diagnosis and prognostic monitoring of prostate cancer. Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and Western blotting, dot blotting, FACS analyses, and the like also may be used.

In one exemplary ELISA, antibodies binding to the biomarkers of the invention are immobilized onto a selected surface exhibiting protein affinity, such as a well in a polystyrene microtiter plate. Then, a test composition suspected of containing the prostate cancer marker antigen, such as a clinical sample, is added to the wells. After binding and washing to remove non-specifically bound immunecomplexes, the bound antigen may be detected. Detection is generally achieved by the addition of a second antibody specific for the target protein, that is linked to a detectable label. This type of ELISA is a simple “sandwich ELISA.” Detection also may be achieved by the addition of a second antibody, followed by the addition of a third antibody that has binding affinity for the second antibody, with the third antibody being linked to a detectable label.

In another exemplary ELISA, the samples suspected of containing the prostate cancer marker antigen are immobilized onto the well surface and then contacted with the anti-biomarker antibodies of the invention. After binding and washing to remove non-specifically bound immunecomplexes, the bound antigen is detected. Where the initial antibodies are linked to a detectable label, the immunecomplexes may be detected directly. Again, the immunecomplexes may be detected using a second antibody that has binding affinity for the first antibody, with the second antibody being linked to a detectable label.

Irrespective of the format employed, ELISAs have certain features in common, such as coating incubating or binding washing to remove non-specifically bound species, and detecting the bound immunecomplexes. These are described as follows.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the control human prostate, cancer and/or clinical or biological sample to be tested under conditions effective to allow immunecomplex (antigen/antibody) formation. Detection of the immunecomplex then requires a labeled secondary binding ligand or antibody, or a secondary binding ligand or antibody in conjunction with a labeled tertiary antibody or third binding ligand.

The phrase “under conditions effective to allow immunecomplex (antigen/antibody) formation” means that the conditions preferably include diluting the antigens and antibodies with solutions such as BSA, bovine gamma globulin (BGG) and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 h, at temperatures preferably on the order of 25 to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immunecomplexes between the test sample and the originally bound material, and subsequent washing, the occurrence of even minute amounts of immunecomplexes may be determined.

To provide a detecting means, the second or third antibody will have an associated label to allow detection. Preferably, this will be an enzyme that will generate color development upon incubating with an appropriate chromogenic substrate. Thus, for example, one will desire to contact and incubate the first or second immunecomplex with a urease, glucose oxidase, alkaline phosphatase or hydrogen peroxidase-conjugated antibody for a period of time and under conditions that favor the development of further immunecomplex formation (e.g., incubation for 2 h at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple. Quantitation is then achieved by measuring the degree of color generation, e.g., using a visible spectra spectrophotometer.

The protein biomarkers of the invention can also be measured, quantitated, detected, and otherwise analyzed using protein mass spectrometry methods and instrumentation. Protein mass spectrometry refers to the application of mass spectrometry to the study of proteins. Although not intending to be limiting, two approaches are typically used for characterizing proteins using mass spectrometry. In the first, intact proteins are ionized and then introduced to a mass analyzer. This approach is referred to as “top-down” strategy of protein analysis. The two primary methods for ionization of whole proteins are electrospray ionization (ESI) and matrix-assisted laser desorption/ionization (MALDI). In the second approach, proteins are enzymatically digested into smaller peptides using a protease such as trypsin. Subsequently these peptides are introduced into the mass spectrometer and identified by peptide mass fingerprinting or tandem mass spectrometry. Hence, this latter approach (also called “bottom-up” proteomics) uses identification at the peptide level to infer the existence of proteins.

Whole protein mass analysis of the biomarkers of the invention can be conducted using time-of-flight (TOF) MS, or Fourier transform ion cyclotron resonance (FT-ICR). These two types of instruments are useful because of their wide mass range, and in the case of FT-ICR, its high mass accuracy. The most widely used instruments for peptide mass analysis are the MALDI time-of-flight instruments as they permit the acquisition of peptide mass fingerprints (PMFs) at high pace (1 PMF can be analyzed in approx. 10 sec). Multiple stage quadrupole-time-of-flight and the quadrupole ion trap also find use in this application.

The protein biomarkers of the invention can also be measured in complex mixtures of proteins and molecules that co-exist in a biological medium or sample, however, fractionation of the sample may be required and is contemplated herein. It will be appreciated that ionization of complex mixtures of proteins can result in situation where the more abundant proteins have a tendency to “drown” or suppress signals from less abundant proteins in the same sample. In addition, the mass spectrum from a complex mixture can be difficult to interpret because of the overwhelming number of mixture components. Fractionation can be used to first separate any complex mixture of proteins prior to mass spectrometry analysis. Two methods are widely used to fractionate proteins, or their peptide products from an enzymatic digestion. The first method fractionates whole proteins and is called two-dimensional gel electrophoresis. The second method, high performance liquid chromatography (LC or HPLC) is used to fractionate peptides after enzymatic digestion. In some situations, it may be desirable to combine both of these techniques. Any other suitable methods known in the art for fractionating protein mixtures are also contemplated herein.

Gel spots identified on a 2D Gel are usually attributable to one protein. If the identity of the protein is desired, usually the method of in-gel digestion is applied, where the protein spot of interest is excised, and digested proteolytically. The peptide masses resulting from the digestion can be determined by mass spectrometry using peptide mass fingerprinting. If this information does not allow unequivocal identification of the protein, its peptides can be subject to tandem mass spectrometry for de novo sequencing.

Characterization of protein mixtures using HPLC/MS may also be referred to in the art as “shotgun proteomics” and MuDPIT (Multi-Dimensional Protein Identification Technology). A peptide mixture that results from digestion of a protein mixture is fractionated by one or two steps of liquid chromatography (LC). The eluent from the chromatography stage can be either directly introduced to the mass spectrometer through electrospray ionization, or laid down on a series of small spots for later mass analysis using MALDI.

The protein biomarkers of the present invention can be identified using MS using a variety of techniques, all of which are contemplated herein. Peptide mass fingerprinting uses the masses of proteolytic peptides as input to a search of a database of predicted masses that would arise from digestion of a list of known proteins. If a protein sequence in the reference list gives rise to a significant number of predicted masses that match the experimental values, there is some evidence that this protein was present in the original sample. It will be further appreciated that the development of methods and instrumentation for automated, data-dependent electrospray ionization (ESI) tandem mass spectrometry (MS/MS) in conjunction with microcapillary liquid chromatography (LC) and database searching has significantly increased the sensitivity and speed of the identification of gel-separated proteins. Microcapillary LC-MS/MS has been used successfully for the large-scale identification of individual proteins directly from mixtures without gel electrophoretic separation (Link et al., 1999; Opitek et al., 1997).

Several recent methods allow for the quantitation of proteins by mass spectrometry. For example, stable (e.g., non-radioactive) heavier isotopes of carbon (¹³C) or nitrogen (¹⁵N) can be incorporated into one sample while the other one can be labeled with corresponding light isotopes (e.g. ¹²C and ¹⁴N). The two samples are mixed before the analysis. Peptides derived from the different samples can be distinguished due to their mass difference. The ratio of their peak intensities corresponds to the relative abundance ratio of the peptides (and proteins). The most popular methods for isotope labeling are SILAC (stable isotope labeling by amino acids in cell culture), trypsin-catalyzed ¹⁸O labeling, ICAT (isotope coded affinity tagging), iTRAQ (isobaric tags for relative and absolute quantitation). “Semi-quantitative” mass spectrometry can be performed without labeling of samples. Typically, this is done with MALDI analysis (in linear mode). The peak intensity, or the peak area, from individual molecules (typically proteins) is here correlated to the amount of protein in the sample. However, the individual signal depends on the primary structure of the protein, on the complexity of the sample, and on the settings of the instrument. Other types of “label-free” quantitative mass spectrometry, uses the spectral counts (or peptide counts) of digested proteins as a means for determining relative protein amounts.

In one embodiment, any one or more of the protein markers of the invention can be identified and quantified from a complex biological sample using mass spectroscopy in accordance with the following exemplary method, which is not intended to limit the invention or the use of other mass spectrometry-based methods.

In the first step of this embodiment, (A) a biological sample, e.g., a biological sample suspected of having prostate cancer, which comprises a complex mixture of protein (including at least one biomarker of interest) is fragmented and labeled with a stable isotope X. (B) Next, a known amount of an internal standard is added to the biological sample, wherein the internal standard is prepared by fragmenting a standard protein that is identical to the at least one target biomarker of interest, and labeled with a stable isotope Y. (C) This sample obtained is then introduced in an LC-MS/MS device, and multiple reaction monitoring (MRM) analysis is performed using MRM transitions selected for the internal standard to obtain an MRM chromatogram. (D) The MRM chromatogram is then viewed to identify a target peptide biomarker derived from the biological sample that shows the same retention time as a peptide derived from the internal standard (an internal standard peptide), and quantifying the target protein biomarker in the test sample by comparing the peak area of the internal standard peptide with the peak area of the target peptide biomarker.

Any suitable biological sample may be used as a starting point for LC-MS/MS/MRM analysis, including biological samples derived blood, urine, saliva, hair, cells, cell tissues, biopsy materials, and treated products thereof; and protein-containing samples prepared by gene recombination techniques.

Each of the above steps (A) to (D) is described further below.

Step (A) (Fragmentation and Labeling). In step (A), the target protein biomarker is fragmented to a collection of peptides, which is subsequently labeled with a stable isotope X. To fragment the target protein, for example, methods of digesting the target protein with a proteolytic enzyme (protease) such as trypsin, and chemical cleavage methods, such as a method using cyanogen bromide, can be used. Digestion by protease is preferable. It is known that a given mole quantity of protein produces the same mole quantity for each tryptic peptide cleavage product if the proteolytic digest is allowed to proceed to completion. Thus, determining the mole quantity of tryptic peptide to a given protein allows determination of the mole quantity of the original protein in the sample. Absolute quantification of the target protein can be accomplished by determining the absolute amount of the target protein-derived peptides contained in the protease digestion (collection of peptides). Accordingly, in order to allow the proteolytic digest to proceed to completion, reduction and alkylation treatments are preferably performed before protease digestion with trypsin to reduce and alkylate the disulfide bonds contained in the target protein.

Subsequently, the obtained digest (collection of peptides, comprising peptides of the target biomarker in the biological sample) is subjected to labeling with a stable isotope X. Examples of stable isotopes X include ¹H and ²H for hydrogen atoms, ¹²C and ¹³C for carbon atoms, and ¹⁴N and ¹⁵N for nitrogen atoms. Any isotope can be suitably selected therefrom. Labeling by a stable isotope X can be performed by reacting the digest (collection of peptides) with a reagent containing the stable isotope. Preferable examples of such reagents that are commercially available include mTRAQ (registered trademark) (produced by Applied Biosystems), which is an amine-specific stable isotope reagent kit. mTRAQ is composed of 2 or 3 types of reagents (mTRAQ-light and mTRAQ-heavy; or mTRAQ-DO, mTRAQ-D4, and mTRAQ-D8) that have a constant mass difference therebetween as a result of isotope-labeling, and that are bound to the N-terminus of a peptide or the primary amine of a lysine residue.

Step (B) (Addition of the Internal Standard). In step (B), a known amount of an internal standard is added to the sample obtained in step (A). The internal standard used herein is a digest (collection of peptides) obtained by fragmenting a protein (standard protein) consisting of the same amino acid sequence as the target protein (target biomarker) to be measured, and labeling the obtained digest (collection of peptides) with a stable isotope Y. The fragmentation treatment can be performed in the same manner as above for the target protein. Labeling with a stable isotope Y can also be performed in the same manner as above for the target protein. However, the stable isotope Y used herein must be an isotope that has a mass different from that of the stable isotope X used for labeling the target protein digest. For example, in the case of using the aforementioned mTRAQ (registered trademark) (produced by Applied Biosystems), when mTRAQ-light is used to label a target protein digest, mTRAQ-heavy should be used to label a standard protein digest.

Step (C) (LC-MS/MS and MRM Analysis). In step (C), the sample obtained in step (B) is first placed in an LC-MS/MS device, and then multiple reaction monitoring (MRM) analysis is performed using MRM transitions selected for the internal standard. By LC (liquid chromatography) using the LC-MS/MS device, the sample (collection of peptides labeled with a stable isotope) obtained in step (B) is separated first by one-dimensional or multi-dimensional high-performance liquid chromatography. Specific examples of such liquid chromatography include cation exchange chromatography, in which separation is conducted by utilizing electric charge difference between peptides; and reversed-phase chromatography, in which separation is conducted by utilizing hydrophobicity difference between peptides. Both of these methods may be used in combination.

Subsequently, each of the separated peptides is subjected to tandem mass spectrometry by using a tandem mass spectrometer (MS/MS spectrometer) comprising two mass spectrometers connected in series. The use of such a mass spectrometer enables the detection of several fmol levels of a target protein. Furthermore, MS/MS analysis enables the analysis of internal sequence information on peptides, thus enabling identification without false positives. Other types of MS analyzers may also be used, including magnetic sector mass spectrometers (Sector MS), quadrupole mass spectrometers (QMS), time-of-flight mass spectrometers (TOFMS), and Fourier transform ion cyclotron resonance mass spectrometers (FT-ICRMS), and combinations of these analyzers.

Subsequently, the obtained data are put through a search engine to perform a spectral assignment and to list the peptides experimentally detected for each protein. The detected peptides are preferably grouped for each protein, and preferably at least three fragments having an m/z value larger than that of the precursor ion and at least three fragments with an m/z value of preferably, 500 or more are selected from each MS/MS spectrum in descending order of signal strength on the spectrum. From these, two or more fragments are selected in descending order of strength, and the average of the strength is defined as the expected sensitivity of the MRR transitions. When a plurality of peptides is detected from one protein, at least two peptides with the highest sensitivity are selected as standard peptides using the expected sensitivity as an index.

Step (D) (Quantification of the Target Protein in the Test Sample). Step (D) comprises identifying, in the MRM chromatogram detected in step (C), a peptide derived from the target protein (a target biomarker of interest) that shows the same retention time as a peptide derived from the internal standard (an internal standard peptide), and quantifying the target protein in the test sample by comparing the peak area of the internal standard peptide with the peak area of the target peptide. The target protein can be quantified by utilizing a calibration curve of the standard protein prepared beforehand.

The calibration curve can be prepared by the following method. First, a recombinant protein consisting of an amino acid sequence that is identical to that of the target biomarker protein is digested with a protease such as trypsin, as described above. Subsequently, precursor-fragment transition selection standards (PFTS) of a known concentration are individually labeled with two different types of stable isotopes (i.e., one is labeled with a stable isomer used to label an internal standard peptide (labeled with IS), whereas the other is labeled with a stable isomer used to label a target peptide (labeled with T). A plurality of samples are produced by blending a certain amount of the IS-labeled PTFS with various concentrations of the T-labeled PTFS. These samples are placed in the aforementioned LC-MS/MS device to perform MRM analysis. The area ratio of the T-labeled PTFS to the IS-labeled PTFS (T-labeled PTFS/IS-labeled PTFS) on the obtained MRM chromatogram is plotted against the amount of the T-labeled PTFS to prepare a calibration curve. The absolute amount of the target protein contained in the test sample can be calculated by reference to the calibration curve.

3. Detection of Nucleic Acids Corresponding to Protein Markers

In certain embodiments, the invention involves the detection of nucleic acid biomarkers, e.g., the corresponding genes or mRNA of the protein markers of the invention, e.g., Tables 13-18.

In various embodiments, the diagnostic/prognostic methods of the present invention generally involve the determination of expression levels of a set of genes in a biological sample. Determination of gene expression levels in the practice of the inventive methods may be performed by any suitable method. For example, determination of gene expression levels may be performed by detecting the expression of mRNA expressed from the genes of interest and/or by detecting the expression of a polypeptide encoded by the genes.

For detecting nucleic acids encoding biomarkers of the invention, any suitable method can be used, including, but not limited to, Southern blot analysis, Northern blot analysis, polymerase chain reaction (PCR) (see, for example, U.S. Pat. Nos. 4,683,195; 4,683,202, and 6,040,166; “PCR Protocols: A Guide to Methods and Applications”, Innis et al. (Eds), 1990, Academic Press: New York), reverse transcriptase PCR (RT-PCT), anchored PCR, competitive PCR (see, for example, U.S. Pat. No. 5,747,251), rapid amplification of cDNA ends (RACE) (see, for example, “Gene Cloning and Analysis: Current Innovations, 1997, pp. 99-115); ligase chain reaction (LCR) (see, for example, EP 01 320 308), one-sided PCR (Ohara et al., Proc. Natl. Acad. Sci., 1989, 86: 5673-5677), in situ hybridization, Taqman-based assays (Holland et al., Proc. Natl. Acad. Sci., 1991, 88: 7276-7280), differential display (see, for example, Liang et al., Nucl. Acid. Res., 1993, 21: 3269-3275) and other RNA fingerprinting techniques, nucleic acid sequence based amplification (NASBA) and other transcription based amplification systems (see, for example, U.S. Pat. Nos. 5,409,818 and 5,554,527), Qbeta Replicase, Strand Displacement Amplification (SDA), Repair Chain Reaction (RCR), nuclease protection assays, subtraction-based methods, Rapid-Scan®, etc.

In other embodiments, gene expression levels of biomarkers of interest may be determined by amplifying complementary DNA (cDNA) or complementary RNA (cRNA) produced from mRNA and analyzing it using a microarray. A number of different array configurations and methods of their production are known to those skilled in the art (see, for example, U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637). Microarray technology allows for the measurement of the steady-state mRNA level of a large number of genes simultaneously. Microarrays currently in wide use include cDNA arrays and oligonucleotide arrays. Analyses using microarrays are generally based on measurements of the intensity of the signal received from a labeled probe used to detect a cDNA sequence from the sample that hybridizes to a nucleic acid probe immobilized at a known location on the microarray (see, for example, U.S. Pat. Nos. 6,004,755; 6,218,114; 6,218,122; and 6,271,002). Array-based gene expression methods are known in the art and have been described in numerous scientific publications as well as in patents (see, for example, M. Schena et al., Science, 1995, 270: 467-470; M. Schena et al., Proc. Natl. Acad. Sci. USA 1996, 93: 10614-10619; J. J. Chen et al., Genomics, 1998, 51: 313-324; U.S. Pat. Nos. 5,143,854; 5,445,934; 5,807,522; 5,837,832; 6,040,138; 6,045,996; 6,284,460; and 6,607,885).

Nucleic acid used as a template for amplification can be isolated from cells contained in the biological sample, according to standard methodologies. (Sambrook et al., 1989) The nucleic acid may be genomic DNA or fractionated or whole cell RNA. Where RNA is used, it may be desired to convert the RNA to a complementary cDNA. In one embodiment, the RNA is whole cell RNA and is used directly as the template for amplification.

Pairs of primers that selectively hybridize to nucleic acids corresponding to any of the prostate cancer biomarker nucleotide sequences identified herein are contacted with the isolated nucleic acid under conditions that permit selective hybridization. Once hybridized, the nucleic acid:primer complex is contacted with one or more enzymes that facilitate template-dependent nucleic acid synthesis. Multiple rounds of amplification, also referred to as “cycles,” are conducted until a sufficient amount of amplification product is produced. Next, the amplification product is detected. In certain applications, the detection may be performed by visual means. Alternatively, the detection may involve indirect identification of the product via chemiluminescence, radioactive scintigraphy of incorporated radiolabel or fluorescent label or even via a system using electrical or thermal impulse signals (Afflymax technology; Bellus, 1994). Following detection, one may compare the results seen in a given patient with a statistically significant reference group of normal patients and prostate, cancer patients. In this way, it is possible to correlate the amount of nucleic acid detected with various clinical states.

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences may be employed. Primers may be provided in double-stranded or single-stranded form, although the single-stranded form is preferred.

A number of template dependent processes are available to amplify the nucleic acid sequences present in a given template sample. One of the best known amplification methods is the polymerase chain reaction (referred to as PCR) which is described in detail in U.S. Pat. Nos. 4,683,195, 4,683,202 and 4,800,159, and in Innis et al., 1990, each of which is incorporated herein by reference in its entirety.

In PCR, two primer sequences are prepared which are complementary to regions on opposite complementary strands of the target nucleic acid sequence. An excess of deoxynucleoside triphosphates are added to a reaction mixture along with a DNA polymerase, e.g., Taq polymerase. If the target nucleic acid sequence is present in a sample, the primers will bind to the target nucleic acid and the polymerase will cause the primers to be extended along the target nucleic acid sequence by adding on nucleotides. By raising and lowering the temperature of the reaction mixture, the extended primers will dissociate from the target nucleic acid to form reaction products, excess primers will bind to the target nucleic acid and to the reaction products and the process is repeated.

A reverse transcriptase PCR amplification procedure may be performed in order to quantify the amount of mRNA amplified. Methods of reverse transcribing RNA into cDNA are well known and described in Sambrook et al., 1989. Alternative methods for reverse transcription utilize thermostable DNA polymerases. These methods are described in WO 90/07641 filed Dec. 21, 1990. Polymerase chain reaction methodologies are well known in the art.

Another method for amplification is the ligase chain reaction (“LCR”), disclosed in European Application No. 320 308, incorporated herein by reference in its entirely. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, described in PCT Application No. PCT/US87/00880, also may be used as still another amplification method in the present invention. In this method, a replicative sequence of RNA which has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[α-thio]-triphosphates in one strand of a restriction site also may be useful in the amplification of nucleic acids in the present invention. Walker et al. (1992), incorporated herein by reference in its entirety.

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases may be added as biotinylated derivatives for easy detection. A similar approach is used in SDA. Target specific sequences also may be detected using a cyclic probe reaction (CPR). In CPR, a probe having 3′ and 5′ sequences of non-specific DNA and a middle sequence of specific RNA is hybridized to DNA which is present in a sample. Upon hybridization, the reaction is treated with RNase H, and the products of the probe identified as distinctive products which are released after digestion. The original template is annealed to another cycling probe and the reaction is repeated.

Still other amplification methods described in GB Application No. 2 202 328, and in PCT Application No. PCT/US89/01025, each of which is incorporated herein by reference in its entirety, may be used in accordance with the present invention. In the former application, “modified” primers are used in a PCR like, template and enzyme dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other contemplated nucleic acid amplification procedures include transcription-based amplification systems (TAS), including nucleic acid sequence based amplification (NASBA) and 3SR. Kwoh et al. (1989); Gingeras et al., PCT Application WO 88/10315, incorporated herein by reference in their entirety. In NASBA, the nucleic acids may be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by a polymerase such as T7 or SP6. In an isothermal cyclic reaction, the RNA's are reverse transcribed into double stranded DNA, and transcribed once against with a polymerase such as T7 or SP6. The resulting products, whether truncated or complete, indicate target specific sequences.

Davey et al., European Application No. 329 822 (incorporated herein by reference in its entirely) disclose a nucleic acid amplification process involving cyclically synthesizing single-stranded RNA (“ssRNA”), ssDNA, and double-stranded DNA (dsDNA), which may be used in accordance with the present invention. The ssRNA is a first template for a first primer oligonucleotide, which is elongated by reverse transcriptase (RNA-dependent DNA polymerase). The RNA is then removed from the resulting DNA RNA duplex by the action of ribonuclease H(RNase H, an RNase specific for RNA in duplex with either DNA or RNA). The resultant ssDNA is a second template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase 1), resulting in a double-stranded DNA (“dsDNA”) molecule, having a sequence identical to that of the original RNA between the primers and having additionally, at one end, a promoter sequence. This promoter sequence may be used by the appropriate RNA polymerase to make many RNA copies of the DNA. These copies may then re-enter the cycle leading to very swift amplification. With proper choice of enzymes, this amplification may be done isothermally without addition of enzymes at each cycle. Because of the cyclical nature of this process, the starting sequence may be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR.” Frohman (1990) and Ohara et al. (1989), each herein incorporated by reference in their entirety.

Methods based on ligation of two (or more) oligonucleotides in the presence of nucleic acid having the sequence of the resulting “di-oligonucleotide”, thereby amplifying the di-oligonucleotide, also may be used in the amplification step of the present invention. Wu et al. (1989), incorporated herein by reference in its entirety.

Oligonucleotide probes or primers of the present invention may be of any suitable length, depending on the particular assay format and the particular needs and targeted sequences employed. In a preferred embodiment, the oligonucleotide probes or primers are at least 10 nucleotides in length (preferably, 10, 11, 12, 13, 14, 15, 16, 17, 18, 19, 20, 21, 22, 23, 24, 25, 26, 27, 28, 29, 30, 31, 32 . . . ) and they may be adapted to be especially suited for a chosen nucleic acid amplification system and/or hybridization system used. Longer probes and primers are also within the scope of the present invention as well known in the art. Primers having more than 30, more than 40, more than 50 nucleotides and probes having more than 100, more than 200, more than 300, more than 500 more than 800 and more than 1000 nucleotides in length are also covered by the present invention. Of course, longer primers have the disadvantage of being more expensive and thus, primers having between 12 and 30 nucleotides in length are usually designed and used in the art. As well known in the art, probes ranging from 10 to more than 2000 nucleotides in length can be used in the methods of the present invention. As for the % of identity described above, non-specifically described sizes of probes and primers (e.g., 16, 17, 31, 24, 39, 350, 450, 550, 900, 1240 nucleotides, . . . ) are also within the scope of the present invention. In one embodiment, the oligonucleotide probes or primers of the present invention specifically hybridize with a filamin A RNA (or its complementary sequence) or a filamin A mRNA. More preferably, the filamin A primers and probes will be chosen to detect a filamin A RNA which is associated with prostate cancer.

In other embodiments, the detection means can utilize a hybridization technique, e.g., where a specific primer or probe is selected to anneal to a target biomarker of interest, e.g., filamin A, and thereafter detection of selective hybridization is made. As commonly known in the art, the oligonucleotide probes and primers can be designed by taking into consideration the melting point of hybridization thereof with its targeted sequence (see below and in Sambrook et al., 1989, Molecular Cloning—A Laboratory Manual, 2nd Edition, CSH Laboratories; Ausubel et al., 1994, in Current Protocols in Molecular Biology, John Wiley & Sons Inc., N.Y.).

To enable hybridization to occur under the assay conditions of the present invention, oligonucleotide primers and probes should comprise an oligonucleotide sequence that has at least 70% (at least 71%, 72%, 73%, 74%), preferably at least 75% (75%, 76%, 77%, 78%, 79%, 80%, 81%, 82%, 83%, 84%, 85%, 86%, 87%, 88%, 89%) and more preferably at least 90% (90%, 91%, 92%, 93%, 94%, 95%, 96%, 97%, 98%, 99%, 100%) identity to a portion of a filamin A or polynucleotide of another biomarker of the invention. Probes and primers of the present invention are those that hybridize under stringent hybridization conditions and those that hybridize to biomarker homologs of the invention under at least moderately stringent conditions. In certain embodiments probes and primers of the present invention have complete sequence identity to the biomarkers of the invention (filamin A, gene sequences (e.g., cDNA or mRNA). It should be understood that other probes and primers could be easily designed and used in the present invention based on the biomarkers of the invention disclosed herein by using methods of computer alignment and sequence analysis known in the art (cf. Molecular Cloning: A Laboratory Manual, Third Edition, edited by Cold Spring Harbor Laboratory, 2000).

4. Antibodies and Labels

In some embodiments, the invention provides methods and compositions that include labels for the highly sensitive detection and quantitation of the markers of the invention. One skilled in the art will recognize that many strategies can be used for labeling target molecules to enable their detection or discrimination in a mixture of particles. The labels may be attached by any known means, including methods that utilize non-specific or specific interactions of label and target. Labels may provide a detectable signal or affect the mobility of the particle in an electric field. In addition, labeling can be accomplished directly or through binding partners.

In some embodiments, the label comprises a binding partner that binds to the biomarker of interest, where the binding partner is attached to a fluorescent moiety. The compositions and methods of the invention may utilize highly fluorescent moieties, e.g., a moiety capable of emitting at least about 200 photons when simulated by a laser emitting light at the excitation wavelength of the moiety, wherein the laser is focused on a spot not less than about 5 microns in diameter that contains the moiety, and wherein the total energy directed at the spot by the laser is no more than about 3 microJoules. Moieties suitable for the compositions and methods of the invention are described in more detail below.

In some embodiments, the invention provides a label for detecting a biological molecule comprising a binding partner for the biological molecule that is attached to a fluorescent moiety, wherein the fluorescent moiety is capable of emitting at least about 200 photons when simulated by a laser emitting light at the excitation wavelength of the moiety, wherein the laser is focused on a spot not less than about 5 microns in diameter that contains the moiety, and wherein the total energy directed at the spot by the laser is no more than about 3 microJoules. In some embodiments, the moiety comprises a plurality of fluorescent entities, e.g., about 2 to 4, 2 to 5, 2 to 6, 2 to 7, 2 to 8, 2 to 9, 2 to 10, or about 3 to 5, 3 to 6, 3 to 7, 3 to 8, 3 to 9, or 3 to 10 fluorescent entities. In some embodiments, the moiety comprises about 2 to 4 fluorescent entities. In some embodiments, the biological molecule is a protein or a small molecule. In some embodiments, the biological molecule is a protein. The fluorescent entities can be fluorescent dye molecules. In some embodiments, the fluorescent dye molecules comprise at least one substituted indolium ring system in which the substituent on the 3-carbon of the indolium ring contains a chemically reactive group or a conjugated substance. In some embodiments, the dye molecules are Alexa Fluor molecules selected from the group consisting of Alexa Fluor 488, Alexa Fluor 532, Alexa Fluor 647, Alexa Fluor 680 or Alexa Fluor 700. In some embodiments, the dye molecules are Alexa Fluor molecules selected from the group consisting of Alexa Fluor 488, Alexa Fluor 532, Alexa Fluor 680 or Alexa Fluor 700. In some embodiments, the dye molecules are Alexa Fluor 647 dye molecules. In some embodiments, the dye molecules comprise a first type and a second type of dye molecules, e.g., two different Alexa Fluor molecules, e.g., where the first type and second type of dye molecules have different emission spectra. The ratio of the number of first type to second type of dye molecule can be, e.g., 4 to 1, 3 to 1, 2 to 1, 1 to 1, 1 to 2, 1 to 3 or 1 to 4. The binding partner can be, e.g., an antibody.

In some embodiments, the invention provides a label for the detection of a biological marker of the invention, wherein the label comprises a binding partner for the marker and a fluorescent moiety, wherein the fluorescent moiety is capable of emitting at least about 200 photons when simulated by a laser emitting light at the excitation wavelength of the moiety, wherein the laser is focused on a spot not less than about 5 microns in diameter that contains the moiety, and wherein the total energy directed at the spot by the laser is no more than about 3 microJoules. In some embodiments, the fluorescent moiety comprises a fluorescent molecule. In some embodiments, the fluorescent moiety comprises a plurality of fluorescent molecules, e.g., about 2 to 10, 2 to 8, 2 to 6, 2 to 4, 3 to 10, 3 to 8, or 3 to 6 fluorescent molecules. In some embodiments, the label comprises about 2 to 4 fluorescent molecules. In some embodiments, the fluorescent dye molecules comprise at least one substituted indolium ring system in which the substituent on the 3-carbon of the indolium ring contains a chemically reactive group or a conjugated substance. In some embodiments, the fluorescent molecules are selected from the group consisting of Alexa Fluor 488, Alexa Fluor 532, Alexa Fluor 647, Alexa Fluor 680 or Alexa Fluor 700. In some embodiments, the fluorescent molecules are selected from the group consisting of Alexa Fluor 488, Alexa Fluor 532, Alexa Fluor 680 or Alexa Fluor 700. In some embodiments, the fluorescent molecules are Alexa Fluor 647 molecules. In some embodiments, the binding partner comprises an antibody. In some embodiments, the antibody is a monoclonal antibody. In other embodiments, the antibody is a polyclonal antibody.

The term “antibody,” as used herein, is a broad term and is used in its ordinary sense, including, without limitation, to refer to naturally occurring antibodies as well as non-naturally occurring antibodies, including for example, single chain antibodies, chimeric, bifunctional and humanized antibodies, as well as antigen-binding fragments thereof Δn “antigen-binding fragment” of an antibody refers to the part of the antibody that participates in antigen binding. The antigen binding site is formed by amino acid residues of the N-terminal variable (“V”) regions of the heavy (“H”) and light (“L”) chains. It will be appreciated that the choice of epitope or region of the molecule to which the antibody is raised will determine its specificity, e.g., for various forms of the molecule, if present, or for total (e.g., all, or substantially all of the molecule).

Methods for producing antibodies are well-established. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)). Monoclonal and polyclonal antibodies to molecules, e.g., proteins, and markers also commercially available (R and D Systems, Minneapolis, Minn.; HyTest, HyTest Ltd., Turku Finland; Abcam Inc., Cambridge, Mass., USA, Life Diagnostics, Inc., West Chester, Pa., USA; Fitzgerald Industries International, Inc., Concord, Mass. 01742-3049 USA; BiosPacific, Emeryville, Calif.).

In some embodiments, the antibody is a polyclonal antibody. In other embodiments, the antibody is a monoclonal antibody.

Antibodies may be prepared by any of a variety of techniques known to those of ordinary skill in the art (see, for example, Harlow and Lane, Antibodies: A Laboratory Manual, Cold Spring Harbor Laboratory, 1988). In general, antibodies can be produced by cell culture techniques, including the generation of monoclonal antibodies as described herein, or via transfection of antibody genes into suitable bacterial or mammalian cell hosts, in order to allow for the production of recombinant antibodies.

Monoclonal antibodies may be prepared using hybridoma methods, such as the technique of Kohler and Milstein (Eur. J. Immunol. 6:511-519, 1976), and improvements thereto. These methods involve the preparation of immortal cell lines capable of producing antibodies having the desired specificity. Monoclonal antibodies may also be made by recombinant DNA methods, such as those described in U.S. Pat. No. 4,816,567. DNA encoding antibodies employed in the disclosed methods may be isolated and sequenced using conventional procedures. Recombinant antibodies, antibody fragments, and/or fusions thereof, can be expressed in vitro or in prokaryotic cells (e.g. bacteria) or eukaryotic cells (e.g. yeast, insect or mammalian cells) and further purified as necessary using well known methods.

More particularly, monoclonal antibodies (MAbs) may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified expressed protein, polypeptide or peptide. The immunizing composition is administered in a manner effective to stimulate antibody producing cells. The methods for generating monoclonal antibodies (MAbs) generally begin along the same lines as those for preparing polyclonal antibodies. Rodents such as mice and rats are preferred animals, however, the use of rabbit, sheep or frog cells is also possible. The use of rats may provide certain advantages (Goding, 1986, pp. 60-61), but mice are preferred, with the BALB/c mouse being most preferred as this is most routinely used and generally gives a higher percentage of stable fusions.

The animals are injected with antigen as described above. The antigen may be coupled to carrier molecules such as keyhole limpet hemocyanin if necessary. The antigen would typically be mixed with adjuvant, such as Freund's complete or incomplete adjuvant. Booster injections with the same antigen would occur at approximately two-week intervals. Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of the animal with the highest antibody titer will be removed and the spleen lymphocytes obtained by homogenizing the spleen with a syringe.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing have high fusion efficiency, and enzyme deficiencies that render then incapable of growing in certain selective media which support the growth of only the desired fused cells (hybridomas).

The selected hybridomas would then be serially diluted and cloned into individual antibody-producing cell lines, which clones may then be propagated indefinitely to provide MAbs. The cell lines may be exploited for MAb production in two basic ways. A sample of the hybridoma may be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, may then be tapped to provide MAbs in high concentration. The individual cell lines also may be cultured in vitro, where the MAbs are naturally secreted into the culture medium from which they may be readily obtained in high concentrations. MAbs produced by either means may be further purified, if desired, using filtration, centrifugation and various chromatographic methods such as HPLC or affinity chromatography.

Large amounts of the monoclonal antibodies of the present invention also may be obtained by multiplying hybridoma cells in vivo. Cell clones are injected into mammals which are histocompatible with the parent cells, e.g., syngeneic mice, to cause growth of antibody-producing tumors. Optionally, the animals are primed with a hydrocarbon, especially oils such as pristane (tetramethylpentadecane) prior to injection.

In accordance with the present invention, fragments of the monoclonal antibody of the invention may be obtained from the monoclonal antibody produced as described above, by methods which include digestion with enzymes such as pepsin or papain and/or cleavage of disulfide bonds by chemical reduction. Alternatively, monoclonal antibody fragments encompassed by the present invention may be synthesized using an automated peptide synthesizer.

Antibodies may also be derived from a recombinant antibody library that is based on amino acid sequences that have been designed in silico and encoded by polynucleotides that are synthetically generated. Methods for designing and obtaining in silico-created sequences are known in the art (Knappik et al., J. Mol. Biol. 296:254:57-86, 2000; Krebs et al., J. Immunol. Methods 254:67-84, 2001; U.S. Pat. No. 6,300,064).

Digestion of antibodies to produce antigen-binding fragments thereof can be performed using techniques well known in the art. For example, the proteolytic enzyme papain preferentially cleaves IgG molecules to yield several fragments, two of which (the “F(ab)” fragments) each comprise a covalent heterodimer that includes an intact antigen-binding site. The enzyme pepsin is able to cleave IgG molecules to provide several fragments, including the “F(ab′).sub.2” fragment, which comprises both antigen-binding sites. “Fv” fragments can be produced by preferential proteolytic cleavage of an IgM, IgG or IgA immunoglobulin molecule, but are more commonly derived using recombinant techniques known in the art. The Fv fragment includes a non-covalent V.sub.H::V.sub.L heterodimer including an antigen-binding site which retains much of the antigen recognition and binding capabilities of the native antibody molecule (Inbar et al., Proc. Natl. Acad. Sci. USA 69:2659-2662 (1972); Hochman et al., Biochem. 15:2706-2710 (1976); and Ehrlich et al., Biochem. 19:4091-4096 (1980)).

Antibody fragments that specifically bind to the protein biomarkers disclosed herein can also be isolated from a library of scFvs using known techniques, such as those described in U.S. Pat. No. 5,885,793.

A wide variety of expression systems are available in the art for the production of antibody fragments, including Fab fragments, scFv, VL and VHs. For example, expression systems of both prokaryotic and eukaryotic origin may be used for the large-scale production of antibody fragments. Particularly advantageous are expression systems that permit the secretion of large amounts of antibody fragments into the culture medium. Eukaryotic expression systems for large-scale production of antibody fragments and antibody fusion proteins have been described that are based on mammalian cells, insect cells, plants, transgenic animals, and lower eukaryotes. For example, the cost-effective, large-scale production of antibody fragments can be achieved in yeast fermentation systems. Large-scale fermentation of these organisms is well known in the art and is currently used for bulk production of several recombinant proteins.

Antibodies that bind to the protein biomarkers employed in the present methods are, in some cases, available commercially or can be obtained without undue experimentation.

In still other embodiments, particularly where oligonucleotides are used as binding partners to detect and hybridize to mRNA biomarkers or other nucleic acid based biomarkers, the binding partners (e.g., oligonucleotides) can comprise a label, e.g., a fluorescent moiety or dye. In addition, any binding partner of the invention, e.g., an antibody, can also be labeled with a fluorescent moiety. The fluorescence of the moiety will be sufficient to allow detection in a single molecule detector, such as the single molecule detectors described herein. A “fluorescent moiety,” as that term is used herein, includes one or more fluorescent entities whose total fluorescence is such that the moiety may be detected in the single molecule detectors described herein. Thus, a fluorescent moiety may comprise a single entity (e.g., a Quantum Dot or fluorescent molecule) or a plurality of entities (e.g., a plurality of fluorescent molecules). It will be appreciated that when “moiety,” as that term is used herein, refers to a group of fluorescent entities, e.g., a plurality of fluorescent dye molecules, each individual entity may be attached to the binding partner separately or the entities may be attached together, as long as the entities as a group provide sufficient fluorescence to be detected.

Typically, the fluorescence of the moiety involves a combination of quantum efficiency and lack of photobleaching sufficient that the moiety is detectable above background levels in a single molecule detector, with the consistency necessary for the desired limit of detection, accuracy, and precision of the assay. For example, in some embodiments, the fluorescence of the fluorescent moiety is such that it allows detection and/or quantitation of a molecule, e.g., a marker, at a limit of detection of less than about 10, 5, 4, 3, 2, 1, 0.1, 0.01, 0.001, 0.00001, or 0.000001 μg/ml and with a coefficient of variation of less than about 20, 15, 14, 13, 12, 11, 10, 9, 8, 7, 6, 5, 4, 3, 2, 1% or less, e.g., about 10% or less, in the instruments described herein. In some embodiments, the fluorescence of the fluorescent moiety is such that it allows detection and/or quantitation of a molecule, e.g., a marker, at a limit of detection of less than about 5, 1, 0.5, 0.1, 0.05, 0.01, 0.005, 0.001 μg/ml and with a coefficient of variation of less than about 10%, in the instruments described herein. “Limit of detection,” or LoD, as those terms are used herein, includes the lowest concentration at which one can identify a sample as containing a molecule of the substance of interest, e.g., the first non-zero value. It can be defined by the variability of zeros and the slope of the standard curve. For example, the limit of detection of an assay may be determined by running a standard curve, determining the standard curve zero value, and adding 2 standard deviations to that value. A concentration of the substance of interest that produces a signal equal to this value is the “lower limit of detection” concentration.

Furthermore, the moiety has properties that are consistent with its use in the assay of choice. In some embodiments, the assay is an immunoassay, where the fluorescent moiety is attached to an antibody; the moiety must have properties such that it does not aggregate with other antibodies or proteins, or experiences no more aggregation than is consistent with the required accuracy and precision of the assay. In some embodiments, fluorescent moieties that are preferred are fluorescent moieties, e.g., dye molecules that have a combination of 1) high absorption coefficient; 2) high quantum yield; 3) high photostability (low photobleaching); and 4) compatibility with labeling the molecule of interest (e.g., protein) so that it may be analyzed using the analyzers and systems of the invention (e.g., does not cause precipitation of the protein of interest, or precipitation of a protein to which the moiety has been attached).

Any suitable fluorescent moiety may be used. Examples include, but are not limited to, Alexa Fluor dyes (Molecular Probes, Eugene, Oreg.). The Alexa Fluor dyes are disclosed in U.S. Pat. Nos. 6,977,305; 6,974,874; 6,130,101; and 6,974,305 which are herein incorporated by reference in their entirety. Some embodiments of the invention utilize a dye chosen from the group consisting of Alexa Fluor 647, Alexa Fluor 488, Alexa Fluor 532, Alexa Fluor 555, Alexa Fluor 610, Alexa Fluor 680, Alexa Fluor 700, and Alexa Fluor 750. Some embodiments of the invention utilize a dye chosen from the group consisting of Alexa Fluor 488, Alexa Fluor 532, Alexa Fluor 647, Alexa Fluor 700 and Alexa Fluor 750. Some embodiments of the invention utilize a dye chosen from the group consisting of Alexa Fluor 488, Alexa Fluor 532, Alexa Fluor 555, Alexa Fluor 610, Alexa Fluor 680, Alexa Fluor 700, and Alexa Fluor 750. Some embodiments of the invention utilize the Alexa Fluor 647 molecule, which has an absorption maximum between about 650 and 660 nm and an emission maximum between about 660 and 670 nm. The Alexa Fluor 647 dye is used alone or in combination with other Alexa Fluor dyes.

In some embodiments, the fluorescent label moiety that is used to detect a biomarker in a sample using the analyzer systems of the invention is a quantum dot. Quantum dots (QDs), also known as semiconductor nanocrystals or artificial atoms, are semiconductor crystals that contain anywhere between 100 to 1,000 electrons and range from 2-10 nm. Some QDs can be between 10-20 nm in diameter. QDs have high quantum yields, which makes them particularly useful for optical applications. QDs are fluorophores that fluoresce by forming excitons, which are similar to the excited state of traditional fluorophores, but have much longer lifetimes of up to 200 nanoseconds. This property provides QDs with low photobleaching. The energy level of QDs can be controlled by changing the size and shape of the QD, and the depth of the QDs' potential. One optical feature of small excitonic QDs is coloration, which is determined by the size of the dot. The larger the dot, the redder, or more towards the red end of the spectrum the fluorescence. The smaller the dot, the bluer or more towards the blue end it is. The bandgap energy that determines the energy and hence the color of the fluoresced light is inversely proportional to the square of the size of the QD. Larger QDs have more energy levels which are more closely spaced, thus allowing the QD to absorb photons containing less energy, i.e., those closer to the red end of the spectrum. Because the emission frequency of a dot is dependent on the bandgap, it is possible to control the output wavelength of a dot with extreme precision. In some embodiments the protein that is detected with the single molecule analyzer system is labeled with a QD. In some embodiments, the single molecule analyzer is used to detect a protein labeled with one QD and using a filter to allow for the detection of different proteins at different wavelengths.

### F. Isolated Biomarkers

1. Isolated Polypeptide Biomarkers

One aspect of the invention pertains to isolated marker proteins and biologically active portions thereof as well as polypeptide fragments suitable for use as immunogens to raise antibodies directed against a marker protein or a fragment thereof. In one embodiment, the native marker protein can be isolated by an appropriate purification scheme using standard protein purification techniques. In another embodiment, a protein or peptide comprising the whole or a segment of the marker protein is produced by recombinant DNA techniques. Alternative to recombinant expression, such protein or peptide can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of a marker protein include polypeptides comprising amino acid sequences sufficiently identical to or derived from the amino acid sequence of the marker protein, which include fewer amino acids than the fill length protein, and exhibit at least one activity of the corresponding full-length protein. Typically, biologically active portions comprise a domain or motif with at least one activity of the corresponding full-length protein. A biologically active portion of a marker protein of the invention can be a polypeptide which is, for example, 10, 25, 50, 100 or more amino acids in length. Moreover, other biologically active portions, in which other regions of the marker protein are deleted, can be prepared by recombinant techniques and evaluated for one or more of the functional activities of the native form of the marker protein.

Preferred marker proteins are encoded by nucleotide sequences provided in the sequence listing. Other useful proteins are substantially identical (e.g., at least about 40%, preferably 50%, 60%, 70%, 80%, 90%, 91%, 92%, 93%, 94%, 95%, 96%, 97%, 98% or 99%) to one of these sequences and retain the functional activity of the corresponding naturally-occurring marker protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. Preferably, the percent identity between the two sequences is calculated using a global alignment. Alternatively, the percent identity between the two sequences is calculated using a local alignment. The percent identity between the two sequences is a function of the number of identical positions shared by the sequences (i.e., % identity=#of identical positions/total #of positions (e.g., overlapping positions)×100). In one embodiment the two sequences are the same length. In another embodiment, the two sequences are not the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the BLASTN and BLASTX programs of Altschul, et al. (1990) J. Mol. Biol. 215:403-410. BLAST nucleotide searches can be performed with the BLASTN program, score=100, wordlength=12 to obtain nucleotide sequences homologous to a nucleic acid molecules of the invention. BLAST protein searches can be performed with the BLASTP program, score=50, wordlength=3 to obtain amino acid sequences homologous to a protein molecules of the invention. To obtain gapped alignments for comparison purposes, a newer version of the BLAST algorithm called Gapped BLAST can be utilized as described in Altschul et al. (1997) Nucleic Acids Res. 25:3389-3402, which is able to perform gapped local alignments for the programs BLASTN, BLASTP and BLASTX. Alternatively, PSI-Blast can be used to perform an iterated search which detects distant relationships between molecules. When utilizing BLAST, Gapped BLAST, and PSI-Blast programs, the default parameters of the respective programs (e.g., BLASTX and BLASTN) can be used. See the NCBI website. Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted.

Another aspect of the invention pertains to antibodies directed against a protein of the invention. In preferred embodiments, the antibodies specifically bind a marker protein or a fragment thereof. The terms “antibody” and “antibodies” as used interchangeably herein refer to immunoglobulin molecules as well as fragments and derivatives thereof that comprise an immunologically active portion of an immunoglobulin molecule, (i.e., such a portion contains an antigen binding site which specifically binds an antigen, such as a marker protein, e.g., an epitope of a marker protein). An antibody which specifically binds to a protein of the invention is an antibody which binds the protein, but does not substantially bind other molecules in a sample, e.g., a biological sample, which naturally contains the protein. Examples of an immunologically active portion of an immunoglobulin molecule include, but are not limited to, single-chain antibodies (scAb), F(ab) and F(ab′)₂ fragments.

An isolated protein of the invention or a fragment thereof can be used as an immunogen to generate antibodies. The full-length protein can be used or, alternatively, the invention provides antigenic peptide fragments for use as immunogens. The antigenic peptide of a protein of the invention comprises at least 8 (preferably 10, 15, 20, or 30 or more) amino acid residues of the amino acid sequence of one of the proteins of the invention, and encompasses at least one epitope of the protein such that an antibody raised against the peptide forms a specific immune complex with the protein. Preferred epitopes encompassed by the antigenic peptide are regions that are located on the surface of the protein, e.g., hydrophilic regions. Hydrophobicity sequence analysis, hydrophilicity sequence analysis, or similar analyses can be used to identify hydrophilic regions. In preferred embodiments, an isolated marker protein or fragment thereof is used as an immunogen.

The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope. Preferred polyclonal and monoclonal antibody compositions are ones that have been selected for antibodies directed against a protein of the invention. Particularly preferred polyclonal and monoclonal antibody preparations are ones that contain only antibodies directed against a marker protein or fragment thereof. Methods of making polyclonal, monoclonal, and recombinant antibody and antibody fragments are well known in the art.

2. Isolated Nucleic Acid Biomarkers

One aspect of the invention pertains to isolated nucleic acid molecules which encode a marker protein or a portion thereof. Isolated nucleic acids of the invention also include nucleic acid molecules sufficient for use as hybridization probes to identify marker nucleic acid molecules, and fragments of marker nucleic acid molecules, e.g., those suitable for use as PCR primers for the amplification of a specific product or mutation of marker nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. In one embodiment, an “isolated” nucleic acid molecule (preferably a protein-encoding sequences) is free of sequences which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. In another embodiment, an “isolated” nucleic acid molecule, such as a cDNA molecule, can be substantially free of other cellular material, or culture medium when produced by recombinant techniques, or substantially free of chemical precursors or other chemicals when chemically synthesized. A nucleic acid molecule that is substantially free of cellular material includes preparations having less than about 30%, 20%, 10%, or 5% of heterologous nucleic acid (also referred to herein as a “contaminating nucleic acid”).

A nucleic acid molecule of the present invention can be isolated using standard molecular biology techniques and the sequence information in the database records described herein. Using all or a portion of such nucleic acid sequences, nucleic acid molecules of the invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N.Y., 1989).

A nucleic acid molecule of the invention can be amplified using cDNA, mRNA, or genomic DNA as a template and appropriate oligonucleotide primers according to standard PCR amplification techniques. The nucleic acid so amplified can be cloned into an appropriate vector and characterized by DNA sequence analysis. Furthermore, nucleotides corresponding to all or a portion of a nucleic acid molecule of the invention can be prepared by standard synthetic techniques, e.g., using an automated DNA synthesizer.

In another preferred embodiment, an isolated nucleic acid molecule of the invention comprises a nucleic acid molecule which has a nucleotide sequence complementary to the nucleotide sequence of a marker nucleic acid or to the nucleotide sequence of a nucleic acid encoding a marker protein. A nucleic acid molecule which is complementary to a given nucleotide sequence is one which is sufficiently complementary to the given nucleotide sequence that it can hybridize to the given nucleotide sequence thereby forming a stable duplex.

Moreover, a nucleic acid molecule of the invention can comprise only a portion of a nucleic acid sequence, wherein the full length nucleic acid sequence comprises a marker nucleic acid or which encodes a marker protein. Such nucleic acids can be used, for example, as a probe or primer. The probe/primer typically is used as one or more substantially purified oligonucleotides. The oligonucleotide typically comprises a region of nucleotide sequence that hybridizes under stringent conditions to at least about 15, more preferably at least about 25, 50, 75, 100, 125, 150, 175, 200, 250, 300, 350, or 400 or more consecutive nucleotides of a nucleic acid of the invention.

Probes based on the sequence of a nucleic acid molecule of the invention can be used to detect transcripts or genomic sequences corresponding to one or more markers of the invention. In certain embodiments, the probes hybridize to nucleic acid sequences that traverse splice junctions. The probe comprises a label group attached thereto, e.g., a radioisotope, a fluorescent compound, an enzyme, or an enzyme co-factor. Such probes can be used as part of a diagnostic test kit or panel for identifying cells or tissues which express or mis-express the protein, such as by measuring levels of a nucleic acid molecule encoding the protein in a sample of cells from a subject, e.g., detecting mRNA levels or determining whether a gene encoding the protein or its translational control sequences have been mutated or deleted.

The invention further encompasses nucleic acid molecules that differ, due to degeneracy of the genetic code, from the nucleotide sequence of nucleic acids encoding a marker protein (e.g., protein having the sequence provided in the sequence listing), and thus encode the same protein.

It will be appreciated by those skilled in the art that DNA sequence polymorphisms that lead to changes in the amino acid sequence can exist within a population (e.g., the human population). Such genetic polymorphisms can exist among individuals within a population due to natural allelic variation and changes known to occur in cancer. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

As used herein, the phrase “allelic variant” refers to a nucleotide sequence which occurs at a given locus or to a polypeptide encoded by the nucleotide sequence.

As used herein, the terms “gene” and “recombinant gene” refer to nucleic acid molecules comprising an open reading frame encoding a polypeptide corresponding to a marker of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

In another embodiment, an isolated nucleic acid molecule of the invention is at least 15, 20, 25, 30, 40, 60, 80, 100, 150, 200, 250, 300, 350, 400, 450, 550, 650, 700, 800, 900, 1000, 1200, 1400, 1600, 1800, 2000, 2200, 2400, 2600, 2800, 3000, 3500, 4000, 4500, or more nucleotides in length and hybridizes under stringent conditions to a marker nucleic acid or to a nucleic acid encoding a marker protein. As used herein, the term “hybridizes under stringent conditions” is intended to describe conditions for hybridization and washing under which nucleotide sequences at least 60% (65%, 70%, preferably 75%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A preferred, non-limiting example of stringent hybridization conditions are hybridization in 6× sodium chloride/sodium citrate (SSC) at about 45° C., followed by one or more washes in 0.2×SSC, 0.1% SDS at 50-65° C.

### G. Biomarker Applications

The invention provides methods for diagnosing an abnormal prostate state, e.g., prostate cancer, in a subject. The invention further provides methods for prognosing or monitoring progression or monitoring response of an abnormal prostate state, e.g., prostate cancer, to a therapeutic treatment during active treatment or watchful waiting.

In one aspect, the present invention constitutes an application of diagnostic information obtainable by the methods of the invention in connection with analyzing, detecting, and/or measuring the prostate cancer biomarkers of the present invention, i.e., the markers of Tables 1-31, which goes well beyond the discovered correlation between prostate cancer and the biomarkers of the invention.

For example, when executing the methods of the invention for detecting and/or measuring an protein biomarker of the present invention, as described herein, one may contact a biological sample with a detection reagent, e.g., a monoclonal antibody, which selectively binds to the biomarker of interest, forming a protein-protein complex, which is then further detected either directly (if the antibody comprises a label) or indirectly (if a secondary detection reagent is used, e.g., a secondary antibody, which in turn is labeled). Thus, the method of the invention transforms the polypeptide markers of the invention to a protein-protein complex that comprises either a detectable primary antibody or a primary and further secondary antibody. Forming such protein-protein complexes is required in order to identify the presence of the biomarker of interest and necessarily changes the physical characteristics and properties of the biomarker of interest as a result of conducting the methods of the invention.

The same principal applies when conducting the methods of the invention for detecting nucleic acids that correspond to the protein biomarkers of the invention. In particular, when amplification methods are used, the process results in the formation of a new population of amplicons, i.e., molecules that are newly synthesized and which were not present in the original biological sample, thereby physically transforming the biological sample. Similarly, when hybridization probes are used to detect a target biomarker, a physical new species of molecules is in effect created by the hybridization of the probes (optionally comprising a label) to the target biomarker mRNA (or other nucleic acid), which is then detected. Such polynucleotide products are effectively newly created or formed as a consequence of carrying out the method of the invention.

The invention provides, in one embodiment, methods for diagnosing an oncological disorder, e.g., prostate cancer. The methods of the present invention can be practiced in conjunction with any other method used by the skilled practitioner to prognose the occurrence or recurrence of an oncologic disorder and/or the survival of a subject being treated for an oncologic disorder. The diagnostic and prognostic methods provided herein can be used to determine if additional and/or more invasive tests or monitoring should be performed on a subject. It is understood that a disease as complex as an oncological disorder is rarely diagnosed using a single test. Therefore, it is understood that the diagnostic, prognostic, and monitoring methods provided herein are typically used in conjunction with other methods known in the art. For example, the methods of the invention may be performed in conjunction with a morphological or cytological analysis of the sample obtained from the subject, imaging analysis, and/or physical exam. Cytological methods would include immunohistochemical or immunofluorescence detection (and quantitation if appropriate) of any other molecular marker either by itself, in conjunction with other markers. Other methods would include detection of other markers by in situ PCR, or by extracting tissue and quantitating other markers by real time PCR. PCR is defined as polymerase chain reaction.

Methods for assessing tumor progression during watchful waiting or the efficacy of a treatment regimen, e.g., chemotherapy, radiation therapy, surgery, hormone therapy, or any other therapeutic approach useful for treating an oncologic disorder in a subject are also provided. In these methods the amount of marker in a pair of samples (a first sample obtained from the subject at an earlier time point or prior to the treatment regimen and a second sample obtained from the subject at a later time point, e.g., at a later time point when the subject has undergone at least a portion of the treatment regimen) is assessed. It is understood that the methods of the invention include obtaining and analyzing more than two samples (e.g., 3, 4, 5, 6, 7, 8, 9, or more samples) at regular or irregular intervals for assessment of marker levels. Pairwise comparisons can be made between consecutive or non-consecutive subject samples. Trends of marker levels and rates of change of marker levels can be analyzed for any two or more consecutive or non-consecutive subject samples.

The invention also provides a method for determining whether an oncologic disorder, e.g., prostate cancer, is aggressive. The method comprises determining the amount of a marker present in a sample and comparing the amount to a control amount of the marker present in one or more control samples, as defined in Definitions, thereby determining whether an oncologic disorder is aggressive. Marker levels can be compared to marker levels in samples obtained at different times from the same subject or marker levels from normal or abnormal prostate state subjects. A rapid increase in the level of marker may be indicative of a more aggressive cancer than a slow increase or no increase or change in the marker level.

The methods of the invention may also be used to select a compound that is capable of modulating i.e., decreasing the aggressiveness of an oncologic disorder, e.g., prostate cancer. In this method, a cancer cell is contacted with a test compound, and the ability of the test compound to modulate the expression and/or activity of a marker in the invention in the cancer cell is determined, thereby selecting a compound that is capable of modulating aggressiveness of an oncologic disorder.

Using the methods described herein, a variety of molecules, may be screened in order to identify molecules which modulate, e.g., increase or decrease the expression and/or activity of a marker of the invention. Compounds so identified can be provided to a subject in order to inhibit the aggressiveness of an oncologic disorder in the subject, to prevent the recurrence of an oncologic disorder in the subject, or to treat an oncologic disorder in the subject.

The present invention pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the present invention relates to diagnostic assays for determining the level of expression of one or more marker proteins or nucleic acids, in order to determine whether an individual is at risk of developing a disease or disorder, such as, without limitation, an oncological disorder, e.g., prostate cancer. Such assays can be used for prognostic or predictive purposes to thereby prophylactically treat an individual prior to the onset of the disorder.

Yet another aspect of the invention pertains to monitoring the influence of agents (e.g., drugs or other therapeutic compounds) on the expression or activity of a biomarker of the invention in clinical trials. These and other applications are described in further detail in the following sections.

1. Diagnostic Assays

An exemplary method for detecting the presence or absence or change of expression level of a marker protein or a corresponding nucleic acid in a biological sample involves obtaining a biological sample (e.g. an oncological disorder-associated body fluid) from a test subject and contacting the biological sample with a compound or an agent capable of detecting the polypeptide or nucleic acid (e.g., mRNA, genomic DNA, or cDNA). The detection methods of the invention can thus be used to detect mRNA, protein, cDNA, or genomic DNA, for example, in a biological sample in vitro as well as in vivo.

Methods provided herein for detecting the presence, absence, change of expression level of a marker protein or corresponding nucleic acid in a biological sample include obtaining a biological sample from a subject that may or may not contain the marker protein or nucleic acid to be detected, contacting the sample with a marker-specific binding agent (i.e., one or more marker-specific binding agents) that is capable of forming a complex with the marker protein or nucleic acid to be detected, and contacting the sample with a detection reagent for detection of the marker-marker-specific binding agent complex, if formed. It is understood that the methods provided herein for detecting an expression level of a marker in a biological sample includes the steps to perform the assay. In certain embodiments of the detection methods, the level of the marker protein or nucleic acid in the sample is none or below the threshold for detection.

The methods include formation of either a transient or stable complex between the marker and the marker-specific binding agent. The methods require that the complex, if formed, be formed for sufficient time to allow a detection reagent to bind the complex and produce a detectable signal (e.g., fluorescent signal, a signal from a product of an enzymatic reaction, e.g., a peroxidase reaction, a phosphatase reaction, a beta-galactosidase reaction, or a polymerase reaction).

In certain embodiments, all markers are detected using the same method. In certain embodiments, all markers are detected using the same biological sample (e.g., same body fluid or tissue). In certain embodiments, different markers are detected using various methods. In certain embodiments, markers are detected in different biological samples.

2. Protein Detection

In certain embodiments of the invention, the marker to be detected is an protein. Proteins are detected using a number of assays in which a complex between the marker protein to be detected and the marker specific binding agent would not occur naturally, for example, because one of the components is not a naturally occurring compound or the marker for detection and the marker specific binding agent are not from the same organism (e.g., human marker proteins detected using marker-specific binding antibodies from mouse, rat, or goat). In a preferred embodiment of the invention, the marker protein for detection is a human marker protein. In certain detection assays, the human markers for detection are bound by marker-specific, non-human antibodies, thus, the complex would not be formed in nature. The complex of the marker protein can be detected directly, e.g., by use of a labeled marker-specific antibody that binds directly to the marker, or by binding a further component to the marker—marker-specific antibody complex. In certain embodiments, the further component is a second marker-specific antibody capable of binding the marker at the same time as the first marker-specific antibody. In certain embodiments, the further component is a secondary antibody that binds to a marker-specific antibody, wherein the secondary antibody preferably linked to a detectable label (e.g., fluorescent label, enzymatic label, biotin). When the secondary antibody is linked to an enzymatic detectable label (e.g., a peroxidase, a phosphatase, a beta-galactosidase), the secondary antibody is detected by contacting the enzymatic detectable label with an appropriate substrate to produce a colorimetric, fluorescent, or other detectable, preferably quantitatively detectable, product. Antibodies for use in the methods of the invention can be polyclonal, however, in a preferred embodiment monoclonal antibodies are used. An intact antibody, or a fragment or derivative thereof (e.g., Fab or F(ab′)2) can be used in the methods of the invention. Such strategies of marker protein detection are used, for example, in ELISA, RIA, western blot, and immuno fluorescence assay methods.

In certain detection assays, the marker present in the biological sample for detection is an enzyme and the detection reagent is an enzyme substrate. For example, the enzyme can be a protease and the substrate can be any protein that includes an appropriate protease cleavage site. Alternatively, the enzyme can be a kinase and the substrate can be any substrate for the kinase. In preferred embodiments, the substrate which forms a complex with the marker enzyme to be detected is not the substrate for the enzyme in a human subject.

In certain embodiments, the marker—marker-specific binding agent complex is attached to a solid support for detection of the marker. The complex can be formed on the substrate or formed prior to capture on the substrate. For example, in an ELISA, RIA, immunoprecipitation assay, western blot, immunofluorescence assay, in gel enzymatic assay the marker for detection is attached to a solid support, either directly or indirectly. In an ELISA, RIA, or immunofluorescence assay, the marker is typically attached indirectly to a solid support through an antibody or binding protein. In a western blot or immunofluorescence assay, the marker is typically attached directly to the solid support. For in-gel enzyme assays, the marker is resolved in a gel, typically an acrylamide gel, in which a substrate for the enzyme is integrated.

3. Nucleic Acid Detection

In certain embodiments of the invention, the marker is a nucleic acid corresponding to a marker protein. Nucleic acids are detected using a number of assays in which a complex between the marker nucleic acid to be detected and a marker-specific probe would not occur naturally, for example, because one of the components is not a naturally occurring compound. In certain embodiments, the analyte comprises a nucleic acid and the probe comprises one or more synthetic single stranded nucleic acid molecules, e.g., a DNA molecule, a DNA-RNA hybrid, a PNA, or a modified nucleic acid molecule containing one or more artificial bases, sugars, or backbone moieties. In certain embodiments, the synthetic nucleic acid is a single stranded is a DNA molecule that includes a fluorescent label. In certain embodiments, the synthetic nucleic acid is a single stranded oligonucleotide molecule of about 12 to about 50 nucleotides in length. In certain embodiments, the nucleic acid to be detected is an mRNA and the complex formed is an mRNA hybridized to a single stranded DNA molecule that is complementary to the mRNA. In certain embodiments, an RNA is detected by generation of a DNA molecule (i.e., a cDNA molecule) first from the RNA template using the single stranded DNA that hybridizes to the RNA as a primer, e.g., a general poly-T primer to transcribe poly-A RNA. The cDNA can then be used as a template for an amplification reaction, e.g., PCR, primer extension assay, using a marker-specific probe. In certain embodiments, a labeled single stranded DNA can be hybridized to the RNA present in the sample for detection of the RNA by fluorescence in situ hybridization (FISH) or for detection of the RNA by northern blot.

For example, in vitro techniques for detection of mRNA include northern hybridizations, in situ hybridizations, and rtPCR. In vitro techniques for detection of genomic DNA include Southern hybridizations. Techniques for detection of mRNA include PCR, northern hybridizations and in situ hybridizations. Methods include both qualitative and quantitative methods.

A general principle of such diagnostic, prognostic, and monitoring assays involves preparing a sample or reaction mixture that may contain a marker, and a probe, under appropriate conditions and for a time sufficient to allow the marker and probe to interact and bind, thus forming a complex that can be removed and/or detected in the reaction mixture. These assays can be conducted in a variety of ways known in the art, e.g., ELISA assay, PCR, FISH.

4. Detection of Expression Levels

Marker levels can be detected based on the absolute expression level or a normalized or relative expression level. Detection of absolute marker levels may be preferable when monitoring the treatment of a subject or in determining if there is a change in the prostate cancer status of a subject. For example, the expression level of one or more markers can be monitored in a subject undergoing treatment for prostate cancer, e.g., at regular intervals, such a monthly intervals. A modulation in the level of one or more markers can be monitored over time to observe trends in changes in marker levels. Expression levels of the biomarkers of the invention in the subject may be higher than the expression level of those markers in a normal sample, but may be lower than the prior expression level, thus indicating a benefit of the treatment regimen for the subject. Similarly, rates of change of marker levels can be important in a subject who is not subject to active treatment for prostate cancer (e.g., watchful waiting). Changes, or not, in marker levels may be more relevant to treatment decisions for the subject than marker levels present in the population. Rapid changes in marker levels in a subject who otherwise appears to have a normal prostate may be indicative of an abnormal prostate state, even if the markers are within normal ranges for the population.

As an alternative to making determinations based on the absolute expression level of the marker, determinations may be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes such as the actin gene, or epithelial cell-specific genes. This normalization allows the comparison of the expression level in one sample, e.g., a patient sample, to another sample, e.g., a non-cancer sample, or between samples from different sources.

Alternatively, the expression level can be provided as a relative expression level as compared to an appropriate control, e.g., population control, adjacent normal tissue control, earlier time point control, etc. Preferably, the samples used in the baseline determination will be from non-cancer cells. The choice of the cell source is dependent on the use of the relative expression level. Using expression found in normal tissues as a mean expression score aids in validating whether the marker assayed is cancer specific (versus normal cells). In addition, as more data is accumulated, the mean expression value can be revised, providing improved relative expression values based on accumulated data. Expression data from cancer cells provides a means for grading the severity of the cancer state.

5. Diagnostic, Prognostic, Monitoring and Treatment Methods

The invention provides methods for diagnosing the presence of prostate cancer in a subject, comprising (a) detecting the level of a prostate cancer marker in a biological sample from the subject, wherein the prostate cancer marker comprises one or more markers selected from Tables 1-31; and (b) comparing the level of the prostate cancer marker in the biological sample with a predetermined threshold value; wherein the level of the prostate cancer marker above or below the predetermined threshold value indicates a diagnosis that prostate cancer is present in the subject.

In another aspect, the invention provides methods for diagnosing the presence of prostate cancer in a subject, comprising: (a) contacting a biological sample with one or more reagents that selectively bind to a prostate cancer marker in the biological sample from the subject, wherein the prostate cancer marker comprises one or more markers selected from Tables 1-31; (b) allowing a complex to form between the one or more reagents and the prostate cancer marker; (c) detecting the level of the complex; and (d) comparing the level of the complex with a predetermined threshold value; wherein the level of the complex above or below the predetermined threshold value indicates a diagnosis that prostate cancer is present in the subject.

In another aspect, the invention provides methods for diagnosing the presence of prostate cancer in a subject selected from a population of Caucasians, comprising (a) detecting the level of a prostate cancer marker in a biological sample from the subject, wherein the prostate cancer marker comprises one or more markers selected from Tables 1, 4, 8, 11, 13, 16, 19, 22, 26, 29 and 30; and (b) comparing the level of the prostate cancer marker in the biological sample with a predetermined threshold value; wherein the level of the prostate cancer marker above or below the predetermined threshold value indicates a diagnosis that prostate cancer is present in the subject.

In still another aspect, the invention further provides methods for diagnosing the presence of prostate cancer in a subject selected from a population of Caucasians, comprising: (a) contacting a biological sample with one or more reagents that selectively bind to a prostate cancer marker in the biological sample from the subject, wherein the prostate cancer marker comprises one or more markers selected from Tables 1, 4, 8, 11, 13, 16, 19, 22, 26, 29 and 30; (b) allowing a complex to form between the one or more reagents and the prostate cancer marker; (c) detecting the level of the complex; and (d) comparing the level of the complex with a predetermined threshold value; wherein the level of the complex above or below the predetermined threshold value indicates a diagnosis that prostate cancer is present in the subject.

In yet another aspect, the invention provides methods for diagnosing the presence of prostate cancer in a subject selected from a population of African Americans, comprising (a) detecting the level of a prostate cancer marker in a biological sample from the subject, wherein the prostate cancer marker comprises one or more markers selected from Tables 2, 5, 9, 12, 14, 17, 20, 23, 27 and 31; and (b) comparing the level of the prostate cancer marker in the biological sample with a predetermined threshold value; wherein the level of the prostate cancer marker above or below the predetermined threshold value indicates a diagnosis that prostate cancer is present in the subject.

In yet another aspect, the invention provides methods for diagnosing the presence of prostate cancer in a subject selected from a population of African Americans, comprising: (a) contacting a biological sample with one or more reagents that selectively bind to a prostate cancer marker in the biological sample from the subject, wherein the prostate cancer marker comprises one or more markers selected from Tables 2, 5, 9, 12, 14, 17, 20, 23, 27 and 31; (b) allowing a complex to form between the one or more reagents and the prostate cancer marker; (c) detecting the level of the complex; and (d) comparing the level of the complex with a predetermined threshold value; wherein the level of the complex above or below the predetermined threshold value indicates a diagnosis that prostate cancer is present in the subject.

In another aspect, the invention provides methods for diagnosing the presence of ERG-positive prostate cancer in a subject, comprising (a) detecting the level of an ERG-positive prostate cancer marker in a biological sample from the subject, wherein the ERG-positive prostate cancer marker comprises one or more markers selected from Tables 6, 30 and 31; and (b) comparing the level of the ERG-positive prostate cancer marker in the biological sample with a predetermined threshold value; wherein the level of the ERG-positive prostate cancer marker above or below the predetermined threshold value indicates a diagnosis that ERG-positive prostate cancer is present in the subject.

In another aspect, the invention provides methods for diagnosing the presence of ERG-positive prostate cancer in a subject, comprising: (a) contacting a biological sample with one or more reagents that selectively bind to an ERG-positive prostate cancer marker in the biological sample from the subject, wherein the ERG-positive prostate cancer marker comprises one or more markers selected from Tables 6, 30 and 31; (b) allowing a complex to form between the one or more reagents and the ERG-positive prostate cancer marker; (c) detecting the level of the complex; and (d) comparing the level of the complex with a predetermined threshold value; wherein the level of the complex above or below the predetermined threshold value indicates a diagnosis that ERG-positive prostate cancer is present in the subject.

In still another aspect, the invention provides methods for diagnosing the presence of prostate cancer in a subject with a BMI index equal or greater than 30, comprising (a) detecting the level of a high BMI prostate cancer marker in a biological sample from the subject, wherein the prostate cancer marker comprises one or more markers selected from Tables 7, 18 and 25; and (b) comparing the level of the high BMI prostate cancer marker in the biological sample with a predetermined threshold value; wherein the level of the high BMI prostate cancer marker above or below the predetermined threshold value indicates a diagnosis that prostate cancer is present in the subject.

In yet another aspect, the invention provides methods for diagnosing the presence of prostate cancer in a subject with a BMI index equal or greater than 30, comprising: (a) contacting a biological sample with one or more reagents that selectively bind to a high BMI prostate cancer marker in the biological sample from the subject, wherein the high BMI prostate cancer marker comprises one or more markers selected from Tables 7, 18 and 25; (b) allowing a complex to form between the one or more reagents and the high BMI prostate cancer marker; (c) detecting the level of the complex; and (d) comparing the level of the complex with a predetermined threshold value; wherein the level of the complex above or below the predetermined threshold value indicates a diagnosis that prostate cancer is present in the subject.

In another aspect, the invention provides methods for diagnosing the presence of ERG-negative prostate cancer in a Caucasian subject with a BMI index equal or greater than 30, comprising (a) detecting the level of mercapto-succinyl-carnitine in a biological sample from the subject; and (b) comparing the level of mercapto-succinyl-carnitine in the biological sample with a predetermined threshold value; wherein the level of mercapto-succinyl-carnitine above the predetermined threshold value indicates a diagnosis that ERG-negative prostate cancer is present in the subject.

In yet another aspect, the invention provides methods for diagnosing the presence of ERG-negative prostate cancer in a Caucasian subject with a BMI index equal or greater than 30, comprising: (a) contacting a biological sample with a reagent that selectively bind to mercapto-succinyl-carnitine in the biological sample from the subject; (b) allowing a complex to form between the reagent and mercapto-succinyl-carnitine; (c) detecting the level of the complex; and (d) comparing the level of the complex with a predetermined threshold value; wherein the level of the complex above or below the predetermined threshold value indicates a diagnosis that ERG-negative prostate cancer is present in the subject.

In still another aspect, the invention provides methods for identifying a subject as being at an increased risk for developing prostate cancer, comprising (a) detecting the level of a prostate cancer marker in a biological sample from the subject, wherein the prostate cancer marker comprises one or more markers selected from Tables 1-31; and (b) comparing the level of the prostate cancer marker in the biological sample with a predetermined threshold value; wherein the level of the prostate cancer marker above or below the predetermined threshold value indicates that the subject is being at an increased risk for developing prostate cancer.

In still another aspect, the invention provides methods for identifying a subject as being at an increased risk for developing prostate cancer, comprising: (a) contacting a biological sample with one or more reagents that selectively bind to a prostate cancer marker in the biological sample from the subject, wherein the prostate cancer marker comprises one or more markers selected from Tables 1-31; (b) allowing a complex to form between the one or more reagents and the prostate cancer marker; (c) detecting the level of the complex; and (d) comparing the level of the complex with a predetermined threshold value; wherein the level of the complex above or below the predetermined threshold value that the subject is being at an increased risk for developing prostate cancer.

In another aspect, the invention provides methods for identifying a Caucasian subject as being at an increased risk for developing prostate cancer, comprising (a) detecting the level of a prostate cancer marker in a biological sample from the subject, wherein the prostate cancer marker comprises one or more markers selected from Tables 1, 4, 8, 11, 13, 16, 19, 22, 26, 29 and 30; and (b) comparing the level of the prostate cancer marker in the biological sample with a predetermined threshold value; wherein the level of the prostate cancer marker above or below the predetermined threshold value indicates that the subject is being at an increased risk for developing prostate cancer.

In yet another aspect, the invention provides methods for identifying a Caucasian subject as being at an increased risk for developing prostate cancer, comprising: (a) contacting a biological sample with one or more reagents that selectively bind to a prostate cancer marker in the biological sample from the subject, wherein the prostate cancer marker comprises one or more markers selected from Tables 1, 4, 8, 11, 13, 16, 19, 22, 26, 29 and 30; (b) allowing a complex to form between the one or more reagents and the prostate cancer marker; (c) detecting the level of the complex; and (d) comparing the level of the complex with a predetermined threshold value; wherein the level of the complex above or below the predetermined threshold value indicates that the subject is being at an increased risk for developing prostate cancer.

In still another aspect, the invention provides methods for identifying an African American subject as being at an increased risk for developing prostate cancer, comprising (a) detecting the level of a prostate cancer marker in a biological sample from the subject, wherein the prostate cancer marker comprises one or more markers selected from Tables 2, 5, 9, 12, 14, 17, 20, 23, 27 and 31; and (b) comparing the level of the prostate cancer marker in the biological sample with a predetermined threshold value; wherein the level of the prostate cancer marker above or below the predetermined threshold value indicates that the subject is being at an increased risk for developing prostate cancer.

The invention provides methods for identifying an African American subject as being at an increased risk for developing prostate cancer, comprising: (a) contacting a biological sample with one or more reagents that selectively bind to a prostate cancer marker in the biological sample from the subject, wherein the prostate cancer marker comprises one or more markers selected from Tables 2, 5, 9, 12, 14, 17, 20, 23, 27 and 31; (b) allowing a complex to form between the one or more reagents and the prostate cancer marker; (c) detecting the level of the complex; and (d) comparing the level of the complex with a predetermined threshold value; wherein the level of the complex above or below the predetermined threshold value indicates that the subject is being at an increased risk for developing prostate cancer.

In another aspect, the invention provides methods for identifying a subject as being at an increased risk for developing ERG-positive prostate cancer, comprising (a) detecting the level of an ERG-positive prostate cancer marker in a biological sample from the subject, wherein the ERG-positive prostate cancer marker comprises one or more markers selected from Tables 6, 30 and 31; and (b) comparing the level of the ERG-positive prostate cancer marker in the biological sample with a predetermined threshold value; wherein the level of the ERG-positive prostate cancer marker above or below the predetermined threshold value indicates that the subject is being at an increased risk for developing ERG-positive prostate cancer.

In yet another aspect, the invention provides methods for identifying a subject as being at an increased risk for developing ERG-positive prostate cancer, comprising: (a) contacting a biological sample with one or more reagents that selectively bind to an ERG-positive prostate cancer marker in the biological sample from the subject, wherein the ERG-positive prostate cancer marker comprises one or more markers selected from Tables 6, 30 and 31; (b) allowing a complex to form between the one or more reagents and the ERG-positive prostate cancer marker; (c) detecting the level of the complex; and (d) comparing the level of the complex with a predetermined threshold value; wherein the level of the complex above or below the predetermined threshold value indicates that the subject is being at an increased risk for developing ERG-positive prostate cancer.

In still another aspect, the invention provides methods for identifying a subject with a BMI index equal or greater than 30 as being at an increased risk for developing prostate cancer, comprising (a) detecting the level of a high BMI prostate cancer marker in a biological sample from the subject, wherein the prostate cancer marker comprises one or more markers selected from Tables 7, 18 and 25; and (b) comparing the level of the high BMI prostate cancer marker in the biological sample with a predetermined threshold value; wherein the level of the high BMI prostate cancer marker above or below the predetermined threshold value indicates that the subject is being at an increased risk for developing prostate cancer.

In still another aspect, the invention provides methods for identifying a subject with a BMI index equal or greater than 30 as being at an increased risk for developing prostate cancer, comprising: (a) contacting a biological sample with one or more reagents that selectively bind to a high BMI prostate cancer marker in the biological sample from the subject, wherein the high BMI prostate cancer marker comprises one or more markers selected from Tables 7, 18 and 25; (b) allowing a complex to form between the one or more reagents and the high BMI prostate cancer marker; (c) detecting the level of the complex; and (d) comparing the level of the complex with a predetermined threshold value; wherein the level of the complex above or below the predetermined threshold value indicates that the subject is being at an increased risk for developing prostate cancer.

In yet another aspect, the invention provides methods for identifying a Caucasian subject with a BMI index equal or greater than 30 as being at an increased risk for developing ERG-negative prostate cancer, comprising (a) detecting the level of mercapto-succinyl-carnitine in a biological sample from the subject; and (b) comparing the level of mercapto-succinyl-carnitine in the biological sample with a predetermined threshold value; wherein the level of mercapto-succinyl-carnitine above the predetermined threshold value indicates that the subject is being at an increased risk for developing ERG-negative prostate cancer.

In another aspect, the invention provides methods for identifying a Caucasian subject with a BMI index equal or greater than 30 as being at an increased risk for developing ERG-negative prostate cancer, comprising: (a) contacting a biological sample with a reagent that selectively bind to mercapto-succinyl-carnitine in the biological sample from the subject; (b) allowing a complex to form between the reagent and mercapto-succinyl-carnitine; (c) detecting the level of the complex; and (d) comparing the level of the complex with a predetermined threshold value; wherein the level of the complex above or below the predetermined threshold value indicates that the subject is being at an increased risk for developing ERG-negative prostate cancer.

The invention provides methods for monitoring prostate cancer in a subject, the method comprising: (1) detecting the level of a prostate cancer marker in a first biological sample obtained at a first time from the subject having prostate cancer, wherein the prostate cancer marker comprises one or more markers selected from Tables 1-31; (2) detecting the level of the prostate cancer marker in a second biological sample obtained from the subject at a second time, wherein the second time is later than the first time; and (3) comparing the level of the prostate cancer marker in the second sample with the level of the prostate cancer marker in the first sample; wherein a change in the level of the prostate cancer marker is indicative of a change in prostate cancer status in the subject.

The invention also provides methods for monitoring prostate cancer in a subject selected from a population of Caucasians, the method comprising: (1) detecting the level of a prostate cancer marker in a first biological sample obtained at a first time from the subject having prostate cancer, wherein the prostate cancer marker comprises one or more markers selected from Tables 1, 4, 8, 11, 13, 16, 19, 22, 26, 29 and 30; (2) detecting the level of the prostate cancer marker in a second biological sample obtained from the subject at a second time, wherein the second time is later than the first time; and (3) comparing the level of the prostate cancer marker in the second sample with the level of the prostate cancer marker in the first sample; wherein a change in the level of the prostate cancer marker is indicative of a change in prostate cancer status in the subject.

The invention further provides methods for monitoring prostate cancer in a subject selected from a population of African Americans, the method comprising: (1) detecting the level of a prostate cancer marker in a first biological sample obtained at a first time from the subject having prostate cancer, wherein the prostate cancer marker comprises one or more markers selected from Tables 2, 5, 9, 12, 14, 17, 20, 23, 27 and 31 in a first biological sample obtained at a first time from a subject having prostate cancer; (2) detecting the level of the prostate cancer marker in a second biological sample obtained from the subject at a second time, wherein the second time is later than the first time; and (3) comparing the level of the prostate cancer marker in the second sample with the level of the prostate cancer marker in the first sample; wherein a change in the level of the prostate cancer marker is indicative of a change in prostate cancer status in the subject.

The invention provides methods for monitoring ERG-positive prostate cancer in a subject, the method comprising: (1) detecting the level of an ERG-positive prostate cancer marker in a first biological sample obtained at a first time from the subject having ERG-positive prostate cancer, wherein the ERG-positive prostate cancer marker comprises one or more markers selected from Tables 6, 30 and 31; (2) detecting the level of the ERG-positive prostate cancer marker in a second biological sample obtained from the subject at a second time, wherein the second time is later than the first time; and (3) comparing the level of the ERG-positive prostate cancer marker in the second sample with the level of the ERG-positive prostate cancer marker in the first sample; wherein a change in the level of the ERG-positive prostate cancer marker is indicative of a change in ERG-positive prostate cancer status in the subject.

The invention also provides methods for monitoring prostate cancer in a subject with a BMI index equal or greater than 30, the method comprising: (1) detecting the level of a high BMI prostate cancer marker in a first biological sample obtained at a first time from the subject having prostate cancer, wherein the high BMI prostate cancer marker comprises one or more markers selected from Tables 7, 18 and 25; (2) detecting the level of the high BMI prostate cancer marker in a second biological sample obtained from the subject at a second time, wherein the second time is later than the first time; and (3) comparing the level of the high BMI prostate cancer marker in the second sample with the level of the high BMI prostate cancer marker in the first sample; wherein a change in the level of the high BMI prostate cancer marker is indicative of a change in prostate cancer status in the subject.

In another aspect, the invention provides methods for monitoring ERG-negative prostate cancer in a subject a Caucasian subject with a BMI index equal or greater than 30, the method comprising: (1) detecting the level of mercapto-succinyl-carnitine in a first biological sample obtained at a first time from a subject having ERG-negative prostate cancer; (2) detecting the level of mercapto-succinyl-carnitine in a second biological sample obtained from the subject at a second time, wherein the second time is later than the first time; and (3) comparing the level of mercapto-succinyl-carnitine in the second sample with the level of the at least one marker in the first sample; wherein a change in the level of mercapto-succinyl-carnitine is indicative of a change in prostate cancer status in the subject.

The invention also provides methods for treating prostate cancer in a subject, comprising administering to the subject a modulator of a prostate cancer marker, wherein the prostate cancer marker comprises one or more markers selected from Tables 1-31.

The invention further provides methods for treating prostate cancer in a subject selected from a population of Caucasians, comprising administering to the subject a modulator of a prostate cancer marker, wherein the prostate cancer marker comprises one or more markers selected from Tables 1, 4, 8, 11, 13, 16, 19, 22, 26, 29 and 30.

The invention also provides methods for treating prostate cancer in a subject selected from a population of African Americans, comprising administering to the subject a modulator of a prostate cancer marker, wherein the prostate cancer marker comprises one or more markers selected from Tables 2, 5, 9, 12, 14, 17, 20, 23, 27 and 31.

The invention also provides methods for treating ERG-positive prostate cancer in a subject, comprising administering to the subject a modulator of an ERG-positive prostate cancer marker, wherein the ERG-positive prostate cancer marker comprises one or more markers selected from Tables 6, 30 and 31.

The invention also provides methods for treating prostate cancer in a subject with a BMI index equal or greater than 30 comprising administering to the subject a modulator of a high BMI prostate cancer marker, wherein the high BMI prostate cancer marker comprises one or more markers selected from Tables 7, 18 and 25.

The invention also provides methods for treating ERG-negative prostate cancer in a Caucasian subject with a BMI index equal or greater than 30 comprising administering to the subject a modulator of mercapto-succinyl-carnitine.

In certain embodiments of the diagnostic, prognostic, monitoring and treatment methods provided herein, one or more marker, e.g., prostate cancer markers, ERG-positive prostate cancer markers or high BMI prostate cancer markers, is two or more markers. In certain embodiments of the diagnostic, prognostic, monitoring and treatment methods provided herein, one or more one or more marker, e.g., prostate cancer markers, ERG-positive prostate cancer markers or high BMI prostate cancer markers, is three or more markers. In certain embodiments of the diagnostic, prognostic, monitoring and treatment methods provided herein, one or more marker, e.g., prostate cancer markers, ERG-positive prostate cancer markers or high BMI prostate cancer markers, is four or more markers. In certain embodiments of the diagnostic, prognostic, monitoring and treatment methods provided herein, one or more marker, e.g., prostate cancer markers, ERG-positive prostate cancer markers or high BMI prostate cancer markers, is five or more markers. In certain embodiments of the diagnostic, prognostic, monitoring and treatment methods provided herein, one or more marker, e.g., prostate cancer markers, ERG-positive prostate cancer markers or high BMI prostate cancer markers, is six or more markers. In certain embodiments of the diagnostic, prognostic, monitoring and treatment methods provided herein, one or more marker, e.g., prostate cancer markers, ERG-positive prostate cancer markers or high BMI prostate cancer markers, is seven or more markers. In certain embodiments of the diagnostic, prognostic, monitoring and treatment methods provided herein, one or more marker, e.g., prostate cancer markers, ERG-positive prostate cancer markers or high BMI prostate cancer markers, is eight or more markers. In certain embodiments of the diagnostic, prognostic, monitoring and treatment methods provided herein, one or more marker, e.g., prostate cancer markers, ERG-positive prostate cancer markers or high BMI prostate cancer markers, is nine or more markers.

In certain embodiments of the diagnostic methods provided herein, an increase or decrease in the level of one or more prostate cancer markers selected from Tables 1-31 in the biological sample as compared to the level of the one or more markers in a normal control sample is an indication that the subject is afflicted with prostate cancer. In certain embodiments of the diagnostic methods provided herein, no increase or decrease in the detected expression level of one or more prostate cancer markers selected from Tables 1-31 in the biological sample as compared to the expression level of the one or more markers in a normal control sample is an indication that the subject is not afflicted with prostate cancer or not predisposed to developing prostate cancer.

In certain embodiments of the diagnostic methods provided herein, an increase or decrease in the level of one or more prostate cancer markers selected from Tables 1-31 in the biological sample as compared to the level of the one or more markers in a normal control sample is an indication that the subject is predisposed to developing prostate cancer.

In certain embodiments of the monitoring methods provided herein, no increase or decrease in the detected level of one or more prostate cancer markers selected from Tables 1-31 in the second sample as compared to the level of the one or more markers in the first sample is an indication that the therapy is efficacious for treating prostate cancer in the subject. In certain embodiments of the monitoring methods provided herein, wherein an increased or decreased expression level of one or more prostate cancer markers selected from Tables 1-31 in the second sample as compared to the expression level in the first sample is an indication that the therapy is not efficacious in the treatment of prostate cancer.

In certain embodiments the monitoring methods provided herein further comprise comparing the level of the one or more cancer markers selected from Tables 1-31 in the first sample or the level of the one or more prostate cancer markers selected from Tables 1-31 in the second sample with the level of the one or more prostate-cancer related markers in a control sample.

In certain embodiments of the monitoring methods provided herein, an increase or decrease in the level of the one or more prostate cancer markers selected from Tables 1-31 in the second sample as compared to the level of the one or more markers in the first sample is an indication for selection of active treatment of prostate cancer in the subject. In certain embodiments of the monitoring methods provided herein, no increase or decrease in the detected level of the one or more prostate cancer markers selected from Tables 1-31 in the second sample as compared to the level of the one or more markers in the first sample is an indication against selection of active treatment of prostate cancer in the subject.

In certain embodiments of the monitoring methods provided herein, modulation of the level of the one or more prostate cancer markers selected from Tables 1-31 in the second sample as compared to the level of the corresponding marker(s) in the first sample is indicative of a change in prostate cancer status in response to treatment of the prostate cancer in the subject. In certain embodiments of the monitoring methods provided herein, the methods further comprise comparing the level of the one or more prostate cancer markers selected from Tables 1-31 in the second sample to the level of the corresponding markers in a normal control sample.

In any of the aforementioned embodiments, the methods may also include a step of determining whether a subject having prostate cancer or who is being treated for prostate cancer is responsive to a particular treatment. Such a step can include, for example, measuring the level of the one or more prostate cancer markers selected from Tables 1-31 prior to administering an anti-prostate cancer treatment, and measuring the level of the one or more prostate cancer markers selected from Tables 1-31 after administering the anti-prostate cancer treatment, and comparing the expression level before and after treatment. Determining that the prostate cancer is responsive to the treatment if the expression level of the one or more markers is higher or lower than before treatment as compared to after treatment. The method may further include the step of adjusting the treatment to a higher dose in order to increase the responsiveness to the treatment, or adjusting the treatment to a lower dose in order to decrease the responsiveness to the treatment.

In any of the aforementioned embodiments, the methods may also include a step of determining whether a subject having prostate cancer or who is being treated for prostate cancer is not responsive to a particular treatment. Such a step can include, for example, measuring the level of the one or more prostate cancer markers selected from Tables 1-31 prior to administering an anti-prostate cancer treatment, and measuring the level of the one or more prostate cancer markers selected from Tables 1-31 after administering the anti-prostate cancer treatment, and comparing the expression level before and after treatment. Determining that the prostate cancer is not responsive to the treatment if the expression level of the one or more markers is higher or lower than before treatment as compared to after treatment. The method may further include the step of adjusting the treatment to a higher dose in order to increase the responsiveness to the treatment.

In certain embodiments, the marker, e.g., a prostate cancer marker, is a structural lipid, for example, a structural lipid listed in Tables 1-7. In some embodiments, the invention also relates to a marker comprising one or more of the structural lipids listed in Tables 1-7. In certain embodiments, the marker, e.g., a prostate cancer marker, is a signaling lipid, for example, a signaling lipid listed in Tables 8-12. In some embodiments, the invention also relates to a marker comprising one or more of the signaling lipids listed in Tables 8-12. In certain embodiments, the marker, e.g., a prostate cancer marker, is a protein, for example, a protein listed in Tables 13-18. In some embodiments, the invention also relates to a marker comprising one or more of the proteins listed in Tables 13-18. In certain embodiments, the marker, e.g., a prostate cancer marker, is a metabolite, for example, a metabolite listed in Tables 19-25. In some embodiments, the invention also relates to a marker comprising one or more of the metabolites listed in Tables 19-25. In certain embodiments, the marker, e.g., a prostate cancer marker, is selected from Tables 26-28. In some embodiments, the invention also relates to a marker comprising one or more of the markers listed in Tables 26-28.

In some embodiments, the marker, e.g., a prostate cancer marker, comprises at least two or more markers, wherein each of the two of more markers are selected from the structural lipids set forth in Tables 1-7, the signaling lipids set forth in Tables 8-12, the proteins set forth in Tables 13-18, the metabolites set forth in Tables 19-25, and/or the markers set forth in Tables 26-28.

In certain embodiments, the level of the marker, e.g., a prostate cancer marker, is increased when compared to the predetermined threshold value in the subject. In some embodiments, the marker, e.g., a prostate cancer marker, is selected from the group consisting of FFA_18:3, TAG_54:7+NH4, TAG_54:6+NH4, PA_18:1/20:2, FFA_18:3, FFA_20:1, TAG_54:7+NH4, TAG_54:6+NH4, PA_18:1/18:3, 6-KETO-PGF1A, TXB2, 13-HOTRE/13-HOTRE(R), 9-HOTRE, TXB2, 12-HEPE, 12-HETE, 13-HODE, APOC, APOB, ADIPOQ, SEPP1, CST3, F5, B2M, nicotinamide, eicosenoic acid, glycerylphosphorylethanolamine, nicotinamide, eicosenoic acid, 3-hydroxybutyric acid and 2-keto-isovalerate. In other embodiments, the marker, e.g., a prostate cancer marker, comprises one or more markers selected from Tables 4-7, 11, 12, 16-18, 22-25, 30 and 31, wherein the one or more markers have a FC ratio greater than 1, or a Log FC value greater than 0. In yet another embodiment, the marker, e.g., a prostate cancer marker, is selected from the group consisting of 13-HOTRE/13-HOTRE(R), nicotinamide, eicosenoic acid, glycerylphosphorylethanolamine, and 3-hydroxybutyric acid.

In other embodiments, the level of the marker, e.g., a prostate cancer marker, is decreased when compared to the predetermined threshold value in the subject. In some embodiments, the marker, e.g., a prostate cancer marker, is selected from the group consisting of CE_22:2+NH4, CE_20:0+NH4, CE_22:3+NH4, DAG_40:1+NH4, CE_20:1+NH4, PI_18:0/20:5, CE_22:1+NH4, TAG_54:0+NH4, PI_18:0/20:4, PI_16:0/18:3, PI_16:0/20:4, CE_20:0+NH4, CE_24:0+NH4, CE_22:2+NH4, DAG 42:2+NH4, PE_36:2, 5-HETE, LXA4, 15-OXOETE, 5-HEPE, 8-HETE, LTB4, 5-HEPE, 5-HETE, LTB4, PGE2/PGD2, GPLD1, SERPING1, C3, A2M, SERPINA6, APOA4, APCS, ITIH2, CLU, APOA2, PPBP, C3, APOA4, C4BPA, MMRN2, APOA2, FGA, ABI3BP, APOA1, PROS1, COMP, CDH5, SERPINA6, glu-leu, 6-ketodecanoylcarnitine, myo-inositol, chenodeoxyglycocholate, 2-hydroxy-2-methylbutanedioic acid, nonanedioic acid, 6-ketodecanoylcarnitine, glu-leu, ethanolamine, and nonanoylcarnitine. In other embodiments, the marker, e.g., a prostate cancer marker, comprises one or more markers selected from Tables 4-7, 11, 12, 16-18, 22-25, 30 and 31, wherein the one or more markers have a FC ratio less than 1, or a Log FC value less than 0. In yet another embodiment, the marker, e.g., a prostate cancer marker, is selected from the group consisting of 15-OXOETE, 5-HEPE, 5-HETE, 6-ketodecanoylcarnitine, glu-leu, ethanolamine, and nonanoylcarnitine.

In certain embodiments, the prostate cancer marker for diagnosis of the presence of prostate cancer in a subject selected from a population of Caucasians comprises one or more markers selected from Tables 1, 4, 8, 11, 13, 16, 19, 22, 26, 29 and 30. In certain embodiments, the prostate cancer marker comprises at least two or more markers, wherein each of the two or more markers are selected from the structural lipids set forth in Tables 1 and 4, the signaling lipids set forth in Tables 8 and 11, the proteins set forth in Tables 13 and 16, the metabolites set forth in Tables 19 and 22, and/or the markers set forth in Table 26. In certain embodiments, the prostate cancer marker for diagnosis of the presence of prostate cancer in a subject selected from a population of Caucasians comprises one or more markers selected from Table 29. In other embodiments, markers for that are predictive of any particular stage or phase of prostate cancer, e.g., Gleason grade 1, grade 2, grade 3, grade 4, or grade 5 prostate cancer, i.e., Gleason score markers, include one or more marker selected from Table 29.

In certain embodiments, the level of the marker, e.g., a prostate cancer marker, for diagnosis of the presence of prostate cancer in a subject selected from a population of Caucasians, is increased when compared to the predetermined threshold value in the subject. In some embodiments, the prostate cancer marker is selected from the group consisting of FFA_18:3, TAG_54:7+NH4, TAG_54:6+NH4, PA_18:1/20:2, 6-KETO-PGF1A, TXB2, APOC, APOB, ADIPOQ, SEPP1, nicotinamide, eicosenoic acid, glycerylphosphorylethanolamine. In other embodiments, the prostate cancer marker comprises one or more markers selected from Tables 4, 11, 16, 22 and 30, wherein the one or more markers have a FC ratio greater than 1, or a Log FC value greater than 0. In certain embodiments, the prostate cancer marker is nicotinamide.

In other embodiments, the level of the marker, e.g., a prostate cancer marker, for diagnosis of the presence of prostate cancer in a subject selected from a population of Caucasians, is decreased when compared to the predetermined threshold value in the subject. In some embodiments, the prostate cancer marker is selected from the group consisting of CE_22:2+NH4, CE_20:0+NH4, CE_22:3+NH4, DAG_40:1+NH4, CE_20:1+NH4, PI_18:0/20:5, CE_22:1+NH4, TAG_54:0+NH4, PI_18:0/20:4, PI_16:0/18:3, PI_16:0/20:4, 5-HETE, LXA4, 15-OXOETE, 5-HEPE, 8-HETE, LTB4, GPLD1, SERPING1, C3, A2M, SERPINA6, APOA4, APCS, ITIH2, CLU, APOA2, PPBP, glu-leu, 6-ketodecanoylcarnitine, myo-inositol, chenodeoxyglycocholate, 2-hydroxy-2-methylbutanedioic acid, nonanedioic acid. In other embodiments, the prostate cancer marker comprises one or more markers selected from Tables 4, 11, 16, 22 and 30, wherein the one or more markers have a FC ratio less than 1, or a Log FC value less than 0. In yet another embodiment, the prostate cancer marker is selected from the group consisting of glu-leu, 5-HETE, 15-OXOETE, 5-HEPE, 8-HETE, and 6-ketodecanoylcarnitine.

In certain embodiments, the prostate cancer marker for diagnosis of the presence of prostate cancer in a subject selected from a population of African Americans comprises one or more markers selected from Tables 2, 5, 9, 12, 14, 17, 20, 23, 27 and 31. In certain embodiments, the prostate cancer marker comprises at least two or more markers, wherein each of the two or more markers are selected from the structural lipids set forth in Table 2, the signaling lipids set forth in Table 9, the proteins set forth in Table 14, the metabolites set forth in Table 20, and/or the markers set forth in Table 27.

In certain embodiments, the level of the marker, e.g., a prostate cancer marker, for diagnosis of the presence of prostate cancer in a subject selected from a population of African Americans, is increased when compared to the predetermined threshold value in the subject. In some embodiments, the prostate cancer marker is selected from the group consisting of FFA_18:3, FFA_20:1, TAG_54:7+NH4, TAG_54:6+NH4, PA_18:1/18:3, 13-HOTRE/13-HOTRE(R), 9-HOTRE, TXB2, 12-HEPE, 12-HETE, 13-HODE, CST3, F5, B2M, nicotinamide, eicosenoic acid, 3-hydroxybutyric acid and 2-keto-isovalerate. In other embodiments, the prostate cancer marker comprises one or more markers selected from Tables 5, 12, 17, 23 and 31, wherein the one or more markers have a FC ratio greater than 1, or a Log FC value greater than 0. In yet another embodiment, the prostate cancer marker is selected from the group consisting of FFA_18:3, 13-HOTRE/13-HOTRE(R), 9-HOTRE, nicotinamide, eicosenoic acid, 3-hydroxybutyric acid, 2-keto-isovalerate and 2-octandioic-carnitine.

In other embodiments, the level of the marker, e.g., a prostate cancer marker, for diagnosis of the presence of prostate cancer in a subject selected from a population of African Americans, is decreased when compared to the predetermined threshold value in the subject. In some embodiments, the prostate cancer marker is selected from the group consisting of CE_20:0+NH4, CE_24:0+NH4, CE_22:2+NH4, DAG_42:2+NH4, PE_36:2, 5-HEPE, 5-HETE, LTB4, PGE2/PGD2, C3, APOA4, C4BPA, MMRN2, APOA2, FGA, ABI3BP, APOA1, PROS1, COMP, CDH5, SERPINA6, 6-ketodecanoylcarnitine, glu-leu, ethanolamine, and nonanoylcarnitine. In other embodiments, the prostate cancer marker comprises one or more markers selected from Tables 5, 12, 17, 23 and 31, wherein the one or more markers have a FC ratio less than 1, or a Log FC value less than 0. In yet another embodiment, the prostate cancer marker is selected from the group consisting of 6-ketodecanoylcarnitine, glu-leu, ethanolamine, and nonanoylcarnitine and propionylcarnitine.

In certain embodiments, the ERG-positive prostate cancer marker for diagnosis of the presence of ERG-positive prostate cancer in a subject comprises one or more markers selected from Tables 6, 30 and 31. In certain embodiments, the ERG-positive prostate cancer marker comprises at least two or more markers, wherein each of the two or more markers are selected from Tables 6, 30 and 31.

In certain embodiments, the level of the marker, e.g., an ERG-positive prostate cancer marker, for diagnosis of the presence of ERG-positive prostate cancer in a subject, is increased when compared to the predetermined threshold value in the subject. In some embodiments, the ERG-positive prostate cancer marker is selected from the group consisting of CE_20:4+NH4, PG_16:1/18:3, D18:0/16:1-MONOHEX, D18:1/22:1-MONOHEX, PG_16:1/20:3. In other embodiments, the ERG-positive prostate cancer marker comprises one or more markers selected from Tables 6, 30 and 31, wherein the one or more markers have a FC ratio greater than 1, or a Log FC value greater than 0.

In other embodiments, the level of the marker, e.g., an ERG-positive prostate cancer marker, for diagnosis of the presence of ERG-positive prostate cancer in a subject, is decreased when compared to the predetermined threshold value in the subject. In some embodiments, the ERG-positive prostate cancer marker is selected from the group consisting of LPC_0-14:1, LPC_22:1, LPC_10:0, LPC_0-22:0, LPC_24:0. In other embodiments, the ERG-positive prostate cancer marker comprises one or more markers selected from Tables 6, 30 and 31, wherein the one or more markers have a FC ratio less than 1, or a Log FC value less than 0.

In certain embodiments, the high BMI prostate cancer marker for diagnosis of the presence of prostate cancer in a subject with a BMI index equal or greater than 30 comprises one or more markers selected from Tables 7, 18 and 25. In certain embodiments, the high BMI prostate cancer marker comprises at least two or more markers, wherein each of the two or more markers are selected from Tables 7, 18 and 25.

In certain embodiments, the level of the marker, e.g., a high BMI prostate cancer marker, for diagnosis of the presence of prostate cancer in a subject with a BMI index equal or greater than 30, is increased when compared to the predetermined threshold value in the subject. In some embodiments, the high BMI prostate cancer marker comprises one or more markers selected from Tables 7, 18 and 25, wherein the one or more markers have a FC ratio greater than 1, or a Log FC value greater than 0.

In other embodiments, the level of the marker, e.g., a high BMI prostate cancer marker, for diagnosis of the presence of prostate cancer in a subject with a BMI index equal or greater than 30, is decreased when compared to the predetermined threshold value in the subject. In some embodiments, the high BMI prostate cancer marker comprises one or more markers selected from Tables 7, 18 and 25, wherein the one or more markers have a FC ratio less than 1, or a Log FC value less than 0.

In certain embodiments, the marker, e.g., a prostate cancer marker, a ERG-positive prostate cancer marker, a ERG-negative prostate cancer marker, a high BMI prostate cancer marker, or a Gleason score marker comprises one or more markers with an increased level when compared to the predetermined threshold value in the subject, and/or one or more markers with a decreased level when compared to the predetermined threshold value in the subject

In certain embodiments the diagnostic methods provided herein further comprise detecting the level of one or more additional markers of prostate cancer in the biological sample and preferably further comprise comparing the level of prostate specific antigen (PSA) in the biological sample to a PSA expression level in a normal control sample. In certain embodiments, the combination of PSA level with one or more of the prostate-cancer maker levels increases the predictive value of the method.

In certain embodiments the monitoring methods provided herein further comprise detecting the level of prostate specific antigen (PSA) in the first sample and the second sample, and preferably further comprising comparing the level of PSA in the first sample with the level of expression of PSA in the second sample. In certain monitoring methods, the change in PSA level in combination with the change in prostate-cancer maker level increases the predictive value of the method.

In certain embodiments the diagnostic and monitoring methods provided herein further comprise comparing the detected level of the one or more prostate markers in the biological samples with one or more control samples wherein the control sample is one or more of a sample from the same subject at an earlier time point than the biological sample, a sample from a subject with benign prostatic hyperplasia (BPH), a sample from a subject with non-metastatic prostate cancer, a sample from a subject with metastatic prostate cancer, a sample from a subject with androgen sensitive prostate cancer, a sample from a subject with androgen insensitive prostate cancer, a sample from a subject with aggressive prostate cancer, and sample obtained from a subject with non-aggressive prostate cancer. Comparison of the marker levels in the biological samples with control samples from subjects with various normal and abnormal prostate states facilitates the differentiation between various prostate states including normal prostate and prostate cancer, benign prostate hyperplasia and prostate cancer, benign prostate hyperplasia and normal prostate, androgen dependent and androgen independent prostate cancer, aggressive prostate cancer and non-aggressive prostate cancer, aggressive prostate cancer and non-aggressive prostate cancer, or between any two or more prostate states including normal prostate, prostate cancer, benign prostate hyperplasia, androgen dependent prostate cancer, androgen independent prostate cancer, aggressive prostate cancer, non-aggressive prostate cancer, metastatic prostate cancer, and non-metastatic prostate cancer.

In certain embodiments the diagnostic and monitoring methods provided herein further comprising detecting the size of the prostate tumor in the subject. In certain embodiments the monitoring methods provided herein further comprise detecting a change in the size or relative aggressiveness of the tumor. In certain embodiments, the size of the prostate tumor in the subject is detected prior to administering the at least a portion of a treatment regimen to the subject. In certain embodiments, the size of the prostate tumor in the subject is detected after administering the at least a portion of a treatment regimen to the subject. Certain monitoring methods, further comprise comparing the size of the prostate tumor in the subject prior to administering the at least a portion of a treatment regimen to the subject to the size of the prostate tumor in the subject after administering the at least a portion of a treatment regimen to the subject. Certain other embodiments of the diagnostic and monitoring methods further comprise determining the particular stage or grade of prostate cancer, e.g., Gleason grade 1, grade 2, grade 3, grade 4, or grade 5 prostate cancer or TNM classifications. Examples of markers for that are predictive of any particular stage or phase of prostate cancer, e.g., Gleason grade 1, grade 2, grade 3, grade 4, or grade 5 prostate cancer include at least one marker selected from Table 29.

In other embodiments, the present invention also involves the analysis and consideration of any clinical and/or patient-related health data, for example, data obtained from an Electronic Medical Record (e.g., collection of electronic health information about individual patients or populations relating to various types of data, such as, demographics, medical history, medication and allergies, immunization status, laboratory test results, radiology images, vital signs, personal statistics like age and weight, and billing information).

In certain embodiments the diagnostic and monitoring methods provided herein further comprise selecting a subject for having or being suspected of having prostate cancer.

In certain embodiments the diagnostic and monitoring methods provided herein further comprising obtaining a biological sample from a subject suspected of having or being at risk of having prostate cancer.

In certain embodiments the diagnostic and monitoring methods provided herein further comprising selecting a treatment regimen for the subject based on the level of the one or more prostate cancer markers selected from Tables 1-31.

In certain embodiments the diagnostic and monitoring methods provided herein further comprising treating the subject with a regimen including one or more treatments selected from the group consisting of surgery, radiation, hormone therapy, antibody therapy, therapy with growth factors, cytokines, and chemotherapy.

In certain embodiments the diagnostic and monitoring methods provided herein further comprise selecting the one or more specific treatment regimens for the subject based on the results of the diagnostic and monitoring methods provided herein. In one embodiment, a treatment regimen known to be effective against prostate cancer having the biomarker signature detected in the subject/sample is selected for the subject. In certain embodiments, the treatment method is started, change, revised, or maintained based on the results from the diagnostic or prognostic methods of the invention, e.g., when it is determined that the subject is responding to the treatment regimen, or when it is determined that the subject is not responding to the treatment regimen, or when it is determined that the subject is insufficiently responding to the treatment regimen. In certain embodiments, the treatment method is changed based on the results from the diagnostic or prognostic methods.

In certain other embodiments the diagnostic and monitoring methods provided herein further comprise introducing one or more specific treatment regimens for the subject based on the results of the diagnostic and monitoring methods provided herein. In one embodiment, a treatment regimen known to be effective against prostate cancer is selected for the subject. In certain embodiments, the treatment method is started, change, revised, or maintained based on the results from the diagnostic or prognostic methods of the invention, e.g., when it is determined that the subject is responding to the treatment regimen, or when it is determined that the subject is not responding to the treatment regimen, or when it is determined that the subject is insufficiently responding to the treatment regimen. In certain embodiments, the treatment method is changed based on the results from the diagnostic or prognostic methods.

In yet other embodiments the diagnostic and monitoring methods provided herein further comprise the step of administering a therapeutically effective amount of an anti-prostate cancer therapy based on the results of the diagnostic and monitoring methods provided herein. In one embodiment, a treatment regimen known to be effective against prostate cancer is selected for the subject. In certain embodiments, the treatment method is administered based on the results from the diagnostic or prognostic methods of the invention, e.g., when it is determined that the subject expresses one or more biomarkers of the invention (i.e., the one or more prostate cancer markers selected from Tables 1-31) above or below some threshold level that is indicative of prostate cancer.

In certain embodiments, a change in the treatment regimen comprises changing a hormone based therapy treatment. In certain embodiments, treatments for prostate cancer include one or more of surgery, radiation, hormone therapy, antibody therapy, therapy with growth factors, cytokines, or chemotherapy based on the results of a method of the present invention for an interval prior to performing a subsequent diagnostic, prognostic, or monitoring method provided herein.

In certain embodiments of the diagnostic and monitoring methods provided herein, the method further comprises isolating a component of the biological sample.

In certain embodiments of the diagnostic and monitoring methods provided herein, the method further comprises labeling a component of the biological sample.

In certain embodiments of the diagnostic and monitoring methods provided herein, the method further comprises amplifying a component of a biological sample.

In certain embodiments of the diagnostic and monitoring methods provided herein, the method comprises forming a complex with a probe and a component of a biological sample. In certain embodiments, forming a complex with a probe comprises forming a complex with at least one non-naturally occurring reagent. In certain embodiments of the diagnostic and monitoring methods provided herein, the method comprises processing the biological sample. In certain embodiments of the diagnostic and monitoring methods provided herein, the method of detecting a level of at least two markers comprises a panel of markers. In certain embodiments of the diagnostic and monitoring methods provided herein, the method of detecting a level comprises attaching the marker to be detected to a solid surface.

The invention provides methods of selecting for administration of active treatment or against administration of active treatment of prostate cancer in a subject comprising: (1) detecting a level of a prostate cancer marker in a first sample obtained from the subject having prostate cancer at a first time wherein the subject has not been actively treated for prostate cancer, wherein the prostate cancer markers comprises one or more markers selected from Tables 1-31; (2) detecting a level of the prostate cancer marker in a second sample obtained from the subject at a second time, e.g., wherein the subject has not been actively treated; (3) comparing the level of the prostate cancer marker in the first sample with the level of the prostate cancer marker in the second sample; wherein selecting for administration of active treatment or against administration of active treatment of prostate cancer is based on the presence or absence of changes in the level of the prostate cancer marker between the first sample and the second sample.

In certain embodiments, the method further comprising obtaining a third sample obtained from the subject at a third time (e.g., wherein the subject has not been actively treated), detecting a level of a prostate cancer marker in the third sample, wherein the prostate cancer markers comprises one or more markers selected from Tables 1-31, and comparing the level the prostate cancer marker in the third sample with the level of the prostate cancer marker in the first sample and/or the one or more markers in the second sample.

In certain embodiments, an increased or decreased level of the prostate cancer marker in the second sample as compared to the level of the prostate cancer marker in the first sample is an indication that the therapy is not efficacious in the treatment of prostate cancer, wherein the prostate cancer markers comprises one or more markers selected from Tables 1-31.

In certain embodiments, an increased or decreased level the prostate cancer marker in the second sample as compared to the prostate cancer marker in the first sample is an indication for selecting active treatment for prostate cancer, wherein the prostate cancer markers comprises one or more markers selected from Tables 1-31.

In certain embodiments, the methods further comprise detecting the level of prostate specific antigen (PSA) in the first sample and the second sample, and then preferably further comprising comparing the level of PSA in the first sample with the level of PSA in the second sample.

In certain embodiments, a decrease in the level of the prostate cancer marker in the second sample as compared to the level of the prostate cancer marker in the first sample in combination with a decrease in the level of PSA in the second sample as compared to the level of PSA in the first sample has greater predictive value that the therapy is efficacious in treating prostate cancer in the subject than analysis of a single marker alone.

In certain embodiments, a decrease in the level of the prostate cancer marker in the second sample as compared to the level of the prostate cancer marker in the first sample in combination with a decrease in the level of PSA in the second sample as compared to the level of PSA in the first sample has greater predictive value for selecting against active treatment for prostate cancer than analysis of a single marker alone.

6. Monitoring Clinical Trials

Monitoring the influence of agents (e.g., drug compounds) on the level of a marker of the invention can be applied not only in basic drug screening or monitoring the treatment of a single subject, but also in clinical trials. For example, the effectiveness of an agent to affect marker expression can be monitored in clinical trials of subjects receiving treatment for an oncological disorder. In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate) comprising the steps of (i) obtaining a pre-administration sample from a subject prior to administration of the agent; (ii) detecting the level of one or more selected markers of the invention in the pre-administration sample; (iii) obtaining one or more post-administration samples from the subject; (iv) detecting the level of the marker(s) in the post-administration samples; (v) comparing the level of the marker(s) in the pre-administration sample with the level of the marker(s) in the post-administration sample or samples; and (vi) altering the administration of the agent to the subject accordingly. For example, increased expression of the lipid marker during the course of treatment may indicate ineffective dosage and the desirability of increasing the dosage. Conversely, decreased expression of the lipid marker may indicate efficacious treatment and no need to change dosage.

### H. Treatment/Therapeutics

The present invention provides methods for treating disease states, e.g., prostate cancer (e.g., ERG positive or ERG negative prostate cancer) in a subject, e.g., a human, e.g., a Caucasian, an African American, or a human with a BMI index equal or greater than 30, using one or more (e.g., 1, 2, 3, 4, 5, 6, 7, 8, 9 or more) markers selected Tables 1-31.

The present invention also provides methods for treating prostate cancer (e.g., ERG positive or ERG negative prostate cancer) with a therapeutic, e.g., a modulator, that modulates (e.g., reduces, or increases) the level of expression or activity of one or more (e.g., 1, 2, 3, 4, 5, 6, 7, 8, 9 or more) markers selected from Tables 1-31.

In certain embodiments, the modulator decreases the level of the marker, e.g., a prostate cancer marker, whose expression level is increased in a subject having prostate cancer. In some embodiments, the prostate cancer marker is selected from the group consisting of FFA_18:3, TAG_54:7+NH4, TAG_54:6+NH4, PA_18:1/20:2, FFA_18:3, FFA_20:1, TAG_54:7+NH4, TAG_54:6+NH4, PA_18:1/18:3, 6-KETO-PGF1A, TXB2, 13-HOTRE/13-HOTRE(R), 9-HOTRE, TXB2, 12-HEPE, 12-HETE, 13-HODE, APOC, APOB, ADIPOQ, SEPP1, CST3, F5, B2M, nicotinamide, eicosenoic acid, glycerylphosphorylethanolamine, nicotinamide, eicosenoic acid, 3-hydroxybutyric acid and 2-keto-isovalerate. In other embodiments, the prostate cancer marker comprises one or more markers selected from Tables 4-7, 11, 12, 16-18, 22-25, 30 and 31, wherein the one or more markers have a FC ratio greater than 1, or a Log FC value greater than 0. In yet another embodiment, the marker, e.g., a prostate cancer marker, is selected from the group consisting of 13-HOTRE/13-HOTRE(R), nicotinamide, eicosenoic acid, glycerylphosphorylethanolamine, and 3-hydroxybutyric acid.

In other embodiments, the modulator increases the level of the marker, e.g., a prostate cancer marker, whose expression level is decreased in a subject having prostate cancer. In some embodiments, the prostate cancer marker is selected from the group consisting of CE_22:2+NH4, CE_20:0+NH4, CE_22:3+NH4, DAG_40:1+NH4, CE_20:1+NH4, PI_18:0/20:5, CE_22:1+NH4, TAG_54:0+NH4, PI_18:0/20:4, PI_16:0/18:3, PI_16:0/20:4, CE_20:0+NH4, CE_24:0+NH4, CE_22:2+NH4, DAG_42:2+NH4, PE_36:2, 5-HETE, LXA4, 15-OXOETE, 5-HEPE, 8-HETE, LTB4, 5-HEPE, 5-HETE, LTB4, PGE2/PGD2, GPLD1, SERPING1, C3, A2M, SERPINA6, APOA4, APCS, ITIH2, CLU, APOA2, PPBP, C3, APOA4, C4BPA, MMRN2, APOA2, FGA, ABI3BP, APOA1, PROS1, COMP, CDH5, SERPINA6, glu-leu, 6-ketodecanoylcarnitine, myo-inositol, chenodeoxyglycocholate, 2-hydroxy-2-methylbutanedioic acid, nonanedioic acid, 6-ketodecanoylcarnitine, glu-leu, ethanolamine, and nonanoylcarnitine. In other embodiments, the prostate cancer marker comprises one or more markers selected from Tables 4-7, 11, 12, 16-18, 22-25, 30 and 31, wherein the one or more markers have a FC ratio less than 1, or a Log FC value less than 0. In yet another embodiment, the marker, e.g., a prostate cancer marker, is selected from the group consisting of 15-OXOETE, 5-HEPE, 5-HETE, 6-ketodecanoylcarnitine, glu-leu, ethanolamine, and nonanoylcarnitine. The present invention provides methods for treating prostate cancer in a subject selected from a population of Caucasians, comprising administering to the subject a modulator of a prostate cancer marker, wherein the prostate cancer marker comprises one or more markers selected from Tables 1, 4, 8, 11, 13, 16, 19, 22, 26, 29 and 30.

In certain embodiments, the modulator decreases the level of the marker, e.g., a prostate cancer marker, whose expression level is increased in a Caucasian subject having prostate cancer. In some embodiments, the prostate cancer marker is selected from the group consisting of FFA_18:3, TAG_54:7+NH4, TAG_54:6+NH4, PA_18:1/20:2, 6-KETO-PGF1A, TXB2, APOC, APOB, ADIPOQ, SEPP1, nicotinamide, eicosenoic acid, glycerylphosphorylethanolamine. In other embodiments, the prostate cancer marker comprises one or more markers selected from Tables 4, 11, 16, 22 and 30, wherein the one or more markers have a FC ratio greater than 1, or a Log FC value greater than 0. In certain embodiments, the prostate cancer marker is nicotinamide.

In other embodiments, the modulator increases the level of the marker, e.g., a prostate cancer marker, whose expression level is decreased in a Caucasian subject having prostate cancer. In some embodiments, the prostate cancer marker is selected from the group consisting of CE_22:2+NH4, CE_20:0+NH4, CE_22:3+NH4, DAG_40:1+NH4, CE_20:1+NH4, PI_18:0/20:5, CE_22:1+NH4, TAG_54:0+NH4, PI_18:0/20:4, PI_16:0/18:3, PI_16:0/20:4, 5-HETE, LXA4, 15-OXOETE, 5-HEPE, 8-HETE, LTB4, GPLD1, SERPING1, C3, A2M, SERPINA6, APOA4, APCS, ITIH2, CLU, APOA2, PPBP, glu-leu, 6-ketodecanoylcarnitine, myo-inositol, chenodeoxyglycocholate, 2-hydroxy-2-methylbutanedioic acid, nonanedioic acid. In other embodiments, the prostate cancer marker comprises one or more markers selected from Tables 4, 11, 16, 22 and 30, wherein the one or more markers have a FC ratio less than 1, or a Log FC value less than 0. In yet another embodiment, the prostate cancer marker is selected from the group consisting of glu-leu, 5-HETE, 15-OXOETE, 5-HEPE, 8-HETE, and 6-ketodecanoylcarnitine.

The present invention provides methods for treating prostate cancer in a subject selected from a population of African Americans, comprising administering to the subject a modulator of a prostate cancer marker, wherein the prostate cancer marker comprises one or more markers selected from Tables 2, 5, 9, 12, 14, 17, 20, 23, 27 and 31.

In certain embodiments, the modulator decreases the level of the marker, e.g., a prostate cancer marker, whose expression level is increased in an African American subject having prostate cancer. In some embodiments, the prostate cancer marker is selected from the group consisting of FFA_18:3, FFA_20:1, TAG_54:7+NH4, TAG_54:6+NH4, PA_18:1/18:3, 13-HOTRE/13-HOTRE(R), 9-HOTRE, TXB2, 12-HEPE, 12-HETE, 13-HODE, CST3, F5, B2M, nicotinamide, eicosenoic acid, 3-hydroxybutyric acid and 2-keto-isovalerate. In other embodiments, the prostate cancer marker comprises one or more markers selected from Tables 5, 12, 17, 23 and 31, wherein the one or more markers have a FC ratio greater than 1, or a Log FC value greater than 0. In yet another embodiment, the prostate cancer marker is selected from the group consisting of FFA_18:3, 13-HOTRE/13-HOTRE(R), 9-HOTRE, nicotinamide, eicosenoic acid, 3-hydroxybutyric acid, 2-keto-isovalerate and 2-octandioic-carnitine.

In other embodiments, the modulator increases the level of the marker, e.g., a prostate cancer marker, whose expression level is decreased in an African American subject having prostate cancer. In some embodiments, the prostate cancer marker is selected from the group consisting of CE_20:0+NH4, CE_24:0+NH4, CE_22:2+NH4, DAG_42:2+NH4, PE_36:2, 5-HEPE, 5-HETE, LTB4, PGE2/PGD2, C3, APOA4, C4BPA, MMRN2, APOA2, FGA, ABI3BP, APOA1, PROS1, COMP, CDH5, SERPINA6, 6-ketodecanoylcarnitine, glu-leu, ethanolamine, and nonanoylcarnitine. In other embodiments, the prostate cancer marker comprises one or more markers selected from Tables 5, 12, 17, 23 and 31, wherein the one or more markers have a FC ratio less than 1, or a Log FC value less than 0. In yet another embodiment, the prostate cancer marker is selected from the group consisting of 6-ketodecanoylcarnitine, glu-leu, ethanolamine, and nonanoylcarnitine and propionylcarnitine.

The present invention provides methods for treating ERG-positive prostate cancer in a subject, comprising administering to the subject a modulator of an ERG-positive prostate cancer marker, wherein the ERG-positive prostate cancer marker comprises one or more markers selected from Tables 6, 30 and 31.

In certain embodiments, the modulator decreases the level of the marker, e.g., an ERG-positive prostate cancer marker, whose expression level is increased in a subject having ERG-positive prostate cancer. In some embodiments, the ERG-positive prostate cancer marker is selected from the group consisting of CE_20:4+NH4, PG_16:1/18:3, D18:0/16:1-MONOHEX, D18:1/22:1-MONOHEX, PG 16:1/20:3. In other embodiments, the ERG-positive prostate cancer marker comprises one or more markers selected from Tables 6, 30 and 31, wherein the one or more markers have a FC ratio greater than 1, or a Log FC value greater than 0.

In other embodiments, the modulator increases the level of the marker, e.g., an ERG-positive prostate cancer marker, whose expression level is decreased in a subject having ERG-positive prostate cancer. In some embodiments, the ERG-positive prostate cancer marker is selected from the group consisting of LPC_0-14:1, LPC_22:1, LPC_10:0, LPC_0-22:0, LPC_24:0. In other embodiments, the ERG-positive prostate cancer marker comprises one or more markers selected from Tables 6, 30 and 31, wherein the one or more markers have a FC ratio less than 1, or a Log FC value less than 0.

The present invention provides methods for treating prostate cancer in a subject with a BMI index equal or greater than 30 comprising administering to the subject a modulator of a high BMI prostate cancer marker, wherein the high BMI prostate cancer marker comprises one or more markers selected from Tables 7, 18 and 25.

In certain embodiments, the modulator decreases the level of the marker, e.g., a high BMI prostate cancer marker, whose expression level is increased in a subject having prostate cancer. In some embodiments, the high BMI prostate cancer marker comprises one or more markers selected from Tables 7, 18 and 25, wherein the one or more markers have a FC ratio greater than 1, or a Log FC value greater than 0.

In other embodiments, the modulator increases the level of the marker, e.g., a high BMI prostate cancer marker, whose expression level is decreased in a subject having prostate cancer. In some embodiments, the high BMI prostate cancer marker comprises one or more markers selected from Tables 7, 18 and 25, wherein the one or more markers have a FC ratio less than 1, or a Log FC value less than 0.

The present invention provides methods for treating ERG-negative prostate cancer in a Caucasian subject with a BMI index equal or greater than 30 comprising administering to the subject a modulator of mercapto-succinyl-carnitine. In some embodiments, the modulator decreases the level or activity of mercapto-succinyl-carnitine.

The invention also provides methods for selection and/or administration of known treatment agents, especially hormone based therapies vs. non-hormone based therapies, and aggressive or active treatment vs. “watchful waiting”, depending on the detection of a change in the level of one or more (e.g., 1, 2, 3, 4, 5, 6, 7, 8, 9 or more) markers selected from Tables 1-31, as compared to a control. The selection of treatment regimens can further include the detection of PSA to assist in selection of the therapeutic methods. Selection of treatment methods can also include other diagnostic considerations and patient characteristics including results from imaging studies, tumor size or growth rates, risk of poor outcomes, disruption of daily activities, and age, Gleason scores (e.g., grade 1, grade 2, grade 3, grade 4, or grade 5 prostate cancer), TNM classifications, clinical and/or patient-related health data (e.g., data obtained from an Electronic Medical Record (e.g., collection of electronic health information about individual patients or populations relating to various types of data, such as, demographics, medical history, medication and allergies, immunization status, laboratory test results, radiology images, vital signs, personal statistics like age and weight, and billing information)).

As used herein, the term “aggressive oncological disorder”, such as aggressive prostate cancer, refers to an oncological disorder involving a fast-growing tumor. An aggressive oncological disorder typically does not respond, responds poorly, or loses response to therapeutic treatment. For example, an prostate cancer may be considered to become an aggressive prostate cancer upon loss of response to hormone therapy, necessitating treatment with chemotherapy, surgery, and/or radiation. As used herein, an aggressive prostate cancer, for example, is one that will likely or has metastasized. As used herein, an aggressive prostate cancer is one that will result in significant changes in quality of life as the tumor grows. Active treatment is therapeutically indicated for an aggressive oncological disorder, e.g., aggressive prostate cancer.

As used herein, the term “non-aggressive oncological disorder”, such as a non-aggressive prostate cancer, refers to an oncological disorder involving a slow-growing tumor. A non-aggressive oncological disorder typically responds favorably or moderately to therapeutic treatment or grows so slowly that immediate treatment is not warranted. A non-aggressive prostate tumor is one that a person skilled in the art, e.g., an oncologist, may decide to not actively treat with routine interventions for the treatment of cancer, e.g., chemotherapy, radiation, surgery, as the active treatment may do more harm than the disease, particularly in an older subject. A non-aggressive prostate tumor is one that a person skilled in the art may decide to monitor with “watchful waiting” rather than subjecting the person to any active therapeutic interventions to alter the presence or growth of the tumor (e.g., radiation, surgery, chemotherapy, hormone therapy).

1. Nucleic Acid Therapeutics

Nucleic acid therapeutics are well known in the art. Nucleic acid therapeutics include both single stranded and double stranded (i.e., nucleic acid therapeutics having a complementary region of at least 15 nucleotides in length that may be one or two nucleic acid strands) nucleic acids that are complementary to a target sequence in a cell. Nucleic acid therapeutics can be delivered to a cell in culture, e.g., by adding the nucleic acid to culture media either alone or with an agent to promote uptake of the nucleic acid into the cell. Nucleic acid therapeutics can be delivered to a cell in a subject, i.e., in vivo, by any route of administration. The specific formulation will depend on the route of administration.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions may include: 400 mM NaCl, 40 mM PIPES pH 6.4, 1 mM EDTA, 50° C. or 70° C. for 12-16 hours followed by washing. Other conditions, such as physiologically relevant conditions as may be encountered inside an organism, can apply. The skilled person will be able to determine the set of conditions most appropriate for a test of complementarity of two sequences in accordance with the ultimate application of the hybridized nucleotides.

Sequences can be “fully complementary” with respect to each when there is base-pairing of the nucleotides of the first nucleotide sequence with the nucleotides of the second nucleotide sequence over the entire length of the first and second nucleotide sequences. However, where a first sequence is referred to as “substantially complementary” with respect to a second sequence herein, the two sequences can be fully complementary, or they may form one or more, but generally not more than 4, 3 or 2 mismatched base pairs upon hybridization, while retaining the ability to hybridize under the conditions most relevant to their ultimate application. However, where two oligonucleotides are designed to form, upon hybridization, one or more single stranded overhangs as is common in double stranded nucleic acid therapeutics, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, may yet be referred to as “fully complementary” for the purposes described herein.

“Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled. Such non-Watson-Crick base pairs includes, but not limited to, G:U Wobble or Hoogstein base pairing.

The terms “complementary,” “fully complementary”, and “substantially complementary” herein may be used with respect to the base matching between the sense strand and the antisense strand of a dsRNA, or between an antisense nucleic acid or the antisense strand of dsRNA and a target sequence, as will be understood from the context of their use.

As used herein, a polynucleotide that is “substantially complementary to at least part of” a messenger RNA (mRNA) refers to a polynucleotide that is substantially complementary to a contiguous portion of the mRNA of interest (e.g., an mRNA encoding filamin B, LY9, a keratin, tubulin-beta 3, or PSA) including a 5′ UTR, an open reading frame (ORF), or a 3′ UTR. For example, a polynucleotide is complementary to at least a part of the mRNA corresponding to the protein markers of Tables 13-18.

Nucleic acid therapeutics typically include chemical modifications to improve their stability and to modulate their pharmacokinetic and pharmacodynamic properties. For example, the modifications on the nucleotides can include, but are not limited to, LNA, HNA, CeNA, 2′-hydroxyl, and combinations thereof.

Nucleic acid therapeutics may further comprise at least one phosphorothioate or methylphosphonate internucleotide linkage. The phosphorothioate or methylphosphonate internucleotide linkage modification may occur on any nucleotide of the sense strand or antisense strand or both (in nucleic acid therapeutics including a sense strand) in any position of the strand. For instance, the internucleotide linkage modification may occur on every nucleotide on the sense strand or antisense strand; each internucleotide linkage modification may occur in an alternating pattern on the sense strand or antisense strand; or the sense strand or antisense strand may contain both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand.

A. Single Stranded Therapeutics

Antisense nucleic acid therapeutic agent single stranded nucleic acid therapeutics, typically about 16 to 30 nucleotides in length and are complementary to a target nucleic acid sequence in the target cell, either in culture or in an organism.

Patents directed to antisense nucleic acids, chemical modifications, and therapeutic uses are provided, for example, in U.S. Pat. No. 5,898,031 related to chemically modified RNA-containing therapeutic compounds, and U.S. Pat. No. 6,107,094 related methods of using these compounds as therapeutic agent. U.S. Pat. No. 7,432,250 related to methods of treating patients by administering single-stranded chemically modified RNA-like compounds; and U.S. Pat. No. 7,432,249 related to pharmaceutical compositions containing single-stranded chemically modified RNA-like compounds. U.S. Pat. No. 7,629,321 is related to methods of cleaving target mRNA using a single-stranded oligonucleotide having a plurality RNA nucleosides and at least one chemical modification. Each of the patents listed in the paragraph are incorporated herein by reference.

B. Double Stranded Therapeutics

In many embodiments, the duplex region is 15-30 nucleotide pairs in length. In some embodiments, the duplex region is 17-23 nucleotide pairs in length, 17-25 nucleotide pairs in length, 23-27 nucleotide pairs in length, 19-21 nucleotide pairs in length, or 21-23 nucleotide pairs in length.

In certain embodiments, each strand has 15-30 nucleotides.

The RNAi agents that are used in the methods of the invention include agents with chemical modifications as disclosed, for example, in Publications WO 2009/073809 and WO/2012/037254, the entire contents of each of which are incorporated herein by reference.

Nucleic acid therapeutic agents for use in the methods of the invention also include double stranded nucleic acid therapeutics. An “RNAi agent,” “double stranded RNAi agent,” double-stranded RNA (dsRNA) molecule, also referred to as “dsRNA agent,” “dsRNA”, “siRNA”, “iRNA agent,” as used interchangeably herein, refers to a complex of ribonucleic acid molecules, having a duplex structure comprising two anti-parallel and substantially complementary, as defined below, nucleic acid strands. As used herein, an RNAi agent can also include dsiRNA (see, e.g., US Patent publication 20070104688, incorporated herein by reference). In general, the majority of nucleotides of each strand are ribonucleotides, but as described herein, each or both strands can also include one or more non-ribonucleotides, e.g., a deoxyribonucleotide and/or a modified nucleotide. In addition, as used in this specification, an “RNAi agent” may include ribonucleotides with chemical modifications; an RNAi agent may include substantial modifications at multiple nucleotides. Such modifications may include all types of modifications disclosed herein or known in the art. Any such modifications, as used in a siRNA type molecule, are encompassed by “RNAi agent” for the purposes of this specification and claims. The RNAi agents that are used in the methods of the invention include agents with chemical modifications as disclosed, for example, in U.S. Provisional Application No. 61/561,710, filed on Nov. 18, 2011, International Application No. PCT/US2011/051597, filed on Sep. 15, 2010, and PCT Publication WO 2009/073809, the entire contents of each of which are incorporated herein by reference. The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop.” Where the two strands are connected covalently by means other than an uninterrupted chain of nucleotides between the 3′-end of one strand and the 5′-end of the respective other strand forming the duplex structure, the connecting structure is referred to as a “linker.” The RNA strands may have the same or a different number of nucleotides. The maximum number of base pairs is the number of nucleotides in the shortest strand of the dsRNA minus any overhangs that are present in the duplex. In addition to the duplex structure, an RNAi agent may comprise one or more nucleotide overhangs. The term “siRNA” is also used herein to refer to an RNAi agent as described above.

In another aspect, the agent is a single-stranded antisense RNA molecule. An antisense RNA molecule is complementary to a sequence within the target mRNA. Antisense RNA can inhibit translation in a stoichiometric manner by base pairing to the mRNA and physically obstructing the translation machinery, see Dias, N. et al., (2002) Mol Cancer Ther 1:347-355. The antisense RNA molecule may have about 15-30 nucleotides that are complementary to the target mRNA. For example, the antisense RNA molecule may have a sequence of at least 15, 16, 17, 18, 19, 20 or more contiguous nucleotides complementary to the mRNA sequences corresponding to the protein markers of Tables 13-18.

The term “antisense strand” refers to the strand of a double stranded RNAi agent which includes a region that is substantially complementary to a target sequence (e.g., a human TTR mRNA). As used herein, the term “region complementary to part of an mRNA encoding transthyretin” refers to a region on the antisense strand that is substantially complementary to part of a TTR mRNA sequence. Where the region of complementarity is not fully complementary to the target sequence, the mismatches are most tolerated in the terminal regions and, if present, are generally in a terminal region or regions, e.g., within 6, 5, 4, 3, or 2 nucleotides of the 5′ and/or 3′ terminus.

The term “sense strand,” as used herein, refers to the strand of a dsRNA that includes a region that is substantially complementary to a region of the antisense strand.

The invention also includes molecular beacon nucleic acids having at least one region which is complementary to a nucleic acid of the invention, such that the molecular beacon is useful for quantitating the presence of the nucleic acid of the invention in a sample. A “molecular beacon” nucleic acid is a nucleic acid comprising a pair of complementary regions and having a fluorophore and a fluorescent quencher associated therewith. The fluorophore and quencher are associated with different portions of the nucleic acid in such an orientation that when the complementary regions are annealed with one another, fluorescence of the fluorophore is quenched by the quencher. When the complementary regions of the nucleic acid are not annealed with one another, fluorescence of the fluorophore is quenched to a lesser degree. Molecular beacon nucleic acids are described, for example, in U.S. Pat. No. 5,876,930.

### I. Drug Screening

As noted above, sets of markers whose expression levels correlate with one or more selected prostate disease characteristics (e.g., prostate cancer progression) are attractive targets for identification of new therapeutic agents via screens to detect compounds or entities that inhibit or enhance expression of these biomarker genes and/or their products. Accordingly, the present invention provides methods for the identification of compounds potentially useful for modulating prostate cancer progression. In particular, the present invention provides methods for the identification of agents or compounds potentially useful for modulating prostate cancer progression wherein the agents or compounds modulate (e.g., increase or decrease) the expression and/or activity of one or more of the markers selected from Tables 1-31. The present invention also provides methods for the identification of agents or compounds potentially useful for modulating ERG-positive prostate cancer progression wherein the agents or compounds modulate (e.g., increase or decrease) the level and/or activity of one or more of the markers selected from Tables 6, 30 and 31.

Such assays typically comprise a reaction between a marker of the invention and one or more assay components. The other components may be either the test compound itself, or a combination of test compounds and a natural binding partner of a marker of the invention. Compounds identified via assays such as those described herein may be useful, for example, for modulating e.g., inhibiting, ameliorating treating or preventing the disease. Compounds identified for modulating the expression level of one or more of the markers selected from Tables 1-31 are preferably further tested for activity useful in the treatment of cancer, preferably prostate cancer, e.g., inhibiting tumor cell growth, inhibiting tumor angiogenesis, inducing tumor cell apoptosis, etc. Compounds identified for modulating the level of one or more of the markers selected from Tables 6, 30 and 31 are preferably further tested for activity useful in the treatment of ERG-positive prostate cancer,

The test compounds used in the screening assays of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad Sci. U.S.A. 90:6909; Erb et al. (1994) Proc. Natl. Acad Sci. USA 91:11422; Zuckermann et al. (1994). J. Med Chem. 37:2678; Cho et al. (1993) Science 261:1303; Carrell et al. (1994) Angew. Chem. Int. Ed Engl. 33:2059; Carell et al. (1994) Angew. Chem. Int. Ed Engl. 33:2061; and in Gallop et al. (1994) J. Med Chem. 37:1233.

Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

The screening methods of the invention comprise contacting a cell, e.g., a diseased cell, especially a prostate cancer cell, with a test compound and determining the ability of the test compound to modulate the expression and/or activity of one or more of the markers selected from Tables 1-31, optionally in combination with PSA, in the cell. The screening methods of the invention also comprise contacting a cell, e.g., a diseased cell, such as an ERG-positive prostate cancer cell, with a test compound and determining the ability of the test compound to modulate the expression and/or activity of one or more of the markers selected from Tables 6, 30 and 31, optionally in combination with PSA, in the cell. The expression and/or activity of one or more of the markers selected from Tables 1-31, optionally in combination with PSA, can be determined using any methods known in the art, such as those described herein.

In another embodiment, the invention provides assays for screening candidate or test compounds which are substrates of a marker of the invention or biologically active portions thereof. In yet another embodiment, the invention provides assays for screening candidate or test compounds which bind to a marker of the invention or biologically active portions thereof. Determining the ability of the test compound to directly bind to a marker can be accomplished, for example, by any method known in the art.

This invention further pertains to novel agents identified by the above-described screening assays. Accordingly, it is within the scope of this invention to further use an agent identified as described herein in an appropriate animal model. For example, an agent capable of modulating the expression and/or activity of a marker of the invention identified as described herein can be used in an animal model to determine the efficacy, toxicity, or side effects of treatment (e.g., of prostate cancer) with such an agent. Alternatively, an agent identified as described herein can be used in an animal model to determine the mechanism of action of such an agent. Furthermore, this invention pertains to uses of novel agents identified by the above-described screening assays for treatment as described above.

In certain embodiments, the screening methods are performed using cells contained in a plurality of wells of a multi-well assay plate. Such assay plates are commercially available, for example, from Stratagene Corp. (La Jolla, Calif.) and Corning Inc. (Acton, Mass.) and include, for example, 48-well, 96-well, 384-well and 1536-well plates.

Reproducibility of the results may be tested by performing the analysis more than once with the same concentration of the same candidate compound (for example, by incubating cells in more than one well of an assay plate). Additionally, since candidate compounds may be effective at varying concentrations depending on the nature of the compound and the nature of its mechanism(s) of action, varying concentrations of the candidate compound may be tested. Generally, candidate compound concentrations from 1 fM to about 10 mM are used for screening. Preferred screening concentrations are generally between about 10 μM and about 100 μM.

The screening methods of the invention will provide “hits” or “leads,” i.e., compounds that possess a desired but not optimized biological activity. Lead optimization performed on these compounds to fulfill all physicochemical, pharmacokinetic, and toxicologic factors required for clinical usefulness may provide improved drug candidates. The present invention also encompasses these improved drug candidates and their use as therapeutics for modulating prostate cancer progression.

### J. Kits/Panels

The invention also provides compositions and kits for diagnosing, prognosing, or monitoring a disease or disorder, recurrence of a disorder, or survival of a subject being treated for a disorder (e.g., an abnormal prostate state, BPH, an oncologic disorder, e.g., prostate cancer). These kits may include one or more of the following: a reagent that specifically binds to a marker of the invention, and a set of instructions for measuring the level of the marker.

The invention also encompasses kits for detecting the presence of a marker protein or nucleic acid in a biological sample. Such kits can be used to determine if a subject has, or is at risk for developing, prostate cancer. For example, the kit can comprise a labeled compound or agent capable of detecting a marker protein or nucleic acid in a biological sample and means for determining the amount of the protein or mRNA in the sample (e.g., an antibody which binds the protein or a fragment thereof, or an oligonucleotide probe which binds to DNA or mRNA encoding the protein). Kits can also include instructions for use of the kit for practicing any of the methods provided herein or interpreting the results obtained using the kit based on the teachings provided herein. The kits can also include reagents for detection of a control protein in the sample not related to the abnormal prostate state, e.g., actin for tissue samples, albumin in blood or blood derived samples for normalization of the amount of the marker present in the sample. The kit can also include the purified marker for detection for use as a control or for quantitation of the assay performed with the kit.

Kits include a panel of reagents for use in a method to diagnose prostate cancer in a subject (or to identify a subject predisposed to developing prostate cancer, etc.), the panel comprising at least two detection reagents, wherein each detection reagent is specific for one prostate cancer-specific protein, wherein said prostate cancer-specific proteins are selected from the prostate cancer-specific protein sets provided herein.

For antibody-based kits, the kit can comprise, for example: (1) a first antibody (e.g., attached to a solid support) which binds to a first marker protein; and, optionally, (2) a second, different antibody which binds to either the first marker protein or the first antibody and is conjugated to a detectable label. In certain embodiments, the kit includes (1) a second antibody (e.g., attached to a solid support) which binds to a second marker protein; and, optionally, (2) a second, different antibody which binds to either the second marker protein or the second antibody and is conjugated to a detectable label. The first and second marker proteins are different. In an embodiment, the first and second markers are markers of the invention, e.g., one or more of the markers selected from Tables 1-31. In certain embodiments, neither the first marker nor the second marker is PSA. In certain embodiments, the kit comprises a third antibody which binds to a third marker protein which is different from the first and second marker proteins, and a second different antibody that binds to either the third marker protein or the antibody that binds the third marker protein wherein the third marker protein is different from the first and second marker proteins.

For oligonucleotide-based kits, the kit can comprise, for example: (1) an oligonucleotide, e.g., a detectably labeled oligonucleotide, which hybridizes to a nucleic acid sequence encoding a marker protein or (2) a pair of primers useful for amplifying a marker nucleic acid molecule. In certain embodiments, the kit can further include, for example: (1) an oligonucleotide, e.g., a second detectably labeled oligonucleotide, which hybridizes to a nucleic acid sequence encoding a second marker protein or (2) a pair of primers useful for amplifying the second marker nucleic acid molecule. The first and second markers are different. In an embodiment, the first and second markers are markers of the invention, e.g., one or more of the markers selected from Tables 1-31. In certain embodiments, the kit can further include, for example: (1) an oligonucleotide, e.g., a third detectably labeled oligonucleotide, which hybridizes to a nucleic acid sequence encoding a third marker protein or (2) a pair of primers useful for amplifying the third marker nucleic acid molecule wherein the third marker is different from the first and second markers. In certain embodiments, the kit includes a third primer specific for each nucleic acid marker to allow for detection using quantitative PCR methods.

For chromatography methods, the kit can include markers, including labeled markers, to permit detection and identification of one or more markers of the invention, e.g., one or more of the markers selected from Tables 1-31, and optionally PSA, by chromatography. In certain embodiments, kits for chromatography methods include compounds for derivatization of one or more markers of the invention. In certain embodiments, kits for chromatography methods include columns for resolving the markers of the method.

Reagents specific for detection of a marker of the invention, e.g., one or more of the markers selected from Tables 1-31, allow for detection and quantitation of the marker in a complex mixture, e.g., serum, tissue sample. In certain embodiments, the reagents are species specific. In certain embodiments, the reagents are not species specific. In certain embodiments, the reagents are isoform specific. In certain embodiments, the reagents are not isoform specific.

In certain embodiments, the kits for the diagnosis, monitoring, or characterization of prostate cancer comprise at least one reagent specific for the detection of the level of one or more of the markers selected from Tables 1-31. In certain embodiments, the kits further comprise instructions for the diagnosis, monitoring, or characterization of prostate cancer based on the level of the at least one marker selected from Tables 1-31. In certain embodiments, the kits further comprise instructions to detect the level of PSA in a sample in which the at least one marker selected from Tables 1-31 is detected. In certain embodiments, the kits further comprise at least one reagent for the specific detection of PSA.

The invention provides kits comprising at least one reagent specific for the detection of a level of at least one marker selected from Tables 1-31 and at least one reagent specific for the detection of a level of PSA.

In certain embodiments, the kits can also comprise, e.g., a buffering agents, a preservative, a protein stabilizing agent, reaction buffers. The kit can further comprise components necessary for detecting the detectable label (e.g., an enzyme or a substrate). The kit can also contain a control sample or a series of control samples which can be assayed and compared to the test sample. The controls can be control serum samples or control samples of purified proteins or nucleic acids, as appropriate, with known levels of target markers. Each component of the kit can be enclosed within an individual container and all of the various containers can be within a single package, along with instructions for interpreting the results of the assays performed using the kit.

The kits of the invention may optionally comprise additional components useful for performing the methods of the invention.

The invention further provides panels of reagents for detection of one or more prostate-related marker in a subject sample and at least one control reagent. In certain embodiments, the prostate cancer marker comprises at least two or more markers, wherein each of the two or more markers are selected from the structural lipids set forth in Tables 1-7, the signaling lipids set forth in Tables 8-12, the proteins set forth in Tables 13-18, the metabolites set forth in Tables 19-25, and/or the markers set forth in Tables 26-28. In other embodiments, the prostate cancer marker comprises at least two or more markers, wherein each of the two or more markers are selected from the structural lipids set forth in Tables 1-3, the signaling lipids set forth in Tables 8-10, the proteins set forth in Tables 13-15, the metabolites set forth in Tables 19-21, and/or the markers set forth in Tables 26-28.

In certain embodiments, the control reagent is to detect the marker for detection in the biological sample wherein the panel is provided with a control sample containing the marker for use as a positive control and optionally to quantitate the amount of marker present in the biological sample. In certain embodiments, the panel includes a detection reagent for a maker not related to an abnormal prostate state that is known to be present or absent in the biological sample to provide a positive or negative control, respectively. The panel can be provided with reagents for detection of a control protein in the sample not related to the abnormal prostate state, e.g., actin for tissue samples, albumin in blood or blood derived samples for normalization of the amount of the marker present in the sample. The panel can be provided with a purified marker for detection for use as a control or for quantitation of the assay performed with the panel.

In certain embodiments, the level of the prostate cancer marker in the panel is increased when compared to a control or a predetermined threshold value. In some embodiments, the prostate cancer marker is selected from the group consisting of FFA_18:3, TAG_54:7+NH4, TAG_54:6+NH4, PA_18:1/20:2, FFA_18:3, FFA_20:1, TAG_54:7+NH4, TAG_54:6+NH4, PA_18:1/18:3, 6-KETO-PGF1A, TXB2, 13-HOTRE/13-HOTRE(R), 9-HOTRE, TXB2, 12-HEPE, 12-HETE, 13-HODE, APOC, APOB, ADIPOQ, SEPP1, CST3, F5, B2M, nicotinamide, eicosenoic acid, glycerylphosphorylethanolamine, nicotinamide, eicosenoic acid, 3-hydroxybutyric acid and 2-keto-isovalerate. In other embodiments, the prostate cancer marker comprises one or more markers selected from Tables 4-7, 11, 12, 16-18, 22-25, 30 and 31, wherein the one or more markers have a FC ratio greater than 1, or a Log FC value greater than 0. In yet another embodiment, the marker, e.g., a prostate cancer marker, is selected from the group consisting of 13-HOTRE/13-HOTRE(R), nicotinamide, eicosenoic acid, glycerylphosphorylethanolamine, and 3-hydroxybutyric acid.

In certain embodiments, the level of the prostate cancer marker in the panel is decreased when compared to a control or a predetermined threshold value. In some embodiments, the prostate cancer marker is selected from the group consisting of CE_22:2+NH4, CE_20:0+NH4, CE_22:3+NH4, DAG_40:1+NH4, CE_20:1+NH4, PI_18:0/20:5, CE_22:1+NH4, TAG_54:0+NH4, PI_18:0/20:4, PI_16:0/18:3, PI_16:0/20:4, CE_20:0+NH4, CE_24:0+NH4, CE_22:2+NH4, DAG_42:2+NH4, PE 36:2, 5-HETE, LXA4, 15-OXOETE, 5-HEPE, 8-HETE, LTB4, 5-HEPE, 5-HETE, LTB4, PGE2/PGD2, GPLD1, SERPING1, C3, A2M, SERPINA6, APOA4, APCS, ITIH2, CLU, APOA2, PPBP, C3, APOA4, C4BPA, MMRN2, APOA2, FGA, ABI3BP, APOA1, PROS1, COMP, CDH5, SERPINA6, glu-leu, 6-ketodecanoylcarnitine, myo-inositol, chenodeoxyglycocholate, 2-hydroxy-2-methylbutanedioic acid, nonanedioic acid, 6-ketodecanoylcarnitine, glu-leu, ethanolamine, and nonanoylcarnitine. In other embodiments, the prostate cancer marker comprises one or more markers selected from Tables 4-7, 11, 12, 16-18, 22-25, 30 and 31, wherein the one or more markers have a FC ratio less than 1, or a Log FC value less than 0. In yet another embodiment, the marker, e.g., a prostate cancer marker, is selected from the group consisting of 15-OXOETE, 5-HEPE, 5-HETE, 6-ketodecanoylcarnitine, glu-leu, ethanolamine, and nonanoylcarnitine.

In some embodiments, the panel comprises one or more prostate cancer markers with an increased level when compared to a control or a predetermined threshold value, and/or one or more prostate cancer markers with a decreased level when compared to a control or a predetermined threshold value.

In a preferred embodiment, the panel includes reagents for detection of two or more markers of the invention (e.g., 2, 3, 4, 5, 6, 7, 8, 9), preferably in conjunction with a control reagent. In the panel, each marker is detected by a reagent specific for that marker. In certain embodiments, the panel further includes a reagent for the detection of PSA. In certain embodiments, the panel includes replicate wells, spots, or portions to allow for analysis of various dilutions (e.g., serial dilutions) of biological samples and control samples. In a preferred embodiment, the panel allows for quantitative detection of one or more markers of the invention.

In certain embodiments, the panel is a protein chip for detection of one or more markers. In certain embodiments, the panel is an ELISA plate for detection of one or more markers. In certain embodiments, the panel is a plate for quantitative PCR for detection of one or more markers.

In certain embodiments, the panel of detection reagents is provided on a single device including a detection reagent for one or more markers of the invention and at least one control sample. In certain embodiments, the panel of detection reagents is provided on a single device including a detection reagent for two or more markers of the invention and at least one control sample. In certain embodiments, multiple panels for the detection of different markers of the invention are provided with at least one uniform control sample to facilitate comparison of results between panels.

### EXAMPLES

This invention is further illustrated by the following examples which should not be construed as limiting. The contents of all references, GenBank Accession and Gene numbers, and published patents and patent applications cited throughout the application are hereby incorporated by reference. Those skilled in the art will recognize that the invention may be practiced with variations on the disclosed structures, materials, compositions and methods, and such variations are regarded as within the ambit of the invention.

### Materials and Methods

### Serum Analysis Identification of Prostate Cancer Markers

These Examples describe an analysis of prostate cancer serum signatures to determine biomarkers for the diagnosis of prostate cancer, including biomarkers for ERG positive or ERG negative tumors, biomarkers associating with African American (AA) or Caucasian American (CA) race, as well as Gleason stratification. As described below, serum lipodomic, proteomic and metabolomic quantitative profiles were assessed for patients treated with radical prostatectomy (N=495) in comparison to serum samples from control subjects (N=205) with no evidence of the disease. Patients in this study included both AA and CA men harboring ERG positive or negative tumors with a range of Gleason scores.

The serum samples from AA and CA men analyzed below were collected at Fort Belvoir in Virginia and Walter Reed Military Medical Center (WRMMC) in Bethesda, Md. The collection followed a consented institutional review board (IRB) protocol that allowed for samples to be contributed to a biobank as part of the WRMMC and Center for Prostate Disease Research (CPDR) biorepository. The sample collection protocol was as follows:
1) Informed consent was obtained from the patient;
2) A patient questionnaire was completed;
3) Serum collection tubes were filled and allowed to clot at room temperature for 30 minutes;
4) Within 1 hour of collection, samples were centrifuged at 1620 g for 15 minutes;
5) Aliquot tubes were labeled with patient ID number and collection date;
6) A minimum of 1 mL of serum was pipetted into each aliquot tube;
7) Aliquots were frozen and stored at −70° C.

Serum samples that demonstrated low PSA levels (PSA<2 ng/mL and normal DRE) under routine examination were collected as negative controls. The negative control patients were subsequently followed over the course of several years to ensure absence of any indication or clinical phenotype of prostate cancer.

AA and CA patients that exhibited elevated PSA had serum samples collected prior to any medical intervention. Following biofluid collection, men under went radical prostectomy, which allowed for the histological analysis of the entire prostate to ensure a positive prostate cancer diagnosis. Following 2009/2010, radical prostectomies underwent a transition where collection of non-hypoxic or non-ischemia tissues was rendered impossible, preventing comprehensive and accurate assessment of the whole prostate. Alternatively, in more conventional procedures several PIN biopsies are performed, however, this does not ensure comprehensive detection of cancer within the prostate throughout the entire organ. Thus, the samples analyzed within this study represent a rare snap shot of comprehensive positive diagnosis of prostate cancer in a population that was monitored for several years as well as the existence of samples that were collected prior to any intervention.

Patients characteristics of interest included: age at RP (years), self-reported race (AA, CA), body mass index (BMI in kg/m²), PSA level (ng/mL) at time of prostate cancer diagnosis, and pathologic Gleason sum (≤6; 3+4; 4+3 8-10). BMI was dichotomized per Centers for Disease Control and Prevention categories of “obese” (BMI≥30 kg/m₂) and “non-obese” (<30 kg/m₂).

Aliquots of serum of 205 age-matched negative controls (i.e., samples that demonstrated low PSA, no clinical features of prostate cancer, and had no subsequent follow up indicating high PSA or prostate cancer), as well as 495 age-matched positive control samples (i.e., samples confirmed for prostate cancer, as well as ERG status by histological analysis of radical prostatectomy samples, and a range of Gleason scores) were analyzed for expression of structural lipidomics, signaling lipidomics, proteomics, and metabolomics.

ERG status was determined by immunohistochemistry in whole-mounted sections of radical prostatectomy specimens using the highly specific anti-ERG antibody, 9FY, which was developed by Center for Prostate Disease Research (CPDR) and can also be commercially purchased from Biocare. Serum samples were analyzed by mass spectrometry for: a) quantitative proteome; b) oxidized signaling lipidome; c) untargeted quantitiave structural lipidome; d) untargeted metabolome; and e) targeted metabolome analysis.

### Statistical Analysis

Random Forest

Collected data were analyzed by both conventional differential analysis and the unbiased Random Forest approach in order to identify novel biomarker panels across all phenomic measures.

Random Forest analysis provides an unbiased selection of predictive diagnostic markers in a statistical fashion. Random Forest is a notion of the general technique of random decision forests that are an ensemble learning method for classification, regression and other tasks, that operate by constructing a multitude of decision trees at training time and outputting the class that is the mode of the classes (classification) or mean prediction (regression) of the individual trees. (see, e.g., Bregiman et al., Breiman and Cutler's Random Forests for Classification and Regression, 2015; Leo Breiman and Adele Cutler, Random Forests, https://www.stat.berkeley.edu/-breiman/RandomForests/cc_home, the contents of which are incorporated by reference herein).

Random Forest starts with a standard machine learning technique called a “decision tree”. In a decision tree, an input variable is entered at the top and as it traverses down the tree and the data gets bucketed into smaller and smaller sets. Each tree give a classification (the tree “votes” for that class), and the forest chooses the classification having the most votes over all the trees in the forest.

When a training set for the current tree is drawn by sampling with replacement, about one third of the cases are left out of the sample. This out-of-bag data is used to get a running unbiased estimate of the classification error as trees are added to the forest, and it is also used to get estimates of variable importance.

At each node of a tree, m input variables are selected at random from all the input variables and the best split on these m variables is used to split the node. At the next node, another m variables are chosen at random from all input variables. The value of m is held constant during the forest growing. Each tree is grown to the largest extent possible, and there is no pruning.

Accordingly, Random Forest corrects for decision trees' habit of over-fitting to their training set. Injecting the right kind of randomness makes them accurate classifiers. In addition, the framework in terms of strength of the individual variables and their correlations gives insight into the ability of the Random Forest to predict. Using out-of-bag estimation makes concrete the otherwise theoretical values of strength and correlation. Furthermore, Random Forest gives results competitive with the conventional boosting and adaptive bagging algorithms, yet does not progressively change the training set.

For Random Forest analysis, raw data was transformed to a mean decrease accuracy and mean decrease Gini index. Mean decrease accuracy and mean decrease Gini index are two metrics used to measure the importance of a variable to a Random Forest model. They measure the average decrease in classification accuracy or Gini index across all classes when a variable is taken out of the model. Accordingly, the higher the values of these measures are, the more important the variable is.

Differential Analysis

For conventional differential analysis, raw data was first normalized and transformed to log 2 based values. For comparison between condition A and B (A vs B):

log FC=average(samples in A)−average(samples in B)

“p” value was computed by linear modeling implemented in limma R package (Smyth, GK (2005). Limma: linear models for microarray data. In: ‘Bioinformatics and Computational Biology Solutions using Rand Bioconductor’. R. Gentleman, V. Carey, S. Dudoit, R. Irizarry, W. Huber (eds), Springer, New York, pages 397-420) for proteomics and lipidomics, and by Wilcoxon rank sum test for metabolomics. FDR was acquired by correcting p value for multiple tests using false discovery rate method.

Therefore, for data based on the conventional differential analysis, if the log FC value herein is positive, the corresponding compound is upregulated in prostate cancer. If the log FC value herein is negative, the corresponding compound is downregulated in prostate cancer. Therefore, for example, a value equal to or higher (i.e., more positive) than the positive log FC would be correlated to the presence of prostate cancer. Likewise, for example, a value equal to or lower (i.e., more negative) than the negative log FC would be correlated to the presence of prostate cancer.

### Example 1: Identification of Structural Lipids as Prostate Cancer Markers

The serum samples were thawed on ice and a 25 μL aliquot of each was taken for analysis. Each aliquot was added to 1:1 (v/v) chloroform/methanol in combination with a cocktail of specific lipid internal standards at designated concentrations for each lipid class specifically designed for serum analysis, which are added to provide accurate quantitation for each molecular species within that class. The internal standards included and their concentrations (nmol/L) were D14:1 PC (0.440), D16:1 PE (0.022), T14:0 CL (0.009), D15:0 PG (0.013), D14:0 PS (0.013), D12:0 PA (0.018), 14:0 LPE (0.004), 17:0 LPC (0.088), T17:1 TAG (0.264), d4-16:0 FFA (0.440), N12:0 SM (0.044), N17:0 Cer (0.003), 13C4-16:0 carnitine (0.00044), D17:1 DAG (0.066), M17:1 MAG (0.066), N15:0 CBS (0.066), NADA-d8 (0.001), and CoQ8 (0.001).

Lipidomic extractions were performed on each aliquot using oxytropic ion pairing based on a modified Bligh and Dyer extraction protocol, as previously described by Kiebish et al. in Journal of Neurochemistry, Vol. 106, pp. 299-312 (2008) and Journal of Lipid Research, Vol. 51, pp. 2153-2170(2010). Lipids were extracted from each aliquot on a Hamilton Robotics STAR series system (Hamilton, Reno, Nev., USA) for automation and enhanced reproducibility. Individual lipid extracts were reconstituted with 1:1 (v/v) chloroform/methanol, flushed with nitrogen, and stored at −20° C. prior to electrospray ionization-MS using an AB SCIEX TripleTOF© 5600+ system coupled to a customized direct injection loop system on an Ekspert™ microLC 200 system (Eksigent, part of AB SCIEX, Framingham, Mass., USA). Individual lipid extracts were diluted 50-fold with 3:3:3:1 (v/v) isopropano/methanol/acetonitrile/2 mM ammonium acetate for optimized ionization efficiency in the positive and negative modes. Individual lipid extracts were analyzed using a customized Data Independent Analysis (DIA) strategy on the TripleTOF© allowing for MS/MS^{ALL} high resolution and high mass accuracy analysis, as previously described by Simons et al. in Metabolites, Vol. 2, pp. 195-213 (2012), with the exception that positive mode is run for 6 minutes and negative mode is run for 8 minutes at a flow rate of 6 μL/min. Data quantitation of all lipid classes was performed using an in-house library with MultiQuant™ Software.

The data generated by the above protocol was assessed for both mean decrease accuracy and mean decrease Gini index generated by the Random Forest analysis (see Tables 1-3), as well as the log FC, pval, and FDR values generated by the conventional differential analysis (see Tables 4-7). The data generated for positive control (i.e., prostate cancer) versus negative control (normal) samples were compared between patients with different races, BMI status or ERG status. For example, Tables 1 and 4 compare Caucasian patients versus negative control. Tables 2 and 5 compare African American patients versus negative control. Table 3 compares combined Caucasian and African American patients versus negative control. Table 6 compares ERG-positive patients versus ERG-negative patients. Table 7 compares obese patients versus non-obese patients.

Tables 1-3 include the top structural lipid markers identified based on Random Forest analysis from Caucasian prostate cancer patients, African American prostate cancer patients, and prostate cancer patients from both races, respectively, versus a negative control.

TABLE 1
Structural Lipid Markers Indicative of Prostate Cancer
for Caucasians Based on Random Forest Analysis

| | | | MeanDe- | MeanDe- |
|---|---|---|---|---|
| lipid | 0 | 1 | creaseAccuracy | creaseGini |
| CE_22:2 + NH4 | 18.41743 | 19.35283 | 21.09324063 | 4.990921 |
| FFA_18:3 | 18.12178 | 17.97784 | 20.32923261 | 5.01386 |
| CE_20:0 + NH4 | 14.18267 | 17.12852 | 17.72573449 | 3.529628 |
| CE_22:3 + NH4 | 8.016416 | 12.91066 | 13.18391106 | 2.097459 |
| DAG_40:1 + NH4 | 11.04664 | 11.04759 | 12.94671581 | 2.259665 |
| CE_20:1 + NH4 | 9.886677 | 11.79352 | 12.59679781 | 1.983804 |
| TAG_54:7 + NH4 | 10.13479 | 9.717018 | 11.87820474 | 2.26447 |
| PI_18:0/20:5 | 9.556637 | 9.078302 | 11.20248987 | 1.711777 |
| CE_22:1 + NH4 | 7.463526 | 8.703051 | 10.07022431 | 1.548294 |
| TAG_54:6 + NH4 | 7.277518 | 8.706495 | 10.0567209 | 1.5596 |
| TAG_54:0 + NH4 | 6.657758 | 7.37787 | 9.503222347 | 1.596983 |
| PI_18:0/20:4 | 6.834374 | 6.891746 | 8.776918493 | 1.018922 |
| PI_16:0/18:3 | 5.629073 | 7.010844 | 8.563682912 | 0.960167 |
| PI_16:0/20:4 | 5.39636 | 7.187911 | 8.314202513 | 0.956174 |
| PA_18:1/20:2 | 8.771356 | 5.222503 | 8.196980241 | 1.53955 |

TABLE 2
Structural Lipid Markers Indicative of Prostate Cancer
for African Americans Based on Random Forest Analysis

| | | | MeanDe- | MeanDe- |
|---|---|---|---|---|
| lipid | 0 | 1 | creaseAccuracy | creaseGini |
| FFA_18:3 | 16.33237 | 15.88045 | 19.4087 | 4.711478 |
| CE_20:0 + NH4 | 6.55772 | 11.51844 | 11.34023 | 1.546749 |
| CE_24:0 + NH4 | 8.028091 | 11.19871 | 11.2658 | 1.305881 |
| FFA_20:1 | 8.801687 | 8.719645 | 11.18966 | 1.992489 |
| TAG_54:7 + NH4 | 7.405216 | 8.846574 | 10.85728 | 2.232282 |
| CE_22:2 + NH4 | 7.618117 | 9.646519 | 9.862301 | 1.161062 |
| TAG_54:6 + NH4 | 6.195354 | 7.440127 | 9.109383 | 1.759237 |
| PA_18:1/18:3 | 5.589583 | 6.5579 | 8.337349 | 1.641482 |
| DAG_42:2 + NH4 | 4.56304 | 7.729075 | 8.039675 | 0.832492 |
| PE_36:2 | 3.631419 | 6.523408 | 7.272858 | 1.201252 |

TABLE 3
Structural Lipid Markers Indicative of Prostate Cancer for Caucasians
and African Americans Based on Random Forest Analysis

| | | | MeanDe- | MeanDe- |
|---|---|---|---|---|
| lipid | 0 | 1 | creaseAccuracy | creaseGini |
| FFA_18:3 | 19.73355 | 20.33917 | 23.13059 | 7.248256 |
| PC_40:10/O-40:3 | 15.67024 | 15.90699 | 18.75597 | 6.042185 |
| FFA_18:2 | 14.46887 | 15.77439 | 17.90717 | 4.797495 |
| LPC_16:0 | 12.91777 | 11.18552 | 15.28207 | 3.867813 |
| PC_O-38:3 | 12.61801 | 10.90802 | 15.11126 | 4.269751 |
| PC_O-40:4 | 11.11717 | 11.32562 | 14.85587 | 3.82895 |
| TAG_54:7 + NH4 | 10.67669 | 12.05411 | 14.29322 | 3.138626 |
| PC_40:2 | 10.65054 | 8.895153 | 13.14588 | 3.155273 |
| AC_10:0 | 9.950454 | 11.00224 | 13.06414 | 2.247635 |
| CE_22:2 + NH4 | 10.58395 | 11.09096 | 13.04074 | 2.85935 |

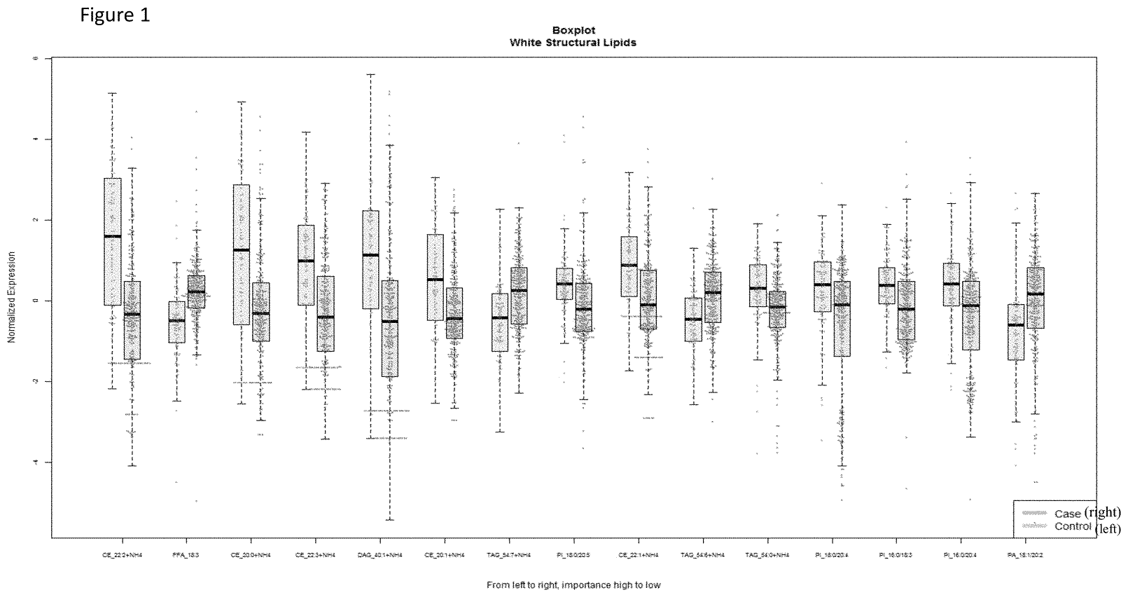

Expression levels of individual markers identified in Tables 1-3 were analyzed. FIGS. 1-3 are box plots depicting a direct comparison of normalized expression levels of individual markers identified in Tables 1-3 between Caucasian prostate cancer patients and negative controls, American African prostate cancer patients and negative controls, and prostate cancer patients from both races and negative controls, respectively. As shown in FIG. 1, expression levels of FFA_18:3, TAG_54:7+NH4, TAG_54:6+NH4, and PA_18:1/20:2 were increased in Caucasian prostate cancer patients when compared to a negative control, whereas other markers listed in Table 1 had a decreased expression level as compared to a negative control.

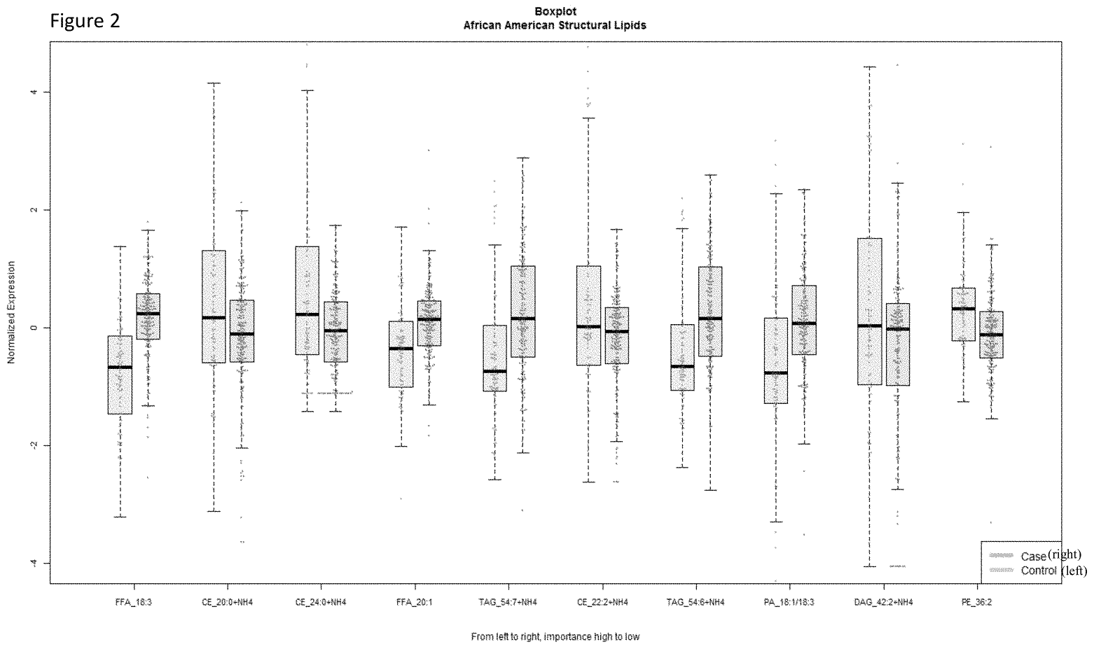

When compared the expression levels of structural lipids in African American prostate cancer patients with negative controls, an increased level for FFA_18:3, FFA_20:1, TAG_54:7+NH4, TAG_54:6+NH4 and PA_18:1/18:3 was observed in African American prostate cancer patients, whereas other markers in Table 2 showed decreased expression level in African American prostate cancer patients as compared to a negative control (FIG. 2).

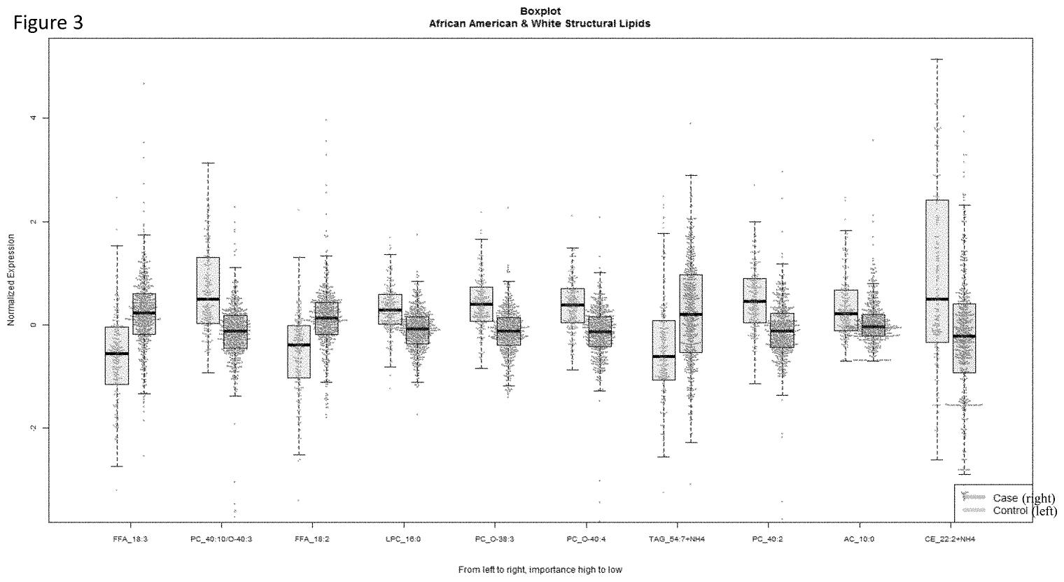

Similar comparison was performed for prostate cancer patients from both races with negative controls. As shown in FIG. 3, an increase expression level of FFA_18:3, FFA_18:2 and TAG_54:7+NH4 was observed in prostate cancer patients from both races, whereas other markers in Table 3 showed a decreased expression level in prostate cancer patients as compared to a negative control.

Receiver operating characteristic (ROC) analysis was also performed for markers identified from the Random Forest analysis. ROC curve is a graphical plot that illustrates the performance of a binary classifier system as its discrimination threshold is varied. The curve is created by plotting the true positive rate (TPR) against the false positive rate (FPR) at various threshold settings. The true-positive rate is also known as sensitivity. The false-positive rate is also known as the fall-out and can be calculated as (1—specificity). The ROC curve is thus the sensitivity as a function of fall-out. ROC analysis provides tools to select possibly optimal models and to discard suboptimal ones independently from (and prior to specifying) the cost context or the class distribution. ROC analysis is related in a direct and natural way to cost/benefit analysis of diagnostic decision making.

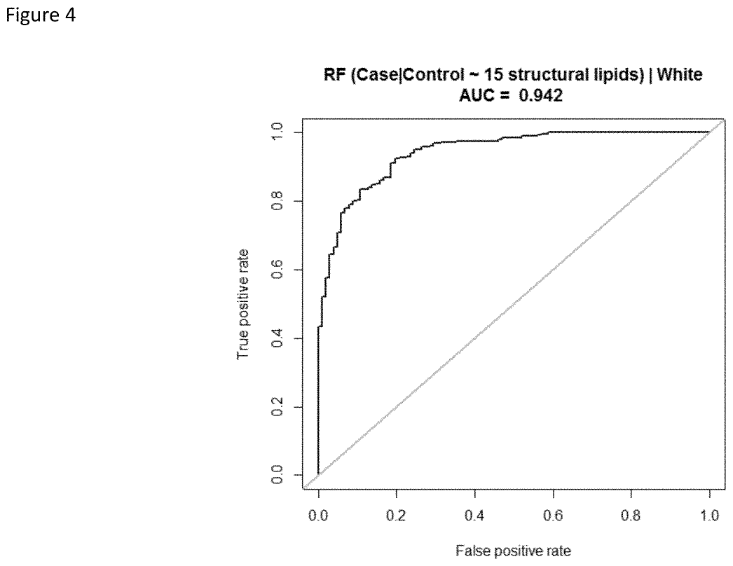

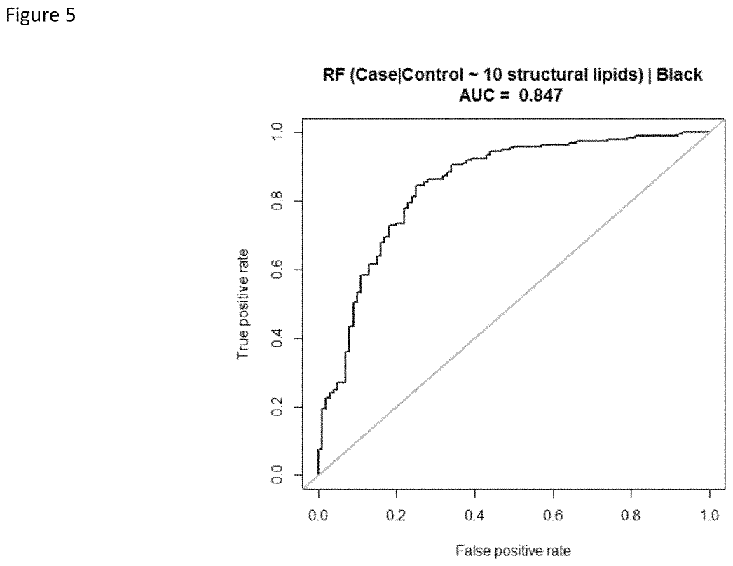

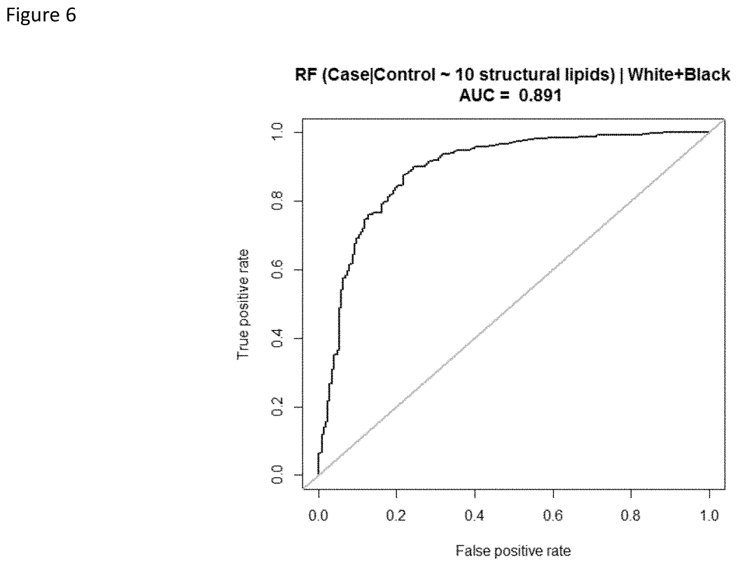

As shown in FIG. 4, the combination of the 15 structural lipids identified in Table 1 has a predictive diagnostic value of 0.942 for Caucasian prostate cancer patients. Similarly, the combination of the 10 structural lipids identified in Table 2 has a predictive diagnostic value of 0.847 for African American prostate cancer patients (FIG. 5), and the combination of the 10 structural lipids identified in Table 3 has a predictive diagnostic value of 0.891 for prostate cancer patients including both Caucasians and African Americans (FIG. 6).

These data indicate that the markers identified in Tables 1-3 may be used as biomarkers for the diagnosis and prognosis of prostate cancer, and to improve the accuracy of prostate cancer detection.

The data generated by the above protocol was also assessed for the log FC, pval, and FDR values generated by the conventional differential analysis (see Tables 4-7). Specifically, Tables 4 and 5 list structural lipid species indicative of prostate cancer in Caucasian and African American patients, respectively. Table 6 includes structural lipid species indicative of positive ERG status. Table 7 is a comparison of structural lipid species indicative of prostate cancer between obese and non-obese patients. These species demonstrated significant changes in amount from negative control to positive control as well as an FDR of less than 0.1. These data indicate that the markers identified in Tables 4-7 may also be used as biomarkers for the diagnosis and prognosis of prostate cancer, and to improve the accuracy of prostate cancer detection.

TABLE 4
Structural Lipid Markers Indicative of Prostate Cancer
for Caucasians Based on Differential Analysis

| | median. | median. | median. | median. |
|---|---|---|---|---|
| lipid | diff | diff. FC | diff. pval | diff. FDR |
| AC_10:0 | −0.25323282 | 0.839014227 | 0 | 0 |
| AC_10:1 | −0.10772729 | 0.928048887 | 0 | 0 |
| CE_16:1 + NH4 | −0.44799694 | 0.733059933 | 0.006 | 0.027337 |
| CE_16:2 + NH4 | −0.40455859 | 0.755467402 | 0.0063 | 0.028251 |
| CE_18:0 + NH4 | −0.45197981 | 0.731038955 | 0.0007 | 0.004733 |
| CE_18:1 + NH4 | −0.38661888 | 0.764920183 | 0.0111 | 0.042791 |
| CE_18:3 + NH4 | −0.41744405 | 0.748749972 | 0.0007 | 0.004733 |
| CE_20:0 + NH4 | −1.56922327 | 0.336989777 | 0 | 0 |
| CE_20:1 + NH4 | −0.97343956 | 0.509290404 | 0 | 0 |
| CE_20:2 + NH4 | −0.51141299 | 0.701535011 | 0.0011 | 0.007047 |
| CE_20:4 + NH4 | −0.36316558 | 0.777456801 | 0.0019 | 0.010721 |
| CE_22:1 + NH4 | −0.98501199 | 0.505221524 | 0 | 0 |
| CE_22:2 + NH4 | −1.92643811 | 0.263077886 | 0 | 0 |
| CE_22:3 + NH4 | −1.39032897 | 0.381477806 | 0 | 0 |
| CE_22:4 + NH4 | −0.4506945 | 0.731690534 | 0.0051 | 0.023875 |
| CE_24:2 + NH4 | −0.54579175 | 0.685015363 | 0.0155 | 0.055255 |
| CE_24:3 + NH4 | −0.64182677 | 0.640900913 | 0.0002 | 0.001563 |
| CER_D18:1/22:0 | −0.2762738 | 0.825720942 | 0.0045 | 0.021784 |
| CER_D18:1/24:0 | −0.17722327 | 0.884403555 | 0.0154 | 0.055129 |
| CER_D18:2/22:2 | 0.50321015 | 1.417363842 | 0.0111 | 0.042791 |
| D18:1/16:0-DIHEX | −0.2000054 | 0.870547305 | 0.0051 | 0.023875 |
| D18:1/16:0- | −0.22433743 | 0.855988055 | 0.0109 | 0.042405 |
| MONOHEX | | | | |
| D18:1/16:0- | −0.61035088 | 0.65503737 | 0.0003 | 0.002262 |
| TRIHEX | | | | |
| D18:1/20:0- | −0.24582037 | 0.843336108 | 0.0178 | 0.0619 |
| MONOHEX | | | | |
| D18:1/20:2-DIHEX | −0.34203439 | 0.788928035 | 0.006 | 0.027337 |
| D18:1/20:3-DIHEX | −0.23955565 | 0.84700615 | 0.0297 | 0.090373 |
| D18:1/22:0-DIHEX | −0.3926772 | 0.761714783 | 0.0003 | 0.002262 |
| D18:1/24:0- | −0.14139103 | 0.906644557 | 0.0263 | 0.08178 |
| MONOHEX | | | | |
| D18:1/24:1- | −0.30037026 | 0.812043963 | 0.0062 | 0.027949 |
| TRIHEX | | | | |
| D18:2/24:0- | −0.24491503 | 0.843865496 | 0.0128 | 0.047007 |
| MONOHEX | | | | |
| DAG_32:1 + NH4 | −0.48145803 | 0.716253391 | 0.0004 | 0.002888 |
| DAG_32:2 + NH4 | −0.4442624 | 0.73495998 | 0.0103 | 0.040817 |
| DAG_34:0 + NH4 | −0.24720844 | 0.842525093 | 0.0229 | 0.075331 |
| DAG_34:1 + NH4 | −0.41135136 | 0.751918728 | 0.0019 | 0.010721 |
| DAG_36:1 + NH4 | −0.19667393 | 0.872559895 | 0.0284 | 0.087353 |
| DAG_36:4 + NH4 | 0.33017483 | 1.257165712 | 0.0002 | 0.001563 |
| DAG_38:1 + NH4 | −0.23679211 | 0.848630179 | 0.0243 | 0.078127 |
| DAG_38:3 + NH4 | −0.27781202 | 0.824841017 | 0.006 | 0.027337 |
| DAG_38:5 + NH4 | −0.44056872 | 0.736844083 | 0.0062 | 0.027949 |
| DAG_40:1 + NH4 | −1.64192602 | 0.320428412 | 0 | 0 |
| DAG_40:3 + NH4 | −0.26861208 | 0.830117762 | 0.0117 | 0.044304 |
| DAG_40:4 + NH4 | −0.20273569 | 0.868901358 | 0.0224 | 0.07426 |
| DES_18:2 + NH4 | −0.45262071 | 0.730714272 | 0.0051 | 0.023875 |
| FFA_16:0 | 0.08057051 | 1.057436118 | 0.0012 | 0.007573 |
| FFA_16:1 | 0.30336559 | 1.23401984 | 0.0005 | 0.00358 |
| FFA_18:1 | 0.25327173 | 1.191907048 | 0 | 0 |
| FFA_18:2 | 0.4845543 | 1.39915355 | 0 | 0 |
| FFA_18:3 | 0.71132394 | 1.637305959 | 0 | 0 |
| FFA_20:1 | 0.51449393 | 1.428492964 | 0 | 0 |
| FFA_20:2 | 0.29267316 | 1.2249078 | 0.0086 | 0.035918 |
| FFA_20:4 | −0.09235321 | 0.937991523 | 0.0311 | 0.0936 |
| FFA_26:1 | −0.30235473 | 0.81092774 | 0.0114 | 0.043361 |
| LPC_14:0 | −0.42213262 | 0.746320583 | 0 | 0 |
| LPC_16:0 | −0.39813861 | 0.758836716 | 0 | 0 |
| LPC_16:1 | −0.41072748 | 0.752243959 | 0 | 0 |
| LPC_18:0 | −0.35349929 | 0.782683377 | 0 | 0 |
| LPC_18:1 | −0.36509895 | 0.776415621 | 0 | 0 |
| LPC_18:2 | −0.27500828 | 0.826445575 | 0.0001 | 0.000819 |
| LPC_18:3 | −0.45141528 | 0.731325068 | 0 | 0 |
| LPC_20:0 | −0.18358176 | 0.880514239 | 0.0109 | 0.042405 |
| LPC_20:1 | −0.16483068 | 0.892033206 | 0.0146 | 0.052708 |
| LPC_20:2 | −0.1785201 | 0.883608927 | 0.0194 | 0.066381 |
| LPC_20:3 | −0.28320502 | 0.821763399 | 0.0006 | 0.004225 |
| LPC_20:4 | −0.35174998 | 0.783632979 | 0 | 0 |
| LPC_20:5 | −0.41438516 | 0.750339202 | 0.0002 | 0.001563 |
| LPC_22:5 | −0.22874405 | 0.853377483 | 0.0123 | 0.045934 |
| LPC_O-16:0 | −0.36562682 | 0.776131589 | 0 | 0 |
| LPC_O-16:1 | −0.30015478 | 0.812165258 | 0.0044 | 0.021545 |
| LPC_O-18:1 | −0.43475115 | 0.739821355 | 0 | 0 |
| LPC_O-20:0 | −0.08230532 | 0.944547124 | 0.0253 | 0.079836 |
| LPC_O-20:1 | −0.32767914 | 0.796817291 | 0.0007 | 0.004733 |
| LPC_O-22:1 | −0.2378204 | 0.848025528 | 0.025 | 0.079478 |
| LPG_18:1 | 0.24600212 | 1.185916242 | 0.0124 | 0.045934 |
| PA_14:0/18:1 | 0.68187664 | 1.604225155 | 0.0007 | 0.004733 |
| PA_14:0/18:2 | 0.67119347 | 1.592389728 | 0.0038 | 0.019157 |
| PA_16:0/18:0 | 0.28541582 | 1.21876149 | 0.0312 | 0.0936 |
| PA_16:0/18:1 | 0.24229367 | 1.182871757 | 0.0272 | 0.084271 |
| PA_16:0/20:0 | 0.30800732 | 1.237996572 | 0.0047 | 0.022624 |
| PA_18:0/18:0 | 0.38308725 | 1.304129603 | 0.004 | 0.01993 |
| PA_18:0/18:1 | 0.54972755 | 1.463809232 | 0 | 0 |
| PA_18:0/18:2 | 0.36450232 | 1.287437431 | 0.0029 | 0.015158 |
| PA_18:0/20:2 | 0.39214538 | 1.312343492 | 0.0079 | 0.033321 |
| PA_18:0/22:4 | 0.36778698 | 1.290371948 | 0.0055 | 0.025467 |
| PA_18:1/18:1 | 0.47417967 | 1.389128124 | 0.01 | 0.040189 |
| PA_18:1/18:3 | 0.49150377 | 1.405909539 | 0.0019 | 0.010721 |
| PA_18:1/20:2 | 0.75619789 | 1.689033438 | 0 | 0 |
| PA_18:1/22:0 | 0.56834581 | 1.482822396 | 0.0001 | 0.000819 |
| PA_20:0/20:5 | −0.41677496 | 0.749097307 | 0.0208 | 0.070046 |
| PA_P18:0/18:1 | 1.41294981 | 2.662810588 | 0 | 0 |
| PC_30:0 | −0.19553534 | 0.8732488 | 0.0019 | 0.010721 |
| PC_30:1 | −0.21152097 | 0.863626268 | 0.0002 | 0.001563 |
| PC_30:2 | −0.23439116 | 0.850043655 | 0.0003 | 0.002262 |
| PC_32:0 | −0.15721526 | 0.896754348 | 0.0011 | 0.007047 |
| PC_32:1 | −0.16472662 | 0.892097549 | 0.031 | 0.0936 |
| PC_32:2 | −0.19980473 | 0.870668401 | 0.0014 | 0.008643 |
| PC_32:3 | −0.23716716 | 0.848409594 | 0.0009 | 0.005898 |
| PC_34:2 | −0.14934732 | 0.901658284 | 0.0001 | 0.000819 |
| PC_34:4 | −0.25295108 | 0.839178092 | 0.0007 | 0.004733 |
| PC_34:5 | −0.27029542 | 0.829149744 | 0.0019 | 0.010721 |
| PC_34:6 | −0.1929273 | 0.874828848 | 0.0065 | 0.028694 |
| PC_36:0/O-38:7 | −0.31588396 | 0.803358611 | 0 | 0 |
| PC_36:1 | −0.22381101 | 0.856300451 | 0.0002 | 0.001563 |
| PC_36:2 | −0.19748925 | 0.872066918 | 0.0001 | 0.000819 |
| PC_36:3 | −0.20386892 | 0.868219108 | 0.0001 | 0.000819 |
| PC_36:4 | −0.15932686 | 0.895442774 | 0.0016 | 0.009533 |
| PC_36:6 | −0.2085344 | 0.865415941 | 0.0124 | 0.045934 |
| PC_36:7 | −0.34060151 | 0.789711985 | 0 | 0 |
| PC_38:0/O-40:7 | −0.24214961 | 0.845484605 | 0 | 0 |
| PC_38:1 | −0.36796759 | 0.774873338 | 0 | 0 |
| PC_38:2 | −0.30426575 | 0.809854282 | 0 | 0 |
| PC_38:3 | −0.17304317 | 0.886969764 | 0.0028 | 0.014817 |
| PC_38:4 | −0.11230848 | 0.925106599 | 0.0329 | 0.097287 |
| PC_38:5 | −0.20841598 | 0.865486979 | 0.002 | 0.011211 |
| PC_38:7/O-38:0 | −0.21198114 | 0.863350845 | 0.0004 | 0.002888 |
| PC_38:8/O-38:1 | −0.47635272 | 0.71879251 | 0 | 0 |
| PC_40:0/O-42:7 | −0.44959272 | 0.732249536 | 0 | 0 |
| PC_40:1 | −0.50899822 | 0.702710217 | 0 | 0 |
| PC_40:10/O-40:3 | −0.91795631 | 0.529258225 | 0 | 0 |
| PC_40:2 | −0.59778965 | 0.660765538 | 0 | 0 |
| PC_40:3 | −0.43975667 | 0.737258947 | 0 | 0 |
| PC_40:4 | −0.26631299 | 0.831441699 | 0.0001 | 0.000819 |
| PC_40:8/O-40:1 | −0.30649817 | 0.808602086 | 0 | 0 |
| PC_40:9/O-40:2 | −0.58056559 | 0.66870157 | 0 | 0 |
| PC_42:0/O-44:7 | −0.36096927 | 0.778641276 | 0 | 0 |
| PC_42:1 | −0.48403236 | 0.714976456 | 0 | 0 |
| PC_42:10/O-42:3 | −0.40631982 | 0.754545696 | 0 | 0 |
| PC_42:11 | −0.40101451 | 0.757325541 | 0 | 0 |
| PC_42:2 | −0.5089338 | 0.702741596 | 0 | 0 |
| PC_42:3 | −0.51236632 | 0.701071591 | 0 | 0 |
| PC_42:4 | −0.63628879 | 0.643365826 | 0 | 0 |
| PC_42:5 | −0.34538567 | 0.787097537 | 0 | 0 |
| PC_42:6 | −0.2357447 | 0.849246516 | 0.001 | 0.006504 |
| PC_42:7/O-42:0 | −0.25824563 | 0.836104035 | 0 | 0 |
| PC_42:8/O-42:1 | −0.41261145 | 0.751262268 | 0 | 0 |
| PC_42:9/O-42:2 | −0.42249752 | 0.74613184 | 0 | 0 |
| PC_44:0 | −0.23353628 | 0.850547504 | 0.003 | 0.015585 |
| PC_44:1 | −0.29194291 | 0.816801313 | 0 | 0 |
| PC_44:10/O-44:3 | −0.45536818 | 0.729324023 | 0 | 0 |
| PC_44:11/O-44:4 | −0.27024576 | 0.829178285 | 0 | 0 |
| PC_44:2 | −0.18313128 | 0.880789222 | 0 | 0 |
| PC_44:3 | −0.48983413 | 0.712106966 | 0 | 0 |
| PC_44:4 | −0.52660464 | 0.69418657 | 0 | 0 |
| PC_44:5 | −0.46443331 | 0.724755705 | 0 | 0 |
| PC_44:6 | −0.70594184 | 0.613042143 | 0 | 0 |
| PC_44:7/O-44:0 | −0.58097888 | 0.668510034 | 0 | 0 |
| PC_44:8/O-44:1 | −0.59245394 | 0.663213859 | 0 | 0 |
| PC_44:9/O-44:2 | −0.53331187 | 0.69096672 | 0 | 0 |
| PC_O-28:0 | −0.2118663 | 0.863419571 | 0.0293 | 0.089475 |
| PC_O-32:0 | −0.17550755 | 0.885455955 | 0.0013 | 0.008144 |
| PC_O-32:1 | −0.20787364 | 0.865812395 | 0.0001 | 0.000819 |
| PC_O-32:2 | −0.10819139 | 0.927750392 | 0.0246 | 0.078794 |
| PC_O-34:0 | −0.17427576 | 0.88621229 | 0.0025 | 0.013396 |
| PC_O-34:1 | −0.11562059 | 0.922985194 | 0.0254 | 0.079855 |
| PC_O-34:3 | −0.26724751 | 0.830903299 | 0 | 0 |
| PC_O-36:0 | −0.35189932 | 0.783551866 | 0 | 0 |
| PC_O-36:1 | −0.39979938 | 0.757963678 | 0 | 0 |
| PC_O-36:3 | −0.13983245 | 0.907624558 | 0.0121 | 0.045415 |
| PC_O-36:4 | −0.22899851 | 0.853226979 | 0.0022 | 0.012171 |
| PC_O-36:5 | −0.25520724 | 0.837866768 | 0.0004 | 0.002888 |
| PC_O-36:6 | −0.19287415 | 0.874861078 | 0.0091 | 0.037275 |
| PC_O-38:2 | −0.47277182 | 0.720578834 | 0 | 0 |
| PC_O-38:3 | −0.58508454 | 0.666610275 | 0 | 0 |
| PC_O-38:4 | −0.21622218 | 0.860816609 | 0.0017 | 0.010058 |
| PC_O-38:5 | −0.16707337 | 0.890647604 | 0.0091 | 0.037275 |
| PC_O-38:6 | −0.15506881 | 0.898089537 | 0.0065 | 0.028694 |
| PC_O-40:4 | −0.6559999 | 0.634635487 | 0 | 0 |
| PC_O-40:5 | −0.47097471 | 0.721476991 | 0 | 0 |
| PC_O-40:6 | −0.15305874 | 0.899341694 | 0.0075 | 0.03195 |
| PC_O-42:4 | −0.39811201 | 0.758850707 | 0 | 0 |
| PC_O-42:5 | −0.45677866 | 0.728611333 | 0 | 0 |
| PC_O-42:6 | −0.33032501 | 0.795357286 | 0 | 0 |
| PC_O-44:6 | −0.18328782 | 0.880693657 | 0.007 | 0.030585 |
| PE_34:1 | 0.20134485 | 1.149769647 | 0.0102 | 0.040609 |
| PE_34:2 | −0.21525307 | 0.861395045 | 0.0213 | 0.071167 |
| PE_36:2 | −0.35429168 | 0.782253612 | 0.0001 | 0.000819 |
| PE_36:3 | −0.46660155 | 0.723667281 | 0 | 0 |
| PE_36:4 | −0.25567671 | 0.837594161 | 0.0048 | 0.022975 |
| PE_38:1 | −0.34924902 | 0.784992611 | 0.022 | 0.073219 |
| PE_38:3 | −0.28325719 | 0.821733683 | 0.0072 | 0.031298 |
| PE_38:4 | −0.17057511 | 0.888488427 | 0.017 | 0.059605 |
| PE_38:5 | −0.25745999 | 0.836559471 | 0.0019 | 0.010721 |
| PE_40:8/O-40:1 | −0.36446909 | 0.776754667 | 0.0165 | 0.058332 |
| PE_40:9/O-40:2 | 0.40381788 | 1.323004419 | 0.0247 | 0.078818 |
| PE_O-36:2 | 0.26872801 | 1.204745164 | 0.0213 | 0.071167 |
| PE_O-36:4 | −0.28603788 | 0.820151377 | 0.033 | 0.097287 |
| PE_O-36:5 | −0.29386653 | 0.815712955 | 0.0283 | 0.087353 |
| PE_O-38:5 | −0.24402869 | 0.844384096 | 0.009 | 0.037223 |
| PE_O-38:6 | −0.22873339 | 0.853383789 | 0.0225 | 0.074302 |
| PE_O-38:7/36:0 | −0.44503441 | 0.734566796 | 0.0149 | 0.053565 |
| PG_14:0/18:1 | 0.21973926 | 1.164523102 | 0.0035 | 0.017856 |
| PG_14:0/22:6 | 0.16450481 | 1.120781316 | 0.026 | 0.081441 |
| PG_16:0/20:1 | 0.18405364 | 1.136071504 | 0.0129 | 0.047171 |
| PG_16:1/18:2 | −0.19252928 | 0.875070235 | 0.0099 | 0.039975 |
| PG_16:1/22:1 | 0.20520196 | 1.15284772 | 0.0157 | 0.055735 |
| PG_18:0/22:0 | 0.25666482 | 1.194713606 | 0.017 | 0.059605 |
| PG_18:1/20:0 | 0.26415407 | 1.200931672 | 0.0083 | 0.034835 |
| PG_18:1/20:1 | 0.1799426 | 1.132838813 | 0.0193 | 0.066305 |
| PG_18:1/22:2 | 0.24420471 | 1.184439665 | 0.0067 | 0.029425 |
| PG_18:2/22:2 | 0.26903478 | 1.205001364 | 0.0112 | 0.042791 |
| PG_A16:0/16:0 | 0.22512035 | 1.168874752 | 0.0171 | 0.05971 |
| PG_P16:0/18:1 | 0.42189141 | 1.339682762 | 0 | 0 |
| PG_P18:0/18:0 | 0.21551615 | 1.161119251 | 0.0196 | 0.066797 |
| PG_P18:0/18:1 | 0.23725647 | 1.178748937 | 0.0233 | 0.07606 |
| PI_14:0/20:3 | −0.31909281 | 0.801573762 | 0.0024 | 0.013108 |
| PI_14:0/22:4 | −0.15785569 | 0.896356356 | 0.0053 | 0.024675 |
| PI_16:0/16:0 | −0.4772162 | 0.718362428 | 0.0014 | 0.008643 |
| PI_16:0/18:1 | −0.48468664 | 0.714652279 | 0 | 0 |
| PI_16:0/18:2 | −0.44953277 | 0.732279965 | 0 | 0 |
| PI_16:0/18:3 | −0.58764537 | 0.66542807 | 0.0001 | 0.000819 |
| PI_16:0/20:3 | −0.50176645 | 0.706241523 | 0.0015 | 0.009129 |
| PI_16:0/20:4 | −0.54752949 | 0.684190753 | 0 | 0 |
| PI_16:0/22:3 | −0.27418149 | 0.826919336 | 0.0253 | 0.079836 |
| PI_16:0/22:4 | −0.29694475 | 0.813974357 | 0.0323 | 0.096184 |
| PI_16:0/22:5 | −0.33977239 | 0.790165964 | 0.0206 | 0.069648 |
| PI_18:0/18:1 | −0.36550841 | 0.776195293 | 0.0015 | 0.009129 |
| PI_18:0/18:2 | −0.35877574 | 0.779826053 | 0.0041 | 0.020192 |
| PI_18:0/18:3 | −0.463228 | 0.725361461 | 0.0001 | 0.000819 |
| PI_18:0/20:3 | −0.32484048 | 0.79838666 | 0.0112 | 0.042791 |
| PI_18:0/20:4 | −0.50276331 | 0.705753699 | 0.0001 | 0.000819 |
| PI_18:0/20:5 | −0.62503222 | 0.648405296 | 0 | 0 |
| PI_18:0/22:2 | −0.44370148 | 0.735245788 | 0.0108 | 0.042404 |
| PI_18:0/22:5 | −0.3694925 | 0.77405474 | 0.0036 | 0.018257 |
| PI_18:1/18:1 | −0.28406048 | 0.821276271 | 0.0263 | 0.08178 |
| PI_18:1/18:2 | −0.24208643 | 0.845521632 | 0.02 | 0.067888 |
| PI_18:1/20:3 | −0.24828581 | 0.84189615 | 0.029 | 0.088878 |
| PI_18:1/20:4 | −0.39320348 | 0.761436968 | 0.0026 | 0.013845 |
| PI_20:1/20:4 | −0.35866559 | 0.779885596 | 0.0119 | 0.044862 |
| PI_A18:0/20:4 | −0.33337372 | 0.793678308 | 0.0324 | 0.096184 |
| PI_P18:0/18:0 | −0.37972182 | 0.768585775 | 0.0074 | 0.031682 |
| PI_P18:0/20:2 | −0.26649894 | 0.831334541 | 0.0318 | 0.095065 |
| PI_P20:0/16:0 | −0.33560865 | 0.792449744 | 0.0137 | 0.049882 |
| PS_18:1/20:1 | 0.25861477 | 1.196329475 | 0.0231 | 0.075697 |
| SM_D16:1/20:0 | −0.3425538 | 0.78864405 | 0.0125 | 0.046104 |
| SM_D16:1/22:0 | −0.43131033 | 0.741587931 | 0.0141 | 0.05112 |
| SM_D18:1/14:0 | −0.4202325 | 0.747304182 | 0.005 | 0.023799 |
| SM_D18:1/16:0 | −0.23971976 | 0.846909807 | 0 | 0 |
| SM_D18:1/18:0 | −0.21792582 | 0.859800694 | 0.0332 | 0.097539 |
| SM_D18:1/22:0 | −0.22936319 | 0.85301133 | 0.0064 | 0.028549 |
| SM_D18:1/24:0 | −0.21730773 | 0.860169135 | 0.03 | 0.090961 |
| SM_D18:1/24:1 | −0.23509425 | 0.849629492 | 0.0104 | 0.041022 |
| SM_D18:2/16:0 | −0.43115376 | 0.741668417 | 0.0001 | 0.000819 |
| SM_D18:2/18:0 | −0.38904778 | 0.763633458 | 0.0079 | 0.033321 |
| TAG_38:0 + NH4 | −0.37247563 | 0.772455843 | 0.0034 | 0.017451 |
| TAG_40:0 + NH4 | −0.32207977 | 0.799915898 | 0.0024 | 0.013108 |
| TAG_40:1 + NH4 | −0.69577986 | 0.617375501 | 0.0003 | 0.002262 |
| TAG_42:0 + NH4 | −0.26530342 | 0.832023729 | 0.0181 | 0.062434 |
| TAG_44:0 + NH4 | −0.93750487 | 0.522135129 | 0 | 0 |
| TAG_44:1 + NH4 | −0.89373106 | 0.538220385 | 0 | 0 |
| TAG_46:0 + NH4 | −0.78770003 | 0.579266834 | 0 | 0 |
| TAG_46:1 + NH4 | −0.94397881 | 0.519797352 | 0 | 0 |
| TAG_46:2 + NH4 | −0.83704279 | 0.559789841 | 0 | 0 |
| TAG_46:3 + NH4 | −0.3252292 | 0.798171571 | 0.0237 | 0.07707 |
| TAG_48:0 + NH4 | −0.30788104 | 0.807827386 | 0.0019 | 0.010721 |
| TAG_48:1 + NH4 | −0.59269669 | 0.663102275 | 0.0008 | 0.005325 |
| TAG_48:2 + NH4 | −0.5108951 | 0.701786889 | 0.0029 | 0.015158 |
| TAG_48:3 + NH4 | −0.5272174 | 0.693891789 | 0.0039 | 0.019546 |
| TAG_48:4 + NH4 | −0.73442882 | 0.601055943 | 0.0001 | 0.000819 |
| TAG_48:5 + NH4 | −0.42891264 | 0.742821439 | 0.0025 | 0.013396 |
| TAG_50:0 + NH4 | −0.2651032 | 0.832139207 | 0.0087 | 0.036158 |
| TAG_50:1 + NH4 | −0.28177342 | 0.822579247 | 0.0073 | 0.031572 |
| TAG_50:2 + NH4 | −0.33093333 | 0.79502199 | 0.0016 | 0.009533 |
| TAG_50:3 + NH4 | −0.3080781 | 0.807717051 | 0.0045 | 0.021784 |
| TAG_50:4 + NH4 | −0.36325388 | 0.777409218 | 0.0021 | 0.011694 |
| TAG_50:5 + NH4 | −0.51436642 | 0.700100324 | 0.0004 | 0.002888 |
| TAG_50:6 + NH4 | −0.33115436 | 0.794900197 | 0.0032 | 0.016524 |
| TAG_52:0 + NH4 | −0.49734333 | 0.708410092 | 0 | 0 |
| TAG_52:1 + NH4 | −0.37225927 | 0.772571697 | 0.0001 | 0.000819 |
| TAG_52:2 + NH4 | −0.256925 | 0.836869747 | 0.0016 | 0.009533 |
| TAG_52:6 + NH4 | −0.28714657 | 0.819521344 | 0.0181 | 0.062434 |
| TAG_52:7 + NH4 | −0.33380763 | 0.793439634 | 0.0092 | 0.037504 |
| TAG_54:0 + NH4 | −0.46622488 | 0.723856246 | 0 | 0 |
| TAG_54:1 + NH4 | −0.63231327 | 0.645141143 | 0 | 0 |
| TAG_54:2 + NH4 | −0.39665852 | 0.759615622 | 0 | 0 |
| TAG_54:3 + NH4 | −0.28368654 | 0.82148917 | 0.0009 | 0.005898 |
| TAG_54:5 + NH4 | 0.25096374 | 1.190001787 | 0.0093 | 0.037731 |
| TAG_54:6 + NH4 | 0.66452344 | 1.585044605 | 0 | 0 |
| TAG_54:7 + NH4 | 0.66615758 | 1.586841001 | 0 | 0 |
| TAG_54:8 + NH4 | 0.2827887 | 1.216544168 | 0.0074 | 0.031682 |
| TAG_56:0 + NH4 | −0.52490178 | 0.695006425 | 0 | 0 |
| TAG_56:1 + NH4 | −0.54122957 | 0.68718499 | 0 | 0 |
| TAG_56:10 + NH4 | −0.38285759 | 0.766917029 | 0.0025 | 0.013396 |
| TAG_56:2 + NH4 | −0.48343794 | 0.715271102 | 0 | 0 |
| TAG_56:3 + NH4 | −0.3619212 | 0.778127677 | 0.0006 | 0.004225 |
| TAG_56:4 + NH4 | −0.27119394 | 0.828633505 | 0.0012 | 0.007573 |
| TAG_56:5 + NH4 | −0.23024891 | 0.852487798 | 0.0102 | 0.040609 |
| TAG_56:6 + NH4 | −0.16497029 | 0.891946887 | 0.0238 | 0.077101 |
| TAG_58:2 + NH4 | −0.26172797 | 0.834088302 | 0.0239 | 0.077132 |
| TAG_58:5 + NH4 | −0.31019011 | 0.806535472 | 0.0041 | 0.020192 |
| TAG_58:6 + NH4 | −0.26282903 | 0.833451972 | 0.0004 | 0.002888 |
| TAG_60:7 + NH4 | −0.39436789 | 0.760822654 | 0.0008 | 0.005325 |

TABLE 5
Structural Lipid Markers Indicative of Prostate Cancer
for African Americans Based on Differential Analysis

| | median. | median. | median. | median. |
|---|---|---|---|---|
| lipid | diff | diff. FC | diff. pval | diff. FDR |
| AC_10:0 | −0.26610049 | 0.831564174 | 0.0004 | 0.003873 |
| AC_10:1 | −0.14395499 | 0.905034698 | 0.0032 | 0.019066 |
| AC_18:1 | 0.19200932 | 1.142353627 | 0.0024 | 0.014817 |
| AC_26:0 | −0.11923399 | 0.920676361 | 0.0184 | 0.072578 |
| CE_18:1 + NH4 | −0.19892903 | 0.871197048 | 0.014 | 0.057903 |
| CE_20:4 + NH4 | −0.17176921 | 0.887753341 | 0.0136 | 0.0568 |
| CE_24:3 + NH4 | −0.22654172 | 0.854681192 | 0.0031 | 0.0186 |
| CER_D18:1/22:1 | 0.26960757 | 1.205479878 | 0.0235 | 0.087052 |
| CER_D18:1/24:0 | −0.18580083 | 0.879160924 | 0.0097 | 0.043497 |
| D18:0/16:1- | −0.41150181 | 0.751840319 | 0.0042 | 0.023856 |
| MONOHEX | | | | |
| D18:0/18:3- | −0.53296356 | 0.69113356 | 0.0003 | 0.00308 |
| MONOHEX | | | | |
| D18:0/20:5- | −0.33318847 | 0.793780227 | 0.0015 | 0.010475 |
| MONOHEX | | | | |
| D18:1/18:0-DIHEX | −0.29035184 | 0.817702615 | 0.0045 | 0.024735 |
| D18:1/18:2-DIHEX | −0.22993761 | 0.852671765 | 0.0251 | 0.092178 |
| D18:1/24:1- | −0.28003147 | 0.823573052 | 0.0047 | 0.025506 |
| TRIHEX | | | | |
| D18:2/24:0- | −0.29464468 | 0.815273101 | 0.0006 | 0.005164 |
| MONOHEX | | | | |
| DAG_36:4 + NH4 | 0.48670972 | 1.401245481 | 0.0001 | 0.001253 |
| DAG_36:5 + NH4 | 0.65122529 | 1.570501466 | 0.0033 | 0.019525 |
| DAG_38:5 + NH4 | −0.39189981 | 0.76212534 | 0.001 | 0.007607 |
| DAG_38:6 + NH4 | −0.32145885 | 0.800260247 | 0.016 | 0.064302 |
| FFA_16:0 | 0.05317311 | 1.037544425 | 0.005 | 0.026625 |
| FFA_16:1 | 0.57381123 | 1.488450486 | 0 | 0 |
| FFA_18:0 | 0.05488579 | 1.038776866 | 0.0011 | 0.00815 |
| FFA_18:1 | 0.35029597 | 1.274822131 | 0 | 0 |
| FFA_18:2 | 0.54322895 | 1.457230352 | 0 | 0 |
| FFA_18:3 | 0.90959909 | 1.878523404 | 0 | 0 |
| FFA_20:1 | 0.48893418 | 1.403407697 | 0 | 0 |
| FFA_20:2 | 0.53061392 | 1.444543771 | 0 | 0 |
| FFA_20:3 | 0.16440923 | 1.120707065 | 0.0009 | 0.006971 |
| FFA_22:0 | −0.13975705 | 0.907671995 | 0.025 | 0.092178 |
| FFA_22:4 | 0.3588393 | 1.282393751 | 0 | 0 |
| FFA_22:5 | 0.35645723 | 1.280278105 | 0.0001 | 0.001253 |
| FFA_26:1 | −0.23256942 | 0.851117712 | 0.006 | 0.030249 |
| LPC_14:0 | −0.27507014 | 0.826410139 | 0 | 0 |
| LPC_16:0 | −0.30453574 | 0.809702737 | 0 | 0 |
| LPC_16:1 | −0.20716025 | 0.866240632 | 0.0068 | 0.033297 |
| LPC_18:0 | −0.28236051 | 0.822244575 | 0 | 0 |
| LPC_18:1 | −0.15996732 | 0.895045345 | 0.0096 | 0.043497 |
| LPC_18:2 | −0.48257951 | 0.715696828 | 0 | 0 |
| LPC_18:3 | −0.42262082 | 0.746068075 | 0 | 0 |
| LPC_20:0 | −0.36795454 | 0.774880347 | 0.0005 | 0.004484 |
| LPC_20:1 | −0.22538353 | 0.855367602 | 0.0043 | 0.024103 |
| LPC_20:2 | −0.25442687 | 0.838320102 | 0.0028 | 0.01704 |
| LPC_20:3 | −0.24649861 | 0.842939731 | 0.0006 | 0.005164 |
| LPC_20:4 | −0.32479705 | 0.798410694 | 0 | 0 |
| LPC_20:5 | −0.31892191 | 0.801668721 | 0.0096 | 0.043497 |
| LPC_22:3 | −0.14901556 | 0.901865651 | 0.0259 | 0.093901 |
| LPC_22:4 | −0.23030877 | 0.852452428 | 0.0042 | 0.023856 |
| LPC_22:6 | −0.18895894 | 0.877238515 | 0.0174 | 0.068953 |
| LPC_O-16:0 | −0.29675091 | 0.814083729 | 0.0005 | 0.004484 |
| LPC_O-16:1 | −0.40201976 | 0.756798031 | 0.0022 | 0.013884 |
| LPC_O-18:1 | −0.1735521 | 0.886656929 | 0.0166 | 0.06609 |
| LPG_18:0 | 0.39742741 | 1.317157083 | 0 | 0 |
| LPG_18:1 | 0.27865438 | 1.213062917 | 0.0018 | 0.012171 |
| LPI_16:0 | 0.19474965 | 1.144525535 | 0.0188 | 0.073814 |
| LPS_16:0 | 0.42049434 | 1.338386075 | 0.0273 | 0.097729 |
| LPS_18:0 | 0.32167982 | 1.249784905 | 0.0034 | 0.019978 |
| PA_14:0/18:1 | 0.52790744 | 1.441836364 | 0.0001 | 0.001253 |
| PA_14:0/18:2 | 0.525564 | 1.439496219 | 0.0016 | 0.010994 |
| PA_16:0/18:0 | 0.4682398 | 1.383420557 | 0.0057 | 0.02908 |
| PA_16:0/18:1 | 0.34881475 | 1.273513939 | 0.0016 | 0.010994 |
| PA_16:0/20:0 | 0.23657014 | 1.178188307 | 0.0276 | 0.098335 |
| PA_16:0/20:2 | 0.26419284 | 1.200963945 | 0.0124 | 0.053089 |
| PA_16:0/20:5 | 0.34577134 | 1.270830253 | 0.0133 | 0.055821 |
| PA_18:0/18:0 | 0.42077604 | 1.338647433 | 0.0057 | 0.02908 |
| PA_18:0/18:1 | 0.33114137 | 1.258008238 | 0.0042 | 0.023856 |
| PA_18:0/18:3 | 0.25174512 | 1.19064648 | 0.0101 | 0.044819 |
| PA_18:0/20:2 | 0.30491737 | 1.235347881 | 0.0254 | 0.092482 |
| PA_18:1/18:1 | 0.34729723 | 1.272175079 | 0.0023 | 0.014409 |
| PA_18:1/18:3 | 0.84407274 | 1.795110608 | 0 | 0 |
| PA_18:1/20:2 | 0.51223011 | 1.426253187 | 0.0005 | 0.004484 |
| PA_20:0/20:3 | −0.35771743 | 0.780398316 | 0.0277 | 0.098335 |
| PA_20:0/20:4 | −0.41432452 | 0.750370741 | 0.0012 | 0.008738 |
| PA_20:0/20:5 | −0.55304261 | 0.681581173 | 0.0014 | 0.009858 |
| PA_P18:0/18:1 | 0.75287927 | 1.685152633 | 0 | 0 |
| PC_30:0 | −0.29960485 | 0.8124749 | 0 | 0 |
| PC_30:1 | −0.20831905 | 0.86554513 | 0.0003 | 0.00308 |
| PC_30:2 | −0.22119554 | 0.857854251 | 0.0007 | 0.005735 |
| PC_30:3 | −0.21157811 | 0.863592063 | 0.0071 | 0.033984 |
| PC_32:0 | −0.21471631 | 0.86171559 | 0.0002 | 0.002185 |
| PC_32:1 | −0.18618211 | 0.878928608 | 0.0097 | 0.043497 |
| PC_32:2 | −0.16820808 | 0.889947367 | 0.0107 | 0.046751 |
| PC_32:4 | −0.1378585 | 0.908867254 | 0.0137 | 0.056939 |
| PC_34:1 | −0.15127888 | 0.900451901 | 0.0045 | 0.024735 |
| PC_34:2 | −0.10429698 | 0.930258144 | 0.0001 | 0.001253 |
| PC_34:4 | −0.3580018 | 0.780244507 | 0 | 0 |
| PC_34:5 | −0.47885148 | 0.717548632 | 0 | 0 |
| PC_34:6 | −0.30303303 | 0.810546562 | 0.0001 | 0.001253 |
| PC_36:0/O-38:7 | −0.22319649 | 0.856665272 | 0.0004 | 0.003873 |
| PC_36:1 | −0.22652879 | 0.854688852 | 0.0002 | 0.002185 |
| PC_36:2 | −0.1371354 | 0.909322906 | 0.0002 | 0.002185 |
| PC_36:3 | −0.21253606 | 0.863018828 | 0.0003 | 0.00308 |
| PC_36:4 | −0.16318645 | 0.893050429 | 0 | 0 |
| PC_36:6 | −0.2658807 | 0.83169087 | 0.002 | 0.013108 |
| PC_36:7 | −0.20433225 | 0.86794032 | 0.0009 | 0.006971 |
| PC_38:0/O-40:7 | −0.22177082 | 0.857512246 | 0.0021 | 0.013352 |
| PC_38:1 | −0.23812061 | 0.847849081 | 0.0006 | 0.005164 |
| PC_38:2 | −0.21904991 | 0.859131033 | 0.0011 | 0.00815 |
| PC_38:3 | −0.21005963 | 0.864501499 | 0.0031 | 0.0186 |
| PC_38:4 | −0.14703399 | 0.903105233 | 0.0037 | 0.021445 |
| PC_38:7/O-38:0 | −0.1609235 | 0.89445233 | 0.0206 | 0.078354 |
| PC_38:8/O-38:1 | −0.21753866 | 0.86003146 | 0.002 | 0.013108 |
| PC_40:0/O-42:7 | −0.3463178 | 0.786589155 | 0 | 0 |
| PC_40:1 | −0.54867777 | 0.683646403 | 0 | 0 |
| PC_40:10/O-40:3 | −0.32312413 | 0.799337052 | 0 | 0 |
| PC_40:2 | −0.50935845 | 0.702534778 | 0 | 0 |
| PC_40:3 | −0.38949585 | 0.763396327 | 0 | 0 |
| PC_40:4 | −0.27863303 | 0.824371749 | 0.0005 | 0.004484 |
| PC_40:8/O-40:1 | −0.31668218 | 0.802914248 | 0 | 0 |
| PC_40:9/O-40:2 | −0.35179917 | 0.783606261 | 0 | 0 |
| PC_42:0/O-44:7 | −0.33624844 | 0.792098395 | 0 | 0 |
| PC_42:1 | −0.36389402 | 0.77706435 | 0 | 0 |
| PC_42:10/O-42:3 | −0.19463004 | 0.873796941 | 0.0059 | 0.029921 |
| PC_42:11 | −0.27728152 | 0.825144378 | 0 | 0 |
| PC_42:2 | −0.42259066 | 0.746083672 | 0 | 0 |
| PC_42:3 | −0.50390632 | 0.70519477 | 0 | 0 |
| PC_42:4 | −0.39768637 | 0.759074625 | 0 | 0 |
| PC_42:5 | −0.33153049 | 0.794692983 | 0.0003 | 0.00308 |
| PC_42:6 | −0.18682436 | 0.878537419 | 0.0097 | 0.043497 |
| PC_42:7/O-42:0 | −0.25053608 | 0.840584011 | 0 | 0 |
| PC_42:8/O-42:1 | −0.28946942 | 0.818202914 | 0 | 0 |
| PC_42:9/O-42:2 | −0.21060865 | 0.864172574 | 0.0014 | 0.009858 |
| PC_44:0 | −0.23253154 | 0.85114006 | 0.0078 | 0.036315 |
| PC_44:1 | −0.28246887 | 0.822182819 | 0 | 0 |
| PC_44:10/O-44:3 | −0.30505709 | 0.809410186 | 0 | 0 |
| PC_44:11/O-44:4 | −0.2466091 | 0.842875177 | 0.0001 | 0.001253 |
| PC_44:12/O-44:5 | −0.22155135 | 0.857642705 | 0.0049 | 0.026257 |
| PC_44:2 | −0.1353445 | 0.910452401 | 0.0011 | 0.00815 |
| PC_44:3 | −0.39937042 | 0.758189078 | 0 | 0 |
| PC_44:4 | −0.44768676 | 0.733217559 | 0 | 0 |
| PC_44:5 | −0.44832712 | 0.732892182 | 0 | 0 |
| PC_44:6 | −0.43649692 | 0.738926657 | 0.0002 | 0.002185 |
| PC_44:7/O-44:0 | −0.36967044 | 0.773959275 | 0 | 0 |
| PC_44:8/O-44:1 | −0.38280479 | 0.766945098 | 0 | 0 |
| PC_44:9/O-44:2 | −0.25666152 | 0.837022599 | 0.0002 | 0.002185 |
| PC_O-32:0 | −0.24041018 | 0.846504604 | 0 | 0 |
| PC_O-32:1 | −0.31804259 | 0.802157485 | 0 | 0 |
| PC_O-32:2 | −0.20749863 | 0.866037481 | 0.0009 | 0.006971 |
| PC_O-34:0 | −0.21450394 | 0.861842447 | 0 | 0 |
| PC_O-34:1 | −0.13990178 | 0.907580942 | 0.0101 | 0.044819 |
| PC_O-34:2 | −0.22669397 | 0.854591001 | 0.0012 | 0.008738 |
| PC_O-34:3 | −0.32432226 | 0.798673494 | 0.0001 | 0.001253 |
| PC_O-34:4 | −0.26657768 | 0.831289169 | 0.002 | 0.013108 |
| PC_O-36:0 | −0.20329207 | 0.868566328 | 0.0009 | 0.006971 |
| PC_O-36:1 | −0.21258917 | 0.862987059 | 0.0007 | 0.005735 |
| PC_O-36:2 | −0.28395882 | 0.821334144 | 0 | 0 |
| PC_O-36:3 | −0.1947976 | 0.873695461 | 0.0053 | 0.027534 |
| PC_O-36:4 | −0.24155182 | 0.845835009 | 0.0001 | 0.001253 |
| PC_O-36:5 | −0.36140417 | 0.77840659 | 0.0001 | 0.001253 |
| PC_O-36:6 | −0.21043048 | 0.864279304 | 0.0002 | 0.002185 |
| PC_O-38:2 | −0.33328818 | 0.793725368 | 0 | 0 |
| PC_O-38:3 | −0.42021183 | 0.747314888 | 0 | 0 |
| PC_O-38:4 | −0.22538775 | 0.8553651 | 0 | 0 |
| PC_O-38:5 | −0.17467333 | 0.885968106 | 0.0074 | 0.034833 |
| PC_O-38:6 | −0.18449592 | 0.87995648 | 0.0018 | 0.012171 |
| PC_O-40:4 | −0.30820425 | 0.807646427 | 0 | 0 |
| PC_O-40:5 | −0.22281312 | 0.856892946 | 0.0003 | 0.00308 |
| PC_O-40:6 | −0.13822162 | 0.908638525 | 0.0144 | 0.05927 |
| PC_O-42:4 | −0.27797578 | 0.824747394 | 0 | 0 |
| PC_O-42:5 | −0.20173494 | 0.869504296 | 0.0008 | 0.00643 |
| PC_O-42:6 | −0.23803002 | 0.847902321 | 0.0005 | 0.004484 |
| PC_O-44:6 | −0.2669645 | 0.831066311 | 0.0005 | 0.004484 |
| PE_36:2 | −0.44244878 | 0.735884483 | 0 | 0 |
| PE_36:3 | −0.57838593 | 0.669712625 | 0 | 0 |
| PE_36:4 | −0.36487084 | 0.776538393 | 0.0001 | 0.001253 |
| PE_36:5 | −0.34937653 | 0.784923234 | 0.007 | 0.033695 |
| PE_38:4 | −0.17394315 | 0.886416628 | 0.0062 | 0.030891 |
| PE_38:5 | −0.29663363 | 0.814149911 | 0.0035 | 0.020425 |
| PE_38:7/O-38:0 | −0.59752156 | 0.660888336 | 0.0002 | 0.002185 |
| PE_40:7/O-40:0 | −0.22708709 | 0.854358165 | 0.0223 | 0.084069 |
| PE_40:8/O-40:1 | −0.34531852 | 0.787134173 | 0.0132 | 0.055675 |
| PE_40:9/O-40:2 | −0.53414122 | 0.690569623 | 0.001 | 0.007607 |
| PE_O-36:5 | −0.35696338 | 0.780806312 | 0.0004 | 0.003873 |
| PE_O-38:4 | −0.18328995 | 0.880692357 | 0.0226 | 0.084084 |
| PE_O-38:6 | −0.27344205 | 0.827343275 | 0.0002 | 0.002185 |
| PE_O-38:7/36:0 | −0.36868233 | 0.774489546 | 0.0002 | 0.002185 |
| PG_14:0/16:0 | 0.20376161 | 1.151697321 | 0.0064 | 0.031702 |
| PG_14:0/18:1 | 0.29730027 | 1.228842714 | 0.0005 | 0.004484 |
| PG_16:0/16:1 | 0.15836363 | 1.116020579 | 0.0193 | 0.075429 |
| PG_16:0/22:2 | 0.17771298 | 1.131089413 | 0.0203 | 0.078261 |
| PG_16:1/18:2 | −0.2686923 | 0.830071606 | 0.0102 | 0.045028 |
| PG_18:0/22:2 | 0.25730571 | 1.195244453 | 0.0045 | 0.024735 |
| PG_18:1/20:3 | 0.23760323 | 1.17903229 | 0.0194 | 0.075474 |
| PG_18:1/20:4 | 0.23736784 | 1.178839935 | 0.0078 | 0.036315 |
| PG_18:1/20:5 | 0.39640507 | 1.316224033 | 0.0085 | 0.039359 |
| PG_18:1/22:2 | 0.25488678 | 1.193242096 | 0.0066 | 0.032504 |
| PG_18:1/22:3 | 0.31095029 | 1.240524553 | 0.0056 | 0.028916 |
| PG_18:1/22:4 | 0.29043011 | 1.223004837 | 0.0157 | 0.063395 |
| PG_18:1/22:5 | 0.23020558 | 1.173002087 | 0.0074 | 0.034833 |
| PG_18:2/20:0 | −0.18170805 | 0.881658556 | 0.0046 | 0.025123 |
| PG_20:3/20:4 | 0.21369982 | 1.159658341 | 0.0164 | 0.0656 |
| PG_20:3/22:4 | 0.27847077 | 1.212908542 | 0.0283 | 0.099635 |
| PG_A16:0/18:0 | 0.23473919 | 1.176693996 | 0.0051 | 0.026989 |
| PG_A18:0/16:0 | 0.23506737 | 1.176961697 | 0.0006 | 0.005164 |
| PG_P16:0/14:0 | 0.18534538 | 1.137089159 | 0.0216 | 0.081792 |
| PG_P16:0/18:0 | 0.26133719 | 1.198589126 | 0.0024 | 0.014817 |
| PG_P16:0/18:1 | 0.1619704 | 1.118814146 | 0.0111 | 0.048251 |
| PG_P18:0/16:0 | 0.18795276 | 1.139146076 | 0.0089 | 0.040988 |
| PG_P20:0/14:0 | 0.31299151 | 1.242280971 | 0.0061 | 0.030572 |
| PI_18:1/20:2 | 0.26963317 | 1.205501269 | 0.0146 | 0.059518 |
| PI_18:1/22:1 | 0.57529502 | 1.489982122 | 0.0007 | 0.005735 |
| PI_18:1/22:2 | 0.3430468 | 1.268432546 | 0.0113 | 0.048871 |
| PI_18:2/22:0 | 0.32472347 | 1.252424356 | 0.0007 | 0.005735 |
| PI_18:2/22:1 | 0.39697705 | 1.316745975 | 0.0043 | 0.024103 |
| PI_18:3/20:0 | 0.14515606 | 1.105850269 | 0.0225 | 0.084084 |
| PI_P20:0/18:2 | 0.37230715 | 1.294421208 | 0.0048 | 0.025884 |
| PS_14:0/18:1 | 0.30794866 | 1.237946236 | 0.0129 | 0.054681 |
| PS_16:0/16:0 | 0.32723005 | 1.254602246 | 0.0196 | 0.075905 |
| PS_16:0/18:1 | 0.3797972 | 1.301158938 | 0.0021 | 0.013352 |
| PS_16:1/16:1 | 0.46898231 | 1.384132744 | 0.0008 | 0.00643 |
| PS_18:1/20:1 | 0.36229379 | 1.285468083 | 0.0107 | 0.046751 |
| PS_18:1/22:3 | 0.40032229 | 1.319802714 | 0.0007 | 0.005735 |
| SM_D16:1/24:1 | −0.35893971 | 0.779737427 | 0.0204 | 0.078292 |
| SM_D18:1/16:0 | −0.13488601 | 0.91074179 | 0.0226 | 0.084084 |
| SM_D18:1/24:0 | −0.16097405 | 0.89442099 | 0.0151 | 0.061263 |
| SM_D18:2/16:0 | −0.39721984 | 0.75932013 | 0.0001 | 0.001253 |
| SM_D18:2/24:0 | −0.25149221 | 0.840027108 | 0.0069 | 0.033593 |
| TAG_40:0 + NH4 | −0.33805054 | 0.791109587 | 0.0052 | 0.02718 |
| TAG_40:1 + NH4 | −0.48853231 | 0.712749827 | 0.0004 | 0.003873 |
| TAG_44:0 + NH4 | −0.86319672 | 0.54973311 | 0 | 0 |
| TAG_44:1 + NH4 | −0.84799402 | 0.555556667 | 0 | 0 |
| TAG_46:0 + NH4 | −0.69546468 | 0.617510392 | 0.0013 | 0.009308 |
| TAG_46:1 + NH4 | −0.87299099 | 0.546013683 | 0.0004 | 0.003873 |
| TAG_46:2 + NH4 | −0.65366182 | 0.635664832 | 0.0021 | 0.013352 |
| TAG_48:1 + NH4 | −0.44898064 | 0.732560268 | 0.0121 | 0.052067 |
| TAG_48:2 + NH4 | −0.3247116 | 0.798457985 | 0.0282 | 0.099635 |
| TAG_48:3 + NH4 | −0.45231334 | 0.730869969 | 0.0074 | 0.034833 |
| TAG_48:4 + NH4 | −0.55376001 | 0.681242331 | 0.0021 | 0.013352 |
| TAG_50:5 + NH4 | −0.32164267 | 0.800158289 | 0.0253 | 0.092482 |
| TAG_50:6 + NH4 | −0.49112124 | 0.711471938 | 0.0002 | 0.002185 |
| TAG_52:8 + NH4 | −0.4251239 | 0.744774767 | 0.0013 | 0.009308 |
| TAG_54:1 + NH4 | −0.29198261 | 0.816778836 | 0.0027 | 0.01655 |
| TAG_54:2 + NH4 | −0.2293474 | 0.853020667 | 0.0264 | 0.095266 |
| TAG_54:5 + NH4 | 0.40648291 | 1.325450608 | 0.0019 | 0.012746 |
| TAG_54:6 + NH4 | 0.82117369 | 1.766842805 | 0 | 0 |
| TAG_54:7 + NH4 | 0.89706381 | 1.862272006 | 0 | 0 |
| TAG_56:10 + NH4 | −0.31806357 | 0.80214582 | 0.0265 | 0.095266 |
| TAG_58:13 + NH4 | 0.36059985 | 1.283959637 | 0.0052 | 0.02718 |
| TAG_58:6 + NH4 | −0.19495351 | 0.873601047 | 0.0127 | 0.054102 |
| TAG_58:9 + NH4 | −0.19273805 | 0.874943614 | 0.0145 | 0.059394 |
| TAG_60:10 + NH4 | −0.24550971 | 0.843517725 | 0.0205 | 0.078323 |
| TAG_60:7 + NH4 | −0.24244729 | 0.845310169 | 0.007 | 0.033695 |

TABLE 6
Structural Lipid Markers Indicative of Positive
ERG Status Based on Differential Analysis

| Lipid | logFC | FC | P. Value | FDR |
|---|---|---|---|---|
| PG_18:3/22:5 | 0.247127 | 1.186841 | 0.000141 | 0.065572 |
| PG_P18:0/16:0 | 0.273413 | 1.208663 | 0.000154 | 0.065572 |

TABLE 7
Comparison of Structural Lipid Markers Indicative of Prostate Cancer
in Obese and Non-Obese Patients Based on Differential Analysis

| Lipid | logFC | FC | P. Value | FDR |
|---|---|---|---|---|
| DAG_30:0 + NH4 | 0.606388908 | 1.522443732 | 0.00223 | 0.079168 |
| DAG_32:2 + NH4 | 0.699272958 | 1.623686334 | 0.000371 | 0.040179 |
| DAG_36:2 + NH4 | 0.325587431 | 1.253174597 | 0.001771 | 0.071859 |
| DAG_36:3 + NH4 | 0.394980671 | 1.314925143 | 0.000767 | 0.046698 |
| DAG_38:5 + NH4 | 0.643668643 | 1.562296892 | 0.000377 | 0.040179 |
| LPC_18:1 | −0.20671144 | 0.866510156 | 0.001224 | 0.054867 |
| LPC_18:2 | −0.19638029 | 0.872737512 | 0.002039 | 0.078965 |
| PC_44:8/O-44:1 | 0.212900104 | 1.159015696 | 0.002879 | 0.084589 |
| TAG_48:1 + NH4 | 0.457474597 | 1.373136071 | 0.002187 | 0.079168 |
| TAG_50:0 + NH4 | 0.316134696 | 1.244990468 | 0.00147 | 0.062618 |
| TAG_50:1 + NH4 | 0.467055319 | 1.382285208 | 0.000241 | 0.040179 |
| TAG_50:2 + NH4 | 0.392235035 | 1.31242505 | 0.000907 | 0.048327 |
| TAG_50:3 + NH4 | 0.444743392 | 1.361072005 | 0.000149 | 0.040179 |
| TAG_50:4 + NH4 | 0.354218518 | 1.278292966 | 0.002694 | 0.083992 |
| TAG_50:5 + NH4 | 0.393721596 | 1.313778076 | 0.001123 | 0.053141 |
| TAG_52:0 + NH4 | 0.297512698 | 1.229023667 | 0.000693 | 0.046698 |
| TAG_52:1 + NH4 | 0.36395124 | 1.286945751 | 0.00024 | 0.040179 |
| TAG_52:2 + NH4 | 0.296161801 | 1.227873384 | 0.0011 | 0.053141 |
| TAG_52:3 + NH4 | 0.295518564 | 1.22732605 | 0.000673 | 0.046698 |
| TAG_52:4 + NH4 | 0.343415477 | 1.268756732 | 0.000755 | 0.046698 |
| TAG_52:5 + NH4 | 0.321352711 | 1.249501567 | 0.002361 | 0.080451 |
| TAG_52:6 + NH4 | 0.409383994 | 1.328118609 | 0.000333 | 0.040179 |
| TAG_54:2 + NH4 | 0.320270363 | 1.248564509 | 0.002501 | 0.081946 |
| TAG_56:5 + NH4 | 0.327715825 | 1.255024759 | 0.000169 | 0.040179 |
| TAG_56:6 + NH4 | 0.305514281 | 1.235859108 | 3.22E−05 | 0.027446 |
| TAG_56:8 + NH4 | 0.291555403 | 1.223959145 | 0.00276 | 0.083992 |
| TAG_58:6 + NH4 | 0.309762127 | 1.239503313 | 0.000627 | 0.046698 |
| TAG_58:7 + NH4 | 0.297867963 | 1.229326353 | 0.000908 | 0.048327 |
| TAG_60:9 + NH4 | 0.362561712 | 1.285706829 | 0.000611 | 0.046698 |

### Example 2: Identification of Signaling Lipids as Prostate Cancer Markers

### Materials

Standards of oxidized lipids and deuterium labeled internal standards were purchased from Cayman Chemical (Ann Arbor, Mich., USA) and Santa Cruz Biotechnology, Inc. (Dallas, Tex., USA). C18 SPE cartridges were purchased from Biotage (Uppsala, Sweden). Organic solvents are acquired from Sigma-Aldrich (St. Louis, Mo., USA), Fisher Scientific (Waltham, Mass., USA), and VWR International (Radnor, Pa., USA).

### Solid Phase Extraction (SPE) of Serum Samples

A 100 μL aliquot of each of the plasma samples thawed on ice was taken for analysis. A mixture of deuterium-labeled internal standards (i.e., d4-9-HODE, d4-9,10-diHOME, d8-5S-HETE, and d4-LTB4-1 ng each) was added to each aliquot, followed by 300 μL of ice cold methanol (MeOH). Each sample was then vortexed for 5 minutes, stored for 2 hours at −20° C., and centrifuged at 14000 g for 10 minutes at 4° C. The supernatant of each sample was then transferred to a secondary tube and 3 mL of acidified H₂O (pH=3.5 with 1N HCl) was added. Each sample was extracted following a modified C18 SPE protocol, as previously described by Powell et al., “Extraction of Eicosanoids” (1999). The methyl formate fractions of each sample were individually collected, dried under N2, and reconstituted in 50 μL 1:1 MeOH/H₂O (v/v). Each reconstituted fraction was analyzed using the High Resolution LC-MS/MS mediator lipidomics platform.

### LC-MS/MS Mediator Lipidomics Platform

Separation of mediator lipids was performed on an Ekspert™ microLC 200 system (Eksigent, part of AB SCIEX, Framingham, USA). A Synergi Fusion-RP capillary C18 column (150×0.5 mm, 4 μm, Phenomenex) was used at a column temperature of 40° C. The flow rate was 20 μL/min and injection volume was 10 μL per sample. Mediator lipids were separated using a mobile phase A (100% H₂O, 0.1% acetic acid) and B (100% MeOH, 0.1% acetic acid) with a gradient starting with B at 60% for 0.5 minutes, linearly increasing to 80% at 5 minutes, and then to 95% at 9 minutes, holding 1 minute, and then reducing to 60% at 12 minutes. The MS analysis was performed on a TripleTOF© 5600+ system (Eksigent, part of AB SCIEX, Framingham, Mass., USA) using the MRM^{HR} strategy, which consists of a TOF MS experiment looped with multiple looped MS/MS experiments. MS spectra were acquired in high resolution mode (>30,000) using 100 milliseconds accumulation time per spectrum. Full scan MS/MS is acquired in high sensitivity mode with an accumulation time optimized per cycle. Collision energy is set at −20 V with a spread of 15 V. MS/MS experiments were used for confirmation of the identity of a compound using PeakView™ Software and for quantitation using MultiQuant™ Software.

The data generated by the above protocol was assessed for both mean decrease accuracy and mean decrease Gini index generated by the Random Forest analysis (see Tables 8-10), as well as the log FC, pval, and FDR values generated by the conventional differential analysis (see Tables 11 and 12). The data generated for positive control (i.e., prostate cancer) versus negative control (normal) samples were compared between patients with different races. For example, Tables 8 and 11 compares Caucasian patients versus negative control. Tables 9 and 12 compares African American patients versus negative control. Table 10 compares combined Caucasian and African American patients versus negative control.

Tables 8-10 include the top signaling lipid markers indicative of prostate cancer based on Random Forest analysis from Caucasian prostate cancer patients, African American prostate cancer patients and prostate cancer patients from both races, respectively.

TABLE 8
Signaling Lipid Markers Indicative of Prostate Cancer
for Caucasians Based on Random Forest Analysis

| | | | MeanDe- | MeanDe- |
|---|---|---|---|---|
| lipid | 0 | 1 | creaseAccuracy | creaseGini |
| 5-HETE | 25.61947 | 30.19905 | 33.51927 | 12.43799 |
| LXA4 | 19.90959 | 28.87182 | 30.07128 | 9.748772 |
| 15-OXOETE | 18.64954 | 24.30217 | 27.92306 | 8.823302 |
| 5-HEPE | 17.86433 | 21.02704 | 24.91468 | 6.477502 |
| 8-HETE | 16.87788 | 22.67696 | 24.41754 | 7.435681 |
| LTB4 | 13.72431 | 21.82691 | 23.39465 | 6.206149 |
| 6-KETO-PGF1A | 22.23932 | 10.23816 | 20.44951 | 3.17379 |
| TXB2 | 16.19323 | 14.706 | 19.25372 | 2.566312 |

TABLE 9
Signaling Lipid Markers Indicative of Prostate Cancer
for African Americans Based on Random Forest Analysis

| | | | MeanDe- | MeanDe- |
|---|---|---|---|---|
| lipid | 0 | 1 | creaseAccuracy | creaseGini |
| 13-HOTRE/13- | 26.56095 | 25.98644 | 32.62468 | 11.26713 |
| HOTRE(R) | | | | |
| 9-HOTRE | 25.88711 | 21.56928 | 29.34095 | 9.627945 |
| TXB2 | 15.09202 | 16.68801 | 20.27862 | 5.271396 |
| 5-HEPE | 14.13717 | 17.86238 | 19.06984 | 4.69424 |
| 5-HETE | 15.2944 | 16.27624 | 18.69237 | 4.258713 |
| 12-HEPE | 13.52171 | 11.36061 | 16.16099 | 3.691962 |
| LTB4 | 11.07846 | 11.697 | 15.32431 | 3.529339 |
| 12-HETE | 11.42849 | 11.20876 | 15.29494 | 3.328766 |
| 13-HODE | 3.659833 | 14.05254 | 14.89252 | 3.503561 |
| PGE2/PGD2 | 13.16735 | 7.96254 | 14.61451 | 3.380849 |

TABLE 10
Signaling Lipid Markers Indicative of Prostate Cancer for Caucasians
and African Americans Based on Random Forest Analysis

| | | | MeanDe- | MeanDe- |
|---|---|---|---|---|
| lipid | 0 | 1 | creaseAccuracy | creaseGini |
| 13-HOTRE/13- | 32.93417 | 31.10003 | 41.2192 | 14.72643 |
| HOTRE(R) | | | | |
| 9-HOTRE | 34.15727 | 28.36684 | 38.62875 | 13.17386 |
| 5-HETE | 27.88277 | 34.03878 | 36.58886 | 18.86015 |
| 6-KETO-PGF1A | 30.76574 | 12.46716 | 32.1093 | 11.35685 |
| TXB2 | 26.11704 | 18.59539 | 29.65059 | 10.77893 |
| 5-HEPE | 20.14658 | 22.00517 | 27.01186 | 10.56602 |
| LTB4 | 15.89734 | 23.49437 | 25.7356 | 11.19078 |
| 15-HETRE | 15.32173 | 21.6176 | 24.20359 | 6.875897 |
| 8-HETE | 18.61353 | 21.82525 | 24.20071 | 11.57804 |
| LXA4 | 8.624506 | 24.6293 | 23.98071 | 9.560588 |
| 12-HETE | 18.62514 | 17.35084 | 23.08142 | 6.153768 |
| 19,20-DIHD | 23.82792 | 11.1303 | 23.00571 | 6.27688 |
| 15-OXOETE | 16.49057 | 19.4136 | 21.96515 | 9.688329 |
| 14-HDHA | 12.77102 | 17.12559 | 20.5661 | 3.763917 |

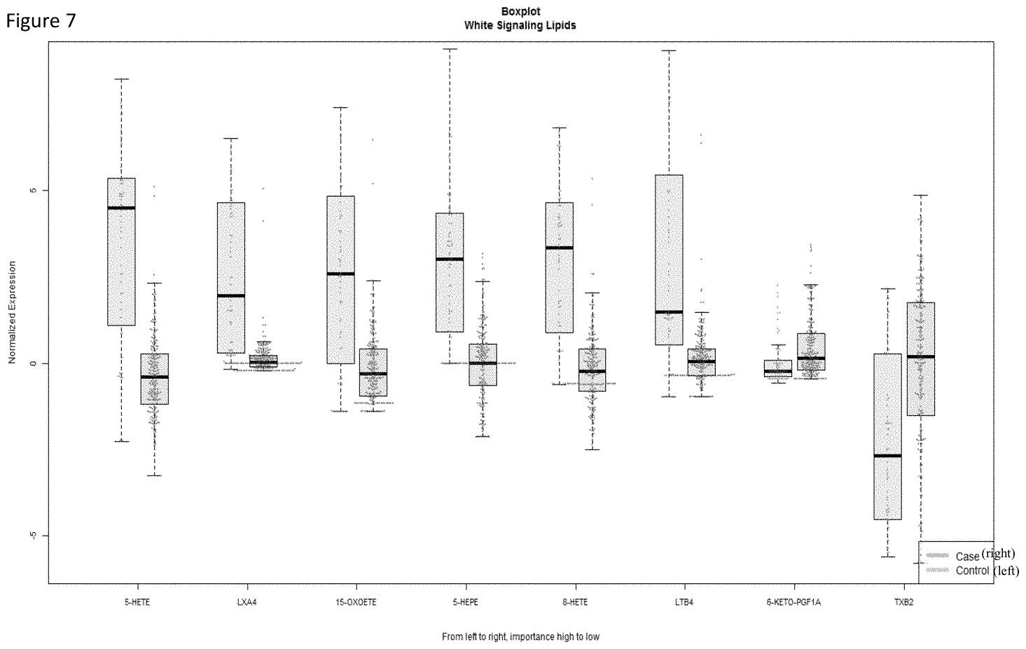

Expression levels of individual markers identified in Tables 8-10 were analyzed. FIGS. 7-9 are box plots depicting a direct comparison of normalized expression levels of individual markers identified in Tables 8-10 between Caucasian prostate cancer patients and negative controls, American African prostate cancer patients and negative controls, and prostate cancer patients from both races and negative controls, respectively. As shown in FIG. 7, expression levels of 6-KETO-PGF1A and TXB2 were increased in Caucasian prostate cancer patients when compared to negative controls, whereas other markers listed in Table 8 had a decreased expression level.

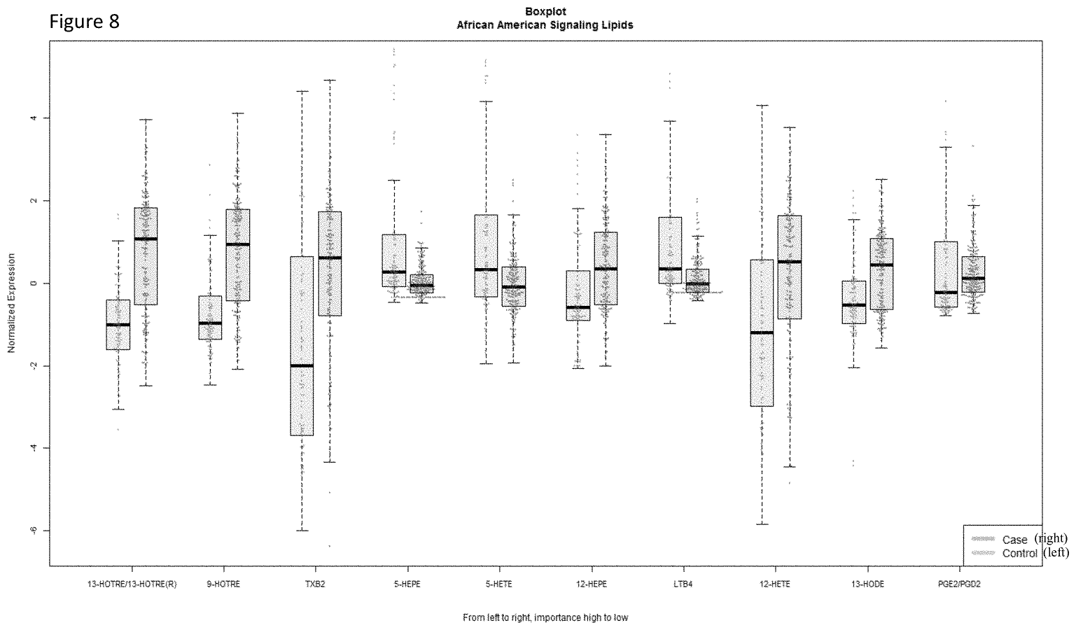

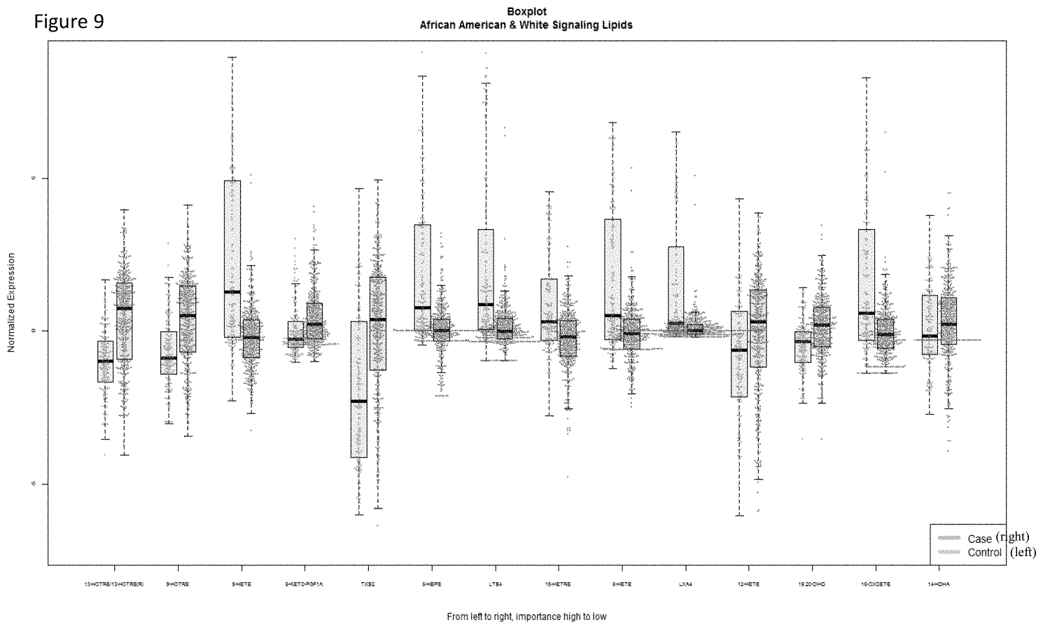

When comparing the expression levels of markers in African American prostate cancer patients with negative controls, an increased level for 13-HOTRE/13-HOTRE(R), 9-HOTRE, TXB2, 12-HEPE, 12-HETE, and 13-HODE was observed in African American prostate cancer patients, whereas other markers in Table 9 showed a decreased expression level in African American prostate cancer patients (FIG. 8). A similar comparison was performed for prostate cancer patients from both races with negative controls. As shown in FIG. 9, an increase expression level of 13-HOTRE/13-HOTRE(R), 9-HOTRE, 6-KETO-PGF1A, TXB2, 12-HETE, 19,20-DID and 14-HDHA was observed in prostate cancer patients from both races, whereas other markers in Table 10 showed a decreased expression level in prostate cancer patients.

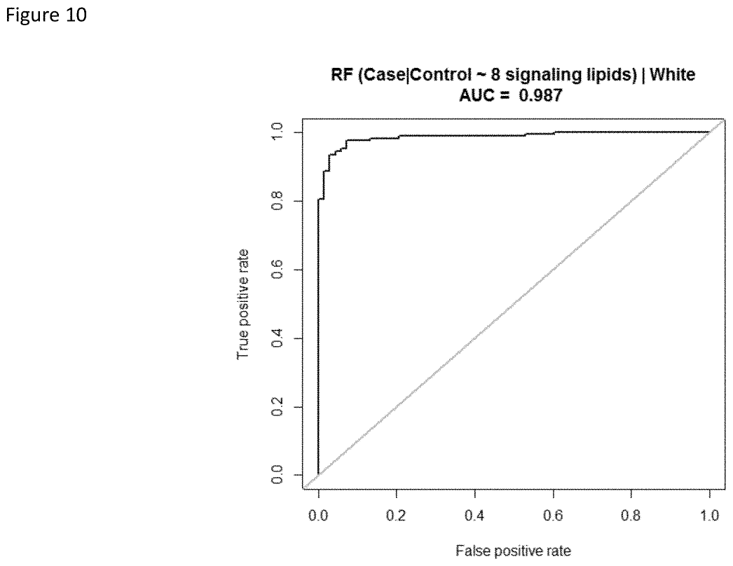

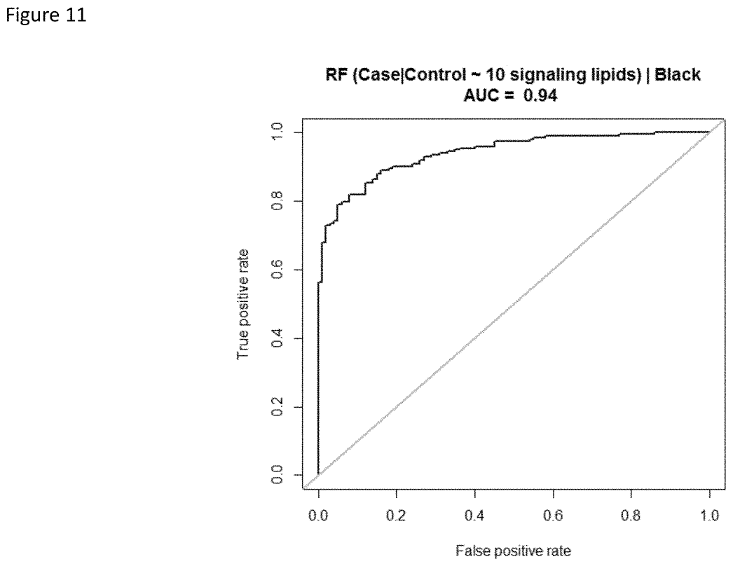

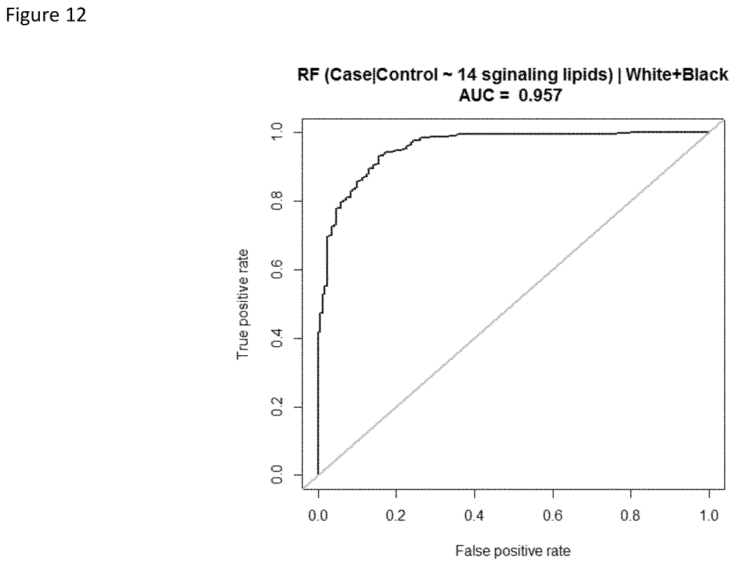

ROC curves were generated for markers identified from the Random Forest analysis. As shown in FIG. 10, the combination of the eight signaling lipids identified in Table 8 has a predictive diagnostic value of 0.987 for Caucasian prostate cancer patients. The combination of the 10 signaling lipids identified in Table 9 has a predictive diagnostic value of 0.94 for African American prostate cancer patients (FIG. 11), and the combination of the 14 signaling lipids identified in Table 10 has a predictive diagnostic value of 0.957 for prostate cancer patients including both Caucasians and African Americans (FIG. 12).

These data indicate that the markers identified in Tables 8-10 may be used as biomarkers for the diagnosis and prognosis of prostate cancer, and to improve the accuracy of prostate cancer detection.

The data generated by the above protocol was also assessed for the log FC, pval, and FDR values generated by the conventional differential analysis (see Tables 11 and 12). Specifically, Tables 11 and 12 list signaling lipid species indicative of prostate cancer in Caucasians and African Americans patients, respectively. These species demonstrated significant changes in amount from negative control to positive control as well as passing an FDR of less than 0.1. These data indicate that the markers identified in Tables 11 and 12 may also be used as biomarkers for the diagnosis and prognosis of prostate cancer, and to improve the accuracy of prostate cancer detection.

TABLE 11
Signaling Lipid Markers Indicative of Prostate Cancer
for Caucasians Based on Differential Analysis

| | median. | median. | median. | median. |
|---|---|---|---|---|
| lipid | diff | diff. FC | diff. pval | diff. FDR |
| TXB2 | 2.87222131 | 7.321916444 | 0 | 0 |
| 13-HOTRE/13- | 1.18948919 | 2.280719762 | 0.0006 | 0.000992 |
| HOTRE(R) | | | | |
| 9,10-DIHOME | 1.00122311 | 2.001696309 | 0.0001 | 0.000195 |
| 9-HOTRE | 0.89396534 | 1.858276704 | 0.0012 | 0.001779 |
| 12,13-DIHOME | 0.70722704 | 1.632663008 | 0.0004 | 0.000688 |
| 9(10)-EPOME | 0.58257036 | 1.497514902 | 0.0013 | 0.001863 |
| 19,20-DIHD | 0.55026064 | 1.464350224 | 0.0009 | 0.001382 |
| 12-HETE | 0.50545511 | 1.419571101 | 0.056 | 0.07297 |
| 6-KETO-PGF1A | 0.39154148 | 1.311794271 | 0.0007 | 0.001115 |
| TXB3 | 0.18409273 | 1.136102286 | 0.038 | 0.051063 |
| 8-ISO-PGF2A | −0.18528312 | 0.879476467 | 0.0002 | 0.000374 |
| PGF2A | −0.22395086 | 0.856217448 | 0 | 0 |
| LXB4 | −0.25729747 | 0.836653715 | 0 | 0 |
| 13,14-DIHYDRO- | −0.27839891 | 0.824505539 | 0 | 0 |
| 15-KETO-PGE2 | | | | |
| 5-IPF2A-VI | −0.32869063 | 0.796258829 | 0 | 0 |
| 8,9-DIHETRE | −0.37919808 | 0.768864844 | 0.0001 | 0.000195 |
| 11(12)-EET | −0.50124679 | 0.706495957 | 0.0003 | 0.000538 |
| 14-HDHA | −0.56582268 | 0.675570071 | 0.034 | 0.047161 |
| 5,6-DIHETRE | −0.78729536 | 0.579429338 | 0 | 0 |
| PGE2/PGD2 | −1.16578757 | 0.445720873 | 0 | 0 |
| LTB4 | −1.41842187 | 0.374121331 | 0 | 0 |
| 10-HDHA | −1.4706539 | 0.360818721 | 0 | 0 |
| 15-HETRE | −1.90230627 | 0.267515378 | 0 | 0 |
| 5-HETRE | −1.92962597 | 0.262497217 | 0 | 0 |
| LXA4 | −1.94162805 | 0.260322506 | 0 | 0 |
| 9-HETE | −2.15927219 | 0.223869177 | 0 | 0 |
| 15-HETE | −2.28154064 | 0.205677995 | 0 | 0 |
| 8-HETRE | −2.34997791 | 0.196149028 | 0 | 0 |
| 11-HETE | −2.89067635 | 0.1348403 | 0 | 0 |
| 15-OXOETE | −2.9023691 | 0.133751864 | 0 | 0 |
| 5-HEPE | −3.00304973 | 0.12473604 | 0 | 0 |
| 8-HETE | −3.56070537 | 0.084746326 | 0 | 0 |
| 5-HETE | −4.88773164 | 0.033778952 | 0 | 0 |

TABLE 12
Signaling Lipid Markers Indicative of Prostate Cancer
for African Americans Based on Differential Analysis

| | median. | median. | median. | median. |
|---|---|---|---|---|
| lipid | diff | diff. FC | diff. pval | diff. FDR |
| TXB2 | 2.6075333 | 6.094607447 | 0 | 0 |
| 13-HOTRE/13- | 2.07821524 | 4.222844842 | 0 | 0 |
| HOTRE(R) | | | | |
| 9-HOTRE | 1.92375912 | 3.794103724 | 0 | 0 |
| 12-HETE | 1.71934282 | 3.292863754 | 0 | 0 |
| 13-HODE | 0.98249187 | 1.97587526 | 0 | 0 |
| 12-HEPE | 0.93929503 | 1.917590983 | 0.0001 | 0.000307 |
| 9-HETE | 0.9262059 | 1.900271951 | 0.003 | 0.00645 |
| 9-HODE | 0.84185993 | 1.792359372 | 0 | 0 |
| 9,10-DIHOME | 0.82362762 | 1.769850647 | 0.0002 | 0.000573 |
| 14-HDHA | 0.81201451 | 1.755661253 | 0 | 0 |
| 9(10)-EPOME | 0.79738185 | 1.737944305 | 0 | 0 |
| 11-HETE | 0.65763046 | 1.577489563 | 0.0072 | 0.013461 |
| 6-KETO-PGF1A | 0.64223213 | 1.560742064 | 0 | 0 |
| 12,13-DIHOME | 0.60631692 | 1.522367766 | 0.0009 | 0.00215 |
| 19,20-DIHD | 0.54618235 | 1.460216564 | 0 | 0 |
| 13-OXOODE | 0.45619815 | 1.371921705 | 0.0001 | 0.000307 |
| PGE2/PGD2 | 0.34613194 | 1.271147935 | 0.0011 | 0.002489 |
| TXB3 | 0.19075408 | 1.141360136 | 0.0094 | 0.016168 |
| 8-ISO-PGF2A | −0.04850156 | 0.96694011 | 0.038 | 0.054467 |
| 5-IPF2A-VI | −0.07677395 | 0.948175519 | 0.0284 | 0.04211 |
| LXA4 | −0.08109769 | 0.945338102 | 0.0032 | 0.006552 |
| 5,6-DIHETRE | −0.08588617 | 0.942205612 | 0.0148 | 0.02357 |
| PGF2A | −0.1092278 | 0.927084149 | 0.0041 | 0.008014 |
| 8,9-DIHETRE | −0.12905769 | 0.914428523 | 0.0103 | 0.017035 |
| 18-HETE | −0.18602955 | 0.879021556 | 0.0207 | 0.031789 |
| 15-HETRE | −0.19717446 | 0.87225722 | 0.0436 | 0.060477 |
| 8-HETRE | −0.25261253 | 0.839375041 | 0.0004 | 0.001075 |
| 15-OXOETE | −0.26565591 | 0.831820468 | 0.0079 | 0.014154 |
| 5-HEPE | −0.3181724 | 0.802085313 | 0 | 0 |
| LTB4 | −0.37539567 | 0.77089396 | 0 | 0 |
| 5-HETE | −0.41642841 | 0.749277269 | 0.0005 | 0.001265 |

### Example 3: Identification of Proteins as Prostate Cancer Markers

### Sample Processing and Top14 Protein Depletion of Serum

Delipidated samples are prepared by adding 1.2 mg of liposorb reagent (PIM-L LIPOSORB resin, EMD Millipore Corporation, Billerica, USA) to a 30 μL aliquot of each serum sample, as per manufacturer protocol. Each delipidated serum sample underwent protein depletion using an HU-14 MARS column (Agilent Technologies, Inc. Santa Clara, USA) on an Agilent 1260 HPLC system according to the manufacturer instruction. A 200 μl aliquot of flow-through fraction was transferred to a clean 2.0 mL microtube.

### Protein Reduction, Alkylation, Precipitation, and Trypsin Digestion

Protein from each sample is reduced (200 mM tris(2-carboxyethyl)phosphine [TCEP], 55° C., 1 hour), alkylated (375 mM iodoacetamide, RT, 30 minutes), precipitated using cold acetone (−20° C., overnight), and digested with Trypsin (1:25 w/w, 200 mM triethylammonium bicarbonate (TEAB), 37° C., 16 hours).

### TMT Labeling

Equal aliquots from each reduced, alkylated, precipitated, and digested sample and the pooled control sample are labeled by TMT 10 Plex reagents according to the manufacturer protocols (Pierce, Rockford, USA). The reactions of each sample are combined, vacuumed to dryness, re-suspended by adding 20 mM ammonium formate (pH 10 resuspended at 1 μg/μL) and analyzed by 2D-LC-MS/MS.

### 2D-LC-MS/MSMS Analysis

Nano-LC-MS/MS was performed on a RP-RP 2D NanoAcquity UPLC system, (Waters, Milford, USA) coupled to a QExactive Plus Orbitrap mass spectrometer (ThermoElectron, Bremen, Germany) and a Nanospray Flex Ion source. A 4 μg sample of tryptic peptides mixture was first loaded onto the 1^{st} dimensional column (300 m×5 cm XBridge™ C18 (5 m), Waters, Milford, USA), then eluted by 10 discontinuous step gradient (mobile phase A: 20 mM ammonium formate pH 10.0; mobile phase B: acetonitrile) to load on a trap column (180 m×2 cm Symmetry C18 (5 μm), Waters, Milford, USA) for concentration and desalting. To maximize sample recovery on the 2^{nd} dimension trap column from the organic-containing fractions, an aqueous flow was delivered with the 2^{nd} dimension pump with a 20 μL/min flow rate and mixed with the eluted fraction with a 2 μL/min flow rate prior to trapping. The trap column was then switched online to directly connect with a 2^{nd} dimensional reversed-phase 75-m I.D.×20 cm column packed with 1.7 m C18 particles (Waters, Milford, USA), and peptides were gradually eluted with a flow rate of 300 nL/min at a 35° C. column temperature using a gradient of 2-40% mobile phase B (H₂O/0.1% FA (mobile phase A) and ACN/0.1% FA (mobile phase B)) for 60 minutes. A steeper gradient was then used to further increase mobile phase B to 85% in the next 5 minutes. Full scan MS spectra (m/z 400-1800) were acquired in the Orbitrap with a resolution of 35,000. Target of automatic gain (AGC) was set at 3×10⁶ ions. The most intense ions (up to 15) were sequentially isolated for fragmentation using High Energy C-Trap Dissociation (HCD) and dynamically excluded for 10 seconds. HCD was conducted with an isolation width of 1.2 Da. The resulting fragment ions were scanned in the Orbitrap with resolution of 35,000.

### Peptide/Protein Identification and Quantification

Peptides and proteins were identified by automated database searching using Proteome Discoverer 1.4 software (Thermo Fisher) with the Mascot and SequestHT search engine against the SwissProt database. Search parameters included 10 ppm for MS tolerance, 0.02 Da for MS² tolerance, and full trypsin digestion allowing for up to 2 missed cleavages. Carbamidomethylation (C) was set as the fixed modification. Oxidation (M), TMT6 (N-terminal, K), and deamidation (NQ) were set as dynamic modifications. Resulting peptide hits were filtered for maximum 1% FDR using the Percolator algorithm. The Proteome Discoverer software applied correction factors on the reporter ions and rejected all quantitation values if not all quantitation channels was present. Relative protein quantitation was achieved by normalization at the mean intensity.

The data generated by the above protocol was assessed for both mean decrease accuracy and mean decrease Gini index generated by the Random Forest analysis (see Tables 13-15), as well as the log FC, pval, and FDR values generated by the conventional differential analysis (see Tables 16-18). The data generated for positive control (i.e., prostate cancer) versus negative control (normal) samples were compared between patients with different races, or BMI status. For example, Tables 13 and 16 compare Caucasian patients versus negative control. Tables 14 and 17 compare African American patients versus negative control. Table 15 compares combined Caucasian and African American patients versus negative control. Table 18 compares obese patients versus non-obese patients.

Tables 13-15 list the protein markers indicative of prostate cancer based on Random Forest analysis from Caucasian prostate cancer patients, African American prostate cancer patients and prostate cancer patients from both races, respectively.

TABLE 13
Protein Markers Indicative of Prostate Cancer for Caucasians Based on Random Forest Analysis

| | | | | | Mean Decrease | Mean Decrease |
|---|---|---|---|---|---|---|
| Accession | Gene | Description | 0 | 1 | Accuracy | Gini |
| P80108 | GPLD1 | Phosphatidyl- | 13.55338 | 20.23704 | 22.0049 | 4.78966 |
| | | inositol-glycan- | | | | |
| | | specific | | | | |
| | | phospholipase D | | | | |
| P02656 | APOC3 | Apolipoprotein | 12.85226 | 13.10194 | 16.20712 | 3.646246 |
| | | C-III | | | | |
| P05155 | SERPING1 | Plasma protease | 1.214796 | 13.46915 | 13.23445 | 2.281121 |
| | | C1 inhibitor | | | | |
| P01024 | C3 | Complement C3 | 2.450765 | 12.21102 | 12.3955 | 1.946765 |
| P01023 | A2M | Alpha-2- | −1.24963 | 12.73167 | 12.26045 | 1.811731 |
| | | macroglobulin | | | | |
| P08185 | SERPINA6 | Corticosteroid- | 3.182902 | 10.74573 | 11.41412 | 1.733628 |
| | | binding globulin | | | | |
| P04114 | APOB | Apolipoprotein | 5.056052 | 8.945619 | 10.17442 | 2.11028 |
| | | B-100 | | | | |
| P06727 | APOA4 | Apolipoprotein | 5.227166 | 9.196746 | 10.09423 | 2.711814 |
| | | A-IV | | | | |
| Q15848 | ADIPOQ | Adiponectin | 7.83319 | 6.071432 | 9.417895 | 2.271707 |
| P02743 | APCS | Serum amyloid | 6.220127 | 5.215185 | 8.550553 | 1.814673 |
| | | P-component | | | | |
| P19823 | ITIH2 | Inter-alpha- | 3.152415 | 7.729316 | 8.34645 | 1.657313 |
| | | trypsin inhibitor | | | | |
| | | heavy chain H2 | | | | |
| P10909 | CLU | Clusterin | −1.78285 | 9.267159 | 8.344048 | 1.37993 |
| P02652 | APOA2 | Apolipoprotein | 4.715723 | 6.913758 | 7.931194 | 1.79465 |
| | | A-II | | | | |
| P49908 | SEPP1 | Selenoprotein P | 3.213534 | 6.732757 | 7.540639 | 1.416148 |
| P02775 | PPBP | Platelet basic | −0.6892 | 7.842576 | 7.048325 | 1.219551 |
| | | protein | | | | |

TABLE 14
Protein Markers Indicative of Prostate Cancer for
African Americans Based on Random Forest Analysis

| | | | | | Mean Decrease | Mean Decrease |
|---|---|---|---|---|---|---|
| Accession | Gene | Description | 0 | 1 | Accuracy | Gini |
| P01024 | C3 | Complement C3 | 14.63661 | 21.53954 | 22.25731 | 4.550463 |
| P06727 | APOA4 | Apolipoprotein | 9.153332 | 9.918799 | 12.72701 | 3.255589 |
| | | A-IV | | | | |
| P04003 | C4BPA | C4b-binding | 7.718607 | 9.49066 | 10.61297 | 1.653199 |
| | | protein alpha | | | | |
| | | chain | | | | |
| Q9H8L6 | MMRN2 | Multimerin-2 | 6.466166 | 6.307338 | 8.291634 | 1.899324 |
| P02652 | APOA2 | Apolipoprotein | 2.656906 | 8.147525 | 7.727353 | 1.604429 |
| | | A-II | | | | |
| P01034 | CST3 | Cystatin-C | 2.936455 | 6.886684 | 7.566153 | 1.343744 |
| P02671 | FGA | Fibrinogen | 3.768516 | 6.677035 | 7.281064 | 1.854925 |
| | | alpha chain | | | | |
| P12259 | F5 | Coagulation | 2.175769 | 8.12205 | 7.183769 | 1.354765 |
| | | factor V | | | | |
| Q7Z7G0 | ABI3BP | Target of | 3.013437 | 6.603629 | 6.992125 | 1.336962 |
| | | Nesh-SH3 | | | | |
| P02647 | APOA1 | Apolipoprotein | −0.13371 | 8.126468 | 6.643086 | 1.276786 |
| | | A-I | | | | |
| P07225 | PROS1 | Vitamin K- | 4.136077 | 5.568667 | 6.632363 | 1.17755 |
| | | dependent | | | | |
| | | protein S | | | | |
| P49747 | COMP | Cartilage | 0.843911 | 7.089962 | 6.607228 | 1.240989 |
| | | oligomeric | | | | |
| | | matrix protein | | | | |
| P61769 | B2M | Beta-2- | 5.199089 | 4.88088 | 6.543872 | 2.274635 |
| | | microglobulin | | | | |
| P33151 | CDH5 | Cadherin-5 | 3.203742 | 3.81518 | 4.773756 | 1.146776 |
| P08185 | SERPINA6 | Corticosteroid- | 2.075369 | 4.026947 | 4.725714 | 0.93987 |
| | | binding globulin | | | | |

TABLE 15
Protein Markers Indicative of Prostate Cancer for Caucasians
and African Americans Based on Random Forest Analysis

| | | | | | MeanDecrease | MeanDecrease |
|---|---|---|---|---|---|---|
| Accession | Gene | Description | 0 | 1 | Accuracy | Gini |
| P01024 | C3 | Complement C3 | 3.626058 | 22.58989 | 23.02246 | 5.716069 |
| P80108 | GPLD1 | Phosphatidy- | 11.78486 | 16.73065 | 19.23702 | 5.514514 |
| | | linositol-glycan- | | | | |
| | | specific | | | | |
| | | phospholipase D | | | | |
| P02656 | APOC3 | Apolipoprotein | 10.70317 | 15.81028 | 18.31597 | 6.096497 |
| | | C-III | | | | |
| P06727 | APOA4 | Apolipoprotein | 8.903969 | 15.29022 | 16.82977 | 6.251579 |
| | | A-IV | | | | |
| P02671 | FGA | Fibrinogen | 1.147825 | 14.19799 | 14.18352 | 3.595781 |
| | | alpha chain | | | | |
| P01023 | A2M | Alpha-2- | 1.199995 | 14.29074 | 14.07488 | 3.350825 |
| | | macroglobulin | | | | |
| P05155 | SERPING1 | Plasma protease | 1.367103 | 13.76712 | 13.38653 | 3.914557 |
| | | C1 inhibitor | | | | |
| P01034 | CST3 | Cystatin-C | 6.106608 | 12.53864 | 13.2599 | 3.970332 |
| P02652 | APOA2 | Apolipoprotein | 2.810881 | 11.86172 | 12.45279 | 3.622326 |
| | | A-II | | | | |
| P08185 | SERPINA6 | Corticosteroid- | 1.435524 | 11.66337 | 11.8972 | 3.305827 |
| | | binding globulin | | | | |
| P04003 | C4BPA | C4b-binding | 7.174154 | 7.940042 | 10.97088 | 3.134159 |
| | | protein alpha | | | | |
| | | chain | | | | |
| P04114 | APOB | Apolipoprotein | 2.442928 | 10.73296 | 10.78669 | 3.929044 |
| | | B-100 | | | | |
| P10909 | CLU | Clusterin | −0.38856 | 11.065 | 10.70325 | 2.554145 |
| O14791 | APOL1 | Apolipoprotein | −0.17479 | 9.708665 | 9.48398 | 2.908765 |
| | | L1 | | | | |
| P02775 | PPBP | Platelet basic | 0.908338 | 9.980571 | 9.290247 | 2.505695 |
| | | protein | | | | |
| P07225 | PROS1 | Vitamin K- | 1.401955 | 8.907813 | 8.506197 | 2.938616 |
| | | dependent | | | | |
| | | protein S | | | | |
| P35542 | SAA4 | Serum amyloid | −2.82293 | 9.186306 | 7.776131 | 1.971727 |
| | | A-4 protein | | | | |
| P05546 | SERPIND1 | Heparin | 4.50291 | 5.397874 | 7.067221 | 3.046714 |
| | | cofactor 2 | | | | |

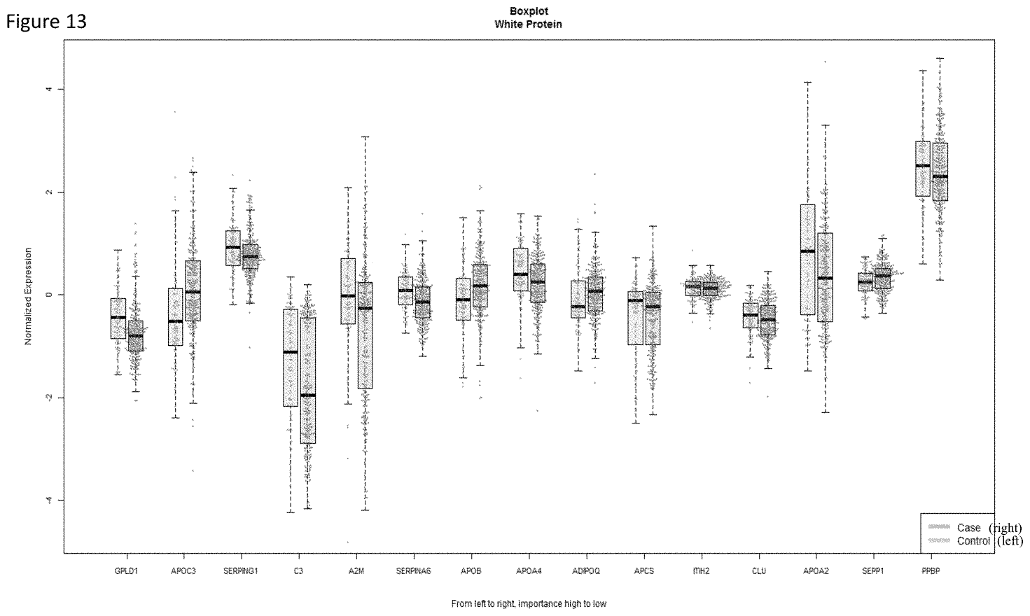

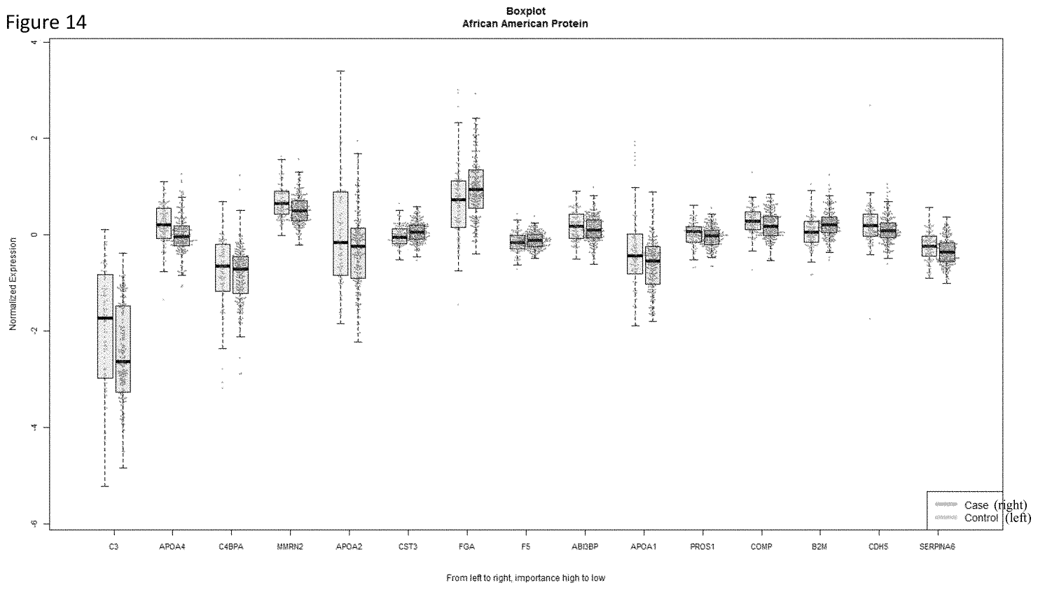

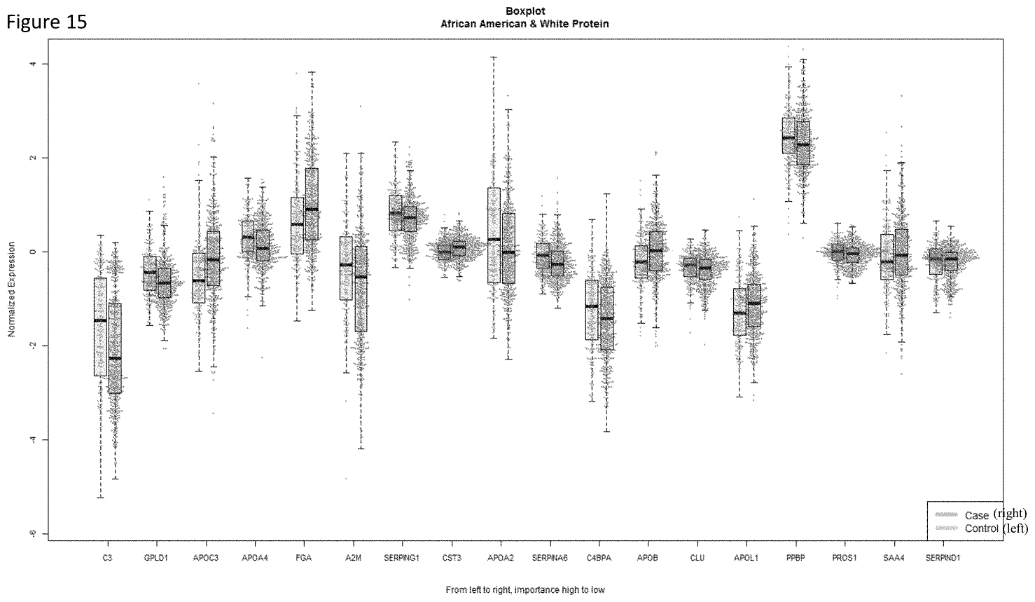

Expression levels of individual markers identified in Tables 13-15 were analyzed. FIGS. 13-15 are box plots depicting a direct comparison of normalized expression levels of individual markers identified in Tables 13-15 between Caucasian prostate cancer patients and negative controls, American African prostate cancer patients and negative controls, and prostate cancer patients from both races and negative controls, respectively. As shown in FIG. 13, expression levels of APOC, APOB, ADIPOQ and SEPP1 were increased in Caucasian prostate cancer patients when compared to negative controls, whereas other markers listed in Table 13 had a decreased expression level. When compared the expression levels of markers in African American prostate cancer patients with negative controls, an increased level for CST3, F5 and B2M was observed in African American prostate cancer patients, whereas other markers in Table 14 showed a decreased expression level in African American prostate cancer patients (FIG. 14). Similar comparison was performed for prostate cancer patients from both races with negative controls. As shown in FIG. 15, an increase expression level of C5, APOC3, FGA, CST3, APOB, APOL1 and SAA4 was observed in prostate cancer patients from both races, whereas other markers in Table 15 showed a decreased expression level in prostate cancer patients.

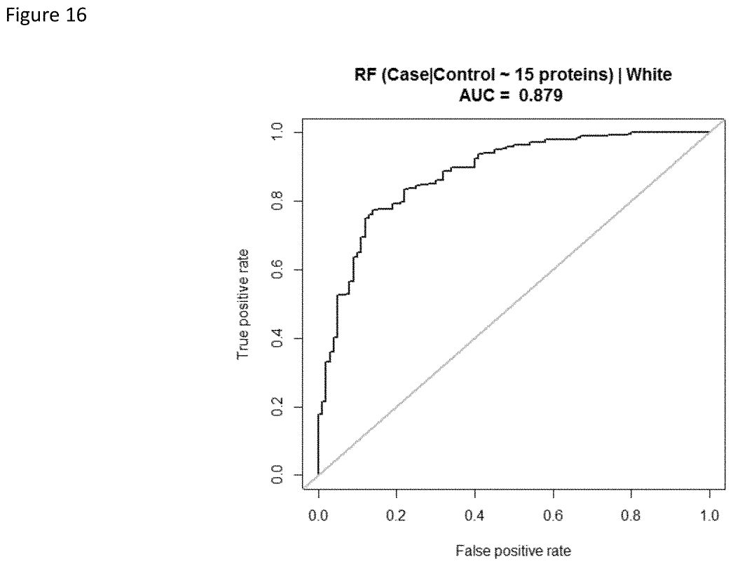

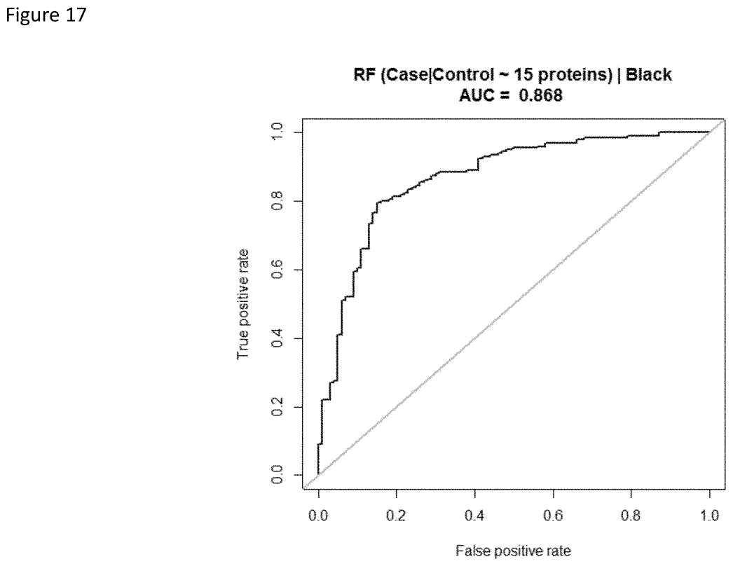

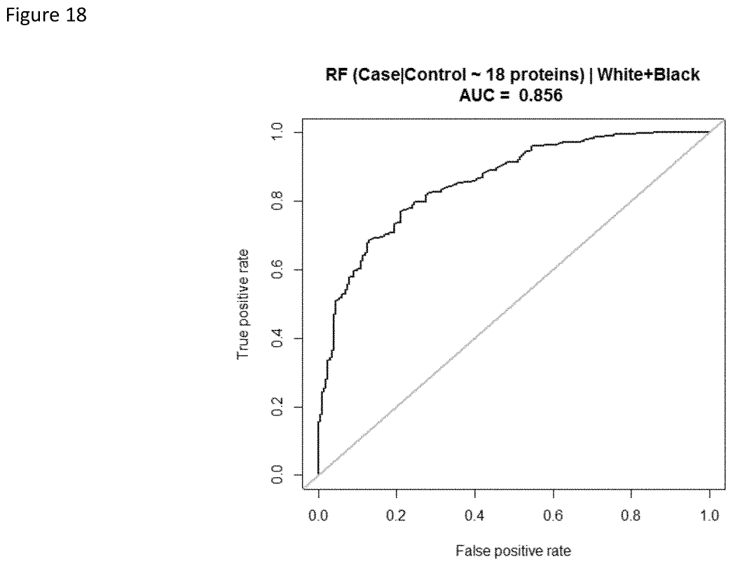

ROC curves were generated for markers identified from the Random Forest analysis. As shown in FIG. 16, the combination of the 15 protein markers identified in Table 13 has a predictive diagnostic value of 0.879 for Caucasian prostate cancer patients. The combination of the 15 protein markers identified in Table 14 has a predictive diagnostic value of 0.868 for African American prostate cancer patients (FIG. 17), and the combination of the 18 protein markers identified in Table 15 has a predictive diagnostic value of 0.856 for prostate cancer patients including both Caucasians and African Americans (FIG. 18). These data indicate that the markers identified in Tables 13-15 may be used as biomarkers for the diagnosis and prognosis of prostate cancer, and to improve the accuracy of prostate cancer detection.

The data generated by the above protocol was also assessed for the log FC, pval, and FDR values generated by the conventional differential analysis (see Tables 16-18). Specifically, Tables 16 and 17 list protein species indicative of prostate cancer in Caucasians and African Americans patients, respectively. Table 18 includes protein species indicative of prostate cancer between obese and non-obese patients. These protein species demonstrated significant changes in amount from negative control to positive control as well as passing an FDR of less than 0.1. These data indicate that the markers identified in Tables 16-18 may also be used as biomarkers for the diagnosis and prognosis of prostate cancer, and to improve the accuracy of prostate cancer detection.

TABLE 16
Protein Markers Indicative of Prostate Cancer
for Caucasians Based on Differential Analysis

| | | | median. | median. | median. | median. |
|---|---|---|---|---|---|---|
| Accession | Gene | Description | diff | diff. FC | diff. pval | diff. FDR |
| P02656 | APOC3 | Apolipoprotein | 0.576105 | 1.490819 | 0 | 0 |
| | | C-III | | | | |
| P05155 | SERPING1 | Plasma protease | −0.18057 | 0.882357 | 0 | 0 |
| | | C1 inhibitor | | | | |
| P80108 | GPLD1 | Phosphatidyl- | −0.35503 | 0.781855 | 0 | 0 |
| | | inositol-glycan- | | | | |
| | | specific | | | | |
| | | phospholipase D | | | | |
| Q15848 | ADIPOQ | Adiponectin | 0.28673 | 1.219872 | 2.00E−04 | 0.0437 |

TABLE 17
Protein Markers Indicative of Prostate Cancer for
African Americans Based on Differential Analysis

| | | | median. | median. | median. | median. |
|---|---|---|---|---|---|---|
| Accession | Gene | Description | diff | FC | dif. pval | diff. FDR |
| P06727 | APOA4 | Apolipoprotein | −0.25482 | 0.83809 | 0 | 0 |
| | | A-IV | | | | |
| P01024 | C3 | Complement C3 | −0.90238 | 0.535002 | 1.00E−04 | 0.029133 |
| P36955 | SERPINF1 | Pigment epithelium- | −0.09029 | 0.939333 | 1.00E−04 | 0.029133 |
| | | derived factor | | | | |
| P01034 | CST3 | Cystatin-C | 0.119625 | 1.086453 | 2.00E−04 | 0.03496 |
| P61769 | B2M | Beta-2- | 0.15835 | 1.11601 | 2.00E−04 | 0.03496 |
| | | microglobulin | | | | |
| P41222 | PTGDS | Prostaglandin-H2 | 0.137705 | 1.100153 | 3.00E−04 | 0.0437 |
| | | D-isomerase | | | | |
| Q9H8L6 | MMRN2 | Multimerin-2 | −0.15452 | 0.898429 | 4.00E−04 | 0.049943 |

TABLE 18
Comparison of Protein Markers Indicative of Prostate Cancer in
Obese and Non-Obese Patients Based on Differential Analysis

| Accession | Gene | Description | logFC | FC | P. Value | FDR |
|---|---|---|---|---|---|---|
| Q15848 | ADIPOQ | Adiponectin | −0.30536 | 0.809241 | 2.52E−06 | 0.001124 |
| P18065 | IGFBP2 | Insulin-like growth factor- | −0.25963 | 0.835305 | 4.65E−06 | 0.001256 |
| | | binding protein 2 | | | | |
| P04278 | SHBG | Sex hormone-binding | −0.19384 | 0.874273 | 3.79E−05 | 0.007687 |
| | | globulin | | | | |
| P49908 | SEPP1 | Selenoprotein P | −0.10087 | 0.932468 | 0.000733 | 0.084879 |
| P35858 | IGFALS | Insulin-like growth factor- | −0.09066 | 0.939094 | 0.000936 | 0.086846 |
| | | binding protein complex | | | | |
| | | acid labile subunit | | | | |
| Q76LX8 | ADAMTS13 | A disintegrin and | 0.110274 | 1.079433 | 0.001018 | 0.086846 |
| | | metalloproteinase with | | | | |
| | | thrombospondin motifs 13 | | | | |
| Q86TH1 | ADAMTSL2 | ADAMTS-like protein 2 | 0.1728 | 1.127244 | 0.000728 | 0.084879 |
| P04003 | C4BPA | C4b-binding | 0.342 | 1.267512 | 0.001071 | 0.086846 |
| | | protein alpha | | | | |
| | | chain | | | | |
| O14798 | TNFRSF10C | Tumor necrosis factor | 0.394067 | 1.314093 | 0.001236 | 0.091091 |
| | | receptor superfamily | | | | |
| | | member 10C | | | | |
| P20742 | PZP | Pregnancy zone protein | 0.464746 | 1.380074 | 0.000294 | 0.047688 |
| P02741 | CRP | C-reactive protein | 0.597515 | 1.513108 | 2.77E−06 | 0.001124 |

### Example 4: Identification of Metabolites as Prostate Cancer Markers

### Serum Sample Preparation for Clinical Metabolomics

Human serum in an amount of 75 μL were placed in pre-chilled at −80° C. 2 ml round bottom Eppendorf tubes having stainless steel ball in it. 420 μl of pre-chilled at −20° C. mixture of acetonitrile, iso-propanol and deionized water in proportion 3:3:2 v.v.v. Samples were vortexed for 5 seconds and stored at −20° C. overnight. Samples were further centrifuged at +4° C. at 12000 rpm for 3 minutes. Clean supernatant was transferred in to LCMS vials and in to 0.5 ml Eppendorf tubes to be dried for GC-TOF-MS analysis. Serum extracts were divided in to three parts: 75 μL for GC-TOF-MS analysis, 150 μL for HILIC-LC/MS analysis, and 150 μL for HILIC-LC/MS/MS analysis.

### Clinical Metabolomics Analysis of the Serum Samples.

Metabolomics analyses were performed using targeted protocols using PEAGSUS-HT GC-TOF-MS, hydrophilic HILIC-LC-MS/MS and hydrophobic (RP)-LC-HRMS instrumentation implementing previously reported methodology modified to the current instrumentation settings (Tolstikov V, et al. PloS one 2014; 9(12): e114019; Urayama S, et al. Rapid communications in mass spectrometry: RCM 2010; 24(5): 613-20; Zou W, et al. Rapid communications in mass spectrometry:RCM 2008; 22(8): 1312-24; Gacias M, et al. eLife 2016; 10.7554/eLife.13442). A standard quality control (QC) sample containing a mixture of amino acids and organic acids was injected daily to monitor mass spectrometer response. The pooled QC sample was obtained by taking an aliquot of the same volume of all samples from the study. The pooled QC sample was injected daily with a batch of analyzed samples. External serum pooled QC sample which not related to current study was injected after each 10^{th} sample daily within a batch of analyzed samples. QCs were used to determine the optimal dilution of the batch samples and to validate metabolite identification and peak integration.

### Sample Derivatization and GC-TOF-MS Analysis

Extracts were dried using SpeedVac Concentrator Savant DNA120 (ThermoScientific, San Jose, Calif.) with the bath temperature set below 30° C. Dried sample derivatization with methoxylamine hydrochloride in pyridine and N-methyl-N-trimethylsilyltrifluoroacetamide was performed at 60° C. during 60 min. 7890B gas chromatograph (Agilent, Palo Alto, Calif.) interfaced to a Pegasus HT TOF mass spectrometer (Leco, St. Joseph, Mich.). Automated injections were performed using an MPS2 programmable robotic multipurpose sampler (Gerstel, Muhlheim an der Ruhr, Germany). The GC system was fitted with a Gerstel temperature-programmed injector, cooled injection system (model CIS 4). An automated liner exchange (ALEX) (Gerstel) was used to eliminate cross-contamination from the sample matrix that was occurring between sample runs. Multiple deactivated baffled liners for the GC inlet were used. Syringe wash was setup with hexane and ethyl acetate consequently prior and after injection. The Gerstel injector was programmed for the following sequence: initial temperature 50° C., hold for 0.1 minute, increase temperature at a rate of 10° Cs⁻¹ to a final temperature of 330° C., and hold time 15 minutes). Injections of 1 μL were made in the splitless mode. Chromatography was performed on an Rtx-5Sil MS column (length: 30 m; ID: 0.25 mm; df 0.25 μm) with an Integra-Guard column (Restek, Bellefonte, Pa.). Helium carrier gas was used at a constant flow of 1 mL min⁻¹. The GC oven temperature was programed for the following sequence: 50° C. initial temperature with a 1-minute hold time and then ramping at 10° C. per minute to a temperature of 140° C., then ramping at 4° C. per minute to a temperature of 240° C., and then ramping at 10° C. per minute to a temperature of 300° C. with an 8-minute hold time. Both the transfer line and the source temperatures were 250° C. Ion source operated at 70 kV filament voltage. After a solvent delay of 500 seconds, mass spectra were acquired at 20 spectra per second with an extraction frequency of 2 kHz and a mass range of 60 to 520 m/z. The standard QCs and pooled sample QCs were used to monitor GC-TOF-MS data acquisition. Mass spectrometer calibration was performed on daily basis using vendor protocol. This included Tune check and Leak check. Data analysis was performed with vendor software ChromaTof (LECO, St. Joseph, Mich.) using the latest NIST-MS database (http//chemdata.nist.gov/).

### HILIC-LC-MS/MS Analysis

Serum extracts were used without any further derivatization. HILIC-LC-MS/MS data acquisition and processing were monitored using standard QCs and pooled sample QCs. NEXERA XR HPLC system (Shimadzu, Columbia, Md.) coupled with the Triple Quadrupole 5500 System (Sciex, Framingham, Mass.). HILIC separations were achieved using a polyamine-bonded polymeric gel column (apHera NH2 Polymer) with a 150×2 mm, 5-μm particle size, equipped with a guard column (apHera NH2 Polymer) with a 10×2 mm, 5-μm particle size (SUPELCO, Bellefonte, Pa.). The mobile phases were acetonitrile (A) and 50 mM ammonium bicarbonate (pH 9.4, adjusted with ammonium hydroxide) (B) at the flow rates of 0.25 mL/minutes at 30° C. After 3-minute isocratic run at 15% B, a gradient to 30% B was concluded at 11 minutes and a gradient to 60% B was concluded at 13 minutes. After that, a gradient to 75% B was concluded at 20 minutes and a gradient to 98% B was completed at 21 minutes. Following column wash, the run was concluded with 98% B at 25 minutes. Column equilibration with a starting buffer took 5 minutes before the next injection. Injection volume was set as 10 μL. Data acquisition was performed using scheduled MRMs. Total list contained more than 450 MRM transitions generated with authentic standards in positive and in negative modes. Data analysis was performed with the vendor software MultiQiant 3.0 (Sciex, Framingham, Mass.). Data extraction was accomplished using MRM transitions values and previously determined retention time with 1 min of the search window.

### RP-LC-HRMS Analysis

Serum extracts were used without any further derivatization. HILIC-LC-MS/MS data acquisition and processing were monitored using standard QCs and pooled sample QCs. RP-LC-HRMS platform consisted of NEXERA XR HPLC system (Shimadzu, Columbia, Md.) coupled with the Triple TOF 5600 System (Sciex, Framingham, Mass.). RP separations were achieved using Kinetex 2.6 u F5 100A column having 150×3 mm dimensions, equipped with a security guard cartridge (Phenomenex, Torrance, Calif.). The mobile phases were 0.1% formic acid (A) and acetonitrile (B) at the flow rates of 0.4 mL/minutes at 40° C. After 0.03-minute isocratic run at 0% B, a gradient to 70% B was concluded at 4 minutes and a gradient to 100% B was concluded at 8.5 minutes. After that, a gradient to 100% B was concluded at 11.5 minutes and a gradient to 0% B was completed at 12 minutes. Following column equilibration, the run was concluded with 0% B at 15 minutes. Injection volume was set as 10 μL. Data analysis was performed with the vendor software MultiQiant 3.0 (Sciex, Framingham, Mass.). Data extraction was accomplished using HRMS window set as 10 amu and retention times previously determined with authentic standards.

### Data Processing

Web based and in house generated HRMS and HRMS/MS databases were used for the elemental composition assignment, spectral data comparisons, and detailed manual interpretation of spectral data in order to assign chemical identity for analytes without authentic standards available commercially and upon request from other institutions/companies.

Peak integration is always manually inspected in order to validate automatic integration quality. Artifacts as well as components having more that 50% of missing values were removed from the list. Refined data was further forwarded to Analytics Group for normalization and statistical analysis. Overlapped metabolite peak areas were selected with the preference given to LC-MS/MS results unless manual inspection finds discrepancies (i.e. poor S/N ratio, poor peak shape, etc.) allowing reassign preference given to choose the obtained data for overlapped metabolites.

The data generated by the above protocol was assessed for both mean decrease accuracy and mean decrease Gini index generated by the Random Forest analysis (see Tables 19-21), as well as the log FC, pval, and FDR values generated by the conventional differential analysis (see Tables 22-25). The data generated for positive control (i.e., prostate cancer) versus negative control (normal) samples were compared between patients with different races, BMI status or ERG status. For example, Tables 19 and 22 compare Caucasian patients versus negative control. Tables 20 and 23 compare African American patients versus negative control. Table 21 compares combined Caucasian and African American patients versus negative control. Table 24 compares Caucasian ERG positive non-obese patients versus Caucasian ERG negative obese patients. Table 25 compares obese patients versus non-obese patients.

Tables 19-21 list the top metabolite markers indicative of prostate cancer based on Random Forest analysis from Caucasian prostate cancer patients, African American prostate cancer patients and prostate cancer patients from both races, respectively.

TABLE 19
Metabolite Markers Indicative of Prostate Cancer
for Caucasians Based on Random Forest Analysis

| | | | MeanDe- | MeanDe- |
|---|---|---|---|---|
| | | | crease | crease |
| all_names | 0 | 1 | Accuracy | Gini |
| nicotinamide | 22.59321 | 25.04004 | 27.11712 | 8.35382 |
| glu-leu | 20.76732 | 24.33626 | 25.6397 | 8.056511 |
| 6-ketodecanoylcarnitine | 17.41196 | 20.71156 | 22.89107 | 5.7859 |
| eicosenoic acid | 16.81194 | 18.14325 | 20.90174 | 5.629215 |
| myo-inositol | 14.18953 | 19.31547 | 19.44699 | 2.936632 |
| chenodeoxyglycocholate | 17.1532 | 15.94717 | 19.2785 | 5.797388 |
| 2-hydroxy-2- | 15.26262 | 16.40938 | 19.14964 | 4.812469 |
| methylbutanedioic acid | | | | |
| nonanedioic acid | 15.60178 | 12.90129 | 17.86514 | 3.921323 |
| glycerylphosphoryl- | 12.501 | 14.49734 | 17.5339 | 3.331343 |
| ethanolamine | | | | |

TABLE 20
Metabolite Markers Indicative of Prostate Cancer for
African Americans Based on Random Forest Analysis

| | | | MeanDe- | MeanDe- |
|---|---|---|---|---|
| | | | crease | crease |
| all_names | 0 | 1 | Accuracy | Gini |
| nicotinamide | 25.88333 | 26.75789 | 28.95601 | 9.970851 |
| 6-ketodecanoylcarnitine | 24.65833 | 26.3123 | 28.67967 | 8.506431 |
| glu-leu | 20.28032 | 23.45286 | 24.45875 | 6.602598 |
| eicosenoic acid | 18.61665 | 19.10165 | 21.77706 | 6.090529 |
| 3-hydroxybutyric acid | 18.62404 | 15.61838 | 21.21881 | 6.431987 |
| ethanolamine | 17.70114 | 14.32458 | 19.16352 | 4.298239 |
| 2-keto-isovalerate | 18.38758 | 10.3084 | 18.74498 | 3.444339 |
| nonanoylcarnitine | 15.39719 | 15.02547 | 18.43379 | 4.685406 |

TABLE 21
Metabolite Markers Indicative of Prostate Cancer for Caucasians
and African Americans Based on Random Forest Analysis

| | | | MeanDe- | MeanDe- |
|---|---|---|---|---|
| | | | crease | crease |
| all_names | 0 | 1 | Accuracy | Gini |
| nicotinamide | 36.60637 | 41.49404 | 44.48802 | 25.85515 |
| 6-ketodecanoylcarnitine | 31.50242 | 34.44189 | 38.4983 | 18.55247 |
| glu-leu | 26.60949 | 33.3398 | 34.7342 | 17.60357 |
| eicosenoic acid | 24.6142 | 27.34694 | 31.08588 | 14.83442 |
| glycerylphosphoryl- | 17.17776 | 20.78308 | 23.63644 | 8.401991 |
| ethanolamine | | | | |
| ethanolamine | 18.83763 | 17.34575 | 23.24224 | 7.698382 |
| 3-hydroxybutyric acid | 17.67366 | 18.46388 | 22.73832 | 9.973339 |
| carnosine | 15.14287 | 19.07504 | 21.18876 | 6.982433 |
| indoxyl sulfate | 14.59567 | 18.0588 | 20.80125 | 5.361017 |

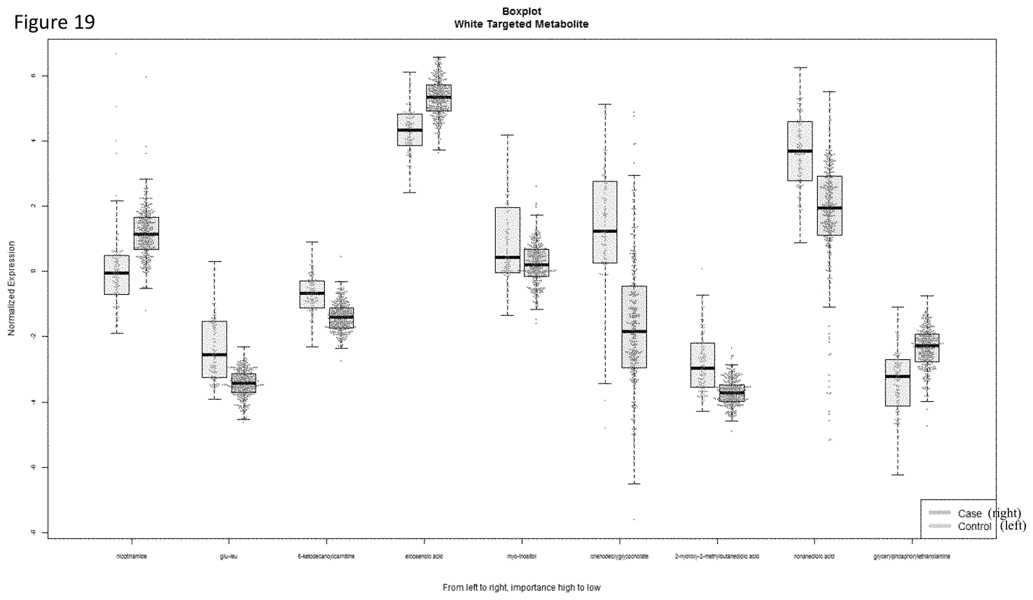

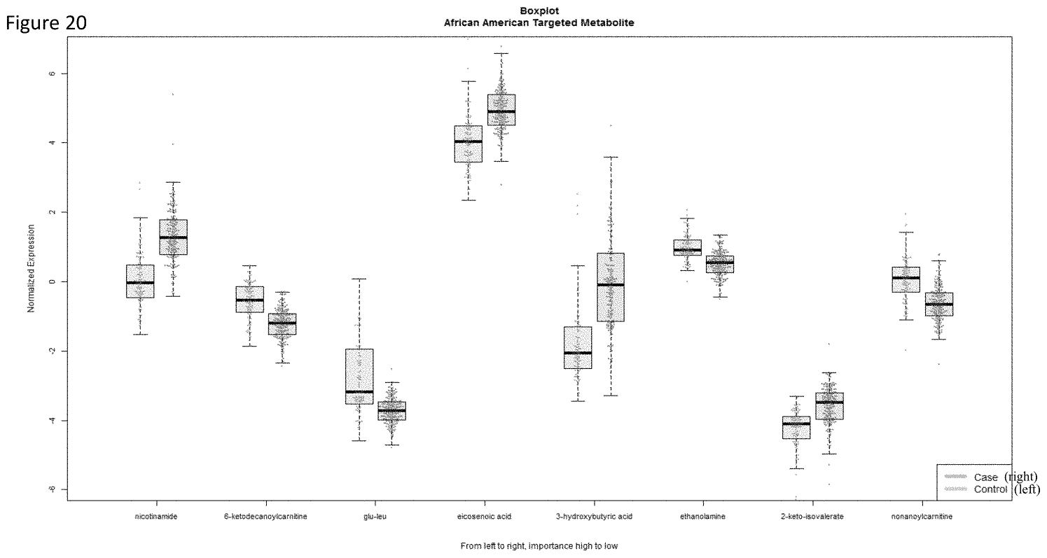

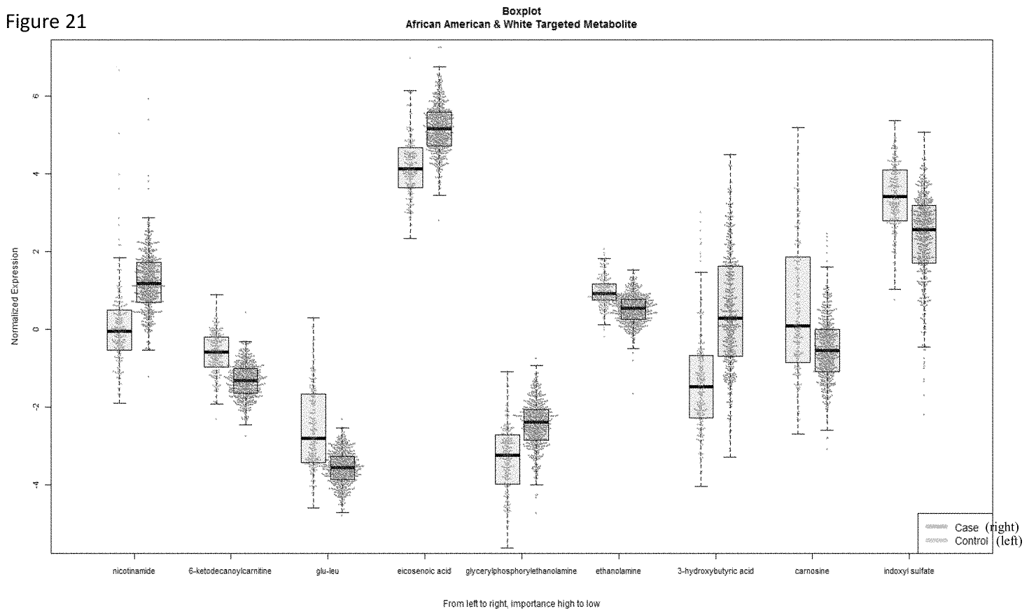

Expression levels of individual markers identified in Tables 19-21 were analyzed. FIGS. 19-21 are box plots depicting a direct comparison of normalized expression levels of individual markers identified in Tables 19-21 between Caucasian prostate cancer patients and negative controls, American African prostate cancer patients and negative controls, and prostate cancer patients from both races and negative controls, respectively. As shown in FIG. 19, expression levels of nicotinamide, eicosenoic acid and glycerylphosphorylethanolamine were increased in Caucasian prostate cancer patients when compared to negative controls, whereas other markers listed in Table 19 were decreased as compared to negative controls. When comparing the expression levels of markers in African American prostate cancer patients with negative controls, an increased level for nicotinamide, eicosenoic acid, 3-hydroxybutyric acid and 2-keto-isovalerate was observed in African American prostate cancer patients, whereas other markers in Table 20 showed a decreased expression level in African American prostate cancer patients as compared to negative controls (FIG. 20). A similar comparison was performed for prostate cancer patients from both races with negative controls. As shown in FIG. 21, an increase expression level of nicotinamide, eicosenoic acid, glycerylphosphorylethanolamine and 3-hydroxybutyric acid was observed in prostate cancer patients from both races, whereas other markers in Table 21 showed a decreased expression level in prostate cancer patients as compared to negative controls.

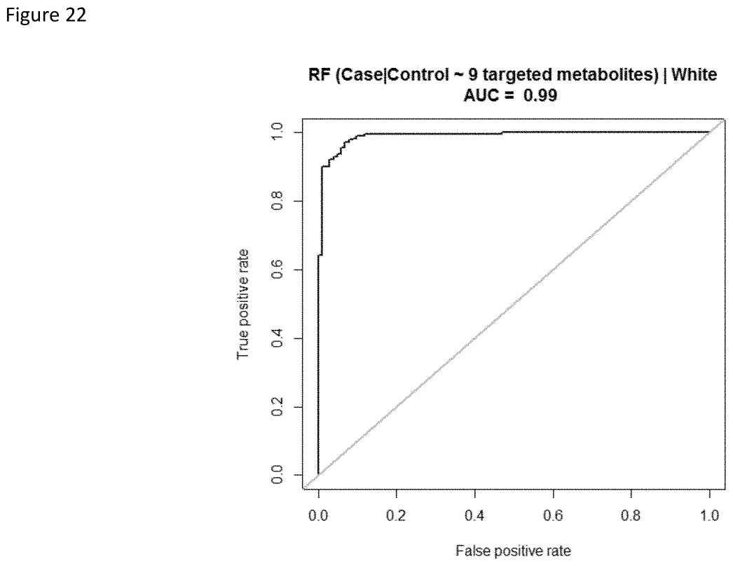

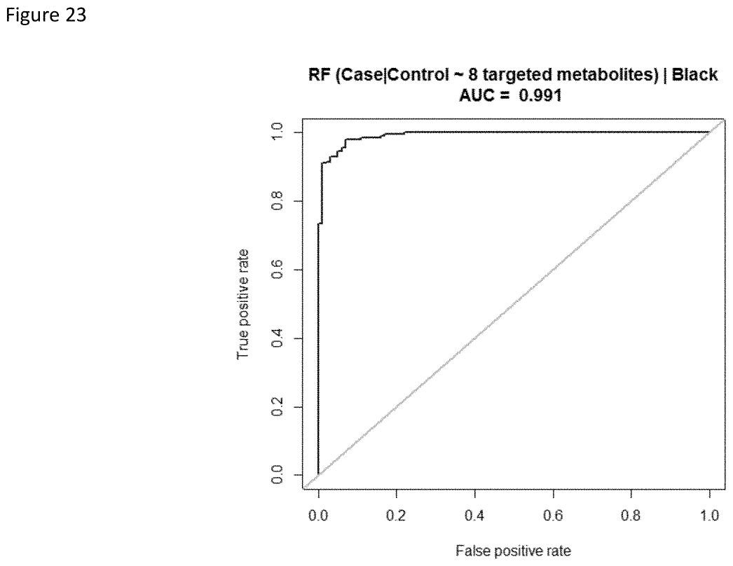

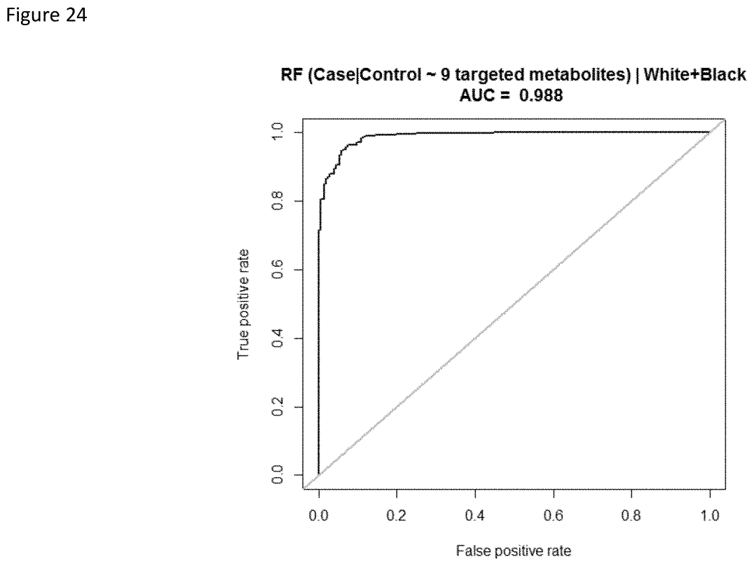

ROC curves were generated for markers identified from the Random Forest analysis. As shown in FIG. 22, the combination of the 9 metabolite markers identified in Table 19 has a predictive diagnostic value of 0.99 for Caucasian prostate cancer patients. The combination of the 8 metabolite markers identified in Table 20 has a predictive diagnostic value of 0.991 for African American prostate cancer patients (FIG. 23), and the combination of the 9 metabolite markers identified in Table 21 has a predictive diagnostic value of 0.988 for prostate cancer patients including both Caucasians and African Americans (FIG. 24).

These data indicate that the markers identified from Tables 19-21 may be used as biomarkers for the diagnosis and prognosis of prostate cancer, and to improve the accuracy of prostate cancer detection.

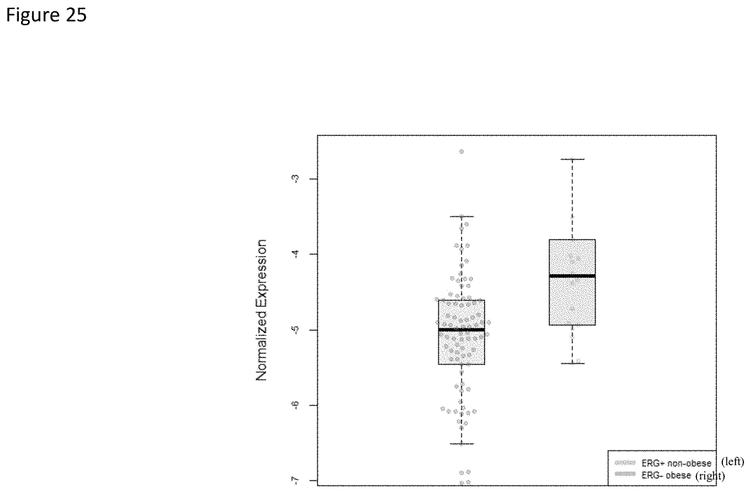

The data generated by the above protocol was also assessed for the log FC, pval, and FDR values generated by the conventional differential analysis (see Tables 22-25). Specifically, Tables 22 and 23 list metabolite species indicative of prostate cancer in Caucasians and African Americans patients, respectively. Table 24 compares Caucasian ERG positive non-obese patients versus Caucasian ERG negative obese patients. As shown in FIG. 25, obese Caucasian patients with ERG negative index prostate cancer have a significantly higher level of mercapto-succinyl-carnitine, which is a TCA cycle intermediate, in comparison to CA patients who are ERG positive and non-obese, indicating that this metabolite is a marker for prognosis associated with ERG and obesity status in CA patients.

Table 25 is a comparison of metabolite species indicative of prostate cancer between obese and non-obese patients. These metabolites demonstrated significant changes in amount from negative control to positive control as well as passing an FDR of less than 0.1. These data indicate that the markers identified from Tables 22-25 may also be used as biomarkers for the diagnosis and prognosis of prostate cancer, and to improve the accuracy of prostate cancer detection.

TABLE 22
Metabolite Markers Indicative of Prostate Cancer
for Caucasians Based on Differential Analysis

| | median. | median. | median. | median. |
|---|---|---|---|---|
| metabolite | diff | diff. FC | diff. pval | diff. FDR |
| 3-hydroxybutyric acid | 1.67409582 | 3.191192903 | 0 | 0 |
| alpha-linolenic acid | 1.49798325 | 2.824476016 | 0 | 0 |
| phosphorylcholine | 1.3635587 | 2.573191271 | 0 | 0 |
| linoleic acid | 1.24565231 | 2.371257456 | 0 | 0 |
| oleic acid | 1.20648807 | 2.307751801 | 0 | 0 |
| nicotinamide | 1.20121938 | 2.299339313 | 0 | 0 |
| stearic acid | 1.04244767 | 2.059719201 | 0 | 0 |
| adenosine | 1.00353711 | 2.004909492 | 0 | 0 |
| eicosenoic acid | 0.99730238 | 1.996263799 | 0 | 0 |
| cysteine-glycine | 0.98630276 | 1.981101449 | 0 | 0 |
| glycerylphosphoryl- | 0.92053741 | 1.892820246 | 0 | 0 |
| ethanolamine | | | | |
| 2-hydroxybutyric acid | 0.8869735 | 1.849292587 | 0 | 0 |
| oleamide | 0.88197356 | 1.842894598 | 0 | 0 |
| stearamide | 0.86819497 | 1.825377647 | 0 | 0 |
| oxo-octadecanoic acid | 0.7019011 | 1.626646882 | 0 | 0 |
| 2-octandioic-carnitine | 0.63608642 | 1.554107629 | 0 | 0 |
| cystine | 0.63185701 | 1.549558273 | 0 | 0 |
| 2-keto-isovalerate | 0.61282946 | 1.529255495 | 0 | 0 |
| cysteine | 0.60014567 | 1.515869617 | 0 | 0 |
| hydroxybutyrylcarnitine | 0.57629589 | 1.491016156 | 0 | 0 |
| 7-methylguanosine | 0.46131941 | 1.376800389 | 0 | 0 |
| uracil | 0.41846971 | 1.336509146 | 0 | 0 |
| s-methyl-cysteine | 0.38331885 | 1.304338975 | 0.0079 | 0.0367 |
| s-methylcysteine | 0.38331885 | 1.304338975 | 0.0079 | 0.0367 |
| succinyladenosine | 0.34357687 | 1.268898674 | 0.0056 | 0.026349 |
| glutamine | 0.30109056 | 1.23207541 | 0 | 0 |
| uridine | 0.28600877 | 1.219262506 | 0 | 0 |
| glutaconylcarnitine | 0.28246443 | 1.21627076 | 0.003 | 0.014878 |
| hexadecandioic acid | 0.27916668 | 1.213493752 | 0.0031 | 0.015169 |
| hexadecanedioic acid | 0.27916668 | 1.213493752 | 0.0031 | 0.015169 |
| oleoylcarnitine | 0.27615084 | 1.210959688 | 0 | 0 |
| amp | 0.26910806 | 1.205062573 | 0.0126 | 0.05505 |
| imidazoleacetic acid | 0.24582331 | 1.185769267 | 0 | 0 |
| glucosamine | 0.24269892 | 1.18320407 | 0 | 0 |
| s-adenosyl-l-methioninamine | 0.21618742 | 1.161659633 | 0.0045 | 0.021448 |
| 1-methyladenosine | 0.17698151 | 1.130516078 | 3.00E−04 | 0.001668 |
| palmitoylcarnitine | 0.17494235 | 1.128919292 | 7.00E−04 | 0.003618 |
| n,n-dimethyl-l-arginine | 0.16505708 | 1.121210438 | 6.00E−04 | 0.003191 |
| acetylcarnitine | 0.13475065 | 1.09790304 | 0.0081 | 0.037159 |
| malonylcarnitine | 0.11732211 | 1.084719567 | 0.014 | 0.060447 |
| tryptophan | −0.13689371 | 0.909475255 | 0.016 | 0.067494 |
| coumaric acid | −0.14098259 | 0.906901273 | 0.0096 | 0.042966 |
| guanidinosuccinic acid | −0.14744973 | 0.902845023 | 0.0213 | 0.087833 |
| kynurenine | −0.15677828 | 0.897026008 | 0.0022 | 0.011214 |
| 3-dehydroxycarnitine | −0.16364187 | 0.892768562 | 7.00E−04 | 0.003618 |
| xanthine | −0.1887628 | 0.877357787 | 0.0149 | 0.063585 |
| gamma-glu-gln | −0.21039742 | 0.86429911 | 4.00E−04 | 0.002191 |
| lpa | −0.21963488 | 0.858782751 | 0.017 | 0.070898 |
| [(2r)-2-(hexadecanoyloxy)- | −0.21963488 | 0.858782751 | 0.017 | 0.070898 |
| 3-ydroxypropoxy]phosphonic acid | | | | |
| lpa(0:0/16:0) | −0.21963488 | 0.858782751 | 0.017 | 0.070898 |
| 2-methylbutyroylcarnitine | −0.25939322 | 0.835439221 | 1.00E−04 | 0.000602 |
| glutamic acid | −0.26349536 | 0.833067118 | 0 | 0 |
| glutamate | −0.26349536 | 0.833067118 | 0 | 0 |
| butyrylcarnitine | −0.2647416 | 0.832347803 | 5.00E−04 | 0.002699 |
| phenylalanine | −0.28156489 | 0.822698153 | 0 | 0 |
| 2-hydroxyglutarate | −0.30044075 | 0.812004287 | 1.00E−04 | 0.000602 |
| 5-hydroxymethyl-2- | −0.32789448 | 0.796698365 | 0.0216 | 0.08808 |
| furoylcarnitine | | | | |
| n-acetylaspartic acid | −0.33505455 | 0.792754161 | 0.0125 | 0.05505 |
| riboflavin | −0.34427593 | 0.787703215 | 0.0041 | 0.019799 |
| n-acetylcarnosine | −0.35293027 | 0.78299214 | 2.00E−04 | 0.001165 |
| 2-aminoethylphosphonate | −0.3603689 | 0.778965371 | 3.00E−04 | 0.001668 |
| n-phenylacetyl-glutamine | −0.37773284 | 0.769646121 | 0.0096 | 0.042966 |
| n-phenylacetylglutamine | −0.37773284 | 0.769646121 | 0.0096 | 0.042966 |
| nonenoylcarnitine | −0.38771079 | 0.764341469 | 0 | 0 |
| ethanolamine | −0.38960991 | 0.763335975 | 0 | 0 |
| propionylcarnitine | −0.41232344 | 0.75141226 | 0 | 0 |
| acadesine | −0.4149841 | 0.750027761 | 3.00E−04 | 0.001668 |
| decenoylcarnitine | −0.5111419 | 0.701666845 | 0 | 0 |
| dodecanoylcarnitine | −0.5169356 | 0.698854681 | 0 | 0 |
| glycerate | −0.53426888 | 0.69050852 | 0 | 0 |
| dodecenoylcarnitine | −0.54443663 | 0.685659099 | 0 | 0 |
| sphingosine-1-phosphate | −0.55493513 | 0.680687664 | 0 | 0 |
| leucine | −0.56221363 | 0.677262194 | 2.00E−04 | 0.001165 |
| 3-methylphenylacetic acid | −0.592083 | 0.663384404 | 0 | 0 |
| guanidinebutyric acid | −0.60220384 | 0.658746894 | 0 | 0 |
| mandeloylcarnitine | −0.60254085 | 0.658593031 | 1.00E−04 | 0.000602 |
| octanoylcarnitine | −0.6110545 | 0.654717978 | 0 | 0 |
| 9-decenoylcarnitine | −0.64536105 | 0.639332771 | 0 | 0 |
| aspartate | −0.67990875 | 0.624204754 | 0 | 0 |
| dhea sulfate | −0.71180097 | 0.610557481 | 0.0024 | 0.012066 |
| nonanoylcarnitine | −0.71356083 | 0.609813152 | 0 | 0 |
| 4-pyridoxic acid | −0.71470484 | 0.609329781 | 0 | 0 |
| allantoin | −0.72835921 | 0.603589992 | 0 | 0 |
| 6-ketodecanoylcarnitine | −0.73791184 | 0.599606596 | 0 | 0 |
| 2-hydroxy-2- | −0.75972416 | 0.590609243 | 0 | 0 |
| methylbutanedioic acid | | | | |
| phe-phe | −0.77659086 | 0.583744574 | 1.00E−04 | 0.000602 |
| cresol | −0.78054978 | 0.582144908 | 1.00E−04 | 0.000602 |
| decanoylcarnitine | −0.82643525 | 0.563920912 | 0 | 0 |
| glu-leu | −0.8826088 | 0.542385757 | 0 | 0 |
| tridecanoyl carnitine | −0.94719231 | 0.518640829 | 0 | 0 |
| indoxyl sulfate | −0.98562979 | 0.505005222 | 0 | 0 |
| p-cresol sulfate | −1.05036878 | 0.482844724 | 1.00E−04 | 0.000602 |
| carnosine | −1.46892607 | 0.361251111 | 0 | 0 |
| nonanedioic acid | −1.7347102 | 0.300469363 | 0 | 0 |
| undecanedioic acid | −2.22554199 | 0.213818414 | 0 | 0 |
| chenodeoxyglycocholate | −3.07998913 | 0.118258097 | 0 | 0 |

TABLE 23
Metabolite Markers Indicative of Prostate Cancer for
African Americans Based on Differential Analysis

| | median. | median. | median. | median. |
|---|---|---|---|---|
| Metabolite | diff | diff. FC | diff. pval | diff. FDR |
| 3-hydroxybutyric acid | 1.95565764 | 3.87892704 | 0 | 0 |
| Nicotinamide | 1.30493876 | 2.470732394 | 0 | 0 |
| 2-octandioic-carnitine | 1.01033629 | 2.014380594 | 0 | 0 |
| eicosenoic acid | 0.85201066 | 1.805014792 | 0 | 0 |
| glycerylphosphoryl- | 0.71164645 | 1.637672015 | 0 | 0 |
| ethanolamine | | | | |
| hydroxybutyrylcarnitine | 0.67874224 | 1.600743598 | 0 | 0 |
| Adenosine | 0.66026112 | 1.580368636 | 9.00E−04 | 0.005505 |
| 2-keto-isovalerate | 0.61309265 | 1.529534502 | 0 | 0 |
| cysteine-glycine | 0.56960302 | 1.484115137 | 0 | 0 |
| 3-methylglutarylcarnitine | 0.46177937 | 1.37723941 | 0 | 0 |
| Cysteine | 0.45573172 | 1.371478228 | 0 | 0 |
| 2-octendioic-carnitine | 0.4271381 | 1.344563695 | 0 | 0 |
| oleoylcarnitine | 0.37236988 | 1.294477492 | 0 | 0 |
| Cysteine | 0.34037399 | 1.266084759 | 0 | 0 |
| 7-methylguanosine | 0.3347514 | 1.261160072 | 0 | 0 |
| pimelylcarnitine | 0.33444797 | 1.26089485 | 2.00E−04 | 0.001562 |
| palmitoylcarnitine | 0.3245953 | 1.252313095 | 0 | 0 |
| s-methyl-cysteine | 0.31734337 | 1.246033944 | 0.0024 | 0.013146 |
| s-methylcysteine | 0.31734337 | 1.246033944 | 0.0024 | 0.013146 |
| Uridine | 0.30983016 | 1.239561765 | 0 | 0 |
| Uracil | 0.30832167 | 1.23826635 | 0 | 0 |
| 1-methylnicotinamide | 0.29614271 | 1.227857136 | 0.0084 | 0.041104 |
| 1,2-dihydroxy-3- | 0.28738502 | 1.220426169 | 4.00E−04 | 0.002878 |
| methylcyclohexa-3,5- | | | | |
| dienecaboxylcarnitine | | | | |
| s-adenosyl-l-methioninamine | 0.26412213 | 1.200905085 | 7.00E−04 | 0.004757 |
| n-acetylalanine | 0.25266501 | 1.191405901 | 0.0164 | 0.076187 |
| Quinolinate | 0.2498039 | 1.189045482 | 0.002 | 0.011469 |
| Glucosamine | 0.23315517 | 1.175402743 | 0 | 0 |
| hexadecandioic acid | 0.21087825 | 1.15739254 | 0.0124 | 0.058344 |
| hexadecanedioic acid | 0.21087825 | 1.15739254 | 0.0124 | 0.058344 |
| o-acetyl-l-serine | 0.20596997 | 1.153461595 | 0.0019 | 0.011068 |
| malonylcarnitine | 0.17711756 | 1.130622694 | 0.0067 | 0.033228 |
| n,n-dimethyl-l-arginine | 0.16392075 | 1.120327671 | 9.00E−04 | 0.005505 |
| Deoxyinosine | 0.1622512 | 1.119031928 | 0 | 0 |
| n-acetyl-neuraminic acid | 0.15107208 | 1.11039431 | 0.0053 | 0.026645 |
| imidazoleacetic acid | 0.15079878 | 1.11018398 | 5.00E−04 | 0.003529 |
| 1-methyladenosine | 0.14965057 | 1.10930076 | 0.0021 | 0.011857 |
| galactosylhydroxylysine | 0.14675921 | 1.107079794 | 4.00E−04 | 0.002878 |
| Acetoacetate | −0.14648806 | 0.903447041 | 0.0029 | 0.015204 |
| 2-oxobutanoate | −0.14683711 | 0.903228485 | 9.00E−04 | 0.005505 |
| Arginine | −0.15708943 | 0.896832565 | 0.0024 | 0.013146 |
| 2-hydroxy-2- | −0.17027029 | 0.888676171 | 0.0106 | 0.050522 |
| methylbutanedioic acid | | | | |
| Lpa | −0.17284277 | 0.887092978 | 0.0091 | 0.043943 |
| [(2r)-2-(hexadecanoyloxy)-3- | −0.17284277 | 0.887092978 | 0.0091 | 0.043943 |
| ydroxypropoxy]phosphonic acid | | | | |
| lpa(0:0/16:0) | −0.17284277 | 0.887092978 | 0.0091 | 0.043943 |
| Tyrosine | −0.17772422 | 0.884096515 | 1.00E−04 | 0.000834 |
| coumaric acid | −0.19615277 | 0.872875156 | 0 | 0 |
| heptanoylcarnitine | −0.200231 | 0.870411185 | 0.0026 | 0.014032 |
| Kynurenine | −0.22239394 | 0.857141955 | 0 | 0 |
| 4-hydroxyphenyllactic | −0.23282409 | 0.850967483 | 9.00E−04 | 0.005505 |
| phenyllactic acid | −0.23604341 | 0.849070698 | 7.00E−04 | 0.004757 |
| gamma-glu-gln | −0.24044652 | 0.846483282 | 4.00E−04 | 0.002878 |
| lpc (16:0/0:0) | −0.24605466 | 0.843199163 | 0.0043 | 0.022227 |
| Lpc | −0.24605466 | 0.843199163 | 0.0043 | 0.022227 |
| 1-hexadecanoyl-sn-glycero- | −0.24605466 | 0.843199163 | 0.0043 | 0.022227 |
| 3-phosphocholine | | | | |
| Phenylalanine | −0.27573121 | 0.826031549 | 0 | 0 |
| Thiamine | −0.28200465 | 0.822447418 | 0.0182 | 0.083493 |
| Tryptophan | −0.29359359 | 0.815867292 | 0 | 0 |
| hydroxyproline | −0.30784908 | 0.807845282 | 9.00E−04 | 0.005505 |
| dodecenoylcarnitine | −0.31887581 | 0.801694338 | 2.00E−04 | 0.001562 |
| 3-dehydroxycarnitine | −0.34816253 | 0.78558401 | 0 | 0 |
| benzoylcarnitine | −0.35635695 | 0.781134589 | 0.0014 | 0.008423 |
| 5-oxoproline | −0.36538824 | 0.77625995 | 0.0016 | 0.009471 |
| Ethanolamine | −0.37018505 | 0.773683252 | 0 | 0 |
| indoleacrylic acid | −0.37069409 | 0.773410314 | 0 | 0 |
| dodecanoylcarnitine | −0.38708924 | 0.764670838 | 2.00E−04 | 0.001562 |
| decenoylcarnitine | −0.40230669 | 0.75664753 | 0 | 0 |
| 2-methylbutyroylcarnitine | −0.42929922 | 0.742622422 | 0 | 0 |
| 5-hydroxymethyl-2- | −0.43496672 | 0.739710818 | 0.0048 | 0.024467 |
| furoylcarnitine | | | | |
| Carnosine | −0.43660121 | 0.738873243 | 8.00E−04 | 0.005338 |
| octanoylcarnitine | −0.45449642 | 0.729764856 | 0 | 0 |
| 4-pyridoxic acid | −0.45789732 | 0.728046589 | 1.00E−04 | 0.000834 |
| Aspartate | −0.45946085 | 0.727257992 | 0 | 0 |
| Tmao | −0.46272625 | 0.725613775 | 4.00E−04 | 0.002878 |
| Acadesine | −0.49247048 | 0.710806865 | 0.0027 | 0.014361 |
| butyrylcarnitine | −0.49258452 | 0.71075068 | 0 | 0 |
| glu-leu | −0.54168395 | 0.686968594 | 0 | 0 |
| propionylcarnitine | −0.60056359 | 0.659496272 | 0 | 0 |
| decanoylcarnitine | −0.64402825 | 0.639923676 | 0 | 0 |
| nonenoylcarnitine | −0.65774971 | 0.63386622 | 0 | 0 |
| 6-ketodecanoylcarnitine | −0.66024591 | 0.632770431 | 0 | 0 |
| mandeloylcarnitine | −0.69045181 | 0.61965976 | 0 | 0 |
| n-phenylacetyl-glutamine | −0.74578381 | 0.596343792 | 1.00E−04 | 0.000834 |
| n-phenylacetylglutamine | −0.74578381 | 0.596343792 | 1.00E−04 | 0.000834 |
| nonanoylcarnitine | −0.76095371 | 0.590106105 | 0 | 0 |
| indoxyl sulfate | −0.84012143 | 0.558596551 | 0 | 0 |
| Sucrose | −1.16721737 | 0.445279355 | 0 | 0 |
| Cresol | −1.27458835 | 0.413343083 | 0 | 0 |

TABLE 24
Metabolite Markers Indicative of Prostate Cancer in
Caucasian ERG positive non-obese patients and ERG negative
obese patients Based on Differential Analysis

| all_names | logFC | FC | P. Value | FDR |
|---|---|---|---|---|
| mercaptosuccinylcarnitine | −1.44804 | 0.36652 | 5.73E−05 | 0.020116 |

TABLE 25
Comparison of Metabolite Markers Indicative of Prostate Cancer
in Obese and Non-Obese Patients Based on Differential Analysis

| all_names | logFC | FC | P. Value | FDR |
|---|---|---|---|---|
| lipoate | 0.988977 | 1.984777 | 0.026673 | 0.091377 |
| n-acetylasparagine | 0.866224 | 1.822885 | 0.000155 | 0.00387 |
| 1-methylnicotinamide | 0.686336 | 1.609192 | 9.14E−05 | 0.003048 |
| tridecanoyl carnitine | 0.675019 | 1.596618 | 0.010531 | 0.056013 |
| n-acetylarginine | 0.666311 | 1.587009 | 4.11E−05 | 0.002156 |
| cyclic-amp | 0.61892 | 1.535725 | 0.000691 | 0.009757 |
| cholesterol | 0.595252 | 1.510737 | 0.002519 | 0.021501 |
| cholesterol, tms derivative | 0.595252 | 1.510737 | 0.002519 | 0.021501 |
| 9-decenoylcarnitine | 0.587602 | 1.502746 | 0.000175 | 0.00387 |
| tetradecadiencarnitine | 0.579215 | 1.494036 | 0.014486 | 0.064079 |
| vanilin-4-sulfate | 0.555517 | 1.469695 | 0.005009 | 0.032843 |
| tetradecenoylcarnitine | 0.551873 | 1.465988 | 0.020291 | 0.077052 |
| orotidine-5-phosphate | 0.488289 | 1.402781 | 0.003402 | 0.024967 |
| n-acetylaspartic acid | 0.470367 | 1.385462 | 0.000352 | 0.005877 |
| eicosatetraenoic acid | 0.463283 | 1.378676 | 0.008744 | 0.05014 |
| 5-amino-3-oxohexanoic | 0.455389 | 1.371153 | 0.00459 | 0.03199 |
| acid | | | | |
| nonenoylcarnitine | 0.433217 | 1.350241 | 2.61E−06 | 0.000895 |
| 5-hydroxylysine | 0.422351 | 1.34011 | 0.025883 | 0.089614 |
| hypusine | 0.412327 | 1.330831 | 0.002449 | 0.0214 |
| mercaptosuccinylcarnitine | 0.396252 | 1.316084 | 0.001509 | 0.015382 |
| 2-octenoylcarnitine | 0.390154 | 1.310533 | 0.015003 | 0.064525 |
| 2-isopropylmalic acid | 0.386707 | 1.307405 | 0.00019 | 0.003876 |
| glucose-1-phosphate | 0.384406 | 1.305323 | 0.001134 | 0.013005 |
| cytidine | 0.353574 | 1.277722 | 0.016006 | 0.066597 |
| quinolinate | 0.335508 | 1.261822 | 0.002608 | 0.021754 |
| octenoylcarnitine | 0.326023 | 1.253553 | 0.00031 | 0.005411 |
| choline | 0.287854 | 1.220823 | 0.00088 | 0.011235 |
| decenoylcarnitine | 0.285278 | 1.218645 | 0.000159 | 0.00387 |
| decanoylcarnitine | 0.262461 | 1.199523 | 0.011276 | 0.058943 |
| n-acetyl-glutamate | 0.262343 | 1.199425 | 0.000934 | 0.011424 |
| octanoylcarnitine | 0.257092 | 1.195067 | 0.007907 | 0.046064 |
| n-acetyl-glucosamine-1- | 0.252733 | 1.191462 | 0.005011 | 0.032843 |
| phosphate | | | | |
| 2-pyrrolidinone | 0.23254 | 1.174901 | 0.000236 | 0.004555 |
| dodecenoylcarnitine | 0.19502 | 1.14474 | 0.011643 | 0.058943 |
| guanidinepropionic acid | 0.185022 | 1.136834 | 0.025882 | 0.089614 |
| nonanoylcarnitine | 0.18017 | 1.133018 | 0.018242 | 0.071986 |
| xanthine | 0.176198 | 1.129902 | 0.014492 | 0.064079 |
| heptanoylcarnitine | 0.168514 | 1.1239 | 0.013353 | 0.062034 |
| cyclohexanoylcarnitine | 0.164604 | 1.120858 | 0.006489 | 0.03969 |
| nadp+ | 0.163587 | 1.120069 | 0.005534 | 0.035017 |
| propionylcarnitine | 0.16035 | 1.117558 | 0.012843 | 0.061394 |
| butyrylcarnitine | 0.151377 | 1.110629 | 0.016955 | 0.06914 |
| methylmalonylcarnitine | 0.149452 | 1.109148 | 0.002034 | 0.018816 |
| 2-methylbutyroylcarnitine | 0.148209 | 1.108193 | 0.006201 | 0.038573 |
| n-acetylputrescine | 0.143826 | 1.104831 | 0.018688 | 0.072961 |
| 6-ketodecanoylcarnitine | 0.126105 | 1.091343 | 0.027629 | 0.093025 |
| tyrosine | 0.115485 | 1.083339 | 0.011833 | 0.058943 |
| coumaric acid | 0.111025 | 1.079995 | 0.011432 | 0.058943 |
| p-hydroxybenzoate | −0.07543 | 0.949056 | 0.028944 | 0.094845 |
| deoxyinosine | −0.09216 | 0.93812 | 0.028646 | 0.094714 |
| asparagine | −0.12045 | 0.919899 | 0.003371 | 0.024967 |
| acetylcarnitine | −0.12367 | 0.917852 | 0.020409 | 0.077052 |
| 7-methylguanosine | −0.13578 | 0.910178 | 0.021105 | 0.078236 |
| glutamine | −0.13717 | 0.909301 | 0.011885 | 0.058943 |
| galactosylhydroxylysine | −0.14185 | 0.906354 | 0.02258 | 0.081949 |
| histidine | −0.14401 | 0.905003 | 0.006723 | 0.040448 |
| n-acetyl-glucosamine | −0.14669 | 0.903324 | 0.02689 | 0.091377 |
| lysine | −0.15051 | 0.900931 | 0.002118 | 0.018956 |
| acetoacetate | −0.16293 | 0.893211 | 0.003632 | 0.026136 |
| guanidinosuccinic acid | −0.17197 | 0.887631 | 0.000426 | 0.006497 |
| n,n-dimethyl-l-arginine | −0.172 | 0.887614 | 0.000443 | 0.006497 |
| serine | −0.17478 | 0.8859 | 0.007871 | 0.046064 |
| o-acetyl-l-serine | −0.17734 | 0.884334 | 0.005336 | 0.034358 |
| 2-oxobutanoate | −0.1819 | 0.881539 | 0.00149 | 0.015382 |
| allantoate | −0.20382 | 0.868247 | 0.000269 | 0.004931 |
| 2-hydroxybutyric acid | −0.2047 | 0.86772 | 0.01512 | 0.064525 |
| aspartate | −0.21344 | 0.862476 | 0.009689 | 0.053072 |
| s1p | −0.21924 | 0.859017 | 0.009162 | 0.051733 |
| glycine | −0.22453 | 0.855876 | 5.06E−05 | 0.002321 |
| gamma-glu-gln | −0.22461 | 0.855828 | 0.000138 | 0.00387 |
| orotate | −0.2288 | 0.853343 | 0.030882 | 0.099293 |
| hexadecandioic acid | −0.2323 | 0.851276 | 0.031114 | 0.099293 |
| hexadecanedioic acid | −0.2323 | 0.851276 | 0.031114 | 0.099293 |
| 2-aminobutyrate | −0.24216 | 0.845481 | 0.002051 | 0.018816 |
| s-adenosyl-l-methionine | −0.25093 | 0.840353 | 0.017683 | 0.071204 |
| carnosine | −0.25136 | 0.840104 | 0.016025 | 0.066597 |
| 3-hydroxy-3- | −0.25149 | 0.840027 | 0.028568 | 0.094714 |
| methylglutarylcarnitine | | | | |
| homocarnosine | −0.26309 | 0.833301 | 0.023772 | 0.084701 |
| lactate | −0.27076 | 0.828881 | 0.017849 | 0.071204 |
| lactic acid | −0.27076 | 0.828881 | 0.017849 | 0.071204 |
| n-carbamoyl-aspartate | −0.27458 | 0.826693 | 0.001245 | 0.013436 |
| 2-octandioic-carnitine | −0.27798 | 0.824743 | 0.022258 | 0.081688 |
| 3-hydroxysuberoylcarnitine | −0.28011 | 0.823529 | 0.025061 | 0.088435 |
| pimelylcarnitine | −0.28538 | 0.820524 | 0.014405 | 0.064079 |
| ornithine | −0.28818 | 0.818935 | 6.09E−05 | 0.002482 |
| allantoin | −0.28841 | 0.818802 | 0.020575 | 0.077052 |
| arginine | −0.29177 | 0.816899 | 8.87E−06 | 0.000895 |
| 5-methyl-thf | −0.30746 | 0.808064 | 0.009602 | 0.053072 |
| glycerate | −0.33843 | 0.790903 | 0.014684 | 0.064156 |
| eicosenoic acid | −0.34493 | 0.787345 | 3.98E−05 | 0.002156 |
| citrulline | −0.34937 | 0.784926 | 0.000179 | 0.00387 |
| pipecolic acid | −0.36052 | 0.778883 | 0.001603 | 0.015901 |
| pyridoxine | −0.38465 | 0.765966 | 0.022776 | 0.081949 |
| acetylcholine | −0.40756 | 0.753896 | 0.012881 | 0.061394 |
| s-methylglutathione | −0.41039 | 0.752419 | 0.002777 | 0.022648 |
| n-carbamoyl-l-aspartate | −0.42543 | 0.744619 | 0.003006 | 0.023469 |
| 3-hydroxyoctanoylcarnitine | −0.45119 | 0.731438 | 0.012319 | 0.060279 |
| indole-3-carboxylic acid | −0.45976 | 0.727106 | 0.001952 | 0.018816 |
| glycerophosphocholine | −0.47861 | 0.717671 | 0.000888 | 0.011235 |
| s-methylcysteine | −0.48361 | 0.715184 | 5.32E−06 | 0.000895 |
| s-methyl-cysteine | −0.48361 | 0.715184 | 5.32E−06 | 0.000895 |
| tetradecanedioic acid | −0.48578 | 0.714111 | 1.77E−05 | 0.001302 |
| nicotinate | −0.48741 | 0.713307 | 0.010136 | 0.054704 |
| 5-oxoproline | −0.50615 | 0.704097 | 9.76E−06 | 0.000895 |
| alpha-linolenic acid | −0.52978 | 0.692659 | 0.020222 | 0.077052 |
| dodecanoic acid | −0.57666 | 0.670513 | 0.00462 | 0.03199 |
| lactose-phosphate | −0.61177 | 0.654395 | 0.001091 | 0.012914 |
| 3-s-methylthiopropionate | −0.61884 | 0.651194 | 0.002937 | 0.023436 |
| undecanedioic acid | −0.62204 | 0.649751 | 0.01615 | 0.066597 |
| 4-sulfophenol | −0.63393 | 0.64442 | 0.004856 | 0.032843 |
| imidazolepropionic acid | −0.70312 | 0.614242 | 0.013886 | 0.063702 |
| 3-hydroxybutyric acid | −0.77985 | 0.582428 | 7.07E−05 | 0.002595 |
| hippuric acid | −0.91433 | 0.530589 | 0.013284 | 0.062034 |
| myristic acid | −0.93736 | 0.522189 | 0.000731 | 0.009942 |
| 2-ketohexanoic acid | −1.08963 | 0.469882 | 0.001227 | 0.013436 |
| purine | −1.10998 | 0.4633 | 0.003281 | 0.024967 |
| n-acetyl-serine | −1.13668 | 0.454804 | 0.000127 | 0.00387 |
| n6-acetyl-l-lysine | −1.39853 | 0.379317 | 0.000434 | 0.006497 |
| indolelactic acid | −1.86536 | 0.274455 | 0.02938 | 0.09542 |

### Example 6: Identification of Markers by Analysis of Omics Data

Random Forest analysis were further performed on data collected from all omics as described above, including proteomic, metabolomics, and lipidomic markers. In addition, ROC curves were generated for these markers that provided predictive diagnostic values for Caucasian prostate cancer patients, African American prostate cancer patients and patients with both races.

Table 26 includes the top omics markers indicative of prostate cancer based on Random Forest analysis for Caucasian prostate cancer patients. Table 27 includes the top omics markers indicative of prostate cancer for African American prostate cancer patients. Table 28 includes the top omics markers indicative of prostate cancer for prostate cancer patients from both races.

TABLE 26
Omics Markers Indicative of Prostate Cancer for
Caucasians Based on Random Forest Analysis

| | | | MeanDe- | MeanDe- |
|---|---|---|---|---|
| | | | crease | crease |
| all_names | 0 | 1 | Accuracy | Gini |
| glu-leu | 11.18831 | 13.14884 | 13.59893 | 2.479597 |
| 5-HETE | 11.43956 | 12.36697 | 12.95483 | 2.892644 |
| nicotinamide | 10.3276 | 11.13171 | 12.24707 | 1.852434 |
| 15-OXOETE | 10.46139 | 11.26275 | 12.15431 | 2.395399 |
| 8-HETE | 10.356 | 10.91333 | 12.01887 | 2.195848 |
| 5-HEPE | 10.07862 | 10.96909 | 11.94164 | 1.788997 |
| 6-ketodecanoylcarnitine | 9.730178 | 10.82643 | 11.8888 | 1.691453 |

TABLE 27
Omics Markers Indicative of Prostate Cancer for
African Americans Based on Random Forest Analysis

| | | | MeanDe- | MeanDe- |
|---|---|---|---|---|
| | | | crease | crease |
| all_names | 0 | 1 | Accuracy | Gini |
| nicotinamide | 15.80606 | 16.10416 | 17.15284 | 4.05556 |
| 6-ketodecanoylcarnitine | 14.27854 | 16.51058 | 16.77471 | 3.326935 |
| glu-leu | 13.1804 | 15.86682 | 16.02967 | 3.009939 |
| eicosenoic acid | 10.14096 | 11.77028 | 12.68854 | 2.409872 |
| 3-hydroxybutyric acid | 11.4501 | 10.56489 | 12.67776 | 2.736529 |
| 13-HOTRE/13- | 12.38331 | 9.505586 | 12.47253 | 2.29986 |
| HOTRE(R) | | | | |
| nonanoylcarnitine | 10.7542 | 10.15264 | 12.02872 | 2.275349 |
| ethanolamine | 10.59498 | 9.434299 | 11.94327 | 1.81696 |
| 2-keto-isovalerate | 10.60109 | 7.719398 | 10.82617 | 1.641836 |
| 9-HOTRE | 10.60145 | 7.97165 | 10.59603 | 1.744069 |
| FFA_18:3 | 8.647684 | 7.789352 | 10.16326 | 1.54314 |
| propionylcarnitine | 8.669351 | 8.492722 | 9.874553 | 1.58087 |
| 2-octandioic-carnitine | 9.587721 | 6.760843 | 9.815937 | 1.595157 |

TABLE 28
Omics Markers Indicative of Prostate Cancer for Caucasians
and African Americans Based on Random Forest Analysis

| | | | MeanDe- | MeanDe- |
|---|---|---|---|---|
| | | | crease | crease |
| all_names | 0 | 1 | Accuracy | Gini |
| 6-ketodecanoylcarnitine | 18.15829 | 20.29193 | 21.59148 | 6.788982 |
| nicotinamide | 19.02384 | 20.25761 | 21.56437 | 8.707283 |
| glu-leu | 14.541 | 19.74797 | 20.01294 | 6.138441 |
| eicosenoic acid | 15.61311 | 15.59213 | 17.43815 | 5.363623 |
| 3-hydroxybutyric acid | 12.55365 | 12.93478 | 14.84568 | 3.874881 |
| nonanoylcarnitine | 12.02789 | 12.06208 | 14.43604 | 3.535041 |
| ethanolamine | 11.09286 | 12.22552 | 14.39039 | 2.863026 |
| 13-HOTRE/13- | 13.3946 | 9.826011 | 13.76729 | 2.685144 |
| HOTRE(R) | | | | |
| 5-HETE | 11.23291 | 12.74447 | 13.71978 | 3.514265 |
| glycerylphosphoryl- | 10.7245 | 12.25798 | 13.48921 | 2.936847 |
| ethanolamine | | | | |
| 5-HEPE | 10.24758 | 11.88669 | 12.706 | 2.453409 |
| 15-OXOETE | 9.369425 | 12.14075 | 12.49056 | 2.750313 |

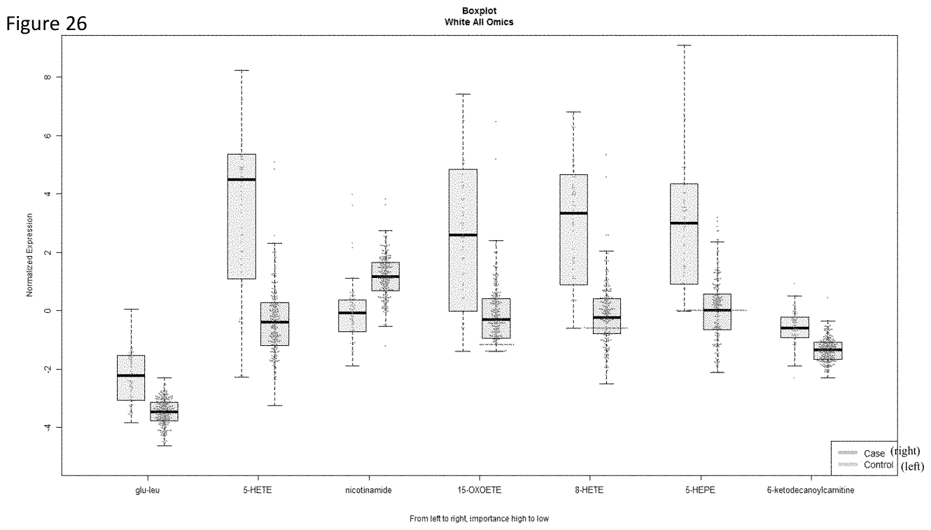

Expression levels of individual markers identified in Tables 26-28 were analyzed. As shown in FIG. 26, the expression level of nicotinamide was increased in Caucasian prostate cancer patients when compared to negative controls, whereas other markers listed in Table 26 had a decreased expression level.

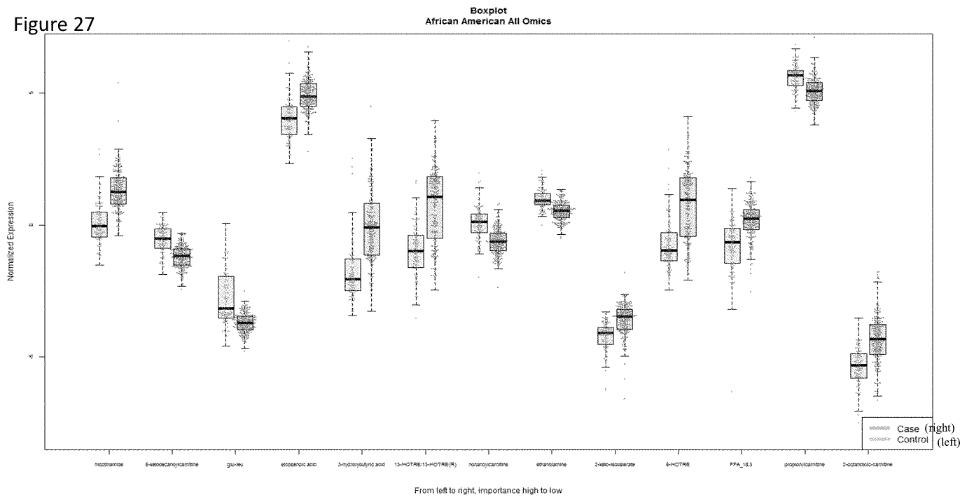

When compared the expression levels of these omic markers in African American prostate cancer patients with negative controls, an increased level for nicotinamide, eicosenoic acid, 3-hydroxybutyric acid, 13-HOTRE/13-HOTRE(R), 2-keto-isovalerate, 9-HOTRE, FFA_18:3 and 2-octandioic-carnitine was observed in African American prostate cancer patients, whereas other markers in Table 27 showed a decreased expression level in African American prostate cancer patients (FIG. 27).

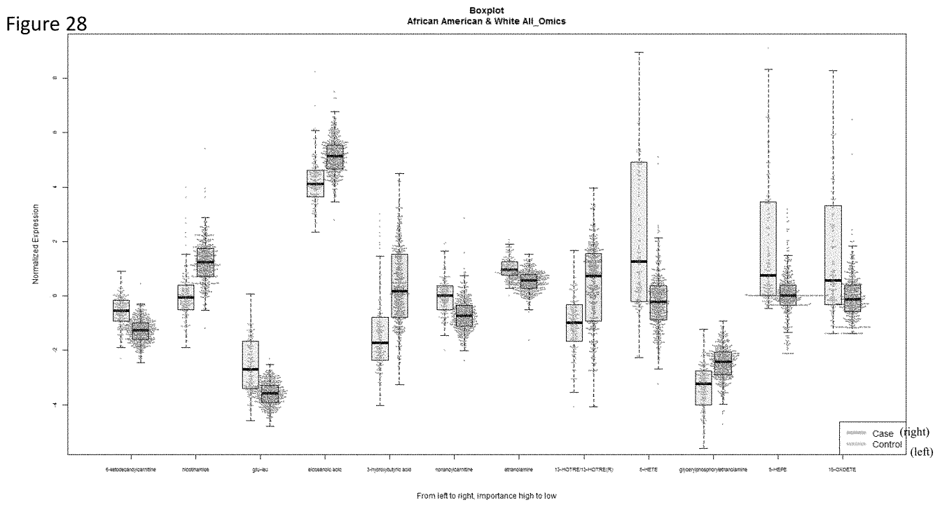

Similar comparison was performed for prostate cancer patients from both races with negative controls. As shown in FIG. 28, an increase expression level of nicotinamide, eicosenoic acid, 3-hydroxybutyric acid, 13-HOTRE/13-HOTRE(R), and glycerylphosphorylethanolamine was observed in prostate cancer patients from both races, whereas other markers in Table 28 showed a decreased expression level in prostate cancer patients.

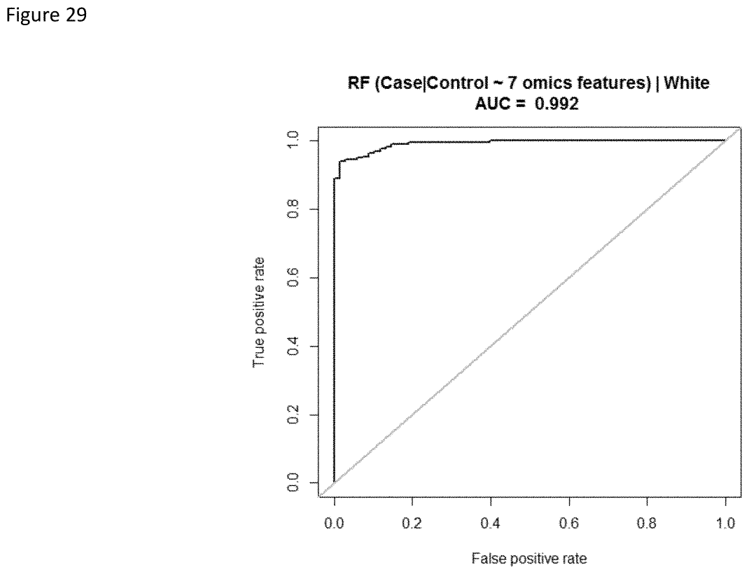

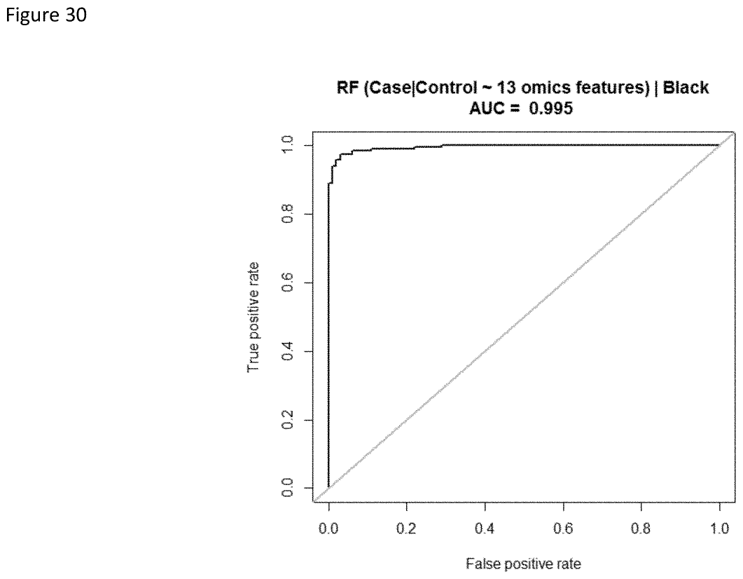

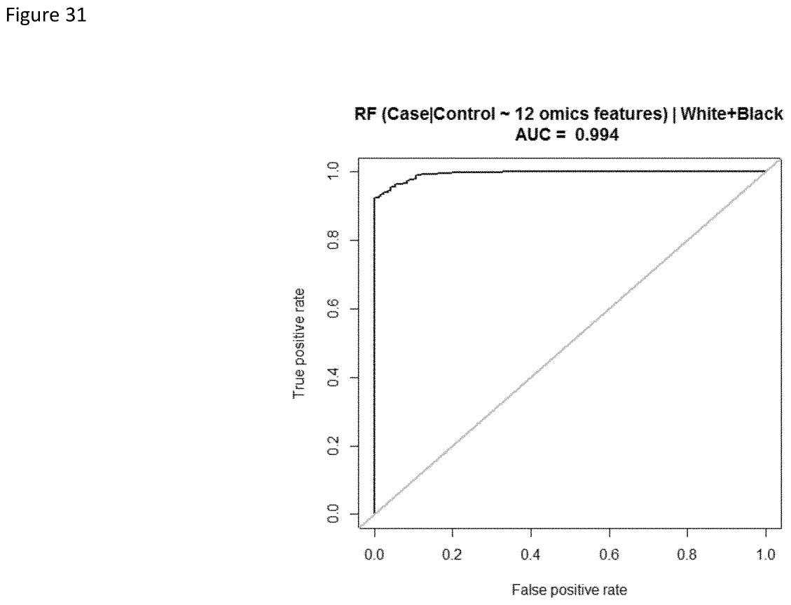

ROC curves were generated for omics marked identified from the Random Forest analysis. As shown in FIG. 29, the combination of the 7 markers identified in Table 26 has a predictive diagnostic value of 0.992 for Caucasian prostate cancer patients. The combination of the 13 markers identified in Table 27 has a predictive diagnostic value of 0.995 for African American prostate cancer patients (FIG. 30), and the combination of the 12 markers identified in Table 28 has a predictive diagnostic value of 0.994 for prostate cancer patients including both Caucasians and African Americans (FIG. 31).

These data indicate that the markers identified in Tables 26-28 may be used as biomarkers for the diagnosis and prognosis of prostate cancer, and to improve the accuracy of prostate cancer detection.

### Example 7: Predictive Analysis for Gleason Scores

To identify markers for prostate cancer diagnosis and treatment, a bootstrapping method was implemented. The method described herein was used to identify omics markers, including proteomic, metabolomics, and lipidomic markers, that had increased or decreased expression between Caucasian prostate cancer patients and negative control groups. This method does not rely on specific data distribution and reduces potential batch effect between patient and control samples and among cohorts. P value of each marker was evaluated by 1000 permutations of sample classes (patient/control) and corrected to FDR for multiple tests.

Briefly, omic markers of Caucasian prostate cancer patients and control populations were normalized using G-log normalization. Markers with missing value were removed from analysis. Sample annotations for different cohorts were combined and normalized. Samples with inconsistent annotation were removed from the analysis.

Patients' Gleason scores were divided into three classes: <=6, 7 and >=8. An ensemble learning method, Random Forest analysis, was used to predict Gleason score class using marker levels. The number of trees used in the ensemble model was tuned. Patients were stratified by race (Caucasian or African American). The importance of each marker in prediction was evaluated by Random Forest model and ranked. The performance of models were evaluated by increasing the number of marker used in model stepwise and ROC analysis. The final model was selected by the best AUC of ROC curve and the least marker used.

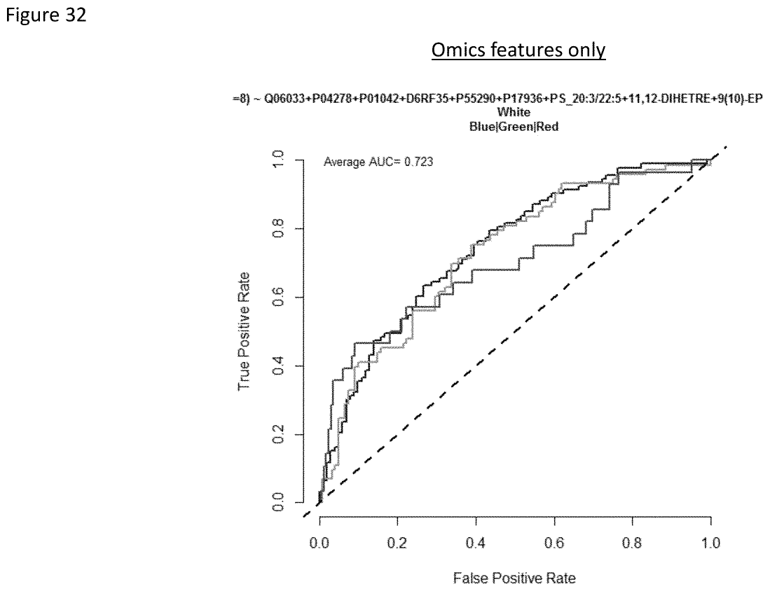

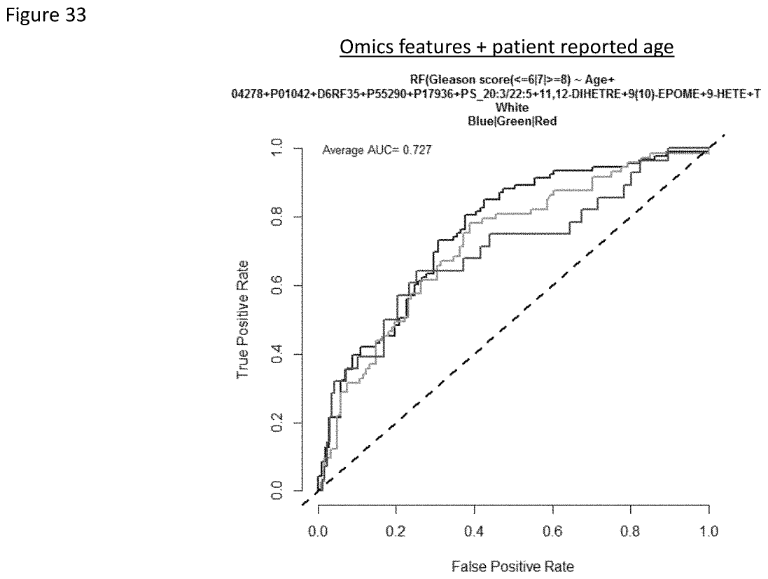

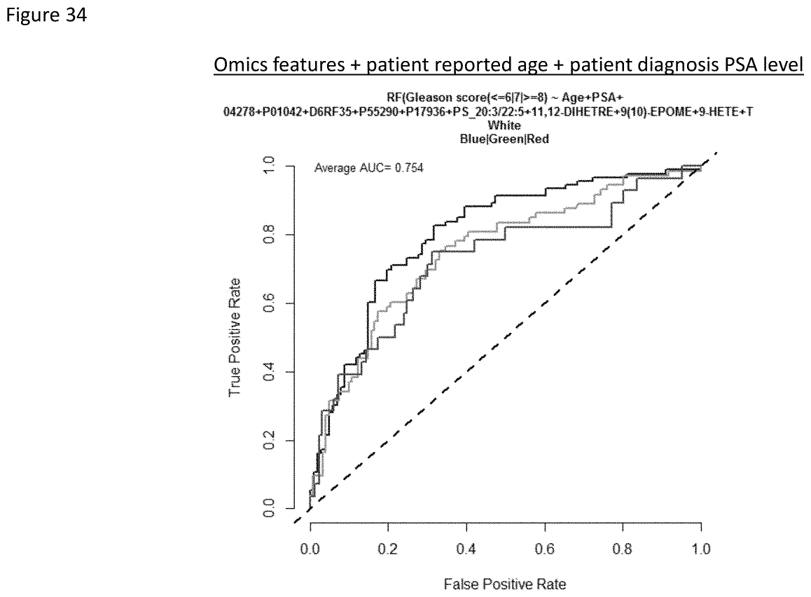

Table 29 includes the omics markers selected for predicting Gleason score class in Caucasian prostate cancer patients. ROC curves were generated for these markers, as well as other variables such as patient's age and PSA levels. As shown in FIG. 32, the combination of all omics markers included in Table 29 generates an AUC value of 0.723. Patient's age alone increases the AUC to 0.727 (FIG. 33), while the addition of patient's PSA level further increases the AUC value to 0.754 (FIG. 34). These data indicate that the markers identified from this analysis and included in Table 29 may be used as biomarkers for the diagnosis and prognosis of prostate cancer, and to improve the accuracy of prostate cancer detection.

TABLE 29
Omics Markers Selected for Gleason Score Class Prediction
in Caucasian Prostate Cancer Patients

| Protein | Accession | Gene | Description |
|---|---|---|---|
| | Q06033 | ITIH3 | Inter-alpha-trypsin inhi- |
| | | | bitor heavy chain H3 |
| | P04278 | SHBG | Sex hormone-binding |
| | | | globulin |
| | P01042 | KNG1 | Kininogen-1 |
| | D6RF35 | GC | Vitamin D-binding protein |
| | P55290 | CHD13 | Cadherin-13 |
| | P17936 | IGFBP3 | Insulin-like growth factor- |
| | | | binding protein 3 |
| Structural | PS-20:3/22:5 | | |
| lipid | | | |
| Signaling | 11,12-DIHETRE | | |
| lipid | 9(10)-EPOME | | |
| | 9-HETE | | |
| | TXB2 | | |
| Targeted | | | |
| metabolite | ID | Name | |
| | BM000536 | Tiglyl- | |
| | | carnitine | |

### Example 8: Lipid Markers Indicative of ERG Status

To identify lipid markers indicative of ERG status in prostate cancer patients, additional analysis were performed. Data were generated as described above, and were assessed for log FC, pval, and FDR values obtained from the conventional differential analysis (see Tables 30 and 31). The data generated for ERG positive versus ERG negative prostate cancer samples were compared between patients with different races. For example, Table 30 compares Caucasian ERG positive patients versus ERG negative patients. Table 31 compares African American ERG positive patients versus ERG negative patients.

TABLE 30
Lipid Markers Indicative of ERG Status for Caucasian Prostate
Cancer Patients Based on Differential Analysis

| Lipid | logFC | P. Value | FDR |
|---|---|---|---|
| LPC_O-14:1 | −0.51773 | 4.32E−05 | 0.02819 |
| LPC_22:1 | −0.31813 | 9.39E−05 | 0.02819 |
| LPC_10:0 | −0.58158 | 9.93E−05 | 0.02819 |
| LPC_O-22:0 | −0.50126 | 0.000396 | 0.084284 |
| LPC_24:0 | −0.28185 | 0.000622 | 0.105995 |
| CE_20:4 + NH4 | 0.379473 | 0.000958 | 0.135971 |

TABLE 31
Lipid Markers Indicative of ERG Status for African American
Prostate Cancer Patients Based on Differential Analysis

| Lipid | logFC | P. Value | FDR |
|---|---|---|---|
| PG_16:1/18:3 | 0.406902 | 1.703E−05 | 0.014513 |
| D18:0/16:1- | 0.5125942 | 9.014E−05 | 0.038399 |
| MONOHEX | | | |
| D18:1/22:1- | 0.5006782 | 0.0003918 | 0.11127 |
| MONOHEX | | | |
| PG_16:1/20:3 | 0.3243435 | 0.0005726 | 0.121966 |

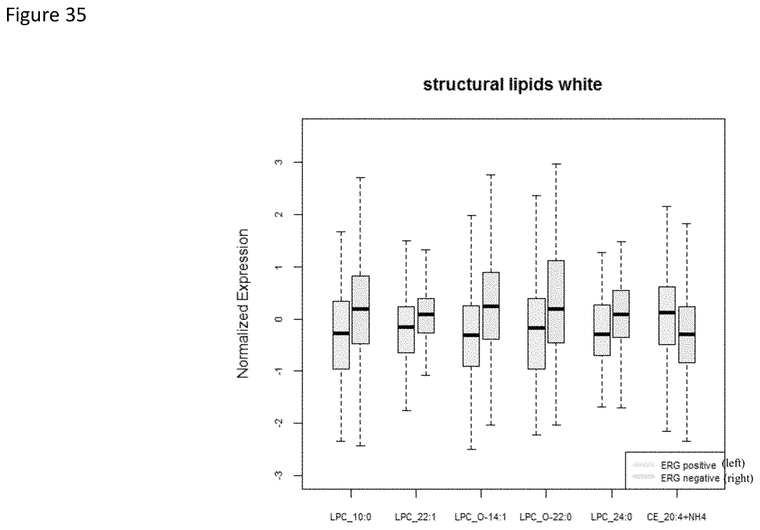

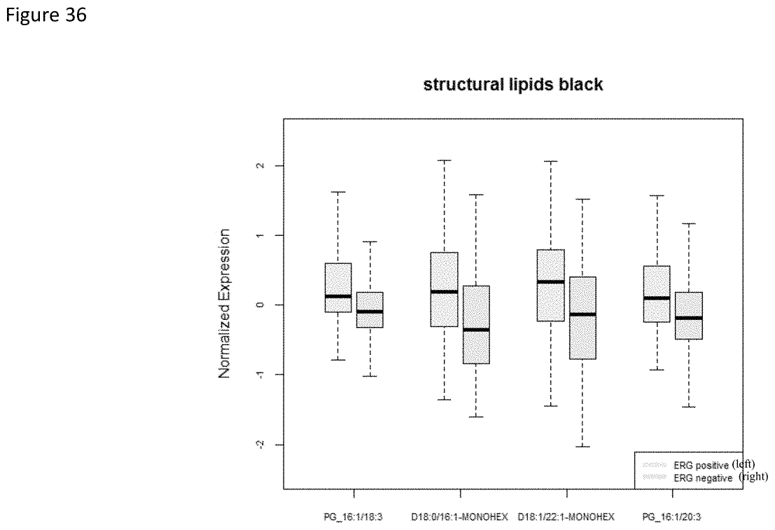

Expression levels of individual markers identified in Tables 30 and 31 were analyzed. FIGS. 32 and 33 are box plots depicting a direct comparison of normalized expression levels of individual markers identified in Tables 30 and 31 between Caucasian ERG positive and ERG negative prostate cancer patients, and between African American ERG positive and ERG negative prostate cancer patients, respectively. As shown in FIG. 35, expression levels of all markers listed in Table 30, with the single exception of CE_20:4+NH4, were decreased in Caucasian ERG positive prostate cancer patients. When comparing the expression levels of markers in African American ERG positive and ERG negative prostate cancer patients, an increased level was observed in all four markers in Table 31 (FIG. 36).

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

It is understood that the detailed examples and embodiments described herein are given by way of example for illustrative purposes only, and are in no way considered to be limiting to the invention. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present invention will be apparent from the appended claims. Moreover, those skilled in the art will recognize, or be able to ascertain using no more than routine experimentation, many equivalents to the specific embodiments of the invention described herein. Such equivalents are intended to be encompassed by the following claims.

1. A method for diagnosing the presence of prostate cancer in a subject, comprising:
(a) detecting the level of a prostate cancer marker in a biological sample from the subject, wherein the prostate cancer marker comprises one or more markers selected from Tables 1-31; and
(b) comparing the level of the prostate cancer marker in the biological sample with a predetermined threshold value;
wherein the level of the prostate cancer marker above or below the predetermined threshold value indicates a diagnosis that prostate cancer is present in the subject.

2. The method of claim 1, wherein the subject is selected from a population of Caucasians, and wherein the prostate cancer marker comprises one or more markers selected from Tables 1, 4, 8, 11, 13, 16, 19, 22, 26, 29 and 30.

3. The method of claim 1, wherein the subject is selected from a population of African Americans, and wherein the prostate cancer marker comprises one or more markers selected from Tables 2, 5, 9, 12, 14, 17, 20, 23, 27 and 31.

4. A method for diagnosing the presence of ERG-positive prostate cancer in a subject, comprising:
(a) detecting the level of an ERG-positive prostate cancer marker in a biological sample from the subject, wherein the ERG-positive prostate cancer marker comprises one or more markers selected from Tables 6, 30 and 31; and
(b) comparing the level of the ERG-positive prostate cancer marker in the biological sample with a predetermined threshold value;
wherein the level of the ERG-positive prostate cancer marker above or below the predetermined threshold value indicates a diagnosis that ERG-positive prostate cancer is present in the subject.

5. A method for diagnosing the presence of prostate cancer in a subject with a BMI index equal or greater than 30, comprising:
(a) detecting the level of a high BMI prostate cancer marker in a biological sample from the subject, wherein the high BMI prostate cancer marker comprises one or more markers selected from Tables 7, 18 and 25; and
(b) comparing the level of the high BMI prostate cancer marker in the biological sample with a predetermined threshold value;
wherein the level of the high BMI prostate cancer marker above or below the predetermined threshold value indicates a diagnosis that prostate cancer is present in the subject.

6. (canceled)

7. The method of claim 1, wherein the prostate cancer marker comprises at least two or more markers, wherein each of the two of more markers are selected from the structural lipids set forth in Tables 1-7, the signaling lipids set forth in Tables 8-12, the proteins set forth in Tables 13-18, the metabolites set forth in Tables 19-25, and the markers set forth in Tables 26-28.

8.-21. (canceled)

22. The method of claim 4, wherein the ERG-positive prostate cancer marker comprises one or more markers with an increased level when compared to the predetermined threshold value in the subject, and/or one or more markers with a decreased level when compared to the predetermined threshold value in the subject.

23.-25. (canceled)

26. The method of claim 5, wherein the high BMI prostate cancer marker comprises one or more markers with an increased level when compared to the predetermined threshold value in the subject, and/or one or more markers with a decreased level when compared to the predetermined threshold value in the subject.

27. (canceled)

28. The method of claim 1, wherein the level of the prostate cancer marker is detected by HPLC/UV-Vis spectroscopy, enzymatic analysis, mass spectrometry, NMR, immunoassay, ELISA, or any combination thereof, or by determining the level of its corresponding mRNA in the biological sample.

29.-31. (canceled)

32. The method of claim 1, further comprising administering a therapeutic anti-cancer treatment where the diagnosis indicates the presence of prostate cancer in the subject.

33. The method of claim 1, further comprising selecting a subject suspected of having or being at risk of having prostate cancer.

34. The method of claim 1, further comprising obtaining a biological sample from a subject suspected of having or being at risk of having prostate cancer.

35. A method for identifying a subject as being at an increased risk for developing prostate cancer, comprising:
(a) detecting the level of a prostate cancer marker in a biological sample from the subject, wherein the prostate cancer comprises one or more markers selected from Tables 1-31; and
(b) comparing the level of the prostate cancer marker in the biological sample with a predetermined threshold value;
wherein the level of the prostate cancer marker above or below the predetermined threshold value indicates that the subject is being at an increased risk for developing prostate cancer.

36. The method of claim 35, wherein the subject is a Caucasian subject and wherein the one or more markers is selected from Tables 1, 4, 8, 11, 13, 16, 19, 22, 26, 29 and 30.

37. The method of claim 35, wherein the subject a an African American subject and wherein the one or more markers is selected from Tables 2, 5, 9, 12, 14, 17, 20, 23, 27 and 31.

38.-40. (canceled)

41. The method of claim 35, wherein the prostate cancer marker comprises at least two or more markers, wherein each of the two of more markers are selected from the structural lipids set forth in Tables 1-7, the signaling lipids set forth in Tables 8-12, the proteins set forth in Tables 13-18, the metabolites set forth in Tables 19-25, and the markers set forth in Tables 26-28.

42.-61. (canceled)

62. The method of claim 35, wherein the level of the prostate cancer marker is detected by HPLC/UV-Vis spectroscopy, enzymatic analysis, mass spectrometry, NMR, immunoassay, ELISA, or any combination thereof, or by determining the level of its corresponding mRNA in the biological sample.

63. (canceled)

64. The method of claim 35, further comprising detecting the level of one or more additional markers of prostate cancer.

65. The method of claim 35, further comprising administering a therapeutic anti-cancer treatment to the subject based on the prognosis.

66.-91. (canceled)
